(19) United States
(12) Patent Application Publication
(10) Pub. No.: US 2024/0076299 A1
(43) Pub. Date: Mar. 7, 2024

(54) TUBERCULATIN ANALOGS AS ANTIVIRAL AGENTS

(71) Applicant: Hong Kong Baptist University, Hong Kong (CN)

(72) Inventors: Hongjie ZHANG, Hong Kong (CN); Yu ZHU, Hong Kong (CN); Nga Yi TSANG, Hong Kong (CN); Yang ZHAO, Hong Kong (CN)

(21) Appl. No.: 18/258,224

(22) PCT Filed: Dec. 7, 2021
(86) PCT No.: PCT/CN2021/136032

§ 371 Date: Jun. 19, 2023

Related U.S. Application Data
(60) Provisional application No. 63/199,329, filed on Dec. 21, 2020.

(51) Int. Cl. C07D 493/04; A61K 31/365; A61K 36/19; A61P 31/14; A61P 31/16; A61P 31/18; C07D 407/14; C07F 7/18

(52) U.S. Cl. CPC C07D 493/04; A61K 31/365; A61K 36/19; A61P 31/14; A61P 31/16; A61P 31/18; C07D 407/14; C07F 7/1804

(57) ABSTRACT

Provided herein are tuberculatin analogs that are useful as antivirals, such as anti-HIV, anti-coronaviral, anti-Ebola viral, and anti-influenza viral agents and methods of use thereof.

### CROSS-REFERENCE TO RELATED APPLICATIONS

This application claims the benefit of priority of U.S. Provisional Application No. 63/199,329, filed on Dec. 21, 2020, the contents of which are hereby incorporated by reference in their entirety for all purposes.

### TECHNICAL FIELD

The present disclosure generally relates to the arylnaphthalene glycoside derivatives, methods for their preparation, and use thereof. More particularly, the present disclosure relates to tuberculatin analogs that are useful as antiviral agents, such as anti-HIV, anti-coronaviral, anti-Ebola viral, anti-Marburg viral, and anti-influenza viral agents. The present disclosure also provides methods for treating viral infections, such as HIV, coronaviruses, Ebola virus, Marburg virus and influenza virus infections.

### BACKGROUND

Viruses are important etiologic agents that cause infectious diseases in humans and other mammals. They differ greatly in size, shape, chemical composition, host range, and effects on hosts. After decades of studies, only a limited number of antiviral agents are available for the treatment and/or prevention of diseases caused by viruses such as HIV, coronaviruses, Ebola, Marburg, influenza A and B and hepatitis C viruses. Because of their toxic effects on a host, many antiviral agents are limited in their application. Drug resistance is often very quickly developed against the antiviral agent, and many viral diseases, such as HIV have no vaccines available to treat or prevent them. Accordingly, there is a need for safe and effective antiviral agents against a wide-spectrum of viruses with no or low toxicity to the host.

AIDS (acquired immunodeficiency syndrome) remains one of the most serious threats to public health. In a UNAIDS (Uniting the world against AIDS) report, about 77 million people have been infected with the human immunodeficiency virus (HIV), and 37.9 million people have died from AIDS-related illnesses since the onset of the HIV epidemic in 1981. Since the first anti-HIV drug zidovudine (AZT) was developed and approved in 1987, more than 40 anti-HIV drugs have been formally approved by the U.S. Food and Drug Administration (FDA) for the treatment of HIV infection. These drugs are categorized as nucleoside reverse transcriptase inhibitors (NRTIs), non-nucleoside reverse transcriptase inhibitors (NNRTIs), protease inhibitors (PIs), entry and fusion inhibitors, and HIV integrase strand transfer inhibitors. Although these drugs have significantly extended the lifespan of HIV-positive people, it is worrisome that the prevalence of HIV drug resistance has increased from 11% to 29% since the global rollout of the antiretroviral therapy (ART) in 2001. In addition, the high cost and limited availability of the current ARTs has excluded patients in developing countries from the benefit of combination therapies. Therefore, there is an urgent need to continuously develop novel, more effective, accessible, and affordable anti-HIV therapeutics.

Influenza, a viral infection of the respiratory system, remains a major threat to human health. The worldwide outbreak of highly pathogenic H5N1 subtype of avian influenza virus (AIV) and the recent appearance of new type human influenza A/H1N1 have heightened public awareness of potential global influenza pandemics. In addition to domestic poultry, AIV can also infect wild birds, pigs, cats, humans, and other animals. Three drugs, Xofluza, zanamivir and oseltamivir phosphate, have been approved for the treatment of influenza. However, the low oral bioavailability and rapid renal elimination of zanamivir, and the rapid emergence of oseltamivir-resistant influenza viruses, have prompted the further development of more potent, longer duration therapeutic drugs to combat potential human influenza pandemics.

Viruses belonging to Filoviridae contain minus-strand RNA as their genome. There are two genera, namely Marburgvirus and Ebolavirus, under the Filoviridae family. Marburg virus is the only member in Marburgvirus genus. There are five members in Ebolavirus genus, namely Zaire ebolavirus, Sudan ebolavirus, Cote d'Ivoire ebolavirus, Reston ebolavirus and Bundibugyo. Owning to their pathogenic potential, high case mortality rate and the lack of effective therapeutics for infected humans, the members of family Filoviridae have been classified as “biosafety level 4” agents. Infection with filovirus may lead to hemorrhagic fever. In fact, both genera contain species that can cause epidemics of serious hemorrhagic fever in humans as well as non-human primates. The outbreak of Ebola virus (EBOV) disease mainly occurred in Democratic Republic of the Congo. There were numerous Ebola outbreaks since 1976. The first outbreak was in 1976 at Yambuku, with 318 cases reported and 88% death rate. Later, there were two large outbreaks in 1995 and 2007, in which over 250 Ebola cases were reported in each outbreak. In 2014-2015, West Africa experienced the largest Ebola outbreak. Over 28,000 cases were reported and the fatality rate reached 40%. Recently, an Ebola outbreak occurred again from Apr. 4, 2018. As of May 30, 2019, a total of 1945 cases have been reported with a death rate of 67%. There is no FDA-approved therapeutic agent specific to treat subjects infected by filovirus. Patients suffering from filovirus infection mainly rely on convalescent whole blood or plasma treatment during Ebola outbreaks. However, this kind of empirical treatment has many limitations, including difficulties in mass-production as well as the compatibility of blood group between donor and recipient. The use of some potential drug candidates, which include Favipiravir, ZMapp and GS-5734, are still under investigation. More clinical data is required to prove the safety and efficacy of these drug candidates in treating filovirus infection.

The emergence of novel coronavirus (SARS-CoV-2) raised international concerns and scientists strive to discover potent inhibitors against novel coronavirus. Coronaviruses (CoVs) are enveloped, single-stranded, positive-sense RNA virus, which include Coronaviridae, Arteriviridae, and Roniviridae families. SARS-CoV-2, that causes the current COVID-19 pandemic, is a β-coronavirus. There have been six CoVs identified as human-susceptible viruses. Two of them, SARS-CoV and MERS-CoV, could lead to severe or even fatal respiratory tract infections. As of Nov. 10, 2020, the COVID-19 epidemic has caused 1,270,573 deaths among over 51.3 million infected cases. Several EBOV inhibitors, such as remdesivir, toremifene, and favipiravir are repurposed as anti-viral agents active against SARS-CoV-2. However, none of them have been highly effective to curb the COVID-19 epidemic. Highly effective viral inhibitors are thus urgently needed to combat the coronaviruses.

Natural products have been a rich source for the discovery of lead compounds in the modern drug discovery. Justicia cf. patentiflora was identified as an anti-HIV plant lead through screening over 3,500 plant extracts. Bioassay-directed fractionation of the methanol extract of the stems and barks of this plant led to the isolation of three ANL (arylnaphthalene) glycoside compounds, which displayed potent inhibitory activity against broad HIV clinical strains with EC₅₀ values in the range of 14-37 nM [Zidovudine (AZT): 77-95 nM]. They also showed significant inhibitory effects against drug-resistance HIV strains.

We further evaluated the anti-HIV activity of the extracts from several other plant species in Justicia genus. Among them, J. procumbens was found as an annual plant that is widely distributed in southern regions of China. Phytochemical separation of the methanol extract of the aerial parts of the plant led to isolation of tuberculatin, the structure scaffold used for the synthesis of the antiviral compounds in the present prevention.

Some arylnaphthalene lignans have been reported to have antiviral activity in the literature. Although some of these compounds showed significant antiviral activities against various virus strains, they were not considered as potential antiviral drug candidates due to their low selectivity indices (SIs).

There thus exists a need to develop improved antiviral agents that address at least some of the aforementioned needs.

### SUMMARY

The present invention is based, at least in part, on the discovery that tuberculatin and congeners isolated from the plant Justicia procumbens L. (Acanthaceae) are effective in the treatment of AIDS and HIV infections. The present disclosure relates to a new class of tuberculatin analogs, the preparation of these compounds and new intermediates, and their use for treatment of viral infections, such as HIV, CoV, EBOV and AIV.

In a first aspect, provided herein is a compound of Formula I:

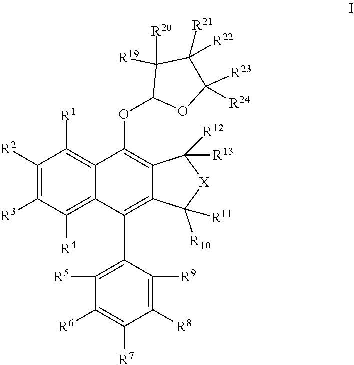

or a pharmaceutically acceptable salt thereof, wherein:
X is oxygen or sulfur;
R¹, R², R³, R⁴, R⁵, R⁶, R⁷, R⁸, and R⁹ are each independently hydrogen, alkyl, akenyl, alkynyl, cycloalkyl, heterocycloalkyl, aryl, aralkyl, heteroaryl, haloalkyl, halogen, cyano, NO₂, —OR²⁶, —C(═O)R²⁷, —C(═O)N(R²⁶)R²⁷, —C(═O)OR²⁶, —OC(═O)R²⁶, —OSi(R²⁵)(R²⁶)R²⁷, —S(═O)₂R²⁶, —S(═O)₂N(R²⁶)R²⁷, —N(R²⁶)R²⁷, —N(R²⁶)N(R²⁶)R²⁷, —N(R²⁶)C(═O)R²⁷, —N(R²⁶)S(═O)₂R²⁷ or a moiety comprising 1 to 30 plural valence atoms selected from the group consisting of carbon, nitrogen, oxygen and sulfur; or R¹ and R², R² and R³, R³ and R⁴, R⁵ and R⁶, R⁶ and R⁷, R⁷ and R⁸, or R⁸ and R⁹ taken together with the carbon atoms to which they are attached to form a cyclic group which is optionally substituted with halogen or a moiety comprising 1 to 30 plural valence atoms selected from the group consisting of carbon, nitrogen, oxygen and sulfur;
R¹⁰ and R¹¹ taken together form oxo; or while one of R¹⁰ and R¹¹ is hydrogen or halogen, the other one of R¹⁰ and R¹¹ is selected from the group consisting of R²⁵, —OR²⁵, —C(═O)R²⁵, and —C(═O)OR²⁵;
R¹² and R¹³ taken together form oxo; or while one of R¹² and R¹³ is hydrogen or halogen, the other one of R¹² and R¹³ is selected from the group consisting of R²⁵, —OR²⁵, —C(═O)R²⁵, and —C(═O)OR²⁵;
R¹⁹ and R²⁰ taken together form oxo; or while one of R¹⁹ and R²⁰ is hydrogen or halogen, the other one of R¹⁹ and R²⁰ is selected from the group consisting of R²⁵, —OR²⁵, —C(═O)R²⁵, —C(═O)OR²⁵, —OC(═O)R²⁵, —OC(═O)N(R²⁵)R²⁵, optionally substituted monosaccharide, optionally substituted disaccharide, optionally substituted trisaccharide, and optionally substituted tetrasaccharide;
R²¹ and R²² taken together form oxo; or while one of R²¹ and R²² is hydrogen, halogen, R²⁵ or —OR²⁵, the other one of R²¹ and R²² is selected from the group consisting of R²⁵, —OR²⁵, —OC(═O)R²⁵, —OC(═O)N(R²⁵)R²⁵, —C(═O)OR²⁵, —CH₂R²⁹, —CH₂OR²⁹, —C(═O)R²⁹; or R¹⁹ and R²¹ taken together with the carbon atoms to which they are attached to form a 5-6 membered heterocyclyl optionally substituted with 1, 2, 3, 4 or 5 group(s) independently selected from R²⁵;
R²³ and R²⁴ taken together form oxo; or while one of R²³ and R²⁴ is hydrogen or halogen, the other one of R²³ and R²⁴ is selected from the group consisting of R²⁵, —OR²⁵, —C(═O)R²⁵, —C(═O)OR²⁵, optionally substituted monosaccharide, optionally substituted disaccharide, optionally substituted trisaccharide, and optionally substituted tetrasaccharide;
R²⁵ for each occurrence is independently selected from hydrogen, halogen, trichloromethyl, trifluoromethyl, cyano, nitro, —OR²⁶, —C(═O)R²⁷, —C(═O)N(R²⁶)R²⁷, —C(═O)OR²⁶, —OC(═O)R²⁶, —OSi(R²⁵)(R²⁶)R²⁷, —S(═O)₂R²⁶, —S(═O)₂N(R²⁶)R²⁷, —N(R²⁶)R²⁷, —N(R²⁶)N(R²⁶)R²⁷, —N(R²⁶)C(═O)R²⁷, —N(R²⁶)S(═O)₂R²⁷, —N3, —OS(═O)₂CF₃, hydrocarbyl optionally substituted with 1, 2, 3, 4 or 5 group(s) independently selected from the group consisting of R²⁸, heterocyclcyl optionally substituted with 1, 2, 3, 4 or 5 group(s) independently selected from the group consisting of R²⁸, and —(CH₂)k-heterocyclyl optionally substituted with 1, 2, 3, 4 or 5 group(s) independently selected from the group consisting of R²⁸, wherein k is an integer between 1 and 6;
R²⁶ and R²⁷ for each occurrence are each independently hydrogen or selected from hydrocarbyl and heterocyclyl, either of which is optionally substituted with 1, 2, 3, 4 or 5 group(s) independently selected from halogen, cyano, amino, hydroxy, C₁₋₆ alkyl and C₁₋₆ alkoxy;
R²⁸ for each occurrence is independently selected from halogen, trichloromethyl, trifluoromethyl, cyano, nitro, oxo, ═NR²⁶, —OR²⁶, —C(═O)R²⁷, —C(═O)N(R²⁶)R²⁷, —C(═O)OR²⁶, —OC(═O)R²⁶, —OSi(R²⁵)(R²⁶)R²⁷, —S(═O)₂R²⁶, —S(═O)₂N(R²⁶)R²⁷, —N(R²⁶)R²⁷, —N(R²⁶)N(R²⁶)R²⁷, —N(R²⁶)C(═O)R²⁷ and —N(R²⁶)S(═O)₂R²⁷; and
R²⁹ for each occurrence is independently selected from hydrogen, halogen, trichloromethyl, trifluoromethyl, cyano, nitro, —OR²⁶, —C(═O)R²⁷, —C(═O)N(R²⁶)R²⁷, —C(═O)OR²⁶, —OC(═O)R²⁶, —OSi(R²⁵)(R²⁶)R²⁷, —S(═O)₂R²⁶, —S(═O)₂N(R²⁶)R²⁷, —N(R²⁶)R²⁷, —N(R²⁶)N(R²⁶)R²⁷, —N(R²⁶)C(═O)R²⁷, —N(R²⁶)S(═O)₂R²⁷, —N3, —OS(═O)₂CF₃, optionally substituted monosaccharide, optionally substituted disaccharide, optionally substituted trisaccharide, optionally substituted tetrasaccharide, hydrocarbyl optionally substituted with 1, 2, 3, 4 or 5 group(s) independently selected from the group consisting of R²⁸, and heterocyclyl optionally substituted with 1, 2, 3, 4 or 5 group(s) independently selected from the group consisting of R²⁸, with the proviso that the compound of Formula I does not include a compound selected from the group consisting of 1, 1-Ac, A1, A2, A3, A4, A5, A6, A7, and A8:

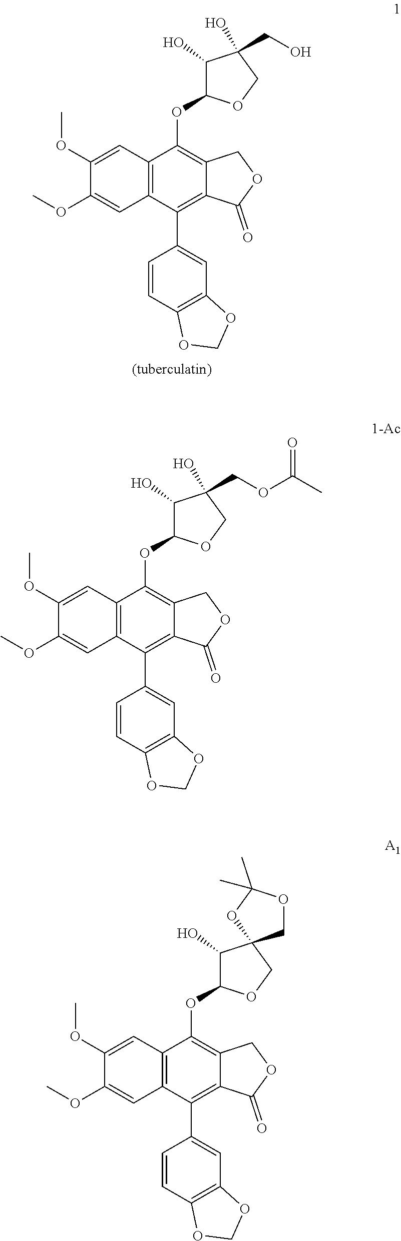

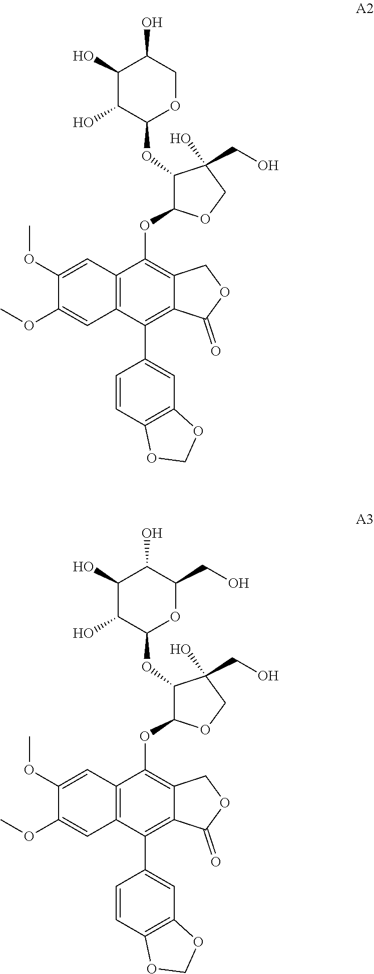 [SMILES: COc1cc2c(O[C@@H]3OC[C@](O)(CO)[C@H]3O[C@@H]3OC[C@H](O)[C@H](O)[C@H]3O)c3c(c(-c4ccc5c(c4)OCO5)c2cc1OC)C(=O)OC3 ; COc1cc2c(O[C@@H]3OC[C@](O)(CO)[C@H]3O[C@@H]3O[C@H](CO)[C@@H](O)[C@H](O)[C@H]3O)c3c(c(-c4ccc5c(c4)OCO5)c2cc1OC)C(=O)OC3]

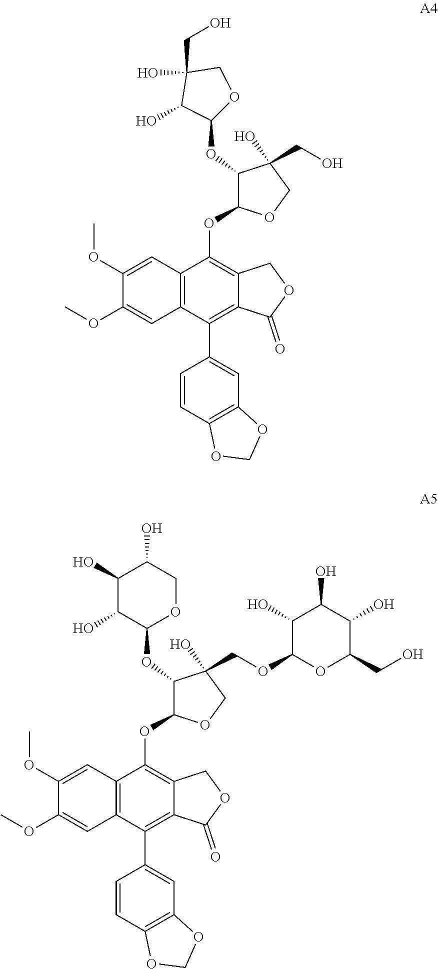 [SMILES: COc1cc2c(O[C@@H]3OC[C@](O)(CO)[C@H]3O[C@@H]3OC[C@](O)(CO)[C@H]3O)c3c(c(-c4ccc5c(c4)OCO5)c2cc1OC)C(=O)OC3 ; COc1cc2c(O[C@@H]3OC[C@](O)(CO[C@@H]4O[C@H](CO)[C@@H](O)[C@H](O)[C@H]4O)[C@H]3O[C@@H]3OC[C@@H](O)[C@H](O)[C@H]3O)c3c(c(-c4ccc5c(c4)OCO5)c2cc1OC)C(=O)OC3]

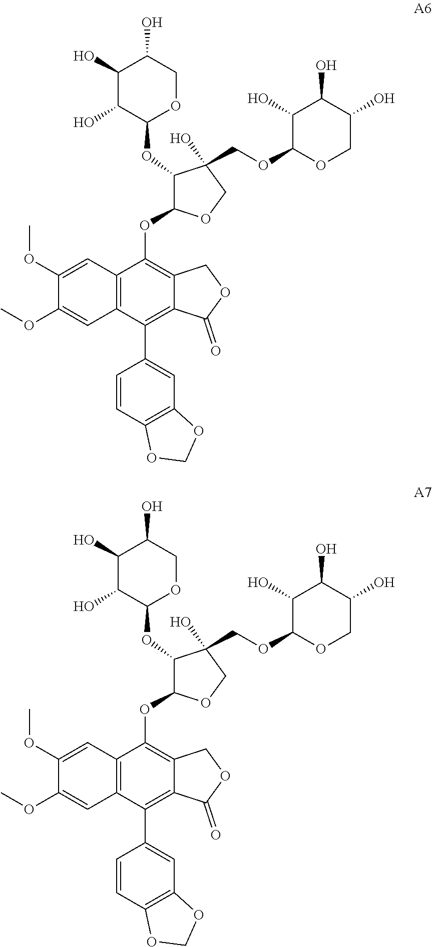 [SMILES: COc1cc2c(O[C@@H]3OC[C@](O)(CO[C@@H]4OC[C@@H](O)[C@H](O)[C@H]4O)[C@H]3O[C@@H]3OC[C@@H](O)[C@H](O)[C@H]3O)c3c(c(-c4ccc5c(c4)OCO5)c2cc1OC)C(=O)OC3 ; COc1cc2c(O[C@@H]3OC[C@](O)(CO[C@@H]4OC[C@@H](O)[C@H](O)[C@H]4O)[C@H]3O[C@@H]3OC[C@H](O)[C@H](O)[C@H]3O)c3c(c(-c4ccc5c(c4)OCO5)c2cc1OC)C(=O)OC3]

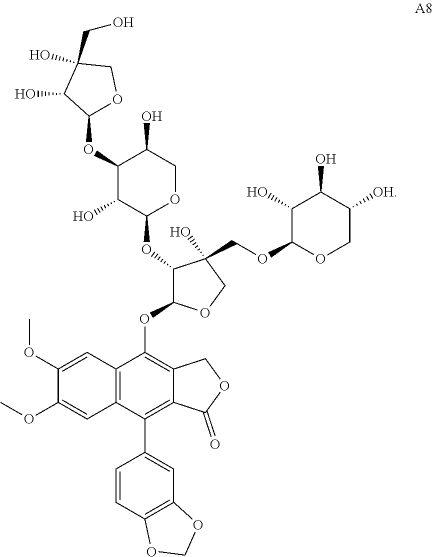 [SMILES: COc1cc2c(O[C@@H]3OC[C@](O)(CO[C@@H]4OC[C@@H](O)[C@H](O)[C@H]4O)[C@H]3O[C@@H]3OC[C@H](O)[C@H](O[C@@H]4OC[C@](O)(CO)[C@H]4O)[C@H]3O)c3c(c(-c4ccc5c(c4)OCO5)c2cc1OC)C(=O)OC3]

In certain embodiments, R²⁰, R²³, and R²⁴ are each hydrogen; R¹⁹ and R²¹ are each independently R²⁹; and R²² is —CH₂R²⁹, —CH₂OR²⁹; or R¹⁹ and R²¹ taken together with the carbon atoms to which they are attached to form a 5-6 membered heterocyclyl optionally substituted with 1, 2, 3, 4 or 5 group(s) independently selected from R²⁵.

In certain embodiments, the compound has Formula II:

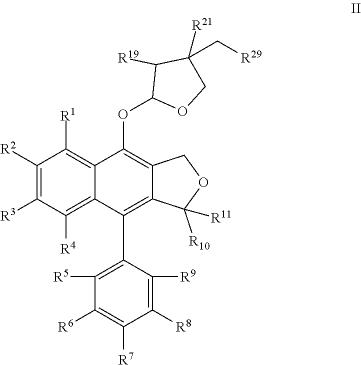

or a pharmaceutically acceptable salt thereof, wherein:
R¹, R², R³, R⁴, R⁵, R⁶, R⁷, R⁸, and R⁹ are each independently hydrogen, alkyl, akenyl, alkynyl, cycloalkyl, heterocycloalkyl, aryl, aralkyl, heteroaryl, haloalkyl, halogen, cyano, NO₂, —OR²⁶, —C(═O)R²⁷, —C(═O)N(R²⁶)R²⁷, —C(═O)OR²⁶, —OC(═O)R²⁶, —OSi(R²⁵)(R²⁶)R²⁷, —S(═O)₂R²⁶, —S(═O)₂N(R²⁶)R²⁷, —N(R²⁶)R²⁷, —N(R²⁶)N(R²⁶)R²⁷, —N(R²⁶)C(═O)R²⁷, —N(R²⁶)S(═O)₂R²⁷ or a moiety comprising 1 to 30 plural valence atoms selected from the group consisting of carbon, nitrogen, oxygen and sulfur; or R¹ and R², R² and R³, R³ and R⁴, R⁵ and R⁶, R⁶ and R⁷, R⁷ and R⁸, or R⁸ and R⁹ taken together with the carbon atoms to which they are attached to form a cyclic group which is optionally substituted with halogen or a moiety comprising 1 to 30 plural valence atoms selected from the group consisting of carbon, nitrogen, oxygen and sulfur;
R¹⁰ and R¹¹ taken together form oxo; or while one of R¹⁰ and R¹¹ is hydrogen, the other one of R¹⁰ and R¹¹ is selected from the group consisting of R²⁵, —OR²⁵, —C(═O)R²⁵, and —C(═O)OR²⁵;
R¹⁹ is selected from the group consisting of R²⁵, —OR²⁵, —OC(═O)R²⁵, —OC(═O)N(R²⁵)R²⁵, optionally substituted monosaccharide, optionally substituted disaccharide, optionally substituted trisaccharide, and optionally substituted tetrasaccharide;
R²¹ is selected from the group consisting of R²⁵, —OR²⁵, —OC(═O)R²⁵, —OC(═O)N(R²⁵)R²⁵, —C(═O)OR²⁵, —CH₂R²⁹, —CH₂OR²⁹, —C(═O)R²⁹; or R¹⁹ and R²¹ taken together with the carbon atoms to which they are attached to form a 5-6 membered heterocyclyl optionally substituted with 1, 2, 3, 4 or 5 group(s) independently selected from R²⁵;
R²⁵ for each occurrence is independently selected from hydrogen, halogen, trichloromethyl, trifluoromethyl, cyano, nitro, —OR²⁶, —C(═O)R²⁷, —C(═O)N(R²⁶)R²⁷, —C(═O)OR²⁶, —OC(═O)R²⁶, —OSi(R²⁵)(R²⁶)R²⁷, —S(═O)₂R²⁶, —S(═O)₂N(R²⁶)R²⁷, —N(R²⁶)R²⁷, —N(R²⁶)N(R²⁶)R²⁷, —N(R²⁶)C(═O)R²⁷, —N(R²⁶)S(═O)₂R²⁷, —N3, —OS(═O)₂CF₃, hydrocarbyl optionally substituted with 1, 2, 3, 4 or 5 group(s) independently selected from the group consisting of R²⁸, heterocyclcyl optionally substituted with 1, 2, 3, 4 or 5 group(s) independently selected from the group consisting of R²⁸, and —(CH₂)ₖ-heterocyclyl optionally substituted with 1, 2, 3, 4 or 5 group(s) independently selected from the group consisting of R²⁸, wherein k is an integer between 1 and 6;
R²⁶ and R²⁷ for each occurrence are each independently hydrogen or selected from hydrocarbyl and heterocyclyl, either of which is optionally substituted with 1, 2, 3, 4 or 5 group(s) independently selected from halogen, cyano, amino, hydroxy, C₁₋₆ alkyl and C₁₋₆ alkoxy;
R²⁸ for each occurrence is independently selected from halogen, trichloromethyl, trifluoromethyl, cyano, nitro, oxo, ═NR²⁶, —OR²⁶, —C(═O)R²⁷, —C(═O)N(R²⁶)R²⁷, —C(═O)OR²⁶, —OC(═O)R²⁶, —OSi(R²⁵)(R²⁶)R²⁷, —S(═O)₂R²⁶, —S(═O)₂N(R²⁶)R²⁷, —N(R²⁶)R²⁷, —N(R²⁶)N(R²⁶)R²⁷, —N(R²⁶)C(═O)R²⁷ and —N(R²⁶)S(═O)₂R²⁷; and
R²⁹ for each occurrence is independently selected from hydrogen, halogen, trichloromethyl, trifluoromethyl, cyano, nitro, —OR²⁶, —C(═O)R²⁷, —C(═O)N(R²⁶)R²⁷, —C(═O)OR²⁶, —OC(═O)R²⁶, —OSi(R²⁵)(R²⁶)R²⁷, —S(═O)₂R²⁶, —S(═O)₂N(R²⁶)R²⁷, —N(R²⁶)R²⁷, —N(R²⁶)N(R²⁶)R²⁷, —N(R²⁶)C(═O)R²⁷, —N(R²⁶)S(═O)₂R²⁷, —N3, —OS(═O)₂CF₃, optionally substituted monosaccharide, optionally substituted disaccharide, optionally substituted trisaccharide, optionally substituted tetrasaccharide, hydrocarbyl optionally substituted with 1, 2, 3, 4 or 5 group(s) independently selected from the group consisting of R²⁸, and heterocyclyl optionally substituted with 1, 2, 3, 4 or 5 group(s) independently selected from the group consisting of R²⁸.

In certain embodiments, R¹, R², R³, R⁴, R⁵, R⁶, R⁷, R⁹, and R⁹ are each independently hydrogen, alkyl, akenyl, alkynyl, cycloalkyl, heterocycloalkyl, aryl, aralkyl, heteroaryl, haloalkyl, halogen, cyano, NO₂, —OR²⁶, —C(═O)R²⁷, —C(═O)N(R²⁶)R²⁷, —C(═O)OR²⁶, —OC(═O)R²⁶, —OSi(R²⁵)(R²⁶)R²⁷, —S(═O)₂R²⁶, —S(═O)₂N(R²⁶)R²⁷, —N(R²⁶)R²⁷, —N(R²⁶)N(R²⁶)R²⁷, —N(R²⁶)C(═O)R²⁷, and —N(R²⁶)S(═O)₂R²⁷; or R¹ and R², R² and R³, R³ and R⁴, R⁵ and R⁶, R⁶ and R⁷, R⁷ and R⁸, or R⁸ and R⁹ taken together with the carbon atoms to which they are attached to form a 5-membered heterocyclyl;
R¹⁰ and R¹¹ taken together form oxo;
R¹⁹ is selected from the group consisting of OR²⁵, —OC(═O)R²⁵, —OC(═O)N(R²⁵)R²⁵, optionally substituted monosaccharide, optionally substituted disaccharide, optionally substituted trisaccharide, and optionally substituted tetrasaccharide; and
R²¹ is selected from the group consisting of —OR²⁵, —OC(═O)R²⁵, —OC(═O)N(R²⁵)R²⁵; or R¹⁹ and R²¹ taken together with the carbon atoms to which they are attached to form a 5 membered heterocyclyl optionally substituted with 1, 2, 3, 4 or 5 group(s) independently selected from R²⁵.

In certain embodiments, the compound has Formula III:

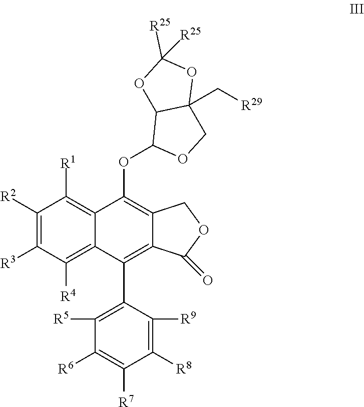

or a pharmaceutically acceptable salt thereof, wherein:
wherein R¹, R², R³, R⁴, R⁵, R⁶, R⁷, R⁸, and R⁹ are each independently hydrogen, alkyl, akenyl, alkynyl, cycloalkyl, heterocycloalkyl, aryl, aralkyl, heteroaryl, haloalkyl, halogen, cyano, NO₂, —OR²⁶, —C(═O)R²⁷, —C(═O)N(R²⁶)R²⁷, —C(═O)OR²⁶, —OC(═O)R²⁶, —OSi(R²⁵)(R²⁶)R²⁷, —S(═O)₂R²⁶, —S(═O)₂N(R²⁶)R²⁷, —N(R²⁶)R²⁷, —N(R²⁶)N(R²⁶)R²⁷, —N(R²⁶)C(═O)R²⁷, —N(R²⁶)S(═O)₂R²⁷; or R¹ and R², R² and R³, R³ and R⁴, R⁵ and R⁶, R⁶ and R⁷, R⁷ and R⁸, or R⁸ and R⁹ taken together with the carbon atoms to which they are attached to form a 5-membered heterocyclyl;
R²⁵ for each occurrence is independently selected from hydrogen and hydrocarbyl optionally substituted with 1, 2, 3, 4 or 5 group(s) independently selected from the group consisting of R²⁸, heterocyclcyl optionally substituted with 1, 2, 3, 4 or 5 group(s) independently selected from the group consisting of R²⁸, and —(CH₂)ₖ-heterocyclyl optionally substituted with 1, 2, 3, 4 or 5 group(s) independently selected from the group consisting of R²⁸, wherein k is an integer between 1 and 6;
R²⁶ and R²⁷ for each occurrence are each independently hydrogen or selected from hydrocarbyl and heterocyclyl, either of which is optionally substituted with 1, 2, 3, 4 or 5 group(s) independently selected from halogen, cyano, amino, hydroxy, C₁₋₆ alkyl and C₁₋₆ alkoxy;
R²⁸ for each occurrence is independently selected from halogen, trichloromethyl, trifluoromethyl, cyano, nitro, oxo, ═NR²⁶, —OR²⁶, —C(═O)R²⁷, —C(═O)N(R²⁶)R²⁷, —C(═O)OR²⁶, —OC(═O)R²⁶, —OSi(R²⁵)(R²⁶)R²⁷, —S(═O)₂R²⁶, —S(═O)₂N(R²⁶)R²⁷, —N(R²⁶)R²⁷, —N(R²⁶)N(R²⁶)R²⁷, —N(R²⁶)C(═O)R²⁷ and —N(R²⁶)S(═O)₂R²⁷; and
R²⁹ is selected from the group consisting of halogen, cyano, —OR²⁶, —OC(═O)R²⁶, —OSi(R²⁵)(R²⁶)R²⁷, —N(R²⁶)R²⁷, —N(R²⁶)N(R²⁶)R²⁷, —N(R²⁶)C(═O)R²⁷, —N(R²⁶)S(═O)₂R²⁷, —N₃, and —OS(═O)₂CF₃.

In certain embodiments, R¹, R⁴, R⁶, and R⁹ are each hydrogen; R², R³, R⁷, and R⁸ are each independently —OR²⁶; and R⁵ is hydrogen or —OR²⁶; or R² and R³ taken together with the carbon atoms to which they are attached to form a 5-membered heterocyclyl; or R⁷ and R⁸ taken together with the carbon atoms to which they are attached to form a 5-membered heterocyclyl.

In certain embodiments, the compound has Formula IV:

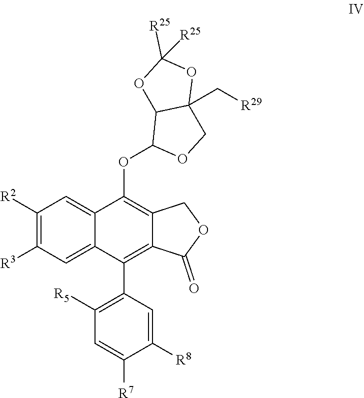

or a pharmaceutically acceptable salt thereof, wherein:
R², R³, R⁷, and R⁸ are each independently —OR²⁶; and R⁵ is hydrogen or —OR²⁶; or R² and R³ taken together form a methylenedioxy group; or R⁷ and R⁸ taken together form a methylenedioxy group;
R²⁵ for each occurrence is independently selected from hydrogen and hydrocarbyl;
R²⁶ for each occurrence is independently hydrogen, hydrocarbyl, or heterocyclyl; and
R²⁹ is selected from the group consisting of halogen, cyano, —OR²⁶, —OC(═O)R²⁶, —OSi(R²⁵)(R²⁶)R²⁷, —N(R²⁶)R²⁷, —N(R²⁶)N(R²⁶)R²⁷, —N(R²⁶)C(═O)R²⁷, —N(R²⁶)S(═O)₂R²⁷, —N₃, and —OS(═O)₂CF₃.

In certain embodiments, the compound has Formula V:

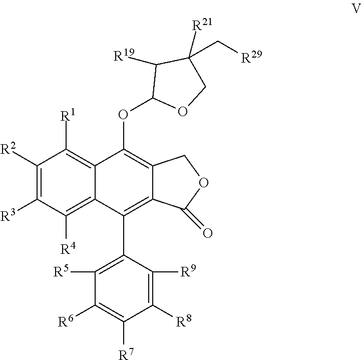

or a pharmaceutically acceptable salt thereof, wherein:
wherein R¹, R², R³, R⁴, R⁵, R⁶, R⁷, R⁹, and R⁹ are each independently hydrogen, alkyl, akenyl, alkynyl, cycloalkyl, heterocycloalkyl, aryl, aralkyl, heteroaryl, haloalkyl, halogen, cyano, NO₂, —OR²⁶, —C(═O)R²⁷, —C(═O)N(R²⁶)R²⁷, —C(═O)OR²⁶, —OC(═O)R²⁶, —OSi(R²⁵)(R²⁶)R²⁷, —S(═O)₂R²⁶, —S(═O)₂N(R²⁶)R²⁷, —N(R²⁶)R²⁷, —N(R²⁶)N(R²⁶)R²⁷, —N(R²⁶)C(═O)R²⁷, —N(R²⁶)S(═O)₂R²⁷; or R¹ and R², R² and R³, R³ and R⁴, R⁵ and R⁶, R⁶ and R⁷, R⁷ and R⁸, or R⁸ and R⁹ taken together with the carbon atoms to which they are attached to form a 5-membered heterocyclyl;
R¹⁹ is selected from the group consisting of —OR²⁵, —OC(═O)R²⁵, —OC(═O)N(R²⁵)R²⁵, optionally substituted monosaccharide, optionally substituted disaccharide, optionally substituted trisaccharide, and optionally substituted tetrasaccharide;
R²¹ is selected from the group consisting of —OR²⁵, —OC(═O)R²⁵, —OC(═O)N(R²⁵)R²⁵, and —C(═O)OR²⁵;
R²⁵ for each occurrence is independently selected from hydrogen and hydrocarbyl optionally substituted with 1, 2, 3, 4 or 5 group(s) independently selected from the group consisting of R²⁸, heterocyclcyl optionally substituted with 1, 2, 3, 4 or 5 group(s) independently selected from the group consisting of R²⁸, and —(CH₂)ₖ-heterocyclyl optionally substituted with 1, 2, 3, 4 or 5 group(s) independently selected from the group consisting of R²⁸, wherein k is an integer between 1 and 6;
R²⁶ and R²⁷ for each occurrence are each independently hydrogen or selected from hydrocarbyl and heterocyclyl, either of which is optionally substituted with 1, 2, 3, 4 or 5 group(s) independently selected from halogen, cyano, amino, hydroxy, C₁₋₆ alkyl and C₁₋₆ alkoxy;
R²⁸ for each occurrence is independently selected from halogen, trichloromethyl, trifluoromethyl, cyano, nitro, oxo, ═NR²⁶, —OR²⁶, —C(═O)R²⁷, —C(═O)N(R²⁶)R²⁷, —C(═O)OR²⁶, —OC(═O)R²⁶, —OSi(R²⁵)(R²⁶)R²⁷, —S(═O)₂R²⁶, —S(═O)₂N(R²⁶)R²⁷, —N(R²⁶)R²⁷, —N(R²⁶)N(R²⁶)R²⁷, —N(R²⁶)C(═O)R²⁷ and —N(R²⁶)S(═O)₂R²⁷; and
R²⁹ is selected from the group consisting of halogen, cyano, —OR²⁶, —OC(═O)R²⁶, —OSi(R²⁵)(R²⁶)R²⁷, —N(R²⁶)R²⁷, —N(R²⁶)N(R²⁶)R²⁷, —N(R²⁶)C(═O)R²⁷, —N(R²⁶)S(═O)₂R²⁷, —N₃, and —OS(═O)₂CF₃, optionally substituted monosaccharide, optionally substituted disaccharide, optionally substituted trisaccharide, and optionally substituted tetrasaccharide.

In certain embodiments, R¹, R⁴, R⁶, and R⁹ are each hydrogen; R², R³, R⁷, and R⁸ are each independently —OR²⁶; and R⁵ is hydrogen or —OR²⁶; or R² and R³ taken together with the carbon atoms to which they are attached to form a 5-membered heterocyclyl; or R⁷ and R⁸ taken together with the carbon atoms to which they are attached to form a 5-membered heterocyclyl.

In certain embodiments, the compound has Formula VI:

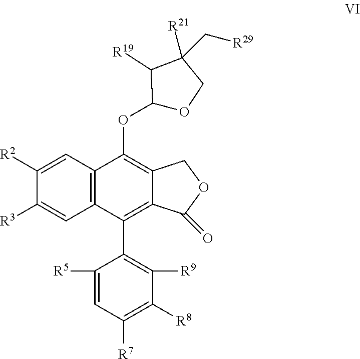

or a pharmaceutically acceptable salt thereof, wherein:
R², R³, R⁷, and R⁸ are each independently —OR²⁶; and R⁵; or R² and R³ taken together form a methylenedioxy group; or R⁷ and R⁸ taken together form a methylenedioxy group;
R⁹ is hydrogen or —OR²⁶;
R¹⁹ is selected from the group consisting of —OR²⁵, —OC(═O)R²⁵, and —OC(═O)N(R²⁵)R²⁵, optionally substituted monosaccharide, optionally substituted disaccharide, optionally substituted trisaccharide, and optionally substituted tetrasaccharide;
R²¹ is selected from the group consisting of —OR²⁵, —OC(═O)R²⁵, —OC(═O)N(R²⁵)R²⁵, and —C(═O)OR²⁵;
R²⁵ for each occurrence is independently selected from hydrogen and hydrocarbyl;
R²⁶ for each occurrence is independently hydrogen, hydrocarbyl, or heterocyclyl; and
R²⁹ is selected from the group consisting of halogen, cyano, —OR²⁶, —OC(═O)R²⁶, —OSi(R²⁵)(R²⁶)R²⁷, —N(R²⁶)R²⁷, —N(R²⁶)N(R²⁶)R²⁷, —N(R²⁶)C(═O)R²⁷, —N(R²⁶)S(═O)₂R²⁷, —N₃, and —OS(═O)₂CF₃, optionally substituted monosaccharide, optionally substituted disaccharide, optionally substituted trisaccharide, and optionally substituted tetrasaccharide.

In certain embodiments, the compound is selected from the group consisting of 8, 9, 10, 11, 12, 16, 17, 18, 19, 20, 21, 23, 24, 25, 26, 27, 28, 29, 30, 31, 32, 42, 43, 44, 45, 46, 47, 48, 49, 50, 51, 52, 53 and 54:

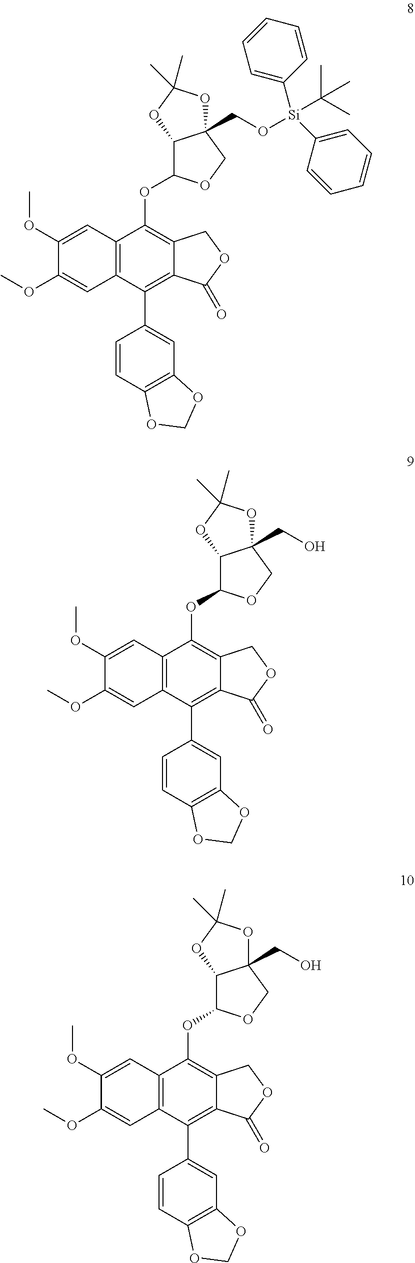 [SMILES: COc1cc2c(OC3OC[C@@]4(CO[Si](c5ccccc5)(c5ccccc5)C(C)(C)C)OC(C)(C)O[C@@H]34)c3c(c(-c4ccc5c(c4)OCO5)c2cc1OC)C(=O)OC3 ; COc1cc2c(O[C@@H]3OC[C@@]4(CO)OC(C)(C)O[C@@H]34)c3c(c(-c4ccc5c(c4)OCO5)c2cc1OC)C(=O)OC3 ; COc1cc2c(O[C@H]3OC[C@@]4(CO)OC(C)(C)O[C@@H]34)c3c(c(-c4ccc5c(c4)OCO5)c2cc1OC)C(=O)OC3]

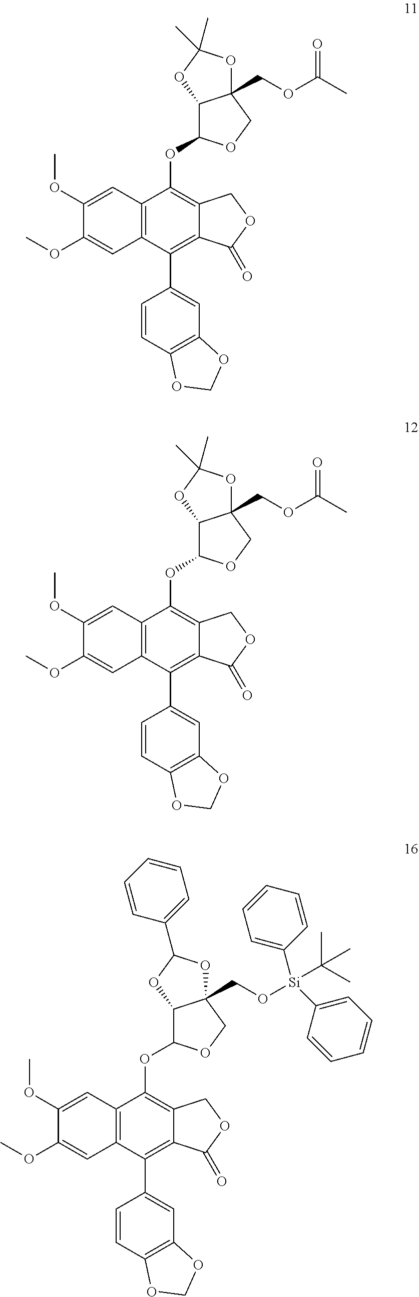

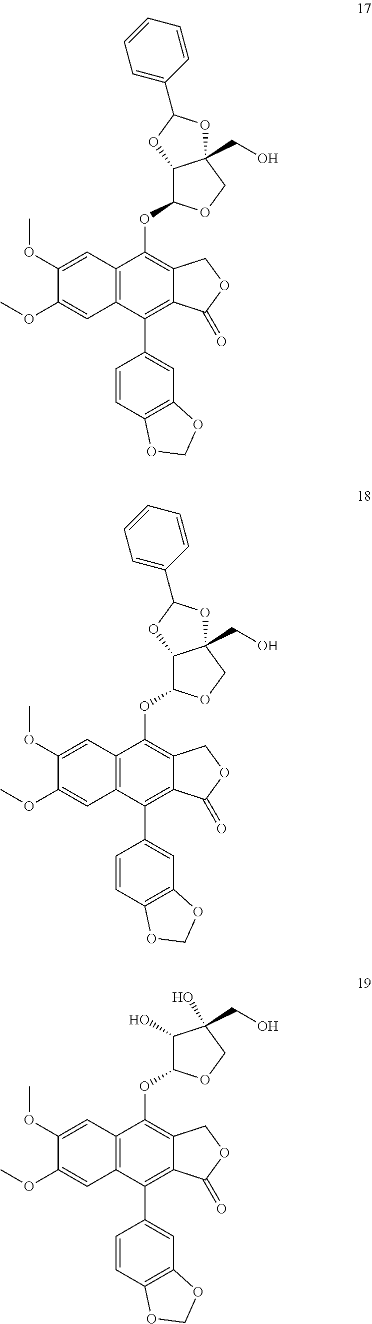

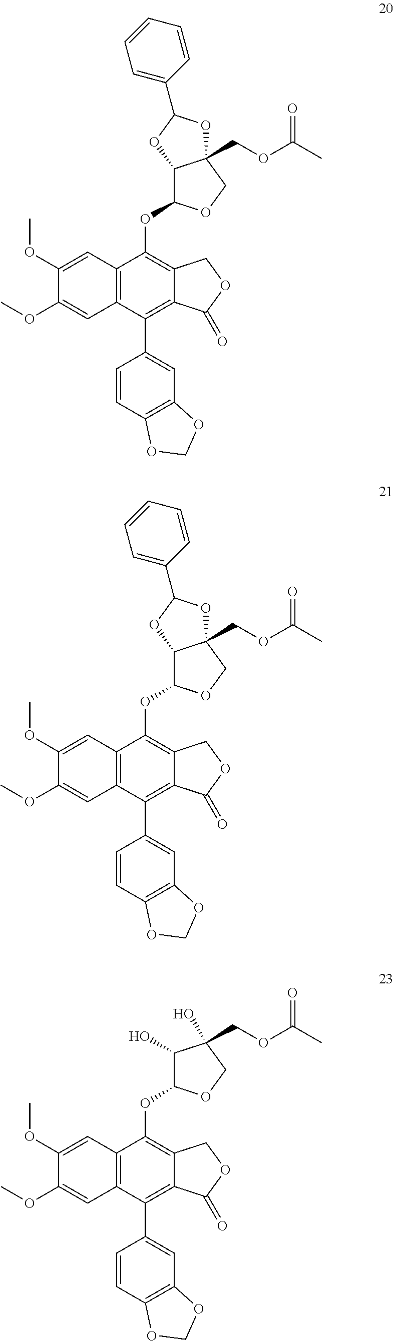

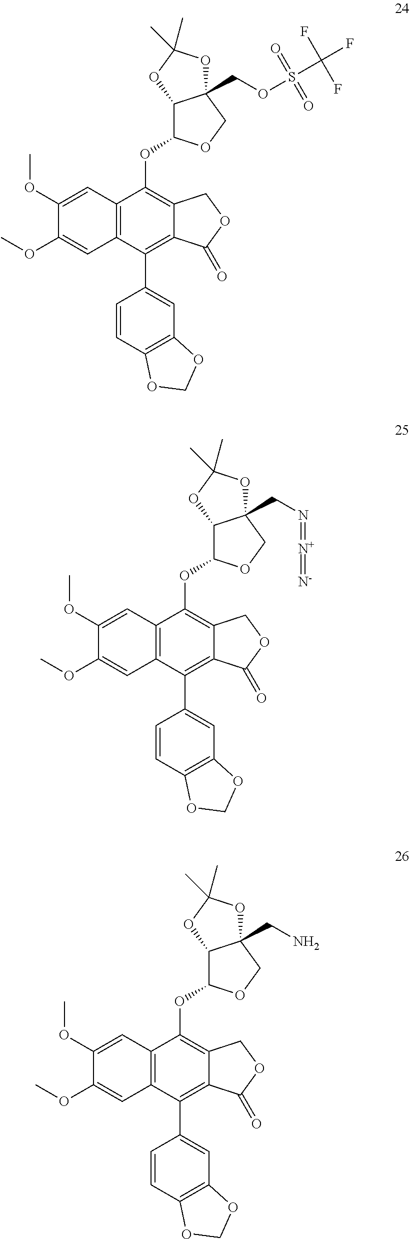 [SMILES: COc1cc2c(O[C@H]3OC[C@@]4(COS(=O)(=O)C(F)(F)F)OC(C)(C)O[C@@H]34)c3c(c(-c4ccc5c(c4)OCO5)c2cc1OC)C(=O)OC3 ; COc1cc2c(O[C@H]3OC[C@@]4(CN=[N+]=[N-])OC(C)(C)O[C@@H]34)c3c(c(-c4ccc5c(c4)OCO5)c2cc1OC)C(=O)OC3 ; COc1cc2c(O[C@H]3OC[C@@]4(CN)OC(C)(C)O[C@@H]34)c3c(c(-c4ccc5c(c4)OCO5)c2cc1OC)C(=O)OC3]

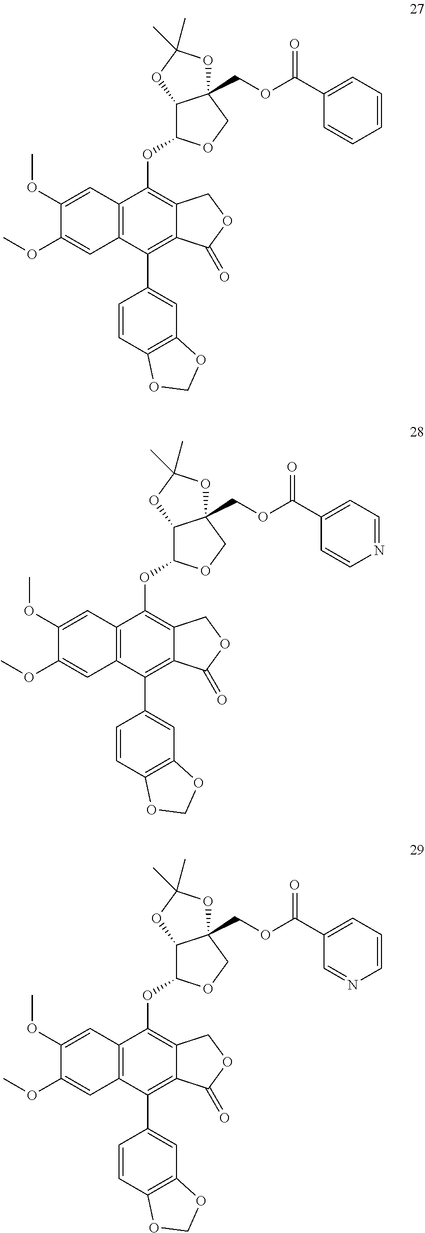

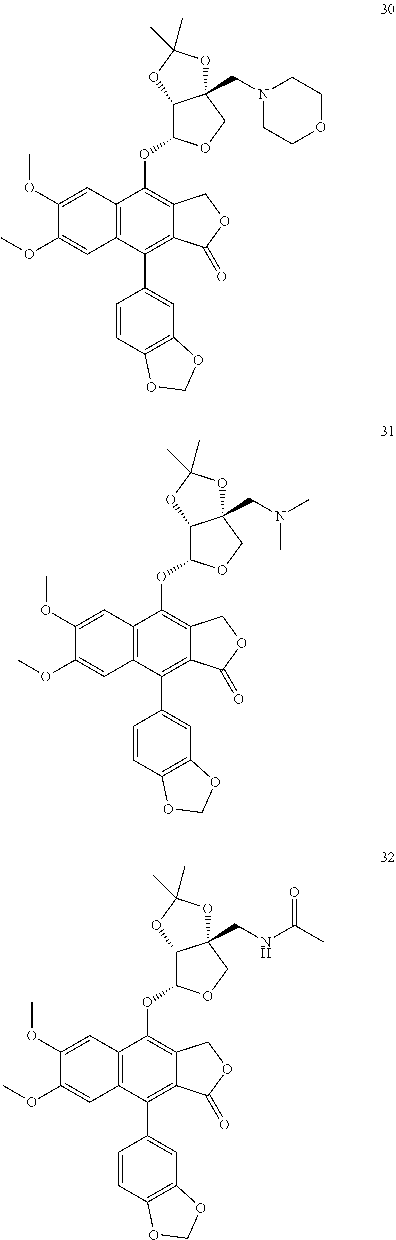

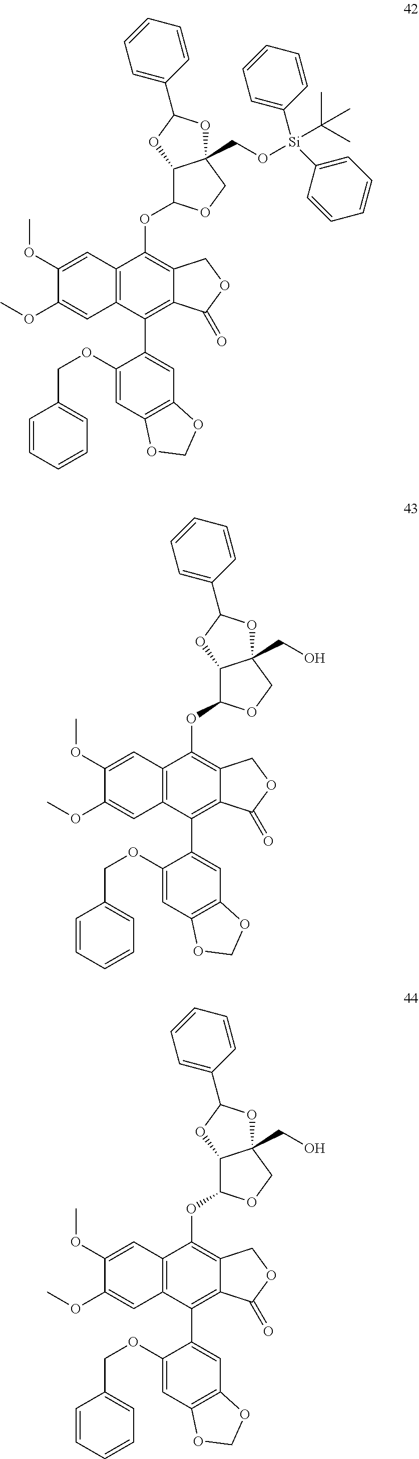

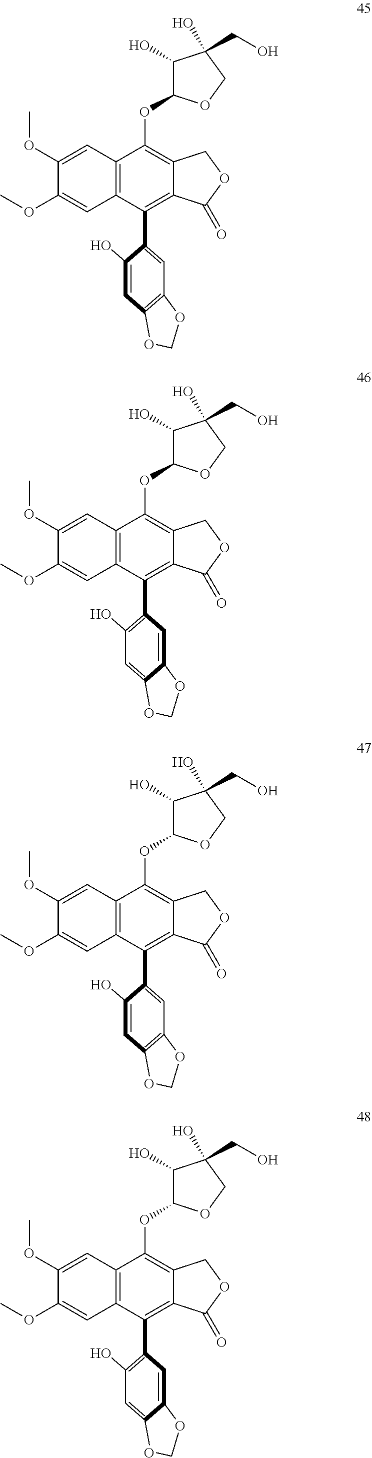

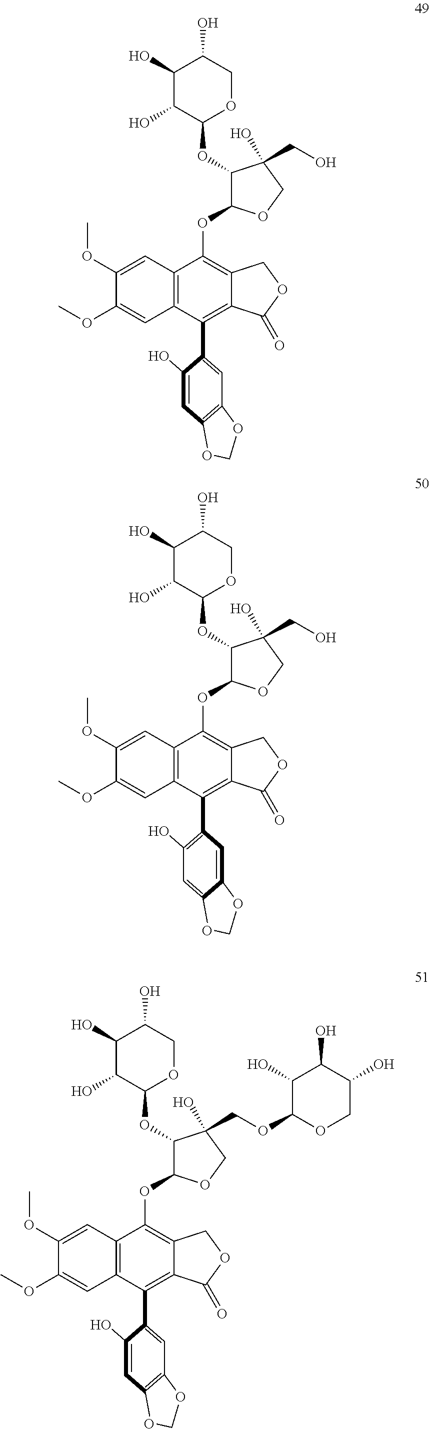

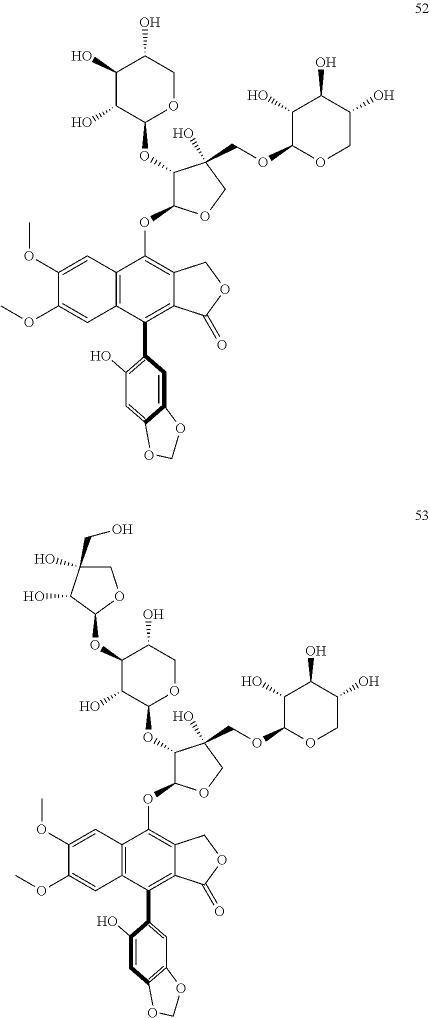

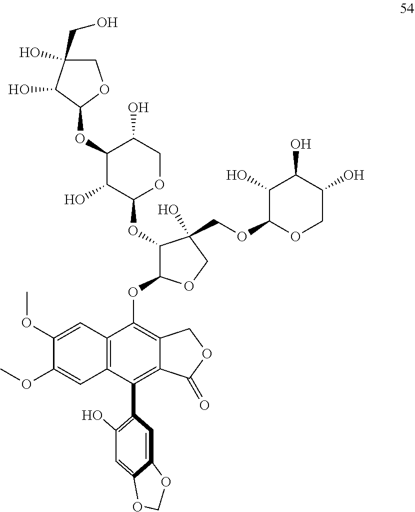

or a pharmaceutically acceptable salt thereof.

In a second aspect, provided herein is a pharmaceutical composition comprising a compound described herein and at least one pharmaceutically acceptable excipient.

In a third aspect provided herein is a compound described herein for use in the treatment, prevention or delay of progression of a viral infection in a subject in need thereof.

In certain embodiments, the viral infection is human immunodeficiency virus (HIV), influenza, vesicular stomatitis virus (VSV), or coronavirus (CoV).

In certain embodiments, the influenza is avian influenza virus (AIV).

In certain embodiments, the AIV is influenza A.

In certain embodiments, the influenza A is H5N₁.

In certain embodiments, the CoV is severe acute respiratory syndrome coronavirus 2 (SARS-CoV-2).

In certain embodiments, the compound inhibits the viral replication.

In certain embodiments, the subject is human.

In certain embodiments, the subject is an animal.

In a fourth aspect, provided herein is a compound for use in treatment, prevention or delay of progression of a viral infection in a subject in need thereof, wherein the compound has the Formula (I):

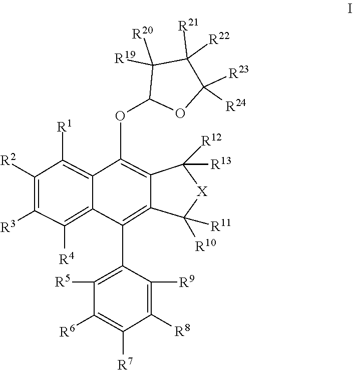

or a pharmaceutically acceptable salt thereof, wherein:
X is oxygen or sulfur;
R¹, R², R³, R⁴, R⁵, R⁶, R⁷, R⁸, and R⁹ are each independently hydrogen, alkyl, akenyl, alkynyl, cycloalkyl, heterocycloalkyl, aryl, aralkyl, heteroaryl, haloalkyl, halogen, cyano, NO₂, —OR²⁶, —C(═O)R²⁷, —C(═O)N(R²⁶)R²⁷, —C(═O)OR²⁶, —OC(═O)R²⁶, —OSi(R²⁵)(R²⁶)R²⁷, —S(═O)₂R²⁶, —S(═O)₂N(R²⁶)R²⁷, —N(R²⁶)R²⁷, —N(R²⁶)N(R²⁶)R²⁷, —N(R²⁶)C(═O)R²⁷, —N(R²⁶)S(═O)₂R²⁷ or a moiety comprising 1 to 30 plural valence atoms selected from the group consisting of carbon, nitrogen, oxygen and sulfur; or R¹ and R², R² and R³, R³ and R⁴, R⁵ and R⁶, R⁶ and R⁷, R⁷ and R⁸, or R⁸ and R⁹ taken together with the carbon atoms to which they are attached to form a cyclic group which is optionally substituted with halogen or a moiety comprising 1 to 30 plural valence atoms selected from the group consisting of carbon, nitrogen, oxygen and sulfur;
R¹⁰ and R¹¹ taken together form oxo; or while one of R¹⁰ and R¹¹ is hydrogen or halogen, the other one of R¹⁰ and R¹¹ is selected from the group consisting of R²⁵, —OR²⁵, —C(═O)R²⁵, and —C(═O)OR²⁵;
R¹² and R¹³ taken together form oxo; or while one of R¹² and R¹³ is hydrogen or halogen, the other one of R¹² and R¹³ is selected from the group consisting of R²⁵, —OR²⁵, —C(═O)R²⁵, and —C(═O)OR²⁵;
R¹⁹ and R²⁰ taken together form oxo; or while one of R¹⁹ and R²⁰ is hydrogen or halogen, the other one of R¹⁹ and R²⁰ is selected from the group consisting of R²⁵, —OR²⁵, —C(═O)R²⁵, —C(═O)OR²⁵, —OC(═O)R²⁵, —OC(═O)N(R²⁵)R²⁵, optionally substituted monosaccharide, optionally substituted disaccharide, optionally substituted trisaccharide, and optionally substituted tetrasaccharide;
R²¹ and R²² taken together form oxo; or while one of R²¹ and R²² is hydrogen, halogen, R²⁵ or —OR²⁵, the other one of R²¹ and R²² is selected from the group consisting of R²⁵, —OR²⁵, —OC(═O)R²⁵, —OC(═O)N(R²⁵)R²⁵, —C(═O)OR²⁵, —CH₂R²⁹, —CH₂OR²⁹, —C(═O)R²⁹; or R¹⁹ and R²¹ taken together with the carbon atoms to which they are attached to form a 5-6 membered heterocyclyl optionally substituted with 1, 2, 3, 4 or 5 group(s) independently selected from R²⁵;
R²³ and R²⁴ taken together form oxo; or while one of R²³ and R²⁴ is hydrogen or halogen, the other one of R²³ and R²⁴ is selected from the group consisting of R²⁵, —OR²⁵, —C(═O)R²⁵, —C(═O)OR²⁵, optionally substituted monosaccharide, optionally substituted disaccharide, optionally substituted trisaccharide, and optionally substituted tetrasaccharide;
R²⁵ for each occurrence is independently selected from hydrogen, halogen, trichloromethyl, trifluoromethyl, cyano, nitro, —OR²⁶, —C(═O)R²⁷, —C(═O)N(R²⁶)R²⁷, —C(═O)OR²⁶, —OC(═O)R²⁶, —OSi(R²⁵)(R²⁶)R²⁷, —S(═O)₂R²⁶, —S(═O)₂N(R²⁶)R²⁷, —N(R²⁶)R²⁷, —N(R²⁶)N(R²⁶)R²⁷, —N(R²⁶)C(═O)R²⁷, —N(R²⁶)S(═O)₂R²⁷, —N₃, —OS(═O)₂CF₃, hydrocarbyl optionally substituted with 1, 2, 3, 4 or 5 group(s) independently selected from the group consisting of R²⁸, heterocyclcyl optionally substituted with 1, 2, 3, 4 or 5 group(s) independently selected from the group consisting of R²⁸, and —(CH₂)ₖ-heterocyclyl optionally substituted with 1, 2, 3, 4 or 5 group(s) independently selected from the group consisting of R²⁸, wherein k is an integer between 1 and 6;
R²⁶ and R²⁷ for each occurrence are each independently hydrogen or selected from hydrocarbyl and heterocyclyl, either of which is optionally substituted with 1, 2, 3, 4 or 5 group(s) independently selected from halogen, cyano, amino, hydroxy, C₁₋₆ alkyl and C₁₋₆ alkoxy;
R²⁸ for each occurrence is independently selected from halogen, trichloromethyl, trifluoromethyl, cyano, nitro, oxo, ═NR²⁶, —OR²⁶, —C(═O)R²⁷, —C(═O)N(R²⁶)R²⁷, —C(═O)OR²⁶, —OC(═O)R²⁶, —OSi(R²⁵)(R²⁶)R²⁷, —S(═O)₂R²⁶, —S(═O)₂N(R²⁶)R²⁷, —N(R²⁶)R²⁷, —N(R²⁶)N(R²⁶)R²⁷, —N(R²⁶)C(═O)R²⁷ and —N(R²⁶)S(═O)₂R²⁷; and
R²⁹ for each occurrence is independently selected from hydrogen, halogen, trichloromethyl, trifluoromethyl, cyano, nitro, —OR²⁶, —C(═O)R²⁷, —C(═O)N(R²⁶)R²⁷, —C(═O)OR²⁶, —OC(═O)R²⁶, —OSi(R²⁵)(R²⁶)R²⁷, —S(═O)₂R²⁶, —S(═O)₂N(R²⁶)R²⁷, —N(R²⁶)R²⁷, —N(R²⁶)N(R²⁶)R²⁷, —N(R²⁶)C(═O)R²⁷, —N(R²⁶)S(═O)₂R²⁷, —N₃, —OS(═O)₂CF₃, optionally substituted monosaccharide, optionally substituted disaccharide, optionally substituted trisaccharide, optionally substituted tetrasaccharide, hydrocarbyl optionally substituted with 1, 2, 3, 4 or 5 group(s) independently selected from the group consisting of R²⁸, and heterocyclyl optionally substituted with 1, 2, 3, 4 or 5 group(s) independently selected from the group consisting of R²⁸.

In certain embodiments, the compound is selected from the group consisting of 1, 1-Ac, A1, A2, A3, A4, A5, A6, A7, A8, 8, 9, 10, 11, 12, 16, 17, 18, 19, 20, 21, 22, 23, 24, 25, 26, 27, 28, 29, 30, 31, 32, 42, 43, 44, 45, 46, 47, 48, 49, 50, 51, 52, 53 and 54:

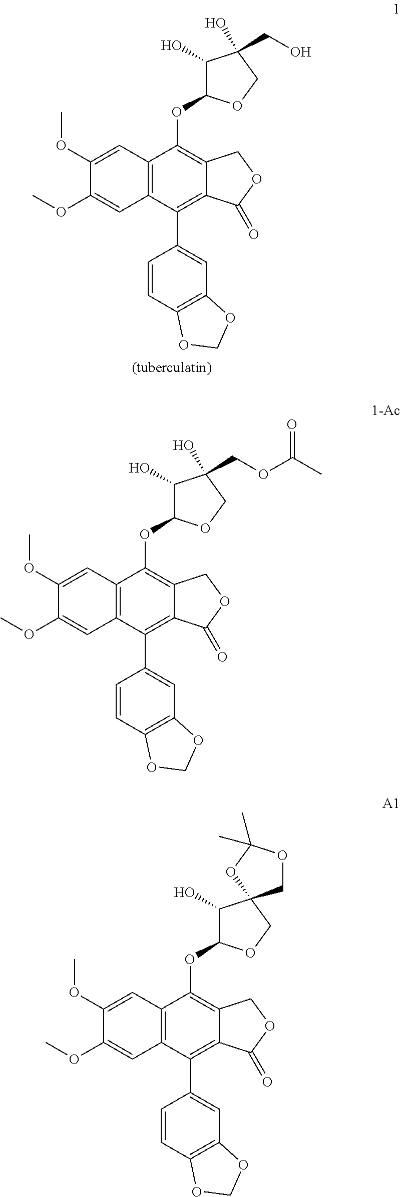

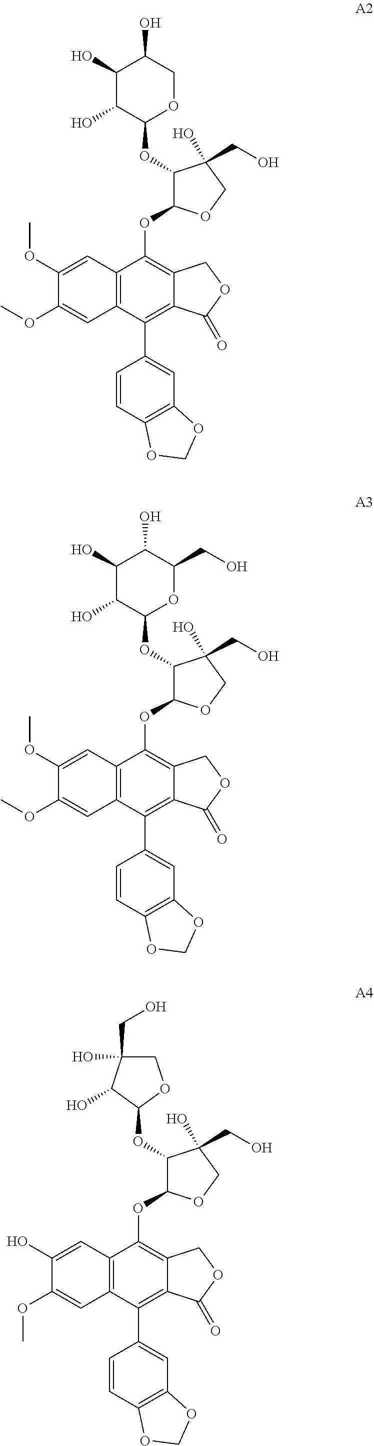

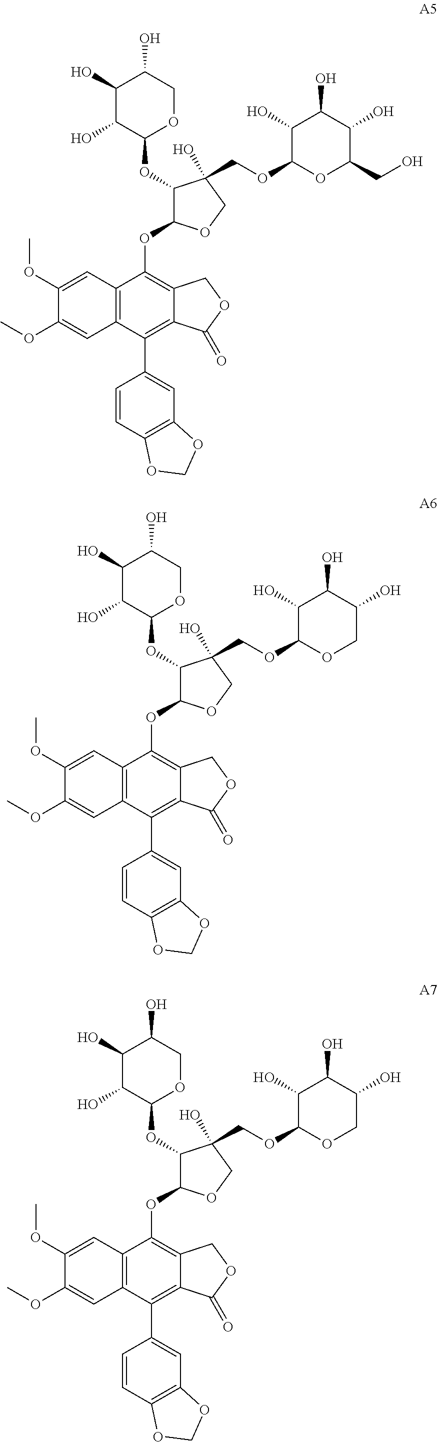

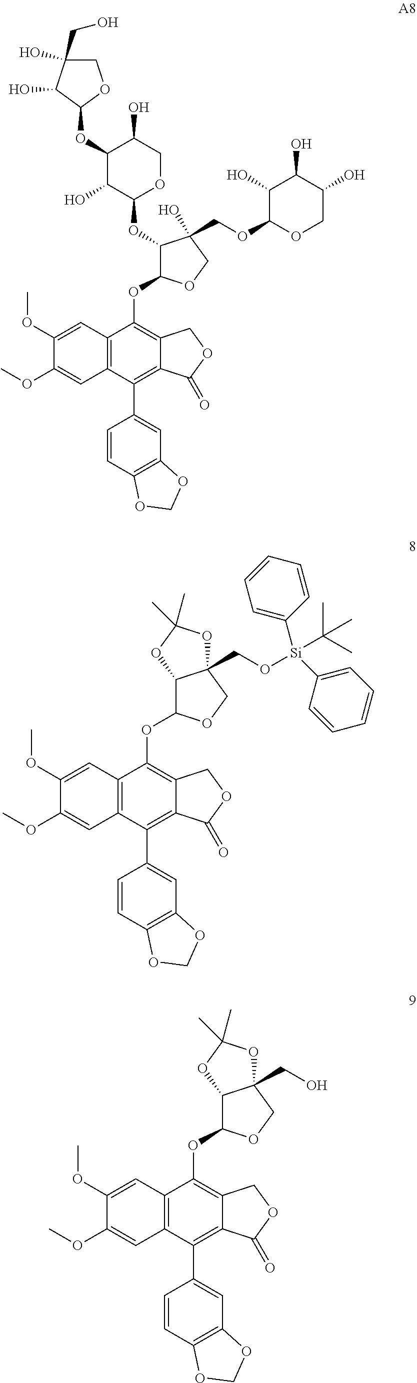

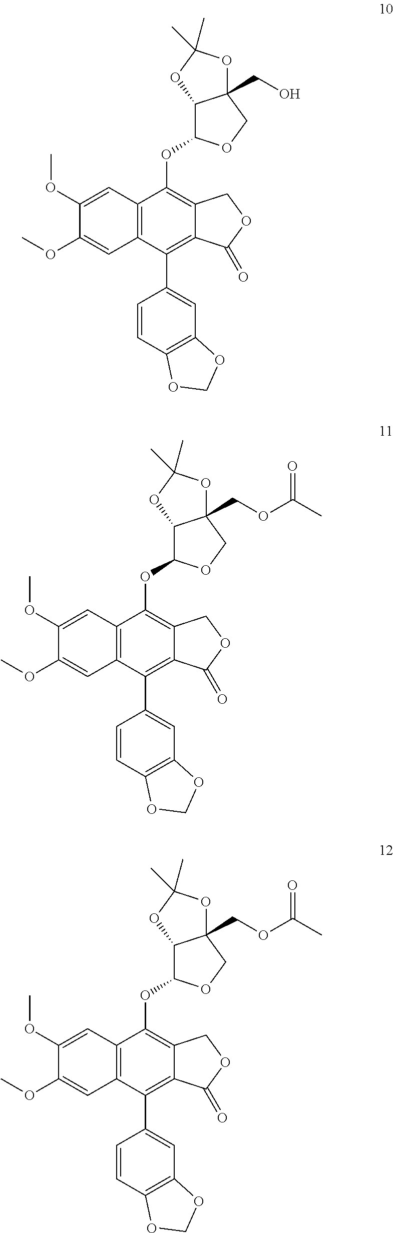

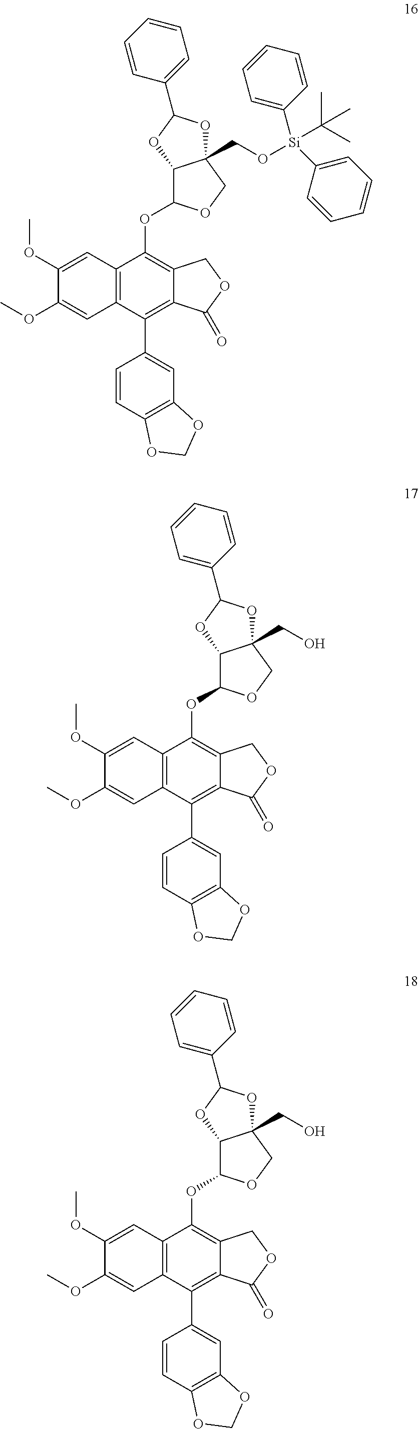

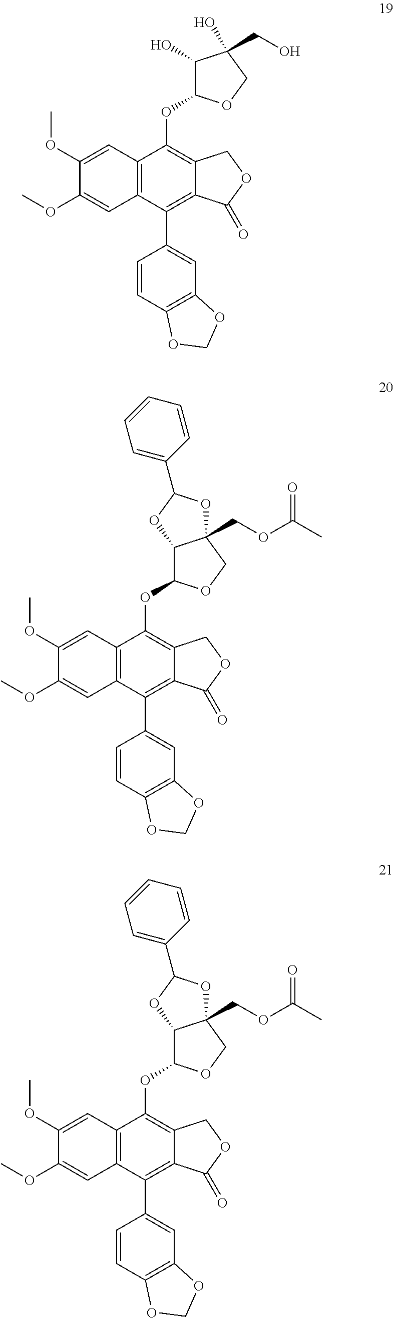

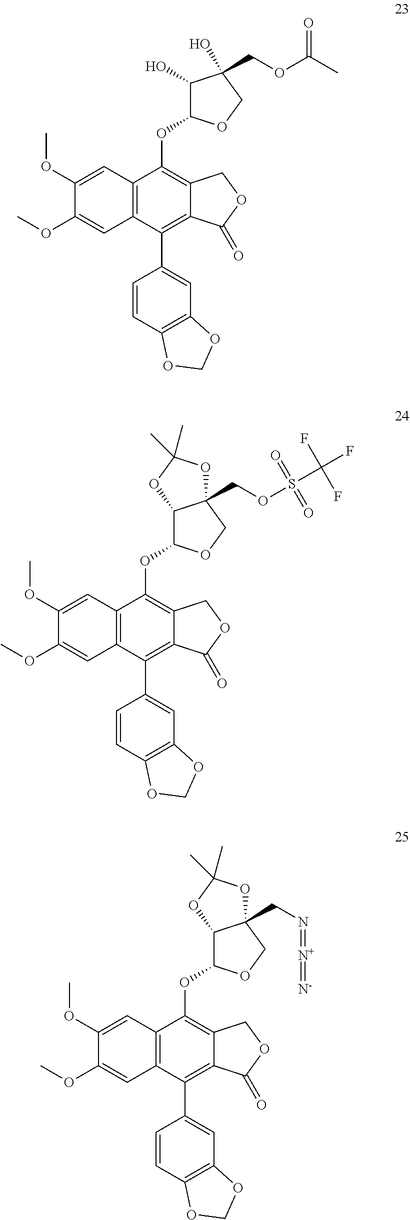

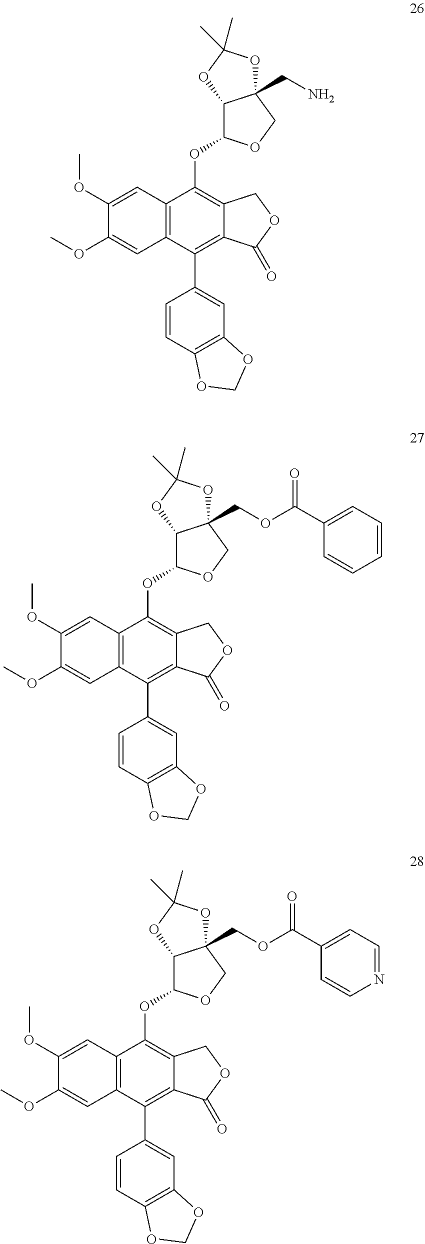 [SMILES: COc1cc2c(O[C@H]3OC[C@@]4(CN)OC(C)(C)O[C@@H]34)c3c(c(-c4ccc5c(c4)OCO5)c2cc1OC)C(=O)OC3 ; COc1cc2c(O[C@H]3OC[C@@]4(COC(=O)c5ccccc5)OC(C)(C)O[C@@H]34)c3c(c(-c4ccc5c(c4)OCO5)c2cc1OC)C(=O)OC3 ; COc1cc2c(O[C@H]3OC[C@@]4(COC(=O)c5ccncc5)OC(C)(C)O[C@@H]34)c3c(c(-c4ccc5c(c4)OCO5)c2cc1OC)C(=O)OC3]

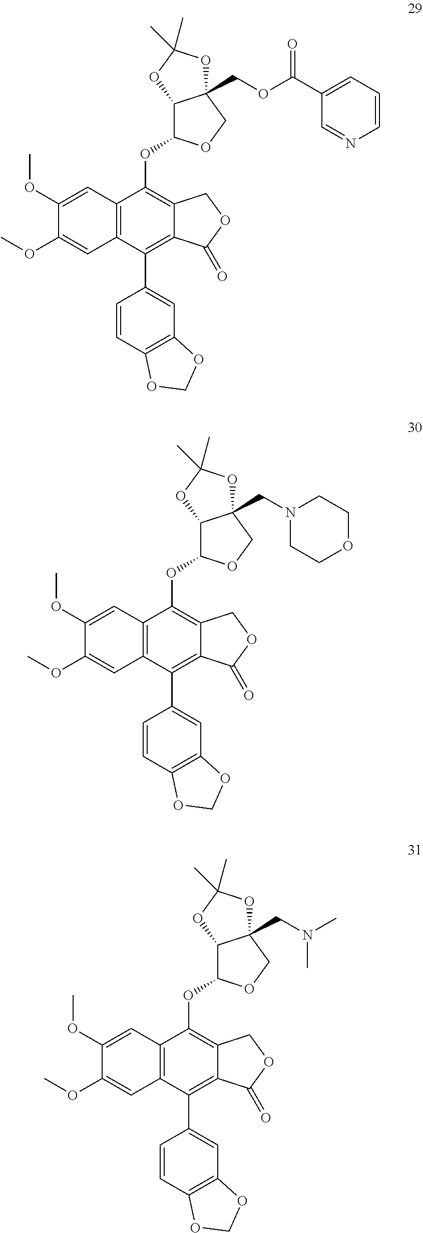

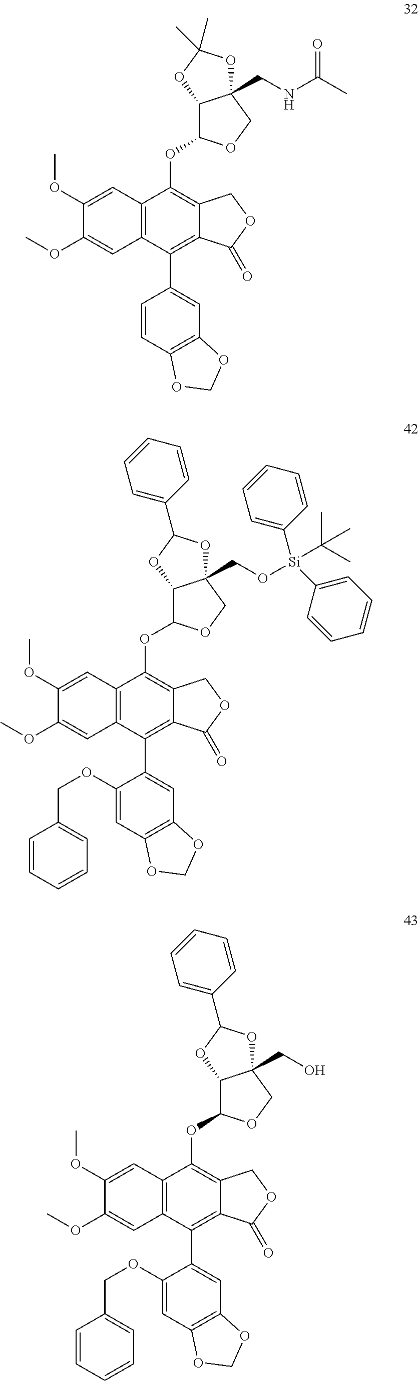

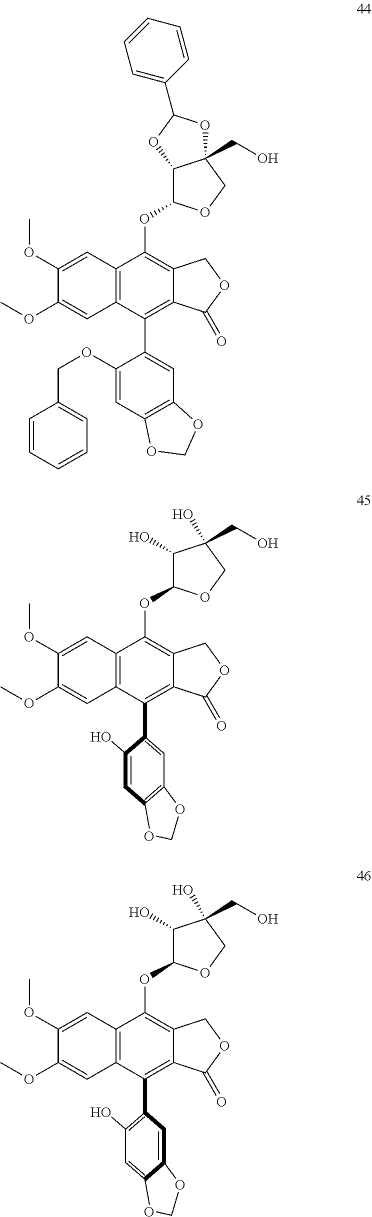

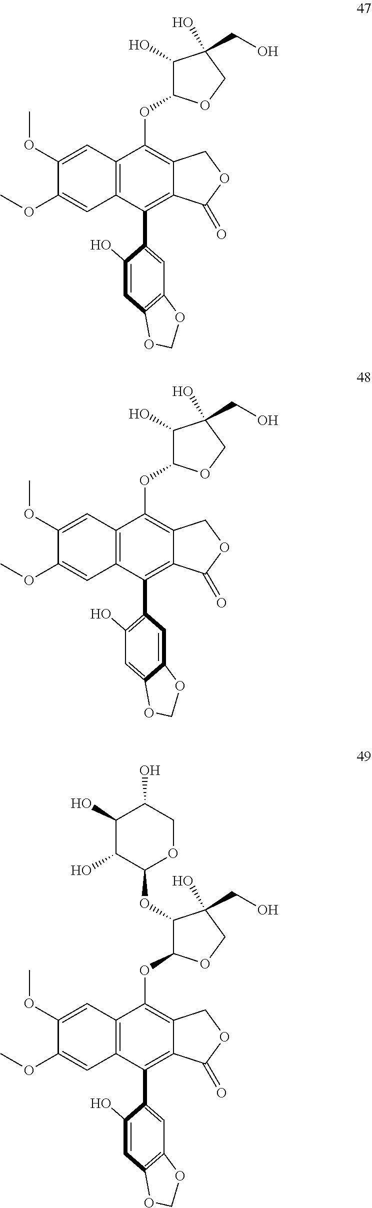

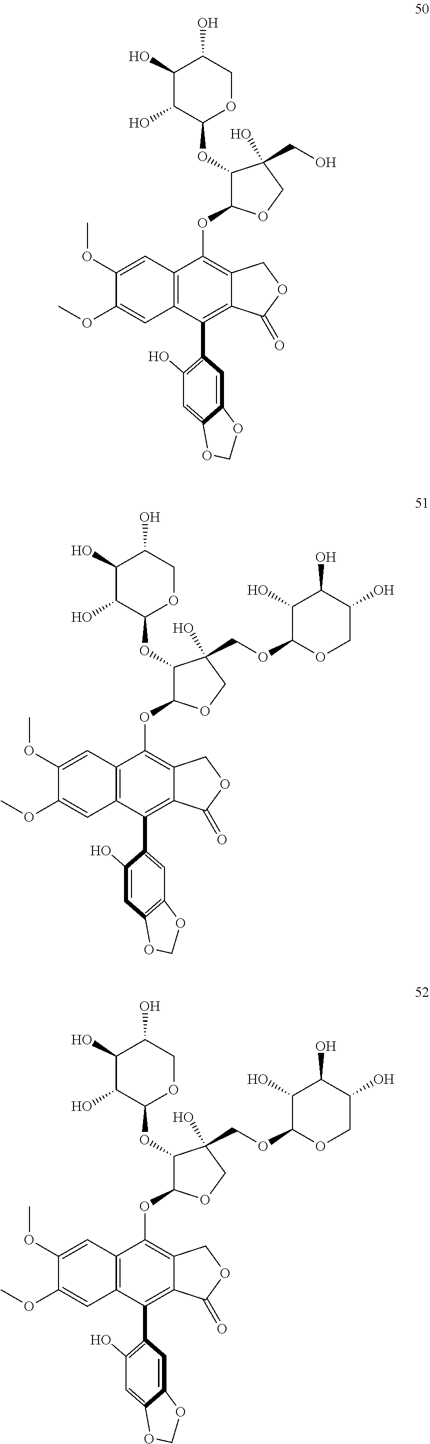

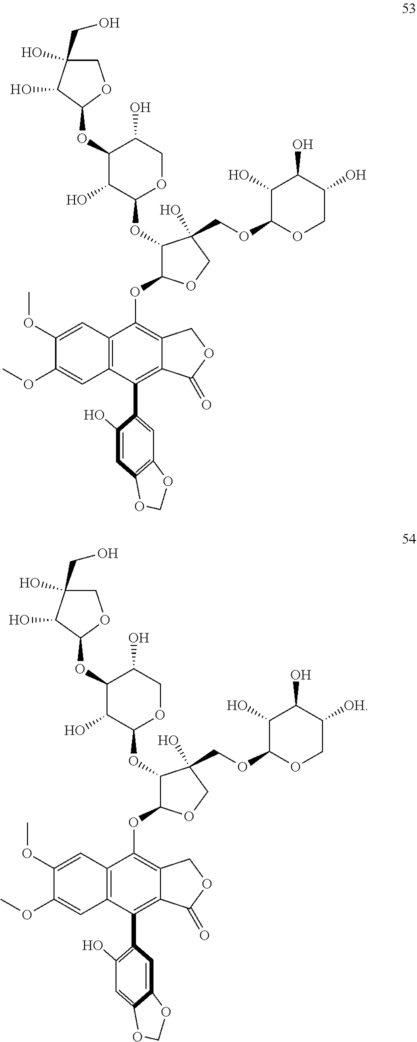

In certain embodiments, the viral infection is HIV, influenza, VSV, or CoV.

In certain embodiments, the influenza is AIV.

In certain embodiments, the AIV is influenza A.

In certain embodiments, the influenza A is H5N₁.

In certain embodiments, the CoV is SARS-CoV-2.

In certain embodiments, the compound inhibits the viral replication.

In certain embodiments, the subject is human.

In certain embodiments, the subject is an animal.

In certain embodiments, the compound is present in a separated extract or fraction from a plant material.

Another aspect of the invention concerns the method to provide synthesis of new arylnaphthalene lignan compounds as well as the intermediate compounds during the synthesis. In addition, the invention is directed to an intermediary compound useful in preparing other compounds of the invention.

Compounds of the invention may exist in different forms, such as free acids, free bases, esters and other prodrugs, salts and tautomers, and the disclosure includes all variant forms of these compounds.

The extent of protection includes counterfeit or fraudulent products which contain or purport to contain a compound of the invention irrespective of whether they do in fact contain such a compound and irrespective of whether any such compound is contained in a therapeutically effective amount.

Included in the scope of protection are packages which include a description or instructions which indicate that the package contains a species or pharmaceutical formulation of the invention and a product which is or comprises, or purports to be or comprise, such a formulation or species. Such packages may be, but are not necessarily, counterfeit or fraudulent.

Features, integers, characteristics, compounds, chemical moieties or groups described in conjunction with a particular aspect, embodiment or example of the invention are to be understood to be applicable to any other aspect, embodiment or example described herein unless incompatible therewith.

### BRIEF DESCRIPTION OF THE DRAWINGS

The above and other objects and features of the present disclosure will become apparent from the following description of the disclosure, when taken in conjunction with the accompanying drawings.

FIG. 1 shows the structure and carbon numbering of tuberculatin (1) and diphyllin (7).

FIG. 2 shows schematic preparation of protected D-apiose (6) (for preparation of compound 3, the reaction reagents and conditions of a: H₂SO₄, acetone, r.t.; for preparation of compounds 4, the reaction reagents and conditions of b: HCHO (aq., 39.5% wt), K₂CO₃, MeOH, reflux; for preparation of compounds 5, the reaction reagents and conditions of c: NaBH₄, H₂O and CH₂Cl₂, r.t., and the reaction reagents and conditions of d: NaIO₄, H₂O, r.t.; for preparation of compounds 6, the reaction reagents and conditions of e: TBDPSCl, imidizole, DMAP, CH₂Cl₂, 0° C.˜r.t.

FIG. 3 shows schematic preparation of tuberculatin analogs 8-12 (for preparation of compound 8, the reaction reagents and conditions of a: compound 6, DIAD, PPh₃, THF, r.t.; for preparation of compounds 9 and 10, the reaction reagents and conditions of b: TBAF, H₂O, THF, 0° C.; for preparation of compounds 11 and 12, the reaction reagents and conditions of c: Ac₂O, Et₃N, DMAP, CH₂Cl₂, r.t.; for the unsuccessful preparation of compound tuberculatin, the reaction reagents and conditions of d: acidic conditions.

FIG. 4 shows schematic preparation of tuberculatin (1) and analogs 16-23 (for preparation of compound 13, the reaction reagents and conditions of a: benzaldehyde, CuSO₄, D-camphorsulfonic acid, DMF, reflux; for preparation of compound 14, the reaction reagents and conditions of b: HCHO (aq., 39.5% wt), K₂CO₃, MeOH, reflux; for preparation of compound 15, the reaction reagents and conditions of c: i) NaBH₄, H₂O, r.t.; ii) NaIO₄, H₂O, r.t., and d: TBDPSCl, imidizole, DMAP, CH₂Cl₂, r.t.; for preparation of compound 16, the reaction reagents and conditions of e: diphyllin (7), DIAD, PPh₃, THF, r.t.; for preparation of compounds 17 and 18, the reaction reagents and conditions of f: TBAF, THF and H₂O, 0° C.˜r.t.; for preparation of tuberculatin (1), compounds 19, 22 and 23, the reaction reagents and conditions of g: Pd(OH)₂ (10% on carbon, dry), H₂ (1 atm), THF and MeOH, r.t.; for preparation of compounds 20 and 21, the reaction reagents and conditions of h: Ac₂O, Et₃N, DMAP, CH₂Cl₂, 0° C.˜r.t.

FIG. 5 shows schematic preparation of tuberculatin analogs 24-32 (for preparation of compound 24, the reaction reagents and conditions of a: (CF₃SO₂)₂₀, pyridine, CH₂Cl₂, −30° C.; for preparation of compound 25, the reaction reagents and conditions of b: NaN₃, DMF, r.t.; for preparation of compounds 26, the reaction reagents and conditions of c: PMe₃ (1M in THF), THF, r.t.; for preparation of compounds 27-29, the reaction reagents and conditions of d: R²C(═O)Cl, Et₃N, DMAP, CH₂Cl₂, r.t.; for preparation of compounds 30-32, the reaction reagents and conditions of e: R³H (amine or amide), Cs₂CO₃, acetone, r.t.

FIG. 6 shows schematic preparation of tuberculatin analogs 42-48 (for preparation of compound 34, the reaction reagents and conditions of a: ethylene glycol, TsOH·H₂O, toluene, reflux; for preparation of compound 36, the reaction reagents and conditions of b: (HCHO)n, anhydrous MgCl₂, Et₃N, THF, 80° C.; for preparation of compound 37, the reaction reagents and conditions of c: BnBr, Cs₂CO₃, THF, 80°; for preparation of compound 38, the reaction reagents and conditions of d: n-BuLi, THF, −78° C.; for preparation of compound 39, the reaction reagents and conditions of e: DMADC, AcOH, DCM, 43° C.; for preparation of compound 40, the reaction reagents and conditions of f: i) NaBH₄, THF, reflux, ii) 3 M HCl, r.t.; for preparation of compound 42, the reaction reagents and conditions of g: PPh₃, DIAD, THF, 0° C.; for preparation of compounds 43 and 44, the reaction reagents and conditions of h: TBAF, THF, r.t.; for preparation of compounds 45-48, the reaction reagents and conditions of i: Pb(OH)₂, H₂, THF/MeOH (1:3), r.t.

FIG. 7 shows the structures and carbon numbering of compounds 49-54.

FIG. 8 shows the structures of compounds Atrop1-3.

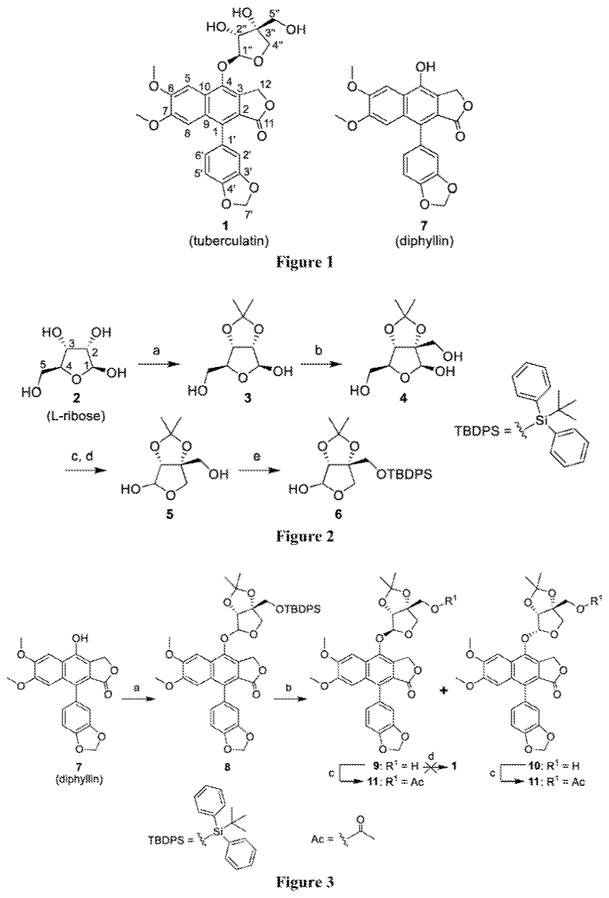

### DETAILED DESCRIPTION

The present disclosure is not to be limited in scope by any of the specific embodiments described herein. The following embodiments are presented for exemplification only.

Throughout the description and claims of this specification the word “comprise” and other forms of the word such as “comprising” and “comprises,” means including but not limited to, and is not intended to exclude, for example, other additives, components, integers, or steps.

As used herein, “comprising” is synonymous with “including.” “containing,” or “characterized by,” and is inclusive or open-ended and does not exclude additional, unrecited elements or method steps. As used herein, “consisting of” excludes any element, step, or ingredient not specified in the claim element. As used herein, “consisting essentially of” does not exclude materials or steps that do not materially affect the basic and novel characteristics of the claim. In each instance herein any of the terms “comprising”, “consisting essentially of” and “consisting of” may be replaced with either of the other two terms.

When a group of materials, compositions, components or compounds are disclosed herein, it is understood that all individual members of those groups and all subgroups thereof are disclosed separately. When a Markush group or other grouping is used herein, all individual members of the group and all combinations and subcombinations possible of the group are intended to be individually included in the disclosure. Every formulation or combination of components described or exemplified herein can be used to practice the invention, unless otherwise stated. Whenever a range is given in the specification, for example, a temperature range, a time range, or a composition range, all intermediate ranges and subranges, as well as all individual values included in the ranges given are intended to be included in the disclosure. In the disclosure and the claims, “and/or” means additionally or alternatively. Moreover, any use of a term in the singular also encompasses plural forms.

As used in the description and the appended claims, the singular forms “a,” “an,” and “the” include plural referents unless the context clearly dictates otherwise. Thus, for example, reference to “a composition” includes mixtures of two or more such compositions, reference to “the compound” includes mixtures of two or more such compounds, reference to “an agent” includes mixture of two or more such agents, and the like.

“Optional” or “optionally” means that the subsequently described event or circumstance can or cannot occur, and that the description includes instances where the event or circumstance occurs and instances where it does not.

As used herein by a “subject” is meant an individual. Thus, the “subject” can include domesticated animals (e.g., cats, dogs, etc.), livestock (e.g., cattle, horses, pigs, sheep, goats, etc.), laboratory animals (e.g., mouse, rabbit, rat, guinea pig, etc.), and birds. “Subject” can also include a mammal, such as a primate or a human.

By “reduce” or other forms of the word, such as “reducing” or “reduction,” is meant lowering of an event or characteristic (e.g., viral replication or transmission). It is understood that this is typically in relation to some standard or expected value, in other words it is relative, but that it is not always necessary for the standard or relative value to be referred to. For example, “reduces tumor growth” means reducing the rate of growth of a tumor relative to a standard or a control.

By “prevent” or other forms of the word, such as “preventing” or “prevention,” is meant to stop a particular event or characteristic, to stabilize or delay the development or progression of a particular event or characteristic, or to minimize the chances that a particular event or characteristic will occur. Prevent does not require comparison to a control as it is typically more absolute than, for example, reduce. As used herein, something could be reduced but not prevented, but something that is reduced could also be prevented. Likewise, something could be prevented but not reduced, but something that is prevented could also be reduced. It is understood that where reduce or prevent are used, unless specifically indicated otherwise, the use of the other word is also expressly disclosed.

By “treat” or other forms of the word, such as “treated” or “treatment,” is meant to administer a composition or to perform a method in order to reduce, prevent, inhibit, or eliminate a particular characteristic or event (e.g., tumor growth or survival). The term “control” is used synonymously with the term “treat.”

The term “antiviral” refers to the ability to inhibit the replication of the particular virus, to inhibit viral transmission, or to prevent the virus from establishing itself in its host, and to ameliorate or alleviate the symptoms of the disease caused by the viral infection. The treatment is considered therapeutic if there is a reduction in viral load, decrease in mortality and/or morbidity.

The term “therapeutically effective” means the amount of the composition used is of sufficient quantity to ameliorate one or more causes or symptoms of a disease or disorder. Such amelioration only requires a reduction or alteration, not necessarily elimination.

As used herein, the term pharmaceutically acceptable salt refers to any salt of the compound of this invention which retains its biological properties and which is not toxic or otherwise undesirable for pharmaceutical use. Such salts may be derived from a variety of organic and inorganic counterions well known in the art and include them. Such salts include: (1) acid addition salts formed with organic or inorganic acids such as hydrochloric, hydrobromic, sulfuric, nitric, phosphoric, sulfamic, acetic, trifluoroacetic, trichloroacetic, propionic, hexanoic, cyclopentylpropionic, glycolic, glutaric, pyruvic, lactic, malonic, succinic, sorbic, ascorbic, malic, maleic, fumaric, tartaric, citric, benzoic, 3-(4-hydroxybenzoyl)benzoic, picric, cinnamic, mandelic, phthalic, lauric, methanesulfonic, ethanesulfonic, 1,2-ethane-disulfonic, 2-hydroxyethanesulfonic, benzenesulfonic, 4-chlorobenzenesulfonic, 2-naphthalenesulfonic, 4-toluenesulfonic, camphoric, camphorsulfonic, 4-methylbicyclo[2.2.2]-oct-2-ene-1-carboxylic, glucoheptonic, 3-phenylpropionic, trimethylacetic, tert-butylacetic, lauryl sulfuric, gluconic, benzoic, glutamic, hydroxynaphthoic, salicylic, stearic, cyclohexylsulfamic, quinic, muconic acid and the like acids; or (2) salts formed when an acidic proton present in the parent compound either (a) is replaced by a metal ion (e.g., an alkali metal ion, an alkaline earth ion or an aluminum ion), or alkali metal or alkaline earth metal hydroxides (e.g., sodium, potassium, calcium, magnesium, aluminum, lithium, zinc, and barium hydroxide), ammonia or (b) coordinates with an organic base, such as aliphatic, alicyclic, or aromatic organic amines, such as ammonia, methylamine, dimethylamine, diethylamine, picoline, ethanolamine, diethanolamine, triethanolamine, ethylenediamine, lysine, arginine, ornithine, choline, N,N′-dibenzylethylene-diamine, chloroprocaine, diethanolamine, procaine, N-benzylphenethylamine, N-methylglucamine piperazine, tris(hydroxymethyl)-aminomethane, tetramethylammonium hydroxide, and the like. In addition, examples of salts include sodium, potassium, calcium, magnesium, ammonium, tetraalkylammonium and the like, and when the compound contains a basic functionality, salts of non-toxic organic or inorganic acids, such as hydrohalides (e.g., hydrochloride and hydrobromide), sulfate, phosphate, sulfamate, nitrate, acetate, trifluoroacetate, trichloroacetate, propionate, hexanoate, cyclopentylpropionate, glycolate, glutarate, pyruvate, lactate, malonate, succinate, sorbate, ascorbate, malate, maleate; fumarate, tartarate, citrate, benzoate, 3-(4-hydroxybenzoyl)benzoate, picrate, cinnamate, mandelate, phthalate, laurate, methanesulfonate (mesylate), ethanesulfonate, 1,2-ethane-disulfonate, 2-hydroxyethanesulfonate, benzenesulfonate (besylate), 4-chlorobenzenesulfonate, 2-naphthalenesulfonate, 4-toluenesulfonate, camphorate, camphorsulfonate, 4-methylbicyclo[2.2.2]-oct-2-ene-1-carboxylate, glucoheptonate, 3-phenylpropionate, trimethylacetate, tert-butylacetate, lauryl sulfate, gluconate, benzoate, glutamate, hydroxynaphthoate, salicylate, stearate, cyclohexylsulfamate, quinate, muconate and the like.

The term “glycoside” or “glycosidic” compound as used herein is interchangeable and includes reference to any of the class of compounds that yield a sugar and an aglycone upon hydrolysis.

The term “ANL” or “aryl naphthalene lignan” or “arylnaphthalene lignan” compound as used herein is interchangeable.

The term “aryl naphthalene lignan” or “arylnaphthalene lignan” or “ANL” as used herein includes reference to a compound comprising the basic structure of 2,3-dimethyl-1-phenyl-naphthalene shown as below:

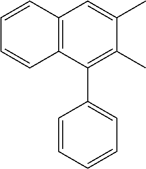 [SMILES: Cc1cc2ccccc2c(-c2ccccc2)c1C]

The carbon numbering of aryl naphthalene lignan molecule as used herein includes reference to a compound comprising numbering system shown as below:

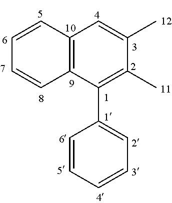 [SMILES: Cc1cc2ccccc2c(-c2ccccc2)c1C]

In one class of the core structure of an aryl naphthalene compound, the two methyl groups are forming a γ-lactone ring to become as aryl naphthofuran-2-one lignan or aryl naphthofuran-3-one lignan shown as below:

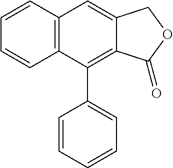 [SMILES: O=C1OCc2cc3ccccc3c(-c3ccccc3)c21]

Aryl naphthofuran-2-one lignan or

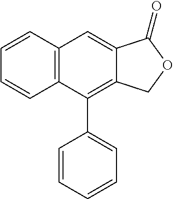 [SMILES: O=C1OCc2c1cc1ccccc1c2-c1ccccc1]

Aryl naphthofuran-3-one lignan

The carbon numbering of an aryl naphthalene lignan glycoside molecule as used herein includes reference to a compound comprising numbering system shown as below:

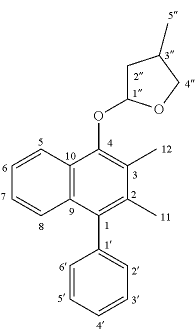 [SMILES: Cc1c(C)c(-c2ccccc2)c2ccccc2c1OC1CC(C)CO1]

The term “hydrocarbyl” as used herein includes reference to a moiety consisting exclusively of hydrogen and carbon atoms; such a moiety may comprise an aliphatic and/or an aromatic moiety. The moiety may comprise 1, 2, 3, 4, 5, 6, 7, 8, 9, 10, 11, 12, 13, 14, 15, 16, 17, 18, 19 or 20 carbon atoms. Examples of hydrocarbyl groups include C₁₋₆ alkyl (e.g. C₁, C₂, C₃ or C₄ alkyl, for example methyl, ethyl, propyl, isopropyl. n-butyl, sec-butyl or tert-butyl); C₁₋₆ alkyl substituted by aryl (e.g. benzyl) or by cycloalkyl (e.g. cyclopropylmethyl); cycloalkyl (e.g. cyclopropyl, cyclobutyl, cyclopentyl or cyclohexyl); aryl (e.g. phenyl, naphthyl or fluorenyl); C₂₋₆ alkenyl (e.g. ethenyl, 2-propenyl or 3-butenyl); C2-6 alkynyl (e.g. ethynyl, 2-propynyl or 3-butynyl) and the like.

The terms “alkyl” and “C₁₋₆ alkyl” as used herein include reference to a straight or branched chain alkyl moiety having 1, 2, 3, 4, 5 or 6 carbon atoms. This term includes reference to groups such as methyl, ethyl, propyl (n-propyl or isopropyl), butyl (n-butyl, sec-butyl or tert-butyl), pentyl, hexyl and the like. In particular, the alkyl moiety may have 1, 2, 3 or 4 carbon atoms.

The terms “alkenyl” as used herein include reference to a straight or branched chain alkyl moiety having 2, 3, 4, 5, 6, 7, 8, 9, 10, 11, 12, 13, 14, 15, 16, 17, 18, 19 or 20 carbon atoms and having, in addition, at least one double bond, of either E or Z stereochemistry where applicable. This term includes reference to groups such as ethenyl, 2-propenyl, 1-butenyl, 2-butenyl, 3-butenyl, 1-pentenyl, 2-pentenyl, 3-pentenyl, 1-hexenyl, 2-hexenyl and 3-hexenyl and the like.

The terms “alkynyl” as used herein include reference to a straight or branched chain alkyl moiety having 2, 3, 4, 5, 6, 7, 8, 9, 10, 11, 12, 13, 14, 15, 16, 17, 18, 19 or 20 carbon atoms and having, in addition, at least one triple bond. This term includes reference to groups such as ethynyl, 2-propynyl, 1-butynyl, 2-butynyl, 3-butynyl, 1-pentynyl, 2-pentynyl, 3-pentynyl, 1-hexynyl, 2-hexynyl and 3-hexynyl and the like.

The terms “alkoxy” and “C₁₋₆ alkoxy” as used herein include reference to —O-alkyl, wherein alkyl is straight or branched chain and comprises 1, 2, 3, 4, 5 or 6 carbon atoms. In one class of embodiments, alkoxy has 1, 2, 3 or 4 carbon atoms. This term includes reference to groups such as methoxy, ethoxy, propoxy, isopropoxy, butoxy, tertbutoxy, pentoxy, hexoxy and the like.

The term “cycloalkyl” as used herein includes reference to an alicyclic moiety having 3, 4, 5, 6, 7 or 8 carbon atoms. The group may be a bridged or polycyclic ring system. More often cycloalkyl groups are monocyclic. This term includes reference to groups such as cyclopropyl, cyclobutyl, cyclopentyl, cyclohexyl, norbomyl, bicyclo[2.2.2]octyl and the like.

The term “aryl” as used herein includes reference to an aromatic ring system comprising 6, 7, 8, 9, 10, 11, 12, 13, 14, 15 or 16 ring carbon atoms. Aryl is often phenyl but may be a polycyclic ring system, having two or more rings, at least one of which is aromatic. This term includes reference to groups such as phenyl, naphthyl, fluorenyl, azulenyl, indenyl, anthryl and the like. The aromatic ring may be substituted at one or more ring positions with such substituents as described above, for example, halogen, azide, alkyl, aralkyl, alkenyl, alkynyl, cycloalkyl, hydroxyl, alkoxyl, amino, nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aromatic or heteroaromatic moieties, —CF₃, —CN, or the like. The term “aryl” also includes polycyclic ring systems having two or more cyclic rings in which two or more carbons are common to two adjoining rings (the rings are “fused rings”) wherein at least one of the rings is aromatic, e.g., the other cyclic rings may be cycloalkyls, cycloalkenyls, cycloalkynyls, aryls and/or heterocyclyls.

The term “aralkyl” is art-recognized and refers to an alkyl group substituted with an aryl group (e.g., an aromatic or heteroaromatic group).

“Cyclic group” means a ring or ring system, which may be unsaturated or partially unsaturated but is usually saturated, typically containing 5 to 13 ring-forming atoms, for example a 5- or 6-membered ring. It includes carbocyclyl and heterocyclyl moeities.

The term “carbocyclyl” as used herein includes reference to a saturated (e.g. cycloalkyl) or unsaturated (e.g. aryl) ring moiety having 3, 4, 5, 6, 7, 8, 9, 10, 11, 12, 13, 14, 15 or 16 carbon ring atoms. In particular, carbocyclyl includes a 3- to 10-membered ring or ring system and, in particular, 5- or 6-membered rings, which may be saturated or unsaturated. A carbocyclic moiety is, for example, selected from cyclopropyl, cyclobutyl, cyclopentyl, cyclohexyl, norbomyl, bicyclo[2.2.2]octyl, phenyl, naphthyl, fluorenyl, azulenyl, indenyl, anthryland the like.

The term “heterocyclyl” as used herein includes reference to a saturated (e.g. heterocycloalkyl) or unsaturated (e.g. heteroaryl) heterocyclic ring moiety having from 3, 4, 5, 6, 7, 8, 9, 10, 11, 12, 13, 14, 15 or 16 ring atoms, at least one of which is selected from boron, nitrogen, oxygen, phosphorus, silicon and sulfur. In particular, heterocyclyl includes a 3- to 10-membered ring or ring system and more particularly a 5 or 6-membered ring, which may be saturated or unsaturated.

A heterocyclic moiety is, for example, selected from oxiranyl, azirinyl, 1.2-oxathiolanyl, imidazolyl, thienyl, furyl, tetrahydrofuryl, pyranyl, thiopyranyl, thianthrenyl, isobenzofuranyl, benzofuranyl, chromenyl, 2H-pyrrolyl pyrrolyl pyrrolinyl, pyrrolidinyl, pyrrolizidinyl, imidazolyl, imidazolidinyl, benzimidazolyl, pyrazolyl pyrazinyl, pyrazolidinyl, thiazolyl, isothiazolyl, dithiazolyl, oxazolyl, isoxazolyl, pyridyl, pyrazinyl, pyrimidinyl, piperidyl, piperazinyl, pyridazinyl, morpholinyl, thiomorpholinyl, especially thiomorpholino, indolizinyl, isoindolyl, 3H-indolyl, indolyl, benzimidazolyl, cumaryl, indazolyl, triazolyl, tetrazolyl, purinyl, 4N-quinolizinyl, isoquinolyl, quinolyl, tetrahydroquinolyl, tetrahydroisoquinolyl, decahydroquinolyl, octahydroisoquinolyl, benzofuranyl, dibenzofuranyl, benzothiophenyl, dibenzothiophenyl, phthalazinyl, naphthyridinyl, quinoxalyl, quinazolinyl, quinazolinyl, cinnolinyl, pteridinyl, carbazolyl, B-carbolinyl, phenanthridinyl, acridinyl, perimidinyl, phenanthrolinyl, furazanyl, phenazinyl, phenothiazinyl, phenoxazinyl, chromenyl, isochromanyl, chromanyl, 1,3,2-dioxaborolane, and the like.

The term “heterocycloalkyl” as used herein includes reference to a saturated heterocyclic moiety having 3, 4, 5, 6 or 7 ring carbon atoms and 1, 2, 3, 4 or 5 ring heteroatoms selected from nitrogen, oxygen, phosphorus and sulfur. The group may be a polycyclic ring system but more often is monocyclic. This term includes reference to groups such as azetidinyl, pyrrolidinyl, tetrahydrofuranyl, piperidinyl, oxiranyl, pyrazolidinyl, imidazolyl, indolizidinyl, piperazinyl, thiazolidinyl, morpholinyl, thiomorpholinyl, quinolizidinyl and the like.

The term “heteroaryl” as used herein includes reference to an aromatic heterocyclic ring system having 5, 6, 7, 8, 9, 10, 11, 12, 13, 14, 15 or 16 ring atoms, at least one of which is selected from nitrogen, oxygen and sulfur. The group may be a polycyclic ring system, having two or more rings, at least one of which is aromatic, but is more often monocyclic. This term includes reference to groups such as pyrimidinyl, furanyl, benzobthiophenyl, thiophenyl, pyrrolyl, imidazolyl, pyrrolidinyl, pyridinyl, benzobfuranyl, pyrazinyl, purinyl, indolyl, benzimidazolyl, quinolinyl, phenothiazinyl, triazinyl, phthalazinyl, 2H-chromenyl, oxazolyl, isoxazolyl, thiazolyl, isoindolylindazolyl, purinyl, isoquinolinyl, quinazolinyl, pteridinyl and the like.

The term “halogen” as used herein includes reference to F, Cl, Br or I.

The expression “halogen containing moiety” as used herein includes reference to a moiety comprising 1 to 30 plural valence atoms selected from carbon, nitrogen, oxygen and sulfur which moiety includes at least one halogen. The moiety may be hydrocarbyl for example C₁₋₆ alkyl or C₁₋₆ alkoxy, or carbocyclyl for example aryl.

The term “substituted” as used herein in reference to a moiety means that one or more, especially up to 5, more especially 1, 2 or 3, of the hydrogen atoms in said moiety are replaced independently of each other by the corresponding number of the described substituents. The term “optionally substituted” refers to a chemical group, such as alkyl, cycloalkyl aryl, and the like, wherein one or more hydrogen may be replaced with a substituent as described herein, for example, halogen, azide, alkyl, aralkyl, alkenyl, alkynyl, cycloalkyl, hydroxyl, alkoxyl, amino, nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aromatic or heteroaromatic moieties, —CF₃, —CN, or the like

Where two or more moieties are described as being “each independently” selected from a list of atoms or groups, this means that the moieties may be the same or different. The identity of each moiety is therefore independent of the identities of the one or more other moieties.

The term “enantiomer” as used herein means one of two stereoisomers that are mirror images of one another.

The term “stereoisomer” as used herein means one of class of isomeric molecules that have the same molecular formula and sequence of bonded atoms, but different three-dimensional orientations of their atoms in space.

The term “tautomer” means isomeric molecules that readily interconvert by a chemical reaction. The reaction commonly results in the migration of a hydrogen atom, which results in a switch of a single bond and adjacent double bond.

A prodrug is a medication that is administered as an inactive (or less than fully active) chemical derivative that is subsequently converted to an active pharmacological agent in the body, often through normal metabolic processes.

CC₅₀ is a cytotoxicity measure of the concentration for a test drug to inhibit cell growth by 50%.

EC₅₀ is an antiviral activity measure of the effective concentration for a test drug to inhibit viral growth by 50%.

The term “selectivity index” or “SI” means a ratio that measures the window between cytotoxicity and antiviral activity by dividing the given CC₅₀ value into the IC₅₀ value (CC₅₀/IC₅₀) of a test drug. The higher SI ratio means more effective and safer a test drug would be for a given viral infection in an in vitro experiment.

The symbol “ [symbol] ” or “ [symbol] ” or “ [symbol] ” or “ [symbol] ” in a chemical structure represents a position from where the specified chemical structure is bonded to another chemical structure.

The symbol “β” in a chemical structure indicates that the bond connection is above (or before) the plane of the paper or screen. The symbol “a” in a chemical structure indicates that the bond connection is below (or behind) the plane of the paper or screen.

A solid wedge in a chemical structure indicates that this bond is above (or before) the plane of the paper or screen toward to the viewer. A hashed (or broken) wedge in a chemical structure indicates that the bond connection is below (or behind) the plane of the paper or screen receding away from the viewer.

Unless stereochemistry is explicitly depicted, a chemical structure is intended to include every possible stereoisomer, both pure or in any possible mixture.

The present disclosure provides a compound of Formula I:

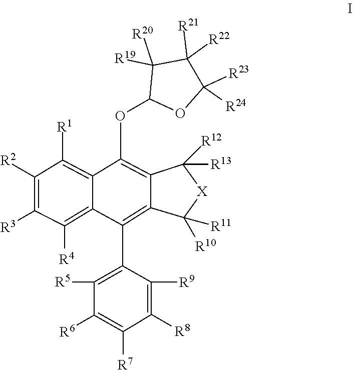

or a pharmaceutically acceptable salt thereof, wherein:
X is oxygen or sulfur;
R¹, R², R³, R⁴, R⁵, R⁶, R⁷, R⁸, and R⁹ are each independently hydrogen, alkyl, akenyl, alkynyl, cycloalkyl, heterocycloalkyl, aryl, aralkyl, heteroaryl, haloalkyl, halogen, cyano, NO₂, —OR²⁶, —C(═O)R²⁷, —C(═O)N(R²⁶)R²⁷, —C(═O)OR²⁶, —OC(═O)R²⁶, —OSi(R²⁵)(R²⁶)R²⁷, —S(═O)₂R²⁶, —S(═O)₂N(R²⁶)R²⁷, —N(R²⁶)R²⁷, —N(R²⁶)N(R²⁶)R²⁷, —N(R²⁶)C(═O)R²⁷, —N(R²⁶)S(═O)₂R²⁷ or a moiety comprising 1 to 30 plural valence atoms selected from the group consisting of carbon, nitrogen, oxygen and sulfur; or R¹ and R², R² and R³, R³ and R⁴, R⁵ and R⁶, R⁶ and R⁷, R⁷ and R⁸, or R⁸ and R⁹ taken together with the carbon atoms to which they are attached to form a cyclic group which is optionally substituted with halogen or a moiety comprising 1 to 30 plural valence atoms selected from the group consisting of carbon, nitrogen, oxygen and sulfur;
R¹⁰ and R¹¹ taken together form oxo; or while one of R¹⁰ and R¹¹ is hydrogen or halogen, the other one of R¹⁰ and R¹¹ is selected from the group consisting of R²⁵, —OR²⁵, —C(═O)R²⁵, and —C(═O)OR²⁵;
R¹² and R¹³ taken together form oxo; or while one of R¹² and R¹³ is hydrogen or halogen, the other one of R¹² and R¹³ is selected from the group consisting of R²⁵, —OR²⁵, —C(═O)R²⁵, and —C(═O)OR²⁵;
R¹⁹ and R²⁰ taken together form oxo; or while one of R¹⁹ and R²⁰ is hydrogen or halogen, the other one of R¹⁹ and R²⁰ is selected from the group consisting of R²⁵, —OR²⁵, —C(═O)R²⁵, —C(═O)OR²⁵, —OC(═O)R²⁵, —OC(═O)N(R²⁵)R²⁵, optionally substituted monosaccharide, optionally substituted disaccharide, optionally substituted trisaccharide, and optionally substituted tetrasaccharide;
R²¹ and R²² taken together form oxo; or while one of R²¹ and R²² is hydrogen, halogen, R²⁵ or —OR²⁵, the other one of R²¹ and R²² is selected from the group consisting of R²⁵, —OR²⁵, —OC(═O)R²⁵, —OC(═O)N(R²⁵)R²⁵, —C(═O)OR²⁵, —CH₂R²⁹, —CH₂OR²⁹, —C(═O)R²⁹; or R¹⁹ and R²¹ taken together with the carbon atoms to which they are attached to form a 5-6 membered heterocyclyl optionally substituted with 1, 2, 3, 4 or 5 group(s) independently selected from R²⁵;
R²³ and R²⁴ taken together form oxo; or while one of R²³ and R²⁴ is hydrogen or halogen, the other one of R²³ and R²⁴ is selected from the group consisting of R²⁵, —OR²⁵, —C(═O)R²⁵, —C(═O)OR²⁵, optionally substituted monosaccharide, optionally substituted disaccharide, optionally substituted trisaccharide, and optionally substituted tetrasaccharide;
R²⁵ for each occurrence is independently selected from hydrogen, halogen, trichloromethyl, trifluoromethyl, cyano, nitro, —OR²⁶, —C(═O)R²⁷, —C(═O)N(R²⁶)R²⁷, —C(═O)OR²⁶, —OC(═O)R²⁶, —OSi(R²⁵)(R²⁶)R²⁷, —S(═O)₂R²⁶, —S(═O)₂N(R²⁶)R²⁷, —N(R²⁶)R²⁷, —N(R²⁶)N(R²⁶)R²⁷, —N(R²⁶)C(═O)R²⁷, —N(R²⁶)S(═O)₂R²⁷, —N₃, —OS(═O)₂CF₃, hydrocarbyl optionally substituted with 1, 2, 3, 4 or 5 group(s) independently selected from the group consisting of R²⁸, heterocyclcyl optionally substituted with 1, 2, 3, 4 or 5 group(s) independently selected from the group consisting of R², and —(CH₂)ₖ-heterocyclyl optionally substituted with 1, 2, 3, 4 or 5 group(s) independently selected from the group consisting of R²⁸, wherein k is an integer between 1 and 6;
R²⁶ and R²⁷ for each occurrence are each independently hydrogen or selected from hydrocarbyl and heterocyclyl, either of which is optionally substituted with 1, 2, 3, 4 or 5 group(s) independently selected from halogen, cyano, amino, hydroxy, C₁₋₆ alkyl and C₁₋₆ alkoxy;
R²⁸ for each occurrence is independently selected from halogen, trichloromethyl, trifluoromethyl, cyano, nitro, oxo, ═NR²⁶, —OR²⁶, —C(═O)R²⁷, —C(═O)N(R²⁶)R²⁷, —C(═O)OR²⁶, —OC(═O)R²⁶, —OSi(R²⁵)(R²⁶)R²⁷, —S(═O)₂R²⁶, —S(═O)₂N(R²⁶)R²⁷, —N(R²⁶)R²⁷, —N(R²⁶)N(R²⁶)R²⁷, —N(R²⁶)C(═O)R²⁷ and —N(R²⁶)S(═O)₂R²⁷; and
R²⁹ for each occurrence is independently selected from hydrogen, halogen, trichloromethyl, trifluoromethyl, cyano, nitro, —OR²⁶, —C(═O)R²⁷, —C(═O)N(R²⁶)R²⁷, —C(═O)OR²⁶, —OC(═O)R²⁶, —OSi(R²⁵)(R²⁶)R²⁷, —S(═O)₂R²⁶, —S(═O)₂N(R²⁶)R²⁷, —N(R²⁶)R²⁷, —N(R²⁶)N(R²⁶)R²⁷, —N(R²⁶)C(═O)R²⁷, —N(R²⁶)S(═O)₂R²⁷, —N₃, —OS(═O)₂CF₃, optionally substituted monosaccharide, optionally substituted disaccharide, optionally substituted trisaccharide, optionally substituted tetrasaccharide, hydrocarbyl optionally substituted with 1, 2, 3, 4 or 5 group(s) independently selected from the group consisting of R²⁸, and heterocyclyl optionally substituted with 1, 2, 3, 4 or 5 group(s) independently selected from the group consisting of R²⁸, with the proviso that the compound of Formula I does not include a compound selected from the group consisting of 1, 1-Ac, A1, A2, A3, A4, A5, A6, A7, and A8:

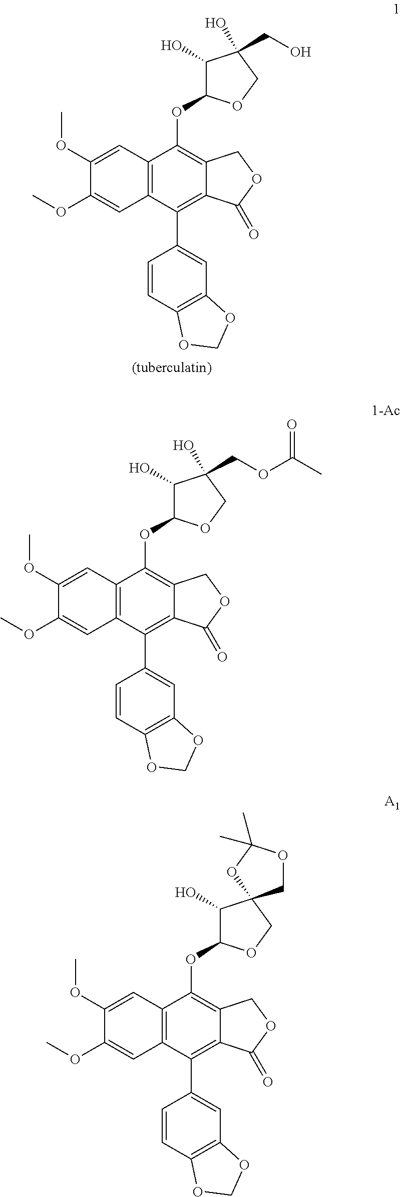

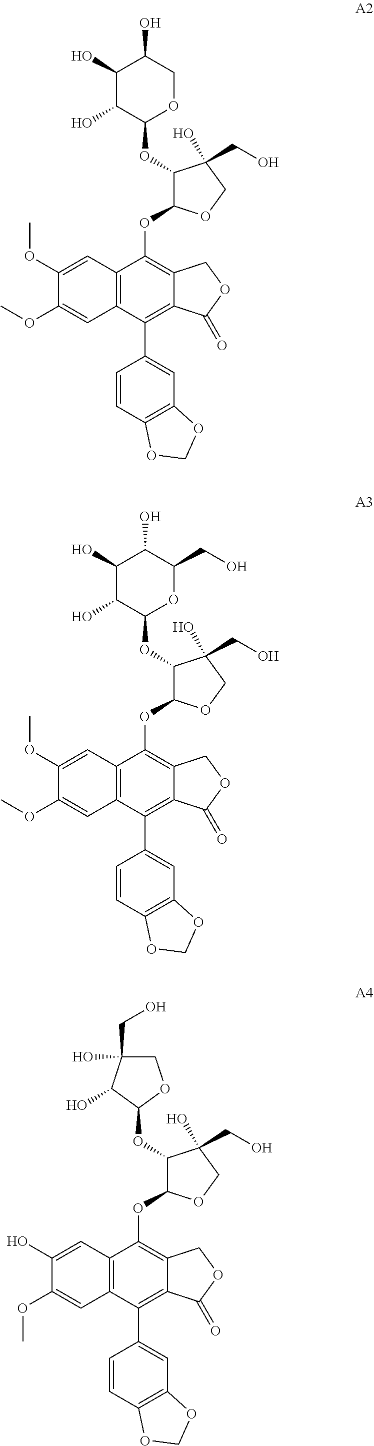

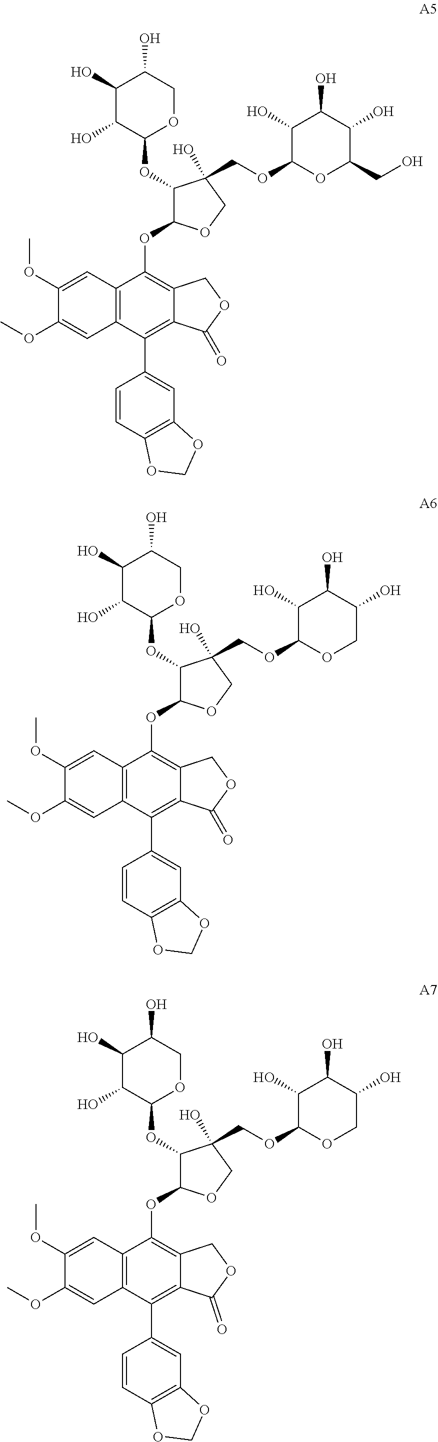

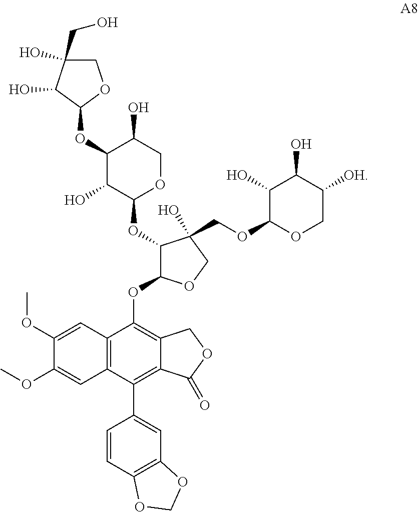

The present disclosure contemplates synthetic and semi-synthetic compounds described herein and excludes naturally occurring compounds in the form in which they occur in nature.

In certain embodiments, R¹, R², R³, R⁴, R⁵, R⁶, R⁷, R⁹, and R⁹ are each independently hydrogen, alkyl, akenyl, alkynyl, cycloalkyl, heterocycloalkyl, aryl, aralkyl, heteroaryl, haloalkyl, halogen, cyano, NO₂, —OR²⁶, —C(═O)R²⁷, —C(═O)N(R²⁶)R²⁷, —C(═O)OR²⁶, —OC(═O)R²⁶, —OSi(R²⁵)(R²⁶)R²⁷, —S(═O)₂R²⁶, —S(═O)₂N(R²⁶)R²⁷, —N(R²⁶)R²⁷, —N(R²⁶)N(R²⁶)R²⁷, —N(R²⁶)C(═O)R²⁷, or —N(R²⁶)S(═O)₂R²⁷; or R¹ and R², R² and R³, R³ and R⁴, R⁵ and R⁶, R⁶ and R⁷, R⁷ and R⁸, or R⁸ and R⁹ taken together with the carbon atoms to which they are attached to form a 5-membered heterocyclyl.

In certain embodiments, R¹, R², R³, R⁴, R⁵, R⁶, R⁷, R⁹, and R⁹ are each independently hydrogen, alkyl, halogen, cyano, NO₂, or —OR²⁶; or R¹ and R², R² and R³, R³ and R⁴, R⁵ and R⁶, R⁶ and R⁷, R⁷ and R⁸, or R⁸ and R⁹ taken together with the carbon atoms to which they are attached to form a 5-membered heterocyclyl.

In certain embodiments, R¹, R⁴, R⁵, and R⁹ are each hydrogen; R², R³, R⁷, and R⁷ are each independently —OR²⁶; and R⁶ is hydrogen or —OR²⁶, wherein R²⁶ is hydrogen, alkyl, cycloalkyl, heterocycloalkyl, aryl, aralkyl (e.g., benzyl), or heteroaryl; or R² and R³ taken together with the carbon atoms to which they are attached to form a 5-membered heterocyclyl; or R⁷ and R⁸ taken together with the carbon atoms to which they are attached to form a 5-membered heterocyclyl.

In certain embodiments, R¹, R⁴, R⁵, R⁶, and R⁹ are each hydrogen; R² and R³ are each —OCH₃; and R⁷ and R⁸ taken together form a methylenedioxy group.

In instances in which R² and R³ or R⁷ and R⁸ taken together with the carbon atoms to which they are attached to form a 5-membered heterocyclyl, the 5-membered heterocyclyl can have the structure:

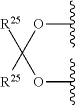

wherein R²⁵ for each occurrence is independently selected from hydrogen, alkyl, cycloalkyl, aryl, and heteroaryl. In certain embodiments, each R²⁵ is hydrogen.

In certain embodiments, R¹⁰ and R¹¹ taken together form oxo (C═O).

In certain embodiments, R¹² and R¹³ taken together form oxo (C═O).

In certain embodiments, R¹⁹ and R²⁰ taken together to form oxo; or R²⁰ is hydrogen, and R¹⁹ is selected from the group consisting of R²⁵, —OR²⁵, —C(═O)R²⁵, —C(═O)OR²⁵, —OC(═O)R²⁵, —OC(═O)N(R²⁵)R²⁵, optionally substituted monosaccharide, optionally substituted disaccharide, optionally substituted trisaccharide, and optionally substituted tetrasaccharide.

In certain embodiments, R²¹ and R²² taken together to form oxo; or R²² is —OR²⁵; and R²¹ is selected from the group consisting of —CH₂R²⁹ and —CH₂OR²⁹; or R¹⁹ and R²¹ taken together with the carbon atoms to which they are attached to form a 5-6 membered heterocyclyl optionally substituted with 1, 2, 3, 4 or 5 group(s) independently selected from R²⁵.

In instances in which one or more of R¹⁹, R²⁰, R²³, R²⁴, and R²⁹ is an optionally substituted monosaccharide, optionally substituted disaccharide, optionally substituted trisaccharide, and optionally substituted tetrasaccharide, the group can comprise any monosaccharide, disaccharide, trisaccharide, or tetrasaccharide. Exemplary glycosidic groups include glucopyranoside, glucofuranoside, galactopyranoside, mannopyranoside, fucopyranoside, arabinopyranoside, arabinofuranoside, glucopyranoside, galactopyranoside, glucuronide, lactopyranoside, xylopyranoside, glucosaminide, galactosaminide, alloside, apioside, lyxoside, taloside, threoside, riboside, fructoside, rhamnoside and guloside groups. More particularly, the glycosidic group may be selected from α-D-glucopyranoside, α-D-galactopyranoside, α-D-mannopyranoside, α-L-fucopyranoside, α-L-arabinopyranoside, β-D-glucopyranoside, β-D-galactopyranoside, β-D-apiofuranoside, β-D-ribofuranoside, β-D-xylofuranoside, β-D-fructofuranoside, β-D-galactofuranoside, 2-deoxy-β-D-erythro-pentofuranoside, α-D-gulofuranoside, α-D-arabinofuranoside, α-D-glucofuranoside, α-L-glucofuranoside, β-D-glucuronide, β-D-lactopyranoside, β-D-xylopyranoside, β-D-glucosaminide, β-D-galactosaminide, β-D-alloside, β-D-lyxoside, β-D-taloside, β-D-threoside, β-D-riboside, β-D-fructoside, β-D-rhamnoside and β-L-guoside groups.

In certain embodiments, R²⁵ for each occurrence is independently selected from the group consisting of hydrogen, alkyl, alkenyl, alkynyl, cycloalkyl, aryl, aralkyl, heterocycloalkyl, and heteraryl.

In certain embodiments, R²⁶ and R²⁷ for each occurrence are each independently selected from the group consisting of hydrogen, alkyl, alkenyl, alkynyl, cycloalkyl, aryl, aralkyl, heterocycloalkyl, and heteraryl.

In certain embodiments, the compound has Formula II:

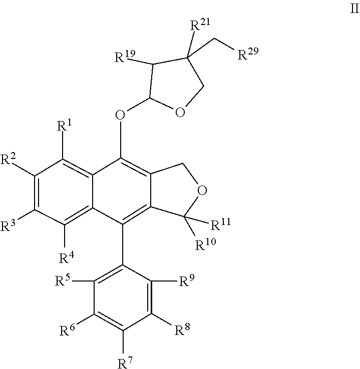

or a pharmaceutically acceptable salt thereof, wherein:
R¹, R², R³, R⁴, R⁵, R⁶, R⁷, R⁸, and R⁹ are each independently hydrogen, alkyl, akenyl, alkynyl, cycloalkyl, heterocycloalkyl, aryl, aralkyl, heteroaryl, haloalkyl, halogen, cyano, NO₂, —OR²⁶, —C(═O)R²⁷, —C(═O)N(R²⁶)R²⁷, —C(═O)OR²⁶, —OC(═O)R²⁶, —OSi(R²⁵)(R²⁶)R²⁷, —S(═O)₂R²⁶, —S(═O)₂N(R²⁶)R²⁷, —N(R²⁶)R²⁷, —N(R²⁶)N(R²⁶)R²⁷, —N(R²⁶)C(═O)R²⁷, —N(R²⁶)S(═O)₂R²⁷ or a moiety comprising 1 to 30 plural valence atoms selected from the group consisting of carbon, nitrogen, oxygen and sulfur; or R¹ and R², R² and R³, R³ and R⁴, R⁵ and R⁶, R⁶ and R⁷, R⁷ and R⁸, or R⁸ and R⁹ taken together with the carbon atoms to which they are attached to form a cyclic group which is optionally substituted with halogen or a moiety comprising 1 to 30 plural valence atoms selected from the group consisting of carbon, nitrogen, oxygen and sulfur;
R¹⁰ and R¹¹ may be taken together to form oxo; or while one of R¹⁰ and R¹¹ is hydrogen, the other one of R¹⁰ and R¹¹ is selected from the group consisting of R²⁵, —OR²⁵, —C(═O)R²⁵, and —C(═O)OR²⁵;
R¹⁹ is selected from the group consisting of R²⁵, —OR²⁵, —OC(═O)R²⁵, —OC(═O)N(R²⁵)R²⁵, optionally substituted monosaccharide, optionally substituted disaccharide, optionally substituted trisaccharide, and optionally substituted tetrasaccharide;
R²¹ is selected from the group consisting of R²⁵, —OR²⁵, —OC(═O)R²⁵, —OC(═O)N(R²⁵)R²⁵, —C(═O)OR²⁵, —CH₂R²⁹, —CH₂OR²⁹, —C(═O)R²⁹; or R¹⁹ and R²¹ taken together with the carbon atoms to which they are attached to form a 5-6 membered heterocyclyl optionally substituted with 1, 2, 3, 4 or 5 group(s) independently selected from R²⁵;
R²⁵ for each occurrence is independently selected from hydrogen, halogen, trichloromethyl, trifluoromethyl, cyano, nitro, —OR²⁶, —C(═O)R²⁷, —C(═O)N(R²⁶)R²⁷, —C(═O)OR²⁶, —OC(═O)R²⁶, —OSi(R²⁵)(R²⁶)R²⁷, —S(═O)₂R²⁶, —S(═O)₂N(R²⁶)R²⁷, —N(R²⁶)R²⁷, —N(R²⁶)N(R²⁶)R²⁷, —N(R²⁶)C(═O)R²⁷, —N(R²⁶)S(═O)₂R²⁷, —N₃, —OS(═O)₂CF₃, hydrocarbyl optionally substituted with 1, 2, 3, 4 or 5 group(s) independently selected from the group consisting of R²⁸, heterocyclcyl optionally substituted with 1, 2, 3, 4 or 5 group(s) independently selected from the group consisting of R²⁸, and —(CH₂)ₖ-heterocyclyl optionally substituted with 1, 2, 3, 4 or 5 group(s) independently selected from the group consisting of R²⁸, wherein k is an integer between 1 and 6;
R²⁶ and R²⁷ for each occurrence are each independently hydrogen or selected from hydrocarbyl and heterocyclyl, either of which is optionally substituted with 1, 2, 3, 4 or 5 group(s) independently selected from halogen, cyano, amino, hydroxy, C₁₋₆ alkyl and C₁₋₆ alkoxy;
R²⁸ for each occurrence is independently selected from halogen, trichloromethyl, trifluoromethyl, cyano, nitro, oxo, ═NR²⁶, —OR²⁶, —C(═O)R²⁷, —C(═O)N(R²⁶)R²⁷, —C(═O)OR²⁶, —OC(═O)R²⁶, —OSi(R²⁵)(R²⁶)R²⁷, —S(═O)₂R²⁶, —S(═O)₂N(R²⁶)R²⁷, —N(R²⁶)R²⁷, —N(R²⁶)N(R²⁶)R²⁷, —N(R²⁶)C(═O)R²⁷ and —N(R²⁶)S(═O)₂R²⁷; and
R²⁹ for each occurrence is independently selected from hydrogen, halogen, trichloromethyl, trifluoromethyl, cyano, nitro, —OR²⁶, —C(═O)R²⁷, —C(═O)N(R²⁶)R²⁷, —C(═O)OR²⁶, —OC(═O)R²⁶, —OSi(R²⁵)(R²⁶)R²⁷, —S(═O)₂R²⁶, —S(═O)₂N(R²⁶)R²⁷, —N(R²⁶)R²⁷, —N(R²⁶)N(R²⁶)R²⁷, —N(R²⁶)C(═O)R²⁷, —N(R²⁶)S(═O)₂R²⁷, —N₃, —OS(═O)₂CF₃, optionally substituted monosaccharide, optionally substituted disaccharide, optionally substituted trisaccharide, optionally substituted tetrasaccharide, hydrocarbyl optionally substituted with 1, 2, 3, 4 or 5 group(s) independently selected from the group consisting of R²⁸, and heterocyclyl optionally substituted with 1, 2, 3, 4 or 5 group(s) independently selected from the group consisting of R²⁸.

The glycoside moiety at carbon 4 of the compounds described herein can be represented a chemical structure selected from the group consisting of:

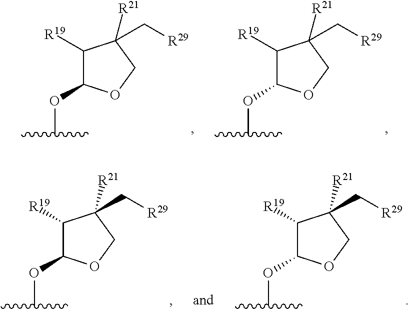

In certain embodiments, of the compound of Formula II, R¹, R⁴, R⁶, and R⁹ are each hydrogen; R⁵ is hydrogen or —OR²⁶; and R², R³, R⁷, and R⁸ are each independently —OR²⁶; or at least one of R² and R³ or R⁷ and R⁸ taken together with the carbon atoms to which they are attached to form a cyclic group which is optionally substituted with halogen or a moiety comprising 1 to 30 plural valence atoms selected from the group consisting of carbon, nitrogen, oxygen and sulfur. In certain embodiments, R²⁶ for each occurrence is independently alkyl, aryl, aralkyl, or cycloalkyl. In certain embodiments, R²⁶ for each occurrence is independently methyl or benzyl.

In instances in which of R² and R³ or R⁷ and R⁸ taken together with the carbon atoms to which they are attached to form a cyclic group which is optionally substituted with halogen or a moiety comprising 1 to 30 plural valence atoms selected from the group consisting of carbon, nitrogen, oxygen and sulfur, the cyclic group can have the structure:

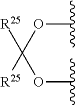

wherein R²⁵ for each occurrence is independently selected from hydrogen, alkyl, cycloalkyl, aryl, and heteroaryl. In certain embodiments, each R²⁵ is hydrogen. In certain embodiments, R⁷ and R⁸ taken together with the carbon atoms to which they are attached have the structure:

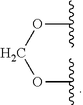 [SMILES: *OCO*]

In instances in which R¹⁹ and R²¹ taken together with the carbon atoms to which they are attached to form a 5-6 membered heterocyclyl, the 5-membered heterocyclyl can have the structure:

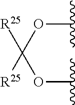

wherein R²⁵ for each occurrence is independently selected from hydrogen, alkyl, cycloalkyl, aryl, and heteroaryl. In certain embodiments, each R²⁵ is alkyl; or one instance of R²⁵ is hydrogen; and one instance R²⁵ is alkyl, cycloalkyl, aryl, or heteroaryl. In certain embodiments, each R²⁵ is methyl; or one instance of R²⁵ is hydrogen; and one instance R²⁵ is phenyl.

In certain embodiments, the compound has Formula III:

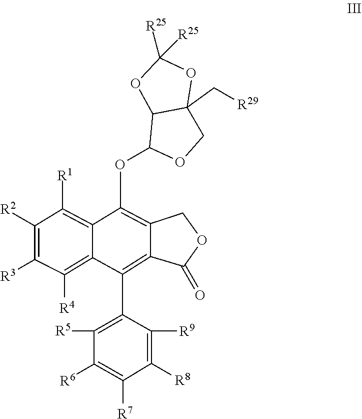

or a pharmaceutically acceptable salt thereof, wherein:
wherein R¹, R², R³, R⁴, R⁵, R⁶, R⁷, R⁸, and R⁹ are each independently hydrogen, alkyl, akenyl, alkynyl, cycloalkyl, heterocycloalkyl, aryl, aralkyl, heteroaryl, haloalkyl, halogen, cyano, NO₂, —OR²⁶, —C(═O)R²⁷, —C(═O)N(R²⁶)R²⁷, —C(═O)OR²⁶, —OC(═O)R²⁶, —OSi(R²⁵)(R²⁶)R²⁷, —S(═O)₂R²⁶, —S(═O)₂N(R²⁶)R²⁷, —N(R²⁶)R²⁷, —N(R²⁶)N(R²⁶)R²⁷, —N(R²⁶)C(═O)R²⁷, —N(R²⁶)S(═O)₂R²⁷; or R¹ and R², R² and R³, R³ and R⁴, R⁵ and R⁶, R⁶ and R⁷, R⁷ and R⁸, or R⁸ and R⁹ taken together with the carbon atoms to which they are attached to form a 5-membered heterocyclyl;
R²⁵ for each occurrence is independently selected from hydrogen and hydrocarbyl optionally substituted with 1, 2, 3, 4 or 5 group(s) independently selected from the group consisting of R²⁸, heterocyclcyl optionally substituted with 1, 2, 3, 4 or 5 group(s) independently selected from the group consisting of R²⁸, and —(CH₂)ₖ-heterocyclyl optionally substituted with 1, 2, 3, 4 or 5 group(s) independently selected from the group consisting of R²⁸, wherein k is an integer between 1 and 6;
R²⁶ and R²⁷ for each occurrence are each independently hydrogen or selected from hydrocarbyl and heterocyclyl, either of which is optionally substituted with 1, 2, 3, 4 or 5 group(s) independently selected from halogen, cyano, amino, hydroxy, C₁₋₆ alkyl and C₁₋₆ alkoxy;
R²⁸ for each occurrence is independently selected from halogen, trichloromethyl, trifluoromethyl, cyano, nitro, oxo, ═NR²⁶, —OR²⁶, —C(═O)R²⁷, —C(═O)N(R²⁶)R²⁷, —C(═O)OR²⁶, —OC(═O)R²⁶, —OSi(R²⁵)(R²⁶)R²⁷, —S(═O)₂R²⁶, —S(═O)₂N(R²⁶)R²⁷, —N(R²⁶)R²⁷, —N(R²⁶)N(R²⁶)R²⁷, —N(R²⁶)C(═O)R²⁷ and —N(R²⁶)S(═O)₂R²⁷; and
R²⁹ is selected from the group consisting of halogen, cyano, —OR²⁶, —OC(═O)R²⁶, —OSi(R²⁵)(R²⁶)R²⁷, —N(R²⁶)R²⁷, —N(R²⁶)N(R²⁶)R²⁷, —N(R²⁶)C(═O)R²⁷, —N(R²⁶)S(═O)₂R²⁷, —N₃, and —OS(═O)₂CF₃.

The glycoside moiety at carbon 4 of the compounds described herein can be represented a chemical structure selected from the group consisting of:

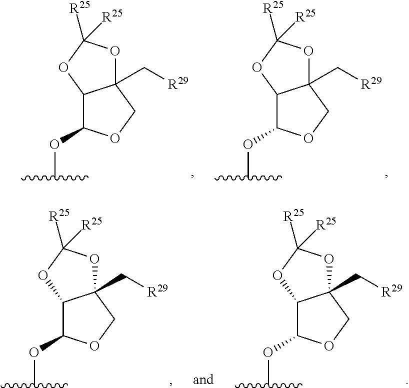

In certain embodiments, of the compound of Formula III, R¹, R⁴, R⁶, and R⁹ are each hydrogen; R⁵ is hydrogen or —OR²⁶; and R², R³, R⁷, and R⁸ are each independently —OR²⁶; or at least one of R² and R³ or R⁷ and R⁸ taken together with the carbon atoms to which they are attached to form a cyclic group which is optionally substituted with halogen or a moiety comprising 1 to 30 plural valence atoms selected from the group consisting of carbon, nitrogen, oxygen and sulfur. In certain embodiments, R²⁶ for each occurrence is independently alkyl, aryl, aralkyl, or cycloalkyl. In certain embodiments, R²⁶ for each occurrence is independently methyl or benzyl.

In instances in which of R² and R³ or R⁷ and R⁸ taken together with the carbon atoms to which they are attached to form a cyclic group which is optionally substituted with halogen or a moiety comprising 1 to 30 plural valence atoms selected from the group consisting of carbon, nitrogen, oxygen and sulfur, the cyclic group can have the structure:

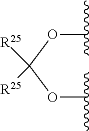

wherein R²⁵ for each occurrence is independently selected from hydrogen, alkyl, cycloalkyl, aryl, and heteroaryl. In certain embodiments, each R²⁵ is hydrogen. In certain embodiments, R⁷ and R⁸ taken together with the carbon atoms to which they are attached have the structure:

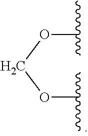 [SMILES: *OCO*]

In instances in which R¹⁹ and R²¹ taken together with the carbon atoms to which they are attached to form a 5-6 membered heterocyclyl, the 5-membered heterocyclyl can have the structure:

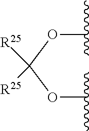

wherein R²⁵ for each occurrence is independently selected from hydrogen, alkyl, cycloalkyl, aryl, and heteroaryl. In certain embodiments, each R²⁵ is alkyl; or one instance of R²⁵ is hydrogen; and one instance R²⁵ is alkyl, cycloalkyl, aryl, or heteroaryl. In certain embodiments, each R²⁵ is methyl; or one instance of R²⁵ is hydrogen; and one instance R²⁵ is phenyl.

In certain embodiments, of the compound of Formula III, R²⁹ is selected from halogen, —OR²⁶, —OC(═O)R²⁶, —OSi(R²⁵)(R²⁶)R²⁷, —N(R²⁶)R²⁷, —N(R²⁶)N(R²⁶)R²⁷, —N(R²⁶)C(═O)R²⁷, —N(R²⁶)S(═O)₂R²⁷, —N₃, and —OS(═O)₂CF₃.

In certain embodiments, the compound has Formula IV:

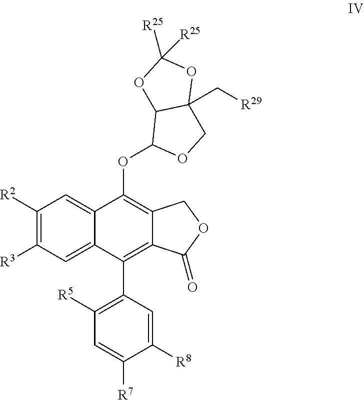

or a pharmaceutically acceptable salt thereof, wherein:
R², R³, R⁷, and R⁸ are each independently —OR²⁶; and R⁵ is hydrogen or —OR²⁶; or R² and R³ taken together form a methylenedioxy group; or R⁷ and R⁸ taken together form a methylenedioxy group;
R²⁵ for each occurrence is independently selected from hydrogen and hydrocarbyl;
R²⁶ for each occurrence is independently hydrogen, hydrocarbyl, or heterocyclyl; and
R²⁹ is selected from the group consisting of halogen, cyano, —OR²⁶, —OC(═O)R²⁶, —OSi(R²⁵)(R²⁶)R²⁷, —N(R²⁶)R²⁷, —N(R²⁶)N(R²⁶)R²⁷, —N(R²⁶)C(═O)R²⁷, —N(R²⁶)S(═O)₂R²⁷, —N₃, and —OS(═O)₂CF₃.

In certain embodiments, the compound has Formula V:

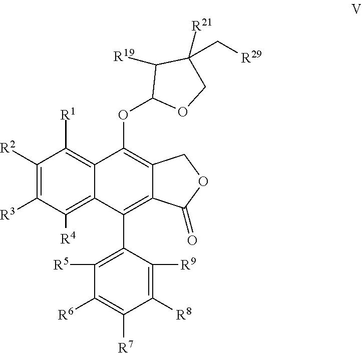

or a pharmaceutically acceptable salt thereof, wherein:
wherein R¹, R², R³, R⁴, R⁵, R⁶, R⁷, R⁸, and R⁹ are each independently hydrogen, alkyl, akenyl, alkynyl, cycloalkyl, heterocycloalkyl, aryl, aralkyl, heteroaryl, haloalkyl, halogen, cyano, NO₂, —OR²⁶, —C(═O)R²⁷, —C(═O)N(R²⁶)R²⁷, —C(═O)OR²⁶, —OC(═O)R²⁶, —OSi(R²⁵)(R²⁶)R²⁷, —S(═O)₂R²⁶, —S(═O)₂N(R²⁶)R²⁷, —N(R²⁶)R²⁷, —N(R²⁶)N(R²⁶)R²⁷, —N(R²⁶)C(═O)R²⁷, —N(R²⁶)S(═O)₂R²⁷; or R¹ and R², R² and R³, R³ and R⁴, R⁵ and R⁶, R⁶ and R⁷, R⁷ and R⁹, or R¹ and R⁹ taken together with the carbon atoms to which they are attached to form a 5-membered heterocyclyl;
R¹⁹ is selected from the group consisting of —OR²⁵, —OC(═O)R²⁵, —OC(═O)N(R²⁵)R²⁵, optionally substituted monosaccharide, optionally substituted disaccharide, optionally substituted trisaccharide, and optionally substituted tetrasaccharide;
R²¹ is selected from the group consisting of —OR²⁵, —OC(═O)R²⁵, —OC(═O)N(R²⁵)R²⁵, and —C(═O)OR²⁵;
R²⁵ for each occurrence is independently selected from hydrogen and hydrocarbyl optionally substituted with 1, 2, 3, 4 or 5 group(s) independently selected from the group consisting of R²⁸, heterocyclcyl optionally substituted with 1, 2, 3, 4 or 5 group(s) independently selected from the group consisting of R²⁸, and —(CH₂)ₖ-heterocyclyl optionally substituted with 1, 2, 3, 4 or 5 group(s) independently selected from the group consisting of R²⁸, wherein k is an integer between 1 and 6;
R²⁶ and R²⁷ for each occurrence are each independently hydrogen or selected from hydrocarbyl and heterocyclyl, either of which is optionally substituted with 1, 2, 3, 4 or 5 group(s) independently selected from halogen, cyano, amino, hydroxy, C₁₋₆ alkyl and C₁₋₆ alkoxy;
R²⁸ for each occurrence is independently selected from halogen, trichloromethyl, trifluoromethyl, cyano, nitro, oxo, ═NR²⁶, —OR²⁶, —C(═O)R²⁷, —C(═O)N(R²⁶)R²⁷, —C(═O)OR²⁶, —OC(═O)R²⁶, —OSi(R²⁵)(R²⁶)R²⁷, —S(═O)₂R²⁶, —S(═O)₂N(R²⁶)R²⁷, —N(R²⁶)R²⁷, —N(R²⁶)N(R²⁶)R²⁷, —N(R²⁶)C(═O)R²⁷ and —N(R²⁶)S(═O)₂R²⁷; and
R²⁹ is independently selected from halogen, cyano, —OR²⁶, —OC(═O)R²⁶, —OSi(R²⁵)(R²⁶)R²⁷, —N(R²⁶)R²⁷, —N(R²⁶)N(R²⁶)R²⁷, —N(R²⁶)C(═O)R²⁷, —N(R²⁶)S(═O)₂R²⁷, —N₃, and —OS(═O)₂CF₃, optionally substituted monosaccharide, optionally substituted disaccharide, optionally substituted trisaccharide, and optionally substituted tetrasaccharide.

In certain embodiments, the compound has Formula VI:

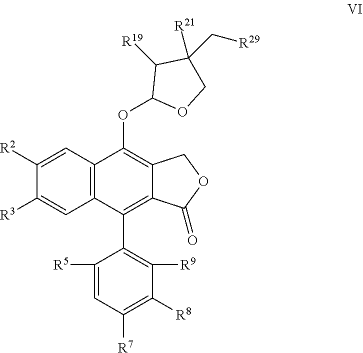

or a pharmaceutically acceptable salt thereof, wherein:
R², R³, R⁷, and R⁸ are each independently —OR 26; and R⁵, and R⁹ is hydrogen or —OR 26; or R² and R³ taken together form a methylenedioxy group; or R⁷ and R⁸ taken together form a methylenedioxy group;
R¹⁹ is selected from the group consisting of —OR 25, —OC(═O)R²⁵, and —OC(═O)N(R²⁵)R²⁵, optionally substituted monosaccharide, optionally substituted disaccharide, optionally substituted trisaccharide, and optionally substituted tetrasaccharide;
R²¹ is selected from the group consisting of —OR 25, —OC(═O)R²⁵, —OC(═O)N(R²⁵)R²⁵, and —C(═O)OR²⁵;
R²⁵ for each occurrence is independently selected from hydrogen and hydrocarbyl;
R²⁶ for each occurrence is independently hydrogen, hydrocarbyl, or heterocyclyl; and
R²⁹ is independently selected from halogen, cyano, —OR²⁶, —OC(═O)R²⁶, —OSi(R²⁵)(R²⁶)R²⁷, —N(R²⁶)R²⁷, —N(R²⁶)N(R²⁶)R²⁷, —N(R²⁶)C(═O)R²⁷, —N(R²⁶)S(═O)₂R²⁷, —N₃, and —OS(═O)₂CF₃, optionally substituted monosaccharide, optionally substituted disaccharide, optionally substituted trisaccharide, and optionally substituted tetrasaccharide.

In instances in which at least one of R⁵ or R⁹ is not hydrogen, atropisomers about the 1-1′ carbons of the compounds described herein can exist. Such atropisomers can be isolated and can be stable (i.e., do not interconvert) at room temperature. In such instances, the compounds can exist in one of two atropisomeric forms as shown below (wherein R⁵ and R⁹; and R⁶ and R⁸ are not the same group):

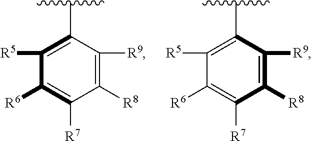

or mixture a thereof.

Surprisingly, it has been discovered when the compounds described herein have the atropisomers about the 1-1′ carbons depicted below:

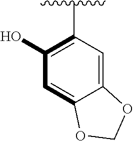

can exhibit improved anti-viral properties.

In certain embodiments, the compound is selected from the group consisting of: 8, 9, 10, 11, 12, 16, 17, 18, 19, 20, 21, 23, 24, 25, 26, 27, 28, 29, 30, 31, 32, 42, 43, 44, 45, 46, 47, 48, 49, 50, 51, 52, 53 and 54:

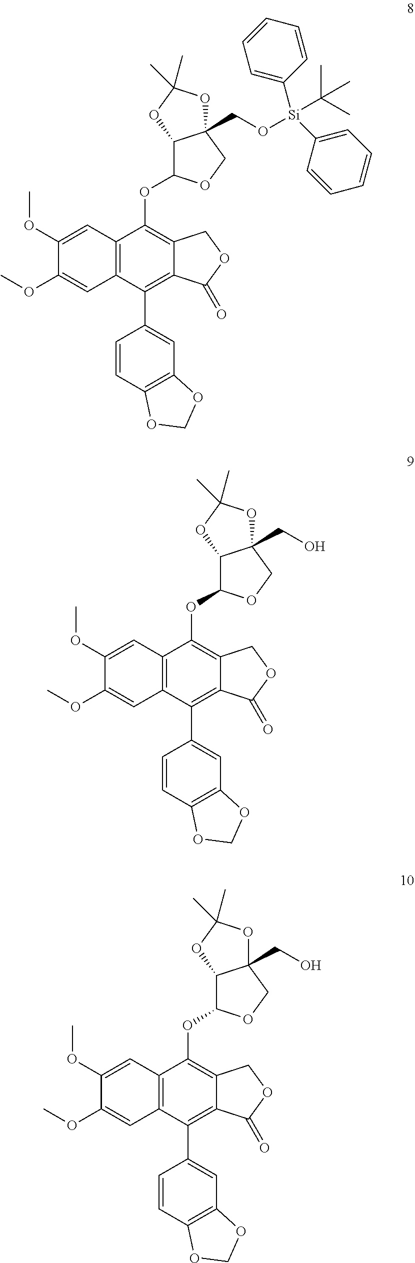 [SMILES: COc1cc2c(OC3OC[C@@]4(CO[Si](c5ccccc5)(c5ccccc5)C(C)(C)C)OC(C)(C)O[C@@H]34)c3c(c(-c4ccc5c(c4)OCO5)c2cc1OC)C(=O)OC3 ; COc1cc2c(O[C@@H]3OC[C@@]4(CO)OC(C)(C)O[C@@H]34)c3c(c(-c4ccc5c(c4)OCO5)c2cc1OC)C(=O)OC3 ; COc1cc2c(O[C@H]3OC[C@@]4(CO)OC(C)(C)O[C@@H]34)c3c(c(-c4ccc5c(c4)OCO5)c2cc1OC)C(=O)OC3]

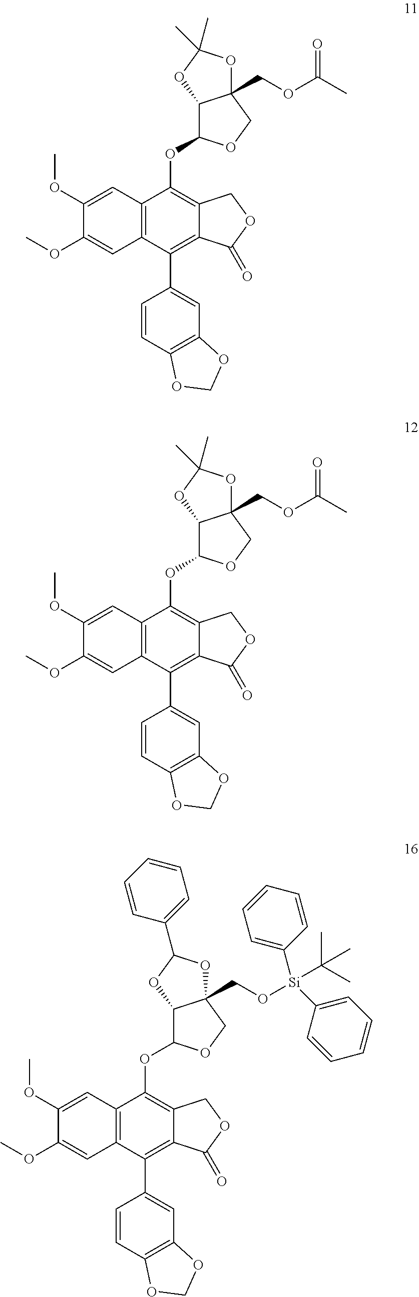

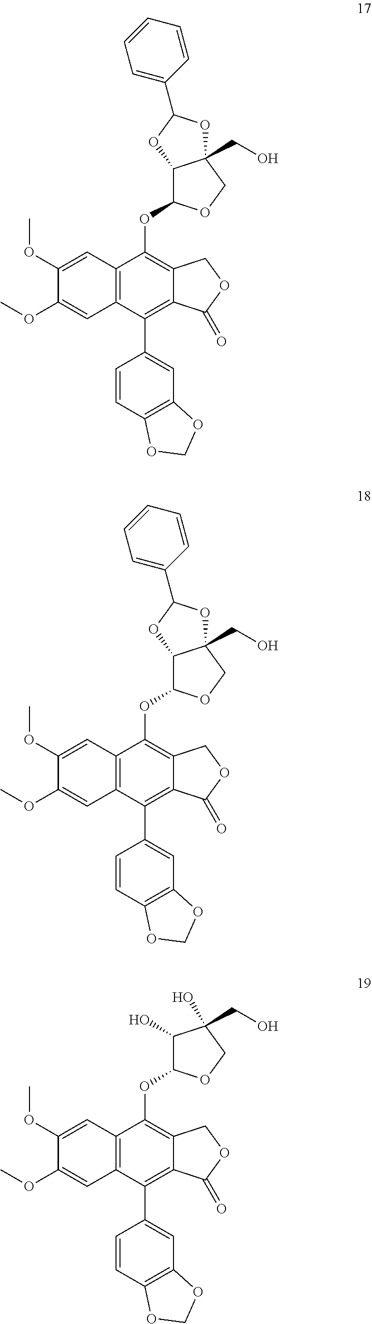

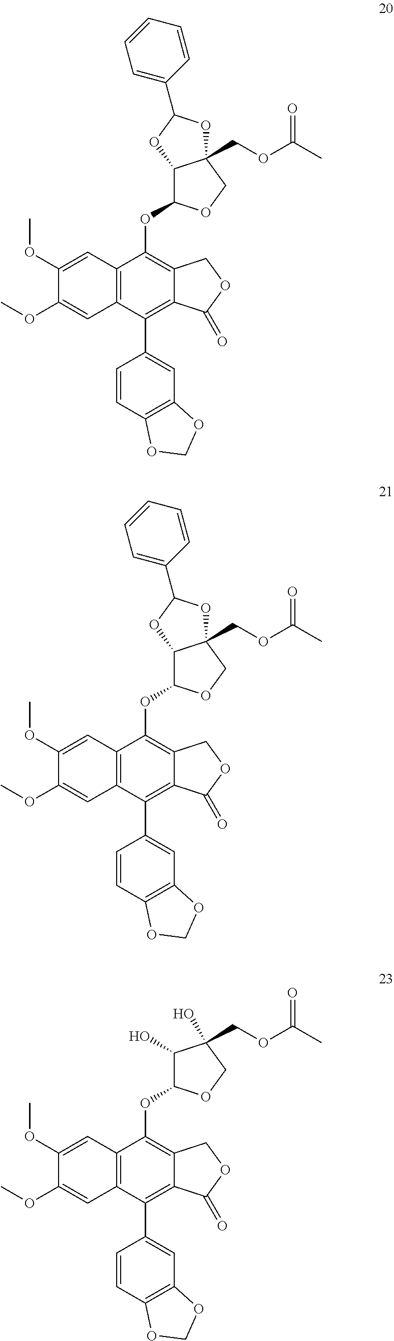

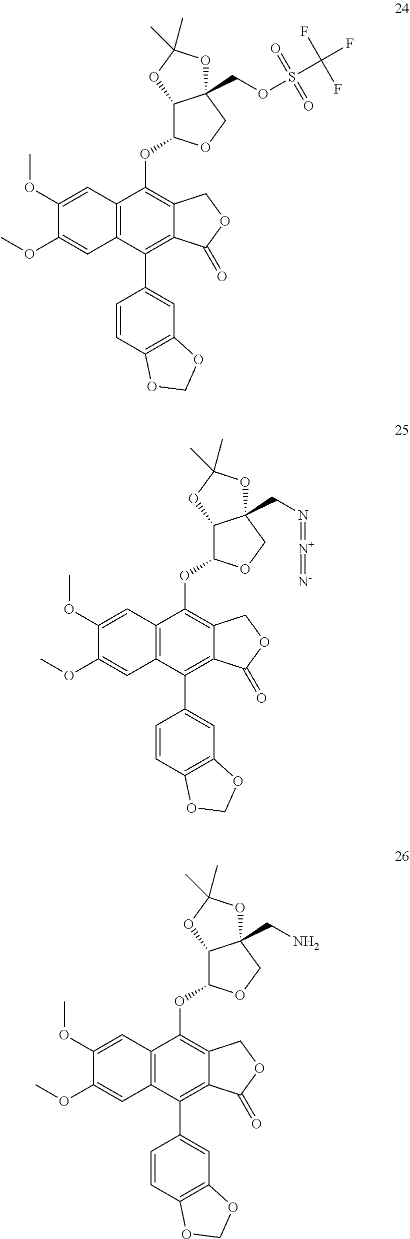 [SMILES: COc1cc2c(O[C@H]3OC[C@@]4(COS(=O)(=O)C(F)(F)F)OC(C)(C)O[C@@H]34)c3c(c(-c4ccc5c(c4)OCO5)c2cc1OC)C(=O)OC3 ; COc1cc2c(O[C@H]3OC[C@@]4(CN=[N+]=[N-])OC(C)(C)O[C@@H]34)c3c(c(-c4ccc5c(c4)OCO5)c2cc1OC)C(=O)OC3 ; COc1cc2c(O[C@H]3OC[C@@]4(CN)OC(C)(C)O[C@@H]34)c3c(c(-c4ccc5c(c4)OCO5)c2cc1OC)C(=O)OC3]

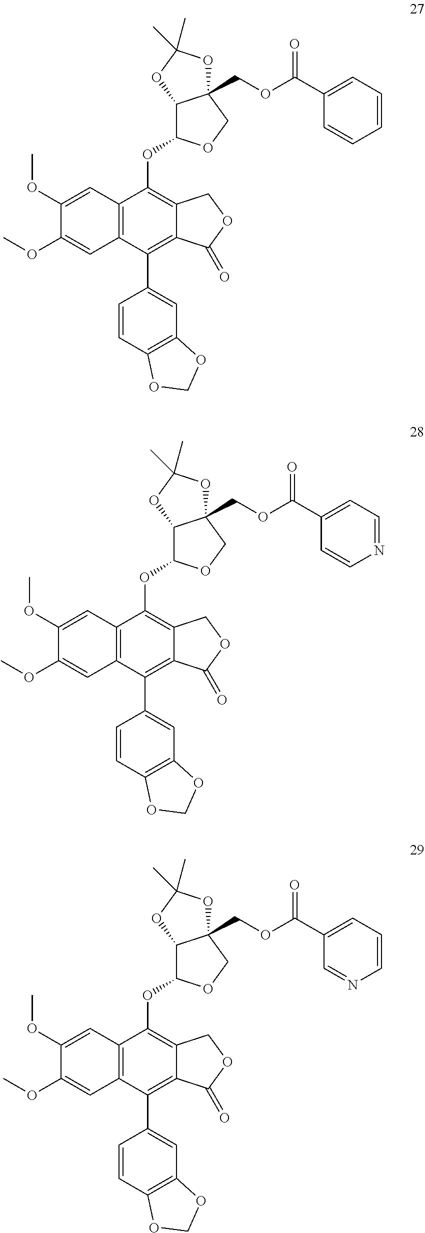

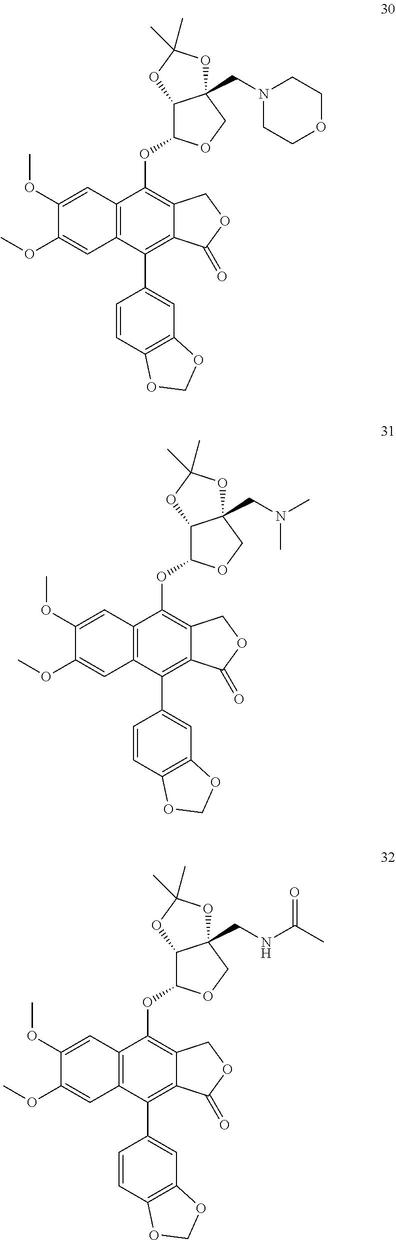

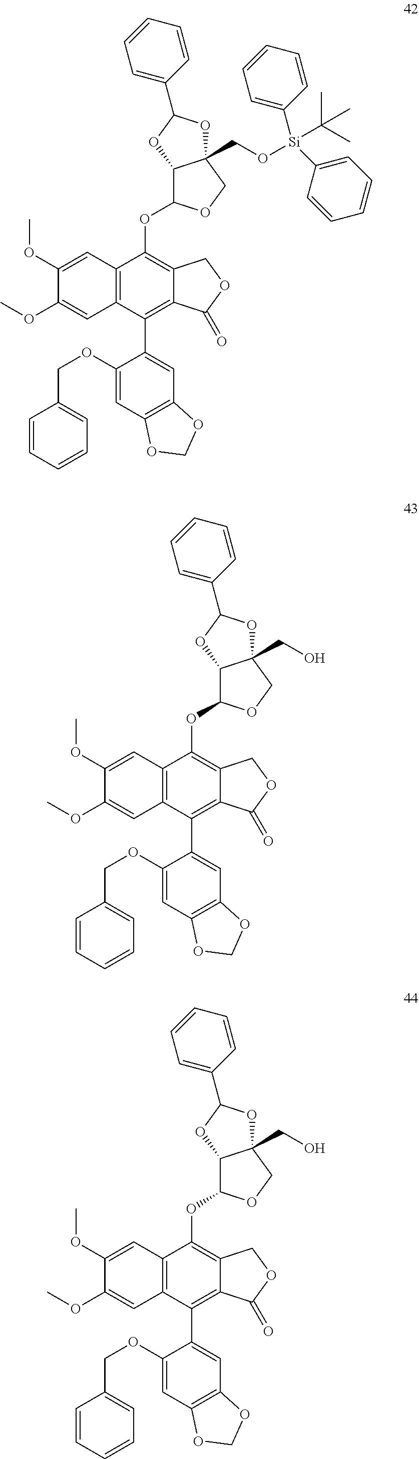

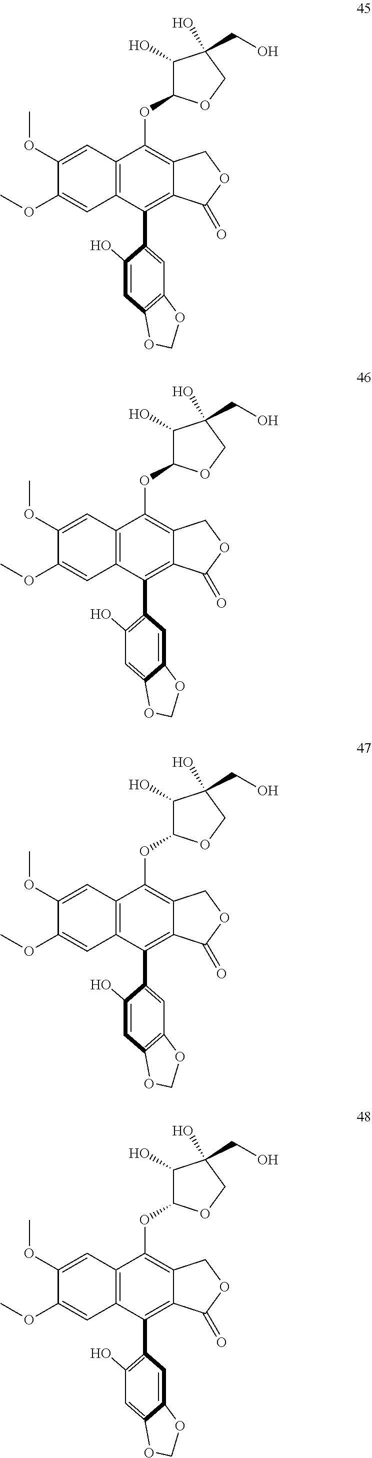

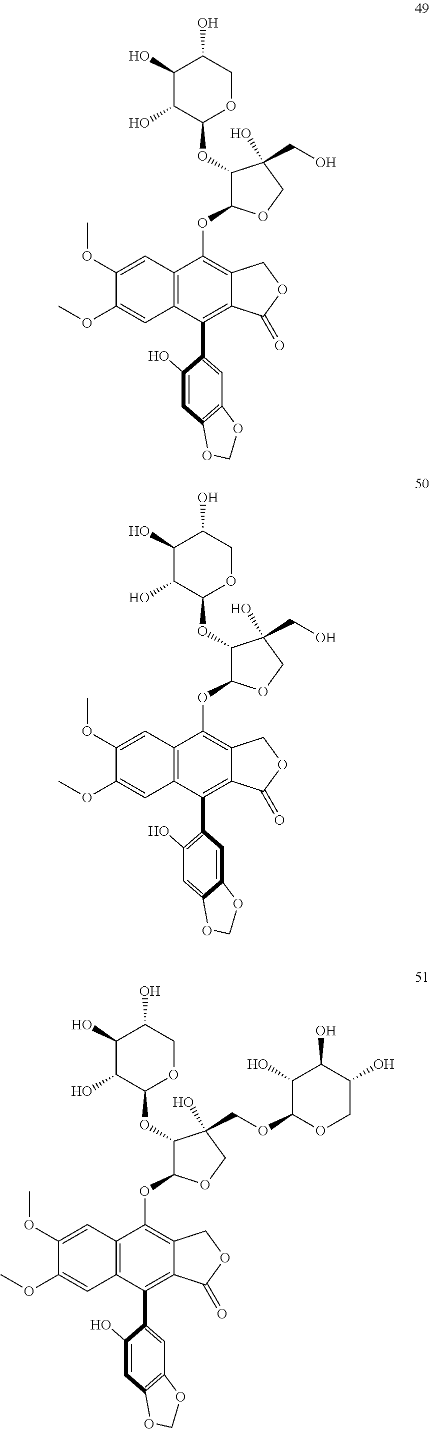

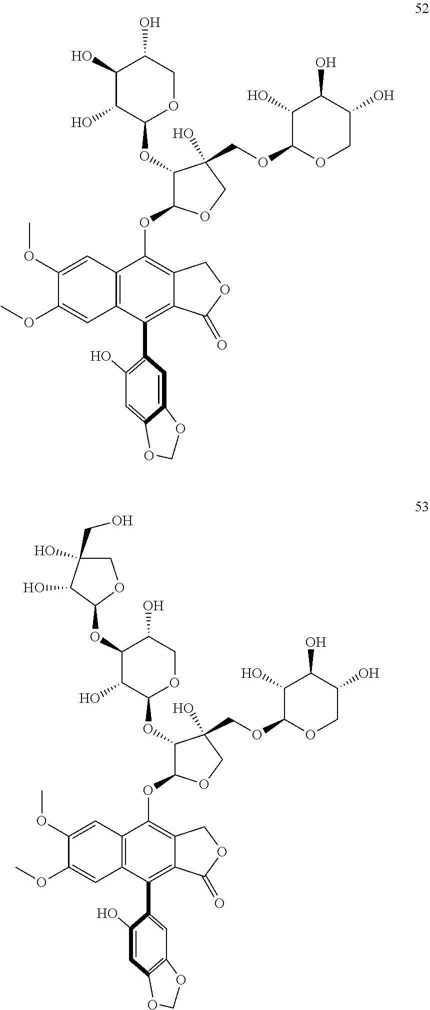

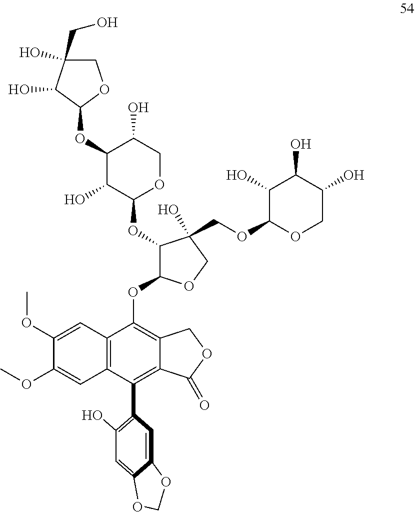

or a pharmaceutically acceptable salt thereof.

Examples of compounds of the present disclosure include those shown below. It will of course be appreciated that, where appropriate, each compound may be in the form of the free compound, an acid or base addition salt, or a prodrug.

The present disclosure also provides a pharmaceutical composition comprising at least one of the compounds described herein and at least one pharmaceutically acceptable excipient.

The compounds described herein and their pharmaceutically acceptable salts can be administered to a subject either alone or in combination with pharmaceutically acceptable, excipients, carriers, and/or diluents in a pharmaceutical composition according to standard pharmaceutical practice. The compounds can be administered orally or parenterally. Parenteral administration includes intravenous, intramuscular, intraperitoneal, subcutaneous and topical, the preferred method being intravenous and topical administrations.

Accordingly, the present disclosure provides pharmaceutically acceptable compositions, which comprise a therapeutically effective amount of one or more of the compounds described herein, formulated together with one or more pharmaceutically, excipients, acceptable carriers (additives) and/or diluents. The pharmaceutical compositions of the present disclosure may be specially formulated for administration in solid or liquid form, including those adapted for the following: (1) parenteral administration, for example, by subcutaneous, intramuscular, intravenous or epidural injection as, for example, a sterile solution or suspension, or sustained-release formulation; and (2) oral administration, for example, drenches (aqueous or non-aqueous solutions or suspensions), tablets, e.g., those targeted for buccal, sublingual, and systemic absorption, boluses, powders, granules, pastes for application to the tongue.

As set out herein, certain embodiments of the compounds described herein may contain a basic functional group, such as amino, and are, thus, capable of forming pharmaceutically acceptable salts with pharmaceutically acceptable acids. The term “pharmaceutically acceptable salts” in this respect, refers to the relatively non-toxic, inorganic and organic acid addition salts of compounds of the present disclosure. These salts can be prepared in situ in the administration vehicle or the dosage form manufacturing process, or by separately reacting a purified compound of the invention in its free base form with a suitable organic or inorganic acid, and isolating the salt thus formed during subsequent purification. Representative salts include the hydrobromide, hydrochloride, sulfate, bisulfate, nitrate, acetate, valerate, oleate, palmitate, stearate, laurate, benzoate, lactate, phosphate, tosylate, citrate, maleate, fumarate, succinate, tartrate, napthylate, mesylate, glucoheptonate, lactobionate, and laurylsulphonate salts and the like.

The pharmaceutically acceptable salts of the compounds of the present disclosure include the conventional non-toxic salts or quaternary ammonium salts of the compounds, e.g., from non-toxic organic or inorganic acids. For example, such conventional non-toxic salts include those derived from inorganic acids such as hydrochloride, hydrobromic, sulfuric, sulfamic, phosphoric, nitric, and the like; and the salts prepared from organic acids such as acetic, propionic, succinic, glycolic, stearic, lactic, malic, tartaric, citric, ascorbic, palmitic, maleic, hydroxymaleic, phenylacetic, glutamic, benzoic, salicyclic, sulfanilic, 2-acetoxybenzoic, fumaric, toluenesulfonic, methanesulfonic, ethane disulfonic, oxalic, isothionic, and the like.

In other cases, the compounds described herein may contain one or more acidic functional groups and, thus, are capable of forming pharmaceutically acceptable salts with pharmaceutically acceptable bases. The term “pharmaceutically acceptable salts” in these instances refers to the relatively non-toxic, inorganic and organic base addition salts of compounds of the present disclosure. These salts can likewise be prepared in situ in the administration vehicle or the dosage form manufacturing process, or by separately reacting the purified compound in its free acid form with a suitable base, such as the hydroxide, carbonate or bicarbonate of a pharmaceutically-acceptable metal cation, with ammonia, or with a pharmaceutically-acceptable organic primary, secondary or tertiary amine. Representative alkali or alkaline earth salts include the lithium, sodium, potassium, calcium, magnesium, and aluminum salts and the like. Representative organic amines useful for the formation of base addition salts include ethylamine, diethylamine, ethylenediamine, ethanolamine, diethanolamine, piperazine and the like.

Wetting agents, emulsifiers and lubricants, such as sodium lauryl sulfate and magnesium stearate, as well as coloring agents, release agents, coating agents, sweetening, flavoring and perfuming agents, preservatives, solubilizing agents, buffers and antioxidants can also be present in the compositions.

Methods of preparing these formulations or compounds include the step of bringing into association a compound described herein with the carrier or excipient and, optionally, one or more accessory ingredients. In general, the formulations are prepared by uniformly and intimately bringing into association a compound of the present disclosure with liquid carriers (liquid formulation), liquid carriers followed by lyophylization (powder formulation for reconstitution with sterile water or the like), or finely divided solid carriers, or both, and then, if necessary, shaping or packaging the product.

Pharmaceutical compositions of the present disclosure suitable for parenteral administration comprise one or more compounds described herein in combination with one or more pharmaceutically-acceptable sterile isotonic aqueous or non-aqueous solutions, dispersions, suspensions or emulsions, or sterile powders which may be reconstituted into sterile injectable solutions or dispersions just prior to use, which may contain sugars, alcohols, antioxidants, buffers, bacteriostats, chelating agents, solutes which render the formulation isotonic with the blood of the intended recipient or suspending or thickening agents.

Examples of suitable aqueous and non-aqueous carriers which may be employed in the pharmaceutical compositions of the disclosure include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants, such as preservatives, wetting agents, emulsifying agents and dispersing agents. Prevention of the action of microorganisms upon the compounds of the present disclosure may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption, such as aluminum monostearate and gelatin.

The compounds of the present disclosure were synthesized and evaluated for their anti-HIV, anti-coronaviral, anti-Ebola viral, anti-Marburg viral, and anti-influenza viral activity.

The present disclosure provides compounds with anti-HIV, anti-coronaviral, anti-Ebola viral, anti-Marburg viral, and anti-influenza virus activity and synthesis thereof. Thus, provided herein is a method of treating a viral infection in a subject in need thereof, the method comprising administering a therapeutically effective amount of a compounds described herein to the subject.

The compounds described herein can be exemplified by 8, 9, 10, 11, 12, 16, 17, 18, 19, 20, 21, 22, 23, 24, 25, 26, 27, 28, 29, 30, 31, 32, 42, 43, 44, 45, 46, 47, 48, 49, 50, 51, 52, 53 and 54.

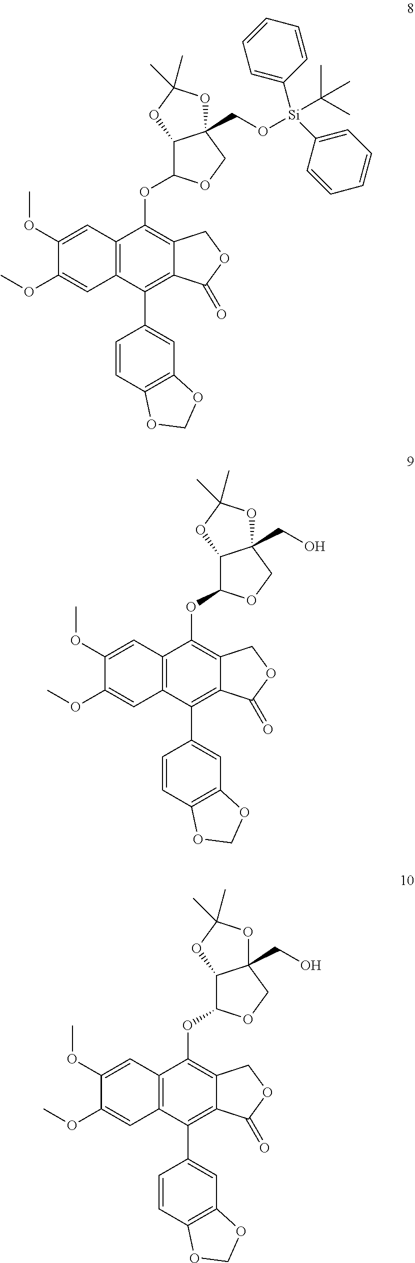 [SMILES: COc1cc2c(OC3OC[C@@]4(CO[Si](c5ccccc5)(c5ccccc5)C(C)(C)C)OC(C)(C)O[C@@H]34)c3c(c(-c4ccc5c(c4)OCO5)c2cc1OC)C(=O)OC3 ; COc1cc2c(O[C@@H]3OC[C@@]4(CO)OC(C)(C)O[C@@H]34)c3c(c(-c4ccc5c(c4)OCO5)c2cc1OC)C(=O)OC3 ; COc1cc2c(O[C@H]3OC[C@@]4(CO)OC(C)(C)O[C@@H]34)c3c(c(-c4ccc5c(c4)OCO5)c2cc1OC)C(=O)OC3]

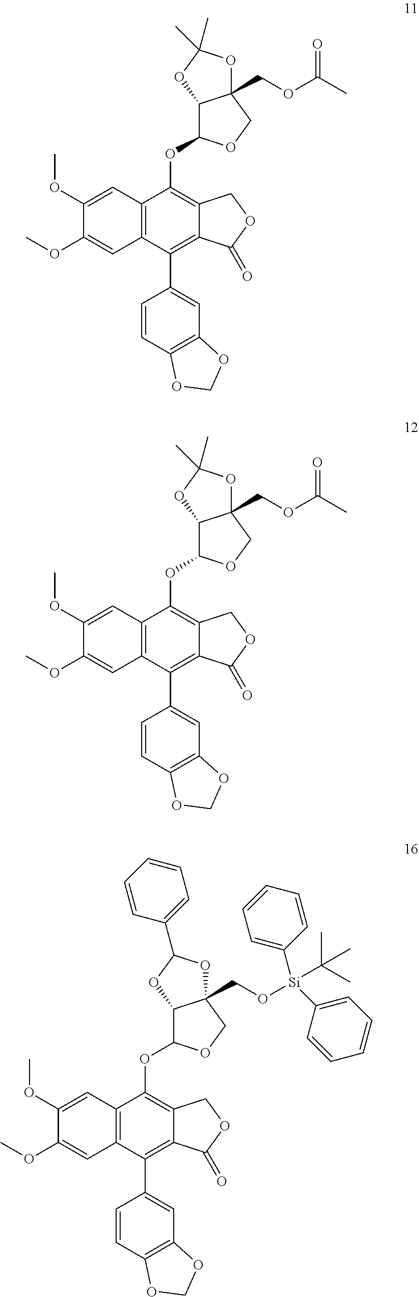

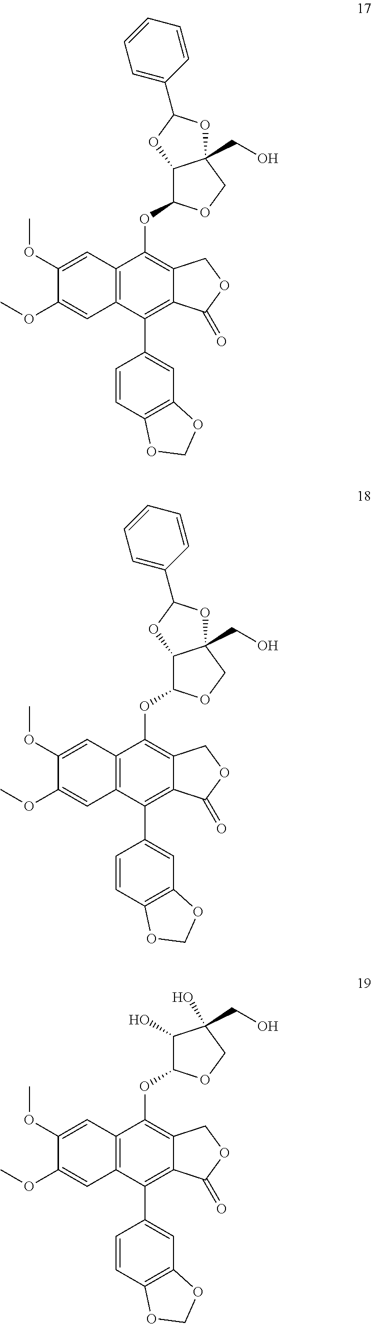

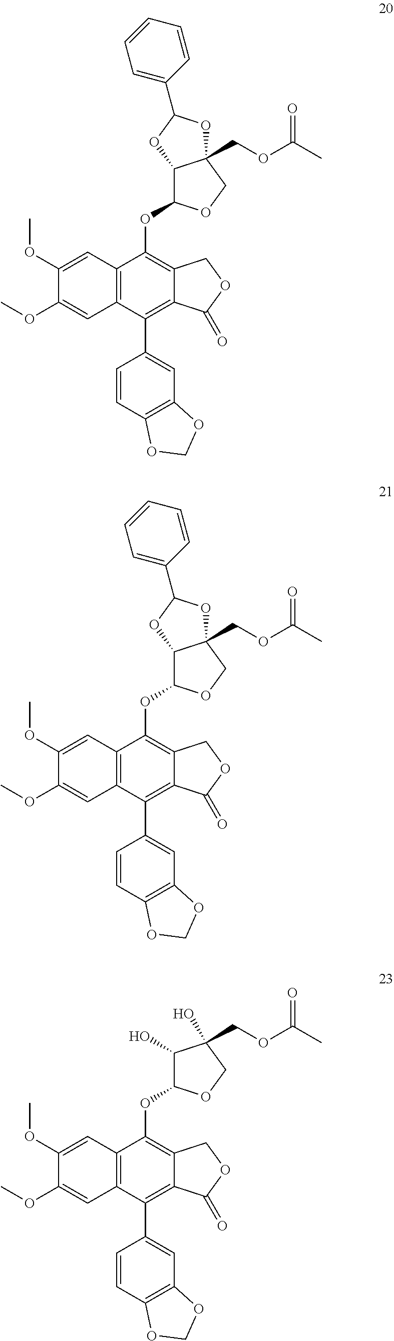

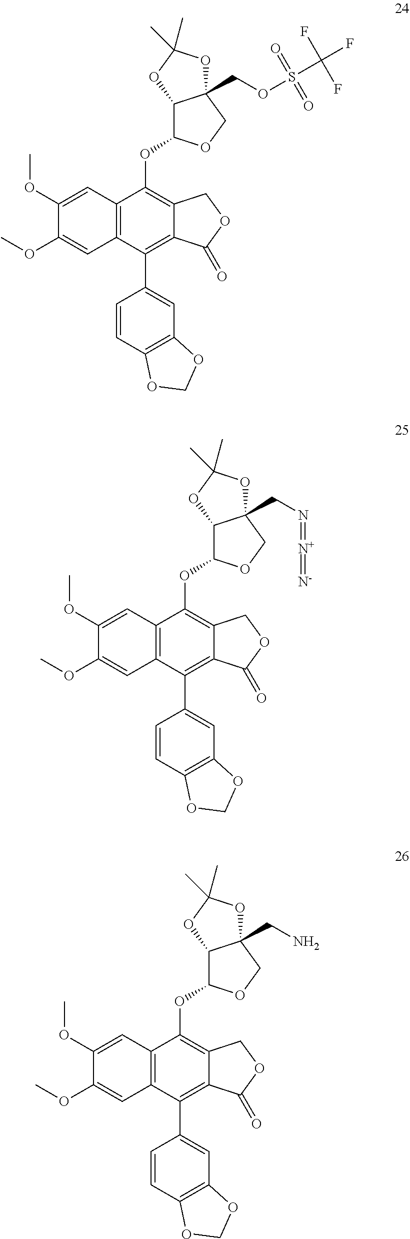 [SMILES: COc1cc2c(O[C@H]3OC[C@@]4(COS(=O)(=O)C(F)(F)F)OC(C)(C)O[C@@H]34)c3c(c(-c4ccc5c(c4)OCO5)c2cc1OC)C(=O)OC3 ; COc1cc2c(O[C@H]3OC[C@@]4(CN=[N+]=[N-])OC(C)(C)O[C@@H]34)c3c(c(-c4ccc5c(c4)OCO5)c2cc1OC)C(=O)OC3 ; COc1cc2c(O[C@H]3OC[C@@]4(CN)OC(C)(C)O[C@@H]34)c3c(c(-c4ccc5c(c4)OCO5)c2cc1OC)C(=O)OC3]

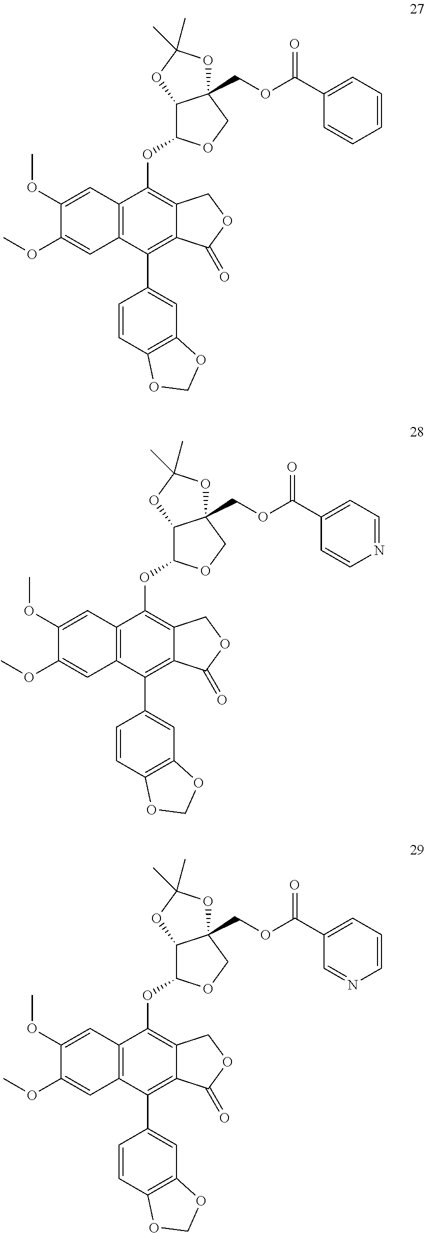

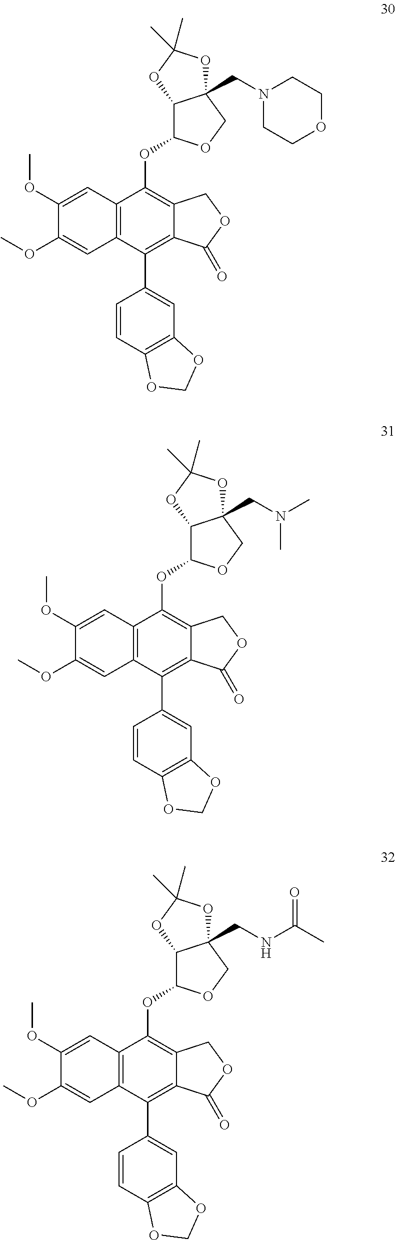

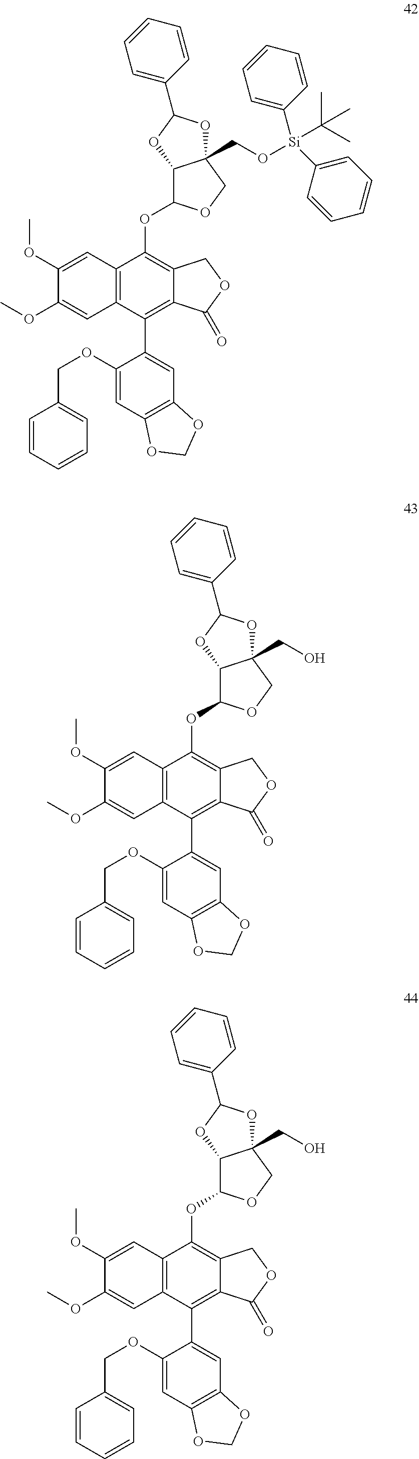

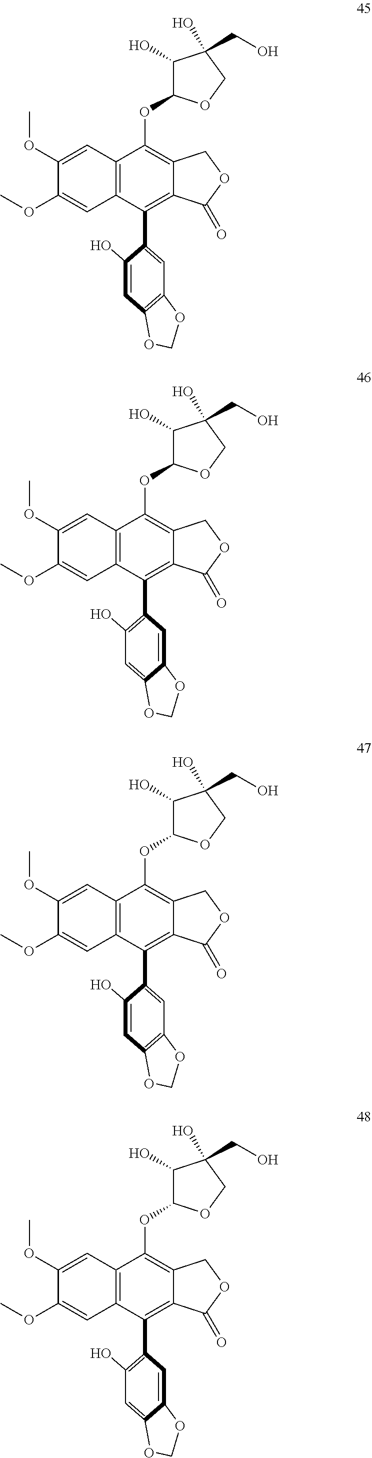

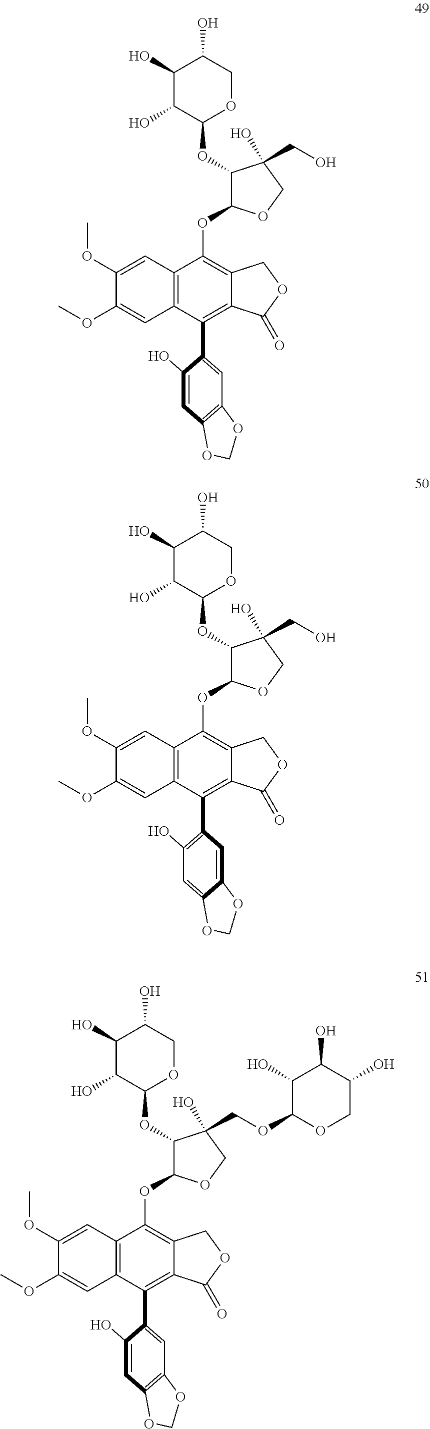

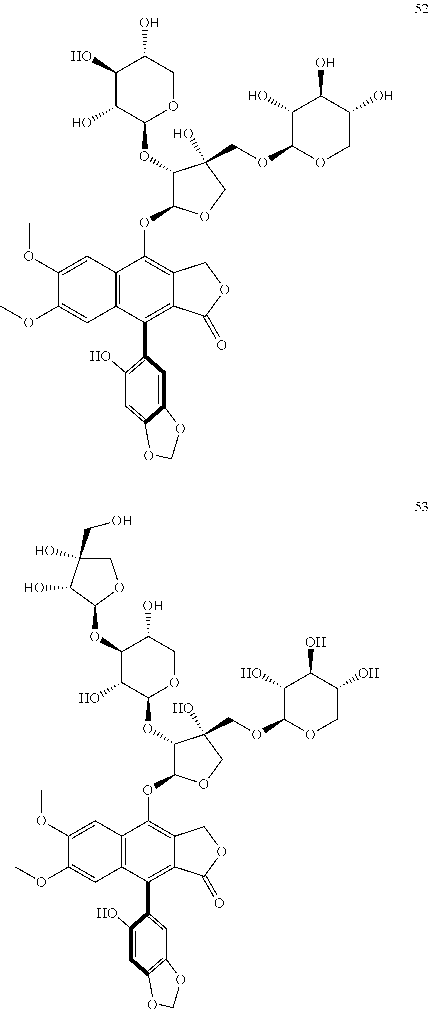

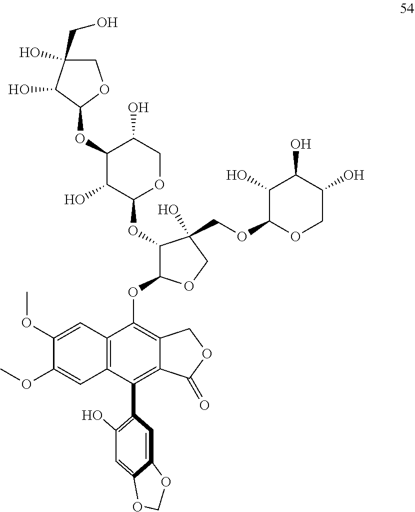

or a pharmaceutically acceptable salt thereof.

### Examples

The following examples are set forth below to illustrate the methods and results according to the disclosed subject matter. These examples are not intended to be inclusive of all aspects of the subject matter disclosed herein, but rather to illustrate representative methods, compositions, and results. These examples are not intended to exclude equivalents and variations of the present invention, which are apparent to one skilled in the art.

Unless indicated otherwise, parts are parts by weight, temperature is ° C. or is at ambient temperature, and pressure is at or near atmospheric. There are numerous variations and combinations of reaction conditions, e.g., component concentrations, temperatures, pressures, and other reaction ranges and conditions that can be used to optimize the product purity and yield obtained from the described process. Only reasonable and routine experimentation will be required to optimize such process conditions.

Examples of compounds of the present disclosure include those shown below. It will of course be appreciated that, where appropriate, each compound may be in the form of the free compound, an acid or base addition salt, or a prodrug.

By using tuberculatin (1) as a structural scaffold, compounds 8, 9, 10, 11, 12, 16, 17, 18, 19, 20, 21, 22, 23, 24, 25, 26, 27, 28, 29, 30, 31, 32, 42, 43, 44, 45, 46, 47 and 48 were synthesized by using reactions illustrated in FIGS. 2-6. Similar to tuberculatin (1), compounds 49-54 were obtained from the medicinal plants Justicia procumbens.

As shown in FIG. 2, a new strategy was applied to construct the apiosidic part. The synthesis started with L-ribose (2). Acetonide protected L-ribose (3) was obtained using concentrated sulfuric acid (H₂SO₄) as catalyst, followed by an addition reaction with formaldehyde (HCHO) by introducing an hydroxymethyl group at C-2 to afford 4. Compound 4 underwent a reduction by sodium borohydride (NaBH₄), followed by a 1,2-diols cleavage reaction with sodium periodate (NaIO₄) to give D-apiose acetonide 5. In order to avoid side reaction, the C-3 hydroxymethyl group was protected by TBDPS (tert-butyldiphenylsilane) to produce 6.

Mitsunobu reaction was than performed between diphyllin (7) and compound 6 to afford 8 s a mixture of α- and β-configured apiosides which were inseparable under silica gel column. The targeted α-configuration was a minor product by containing 13% in the mixture. Without separation of the mixture, TBDPS group was deprotected to yield two compounds (9 and 10) in excellent yields, which reacted with acetic anhydride (Ac₂O) to give the acetylated derivatives 11 and 12, respectively. However, because the apiosidic bond is very sensitive to acidic condition as an acetonide group, the final step to deprotect the acetal group only failed to yield tuberculatin (1) (FIG. 3).

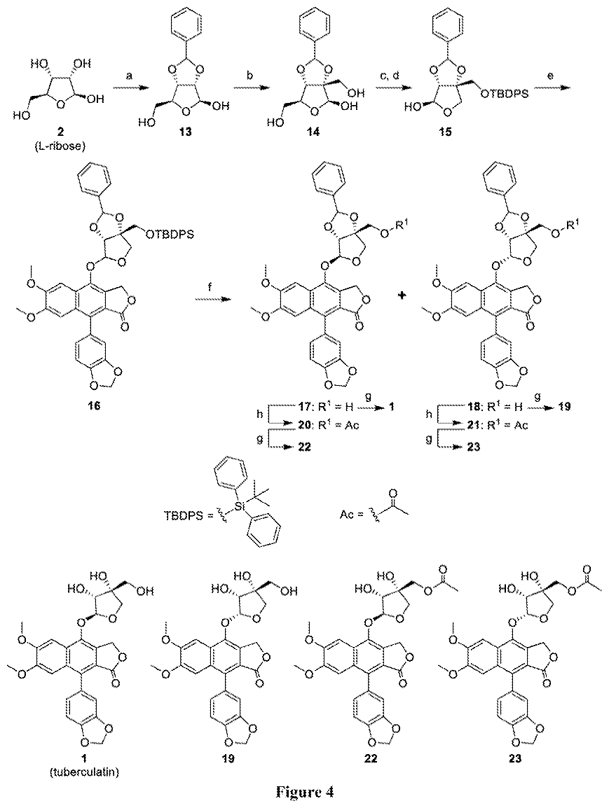

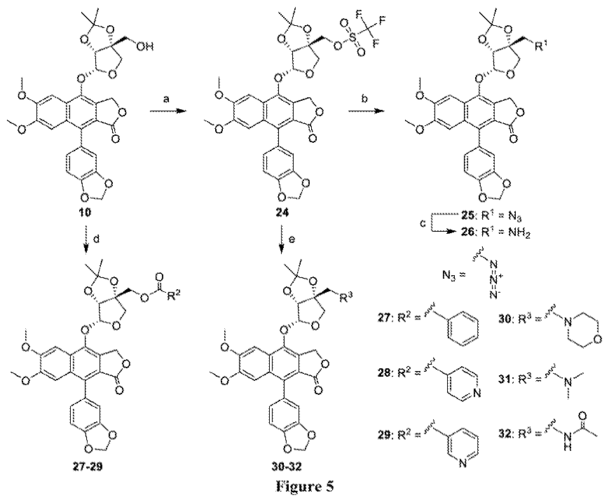

As shown in FIG. 4, benzylidene acetal group was used to protect the 2, 3-diol on L-ribose, which could be easily removed under a neutral condition by hydrogenolysis. Using anhydrous copper sulfate (CuSO₄) as desiccant and D-camphorsulfonic acid as catalyst, compound 13 was obtained by the condensation of the fresh distilled benzaldehyde and L-ribose with a moderate yield. Using the same method described in FIG. 2, 13 was converted to 14 by the addition reaction promoted by K₂CO₃. Followed by a reduction with NaBH₄ and oxidation with NaIO₄, compound 15 was formed from 14. After the protection and deprotection of the primary alcohol group with TBDPS, 15 was converted smoothyl to the mixture compounds of 17 and 18 by carrying out the key step of Mitsunobu reaction. The compound mixture was separated by a preparative TLC to afford pure 17 and 18, which were hydrogenated with palladium hydroxide [Pd(OH)₂] to afford 1 and 19, respectively. Compound 1 was identified as the target molecule tuberculatin, which showed the same ¹H and ¹³C NMR spectroscopic data as the natural isolate. Acetylation of 17 and 18 with Ac₂O provided 20 and 21, respectively, which were hydrogenated by removing the benzylidene acetal group to afford diphyllin acetylapiosides 22 and 23, respectively.

To understand if the other functional groups on the apiose may affect the antiviral activity, derivatization of compound 10 focusing on the hydroxymethyl group was carried out (FIG. 5). Esterification of 10 with carbonyl chlorides provided compounds 26, 27 and 28 in excellent yields, respectively. The nitrogen containing derivatives 25, 30, 31 and 32 were also synthesized through the coupling reaction of an azide, an amine or an amide with the activated intermediate 24 in moderate yields, respectively. Further reduction of the azide derivative 25 by trimethylphosphine (PMe₃) solution afforded aza-diphyllin β-apioside 26.

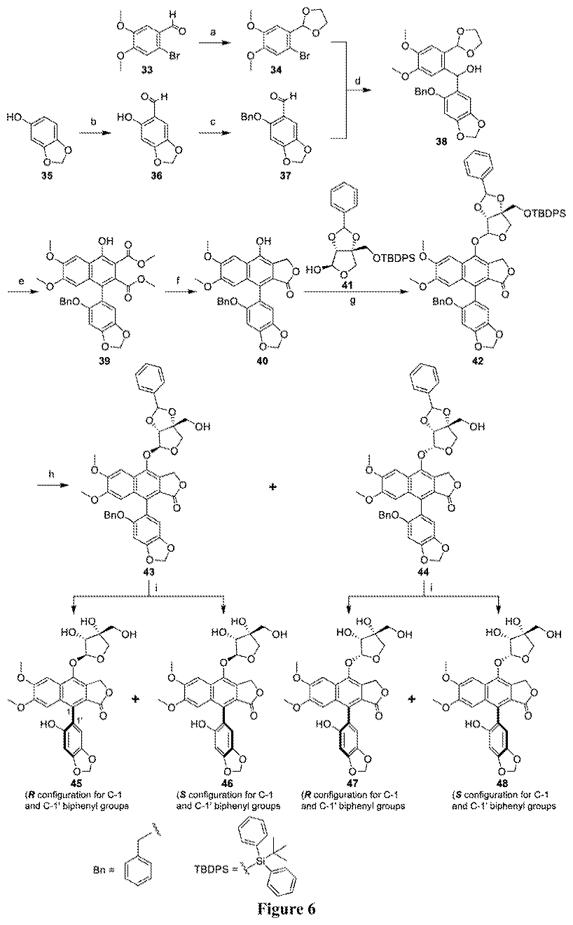

FIG. 6 shown synthesis of several atropisomer analogs (45, 46, 47 and 48) of tuberculatin (1). Arylnaphthalene lignans are known to have axial chirality due to the biphenyl skeleton with a hindered rotation around the sp²-sp² σ bond, which leads to formation of P- and M-configured isomers (i.e. atropisomers). To understand if the atropisomerism may affect the antiviral activity of arylnaphthalene lignan compounds, we synthesized a number of atropisomer analogs of tuberculatin (1). Starting from 6-bromoveratraldehyde (33) and sesamol (35), the aglycone 6′-benzyloxy diphyllin (40) was synthesized. By attaching the sugar unit D-apiose, 40 was converted to compounds 43 and 44 through the intermediate 42. Compounds 43 and 44 were hydrolyzed separately by removing the protection group to afford the pair of atropisomers 45 and 46, and the atropisomers 47 and 48, respectively. All the four synthesized atropisomers (45, 46, 47 and 48) were obtained as stable forms of pure compounds at room temperature.

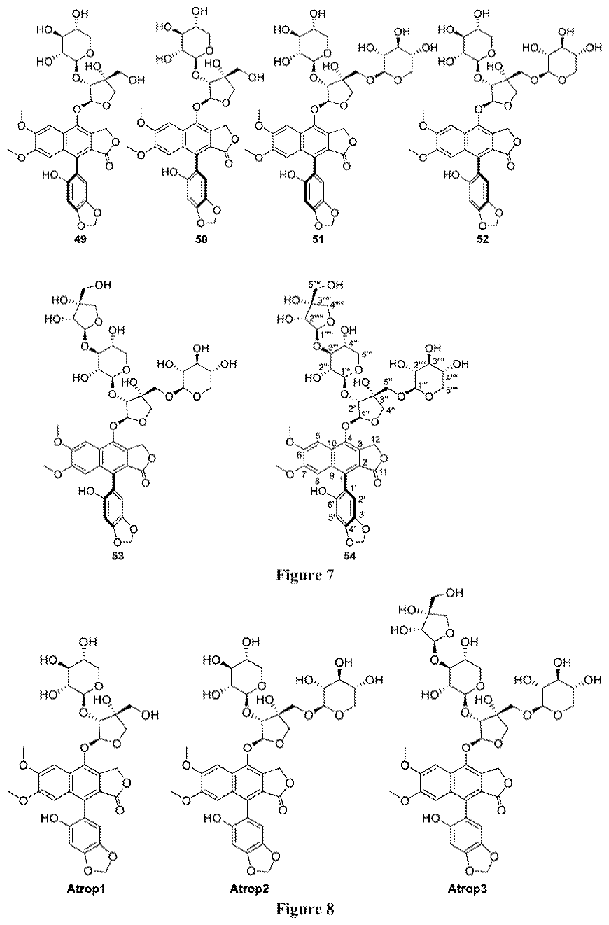

FIG. 7 shown the structures of compounds 49-54, which were obtained as three pairs of stable atropisomers through a chiral separation of the respective compounds Atrop1, Atrop2 and Atrop3 (FIG. 8) from Justicia procumbens.

The compounds described herein can exhibit broad antiviral properties. According to methods of the present disclosure, compounds of Formula (I) and Formula (II) are administered to a patient to inhibit replication of or reduce cytopathic effects of viruses, such as HIV, coronaviruses, Ebola virus, Marburg virus or influenza viruses. Other viruses that may be inhibited by compounds of Formula (I) and Formula (II) include, but are not limited to, cytomegalovirus (CMV), HSV-1 (herpes simplex virus type 1), HSV-2 (herpes simplex virus type 2), HBV (hepatitis B virus), HCV (hepatitis C virus), HPV (human papilloma virus), influenza A, influenza B, RSV (respiratory syncitial virus), RV (rhinovirus), AV (adenovirus), PIV (human parainfluenza viruses), Epstein-Barr virus (EBV), varicella zoster virus (VZV), dengue virus and Zika virus.

The compounds, pharmaceutical compositions, and therapeutic methods described herein are useful for preventing or treating or ameliorating HIV infections.

The compounds, pharmaceutical compositions, and therapeutic methods described herein are useful for preventing, treating, or ameliorating infections caused by influenza viruses, including but not limited to: any of the subtypes of influenza A, influenza B, or influenza C.

In certain embodiments, the compounds, pharmaceutical compositions, and therapeutic methods disclosed herein are useful for preventing, treating, or ameliorating infections caused by influenza A viruses, including but not limited to, any of the strains of H1N1, H1N2, H1N3, H1N4, H1N5, H1N6, H1N7, H1N8, H1N9, H2N1, H2N2, H2N3, H2N4, H2N5, H2N6, H2N7, H2N8, H2N9, H3N1, H3N2, H3N3, H3N4, H3N5, H3N6, H3N7, H3N8, H3N9, H4N1, H5N2, H5N3, H5N4, H5N5, H5N6, H5N7, H5N8, H5N9, H6N1, H6N2, H6N3, H6N4, H6N5, H6N6, H6N7, H6N8, H6N9, H7N1, H7N2, H7N3, H7N4, H7N5, H7N6, H7N7, H7N8, H7N9, H8N1, H8N2, H8N3, H8N4, H8N5, H8N6, H8N7, H8N8, H8N9, H9N1, H9N2, H9N3, H9N4, H9N5, H9N6, H9N7, H9N8, H9N9, H10N1, H10N2, H10N3, H10N4, H10N5, H10N6, H10N7, H10N8, H10N9, H11N1, H11N2, H11N3, H11N4, H11N5, H11N6, H11N7, H11N8, H11N9, H12N1, H12N2, H12N3, H12N4, H12N5, H12N6, H12N7, H12N8, H12N9, H13N1, H13N2, H13N3, H13N4, H13N5, H13N6, H13N7, H13N8, H13N9, H14N1, H14N2, H14N3, H14N4, H14N5, H14N6, H14N7, H14N8, H14N9, H15N1, H15N2, H15N3, H15N4, H15N5, H15N6, H1N7, H15N8, and H15N9.

In certain embodiments, the compounds, pharmaceutical compositions, and therapeutic methods disclosed herein are useful for preventing, treating, or ameliorating infections caused by influenza A virus strains having a type 5 hemagglutinin protein. In certain embodiments, the influenza A virus strain has a type 5 hemagglutinin protein and neuraminidase protein selected from types 1 to 11. In certain embodiments, the influenza A virus is selected from the group consisting of H5N1 and H5N2.

In certain embodiments, the compounds, pharmaceutical compositions, and therapeutic methods disclosed herein are useful for preventing, treating, or ameliorating infections caused by H5N1.

The term “HIV”, as used herein, refers to the human immunodeficiency virus and includes HIV-1, HIV-2 and SIV. In certain embodiments, HIV refers to HIV-1 and/or HIV-2. “HIV-1” means the human immunodeficiency virus type-1. HIV-1 can include but is not limited to extracellular virus particles and the forms of HIV-1 associated with HIV-1 infected cells. The HIV-1 virus can include any of the known major subtypes (classes A, B, C, D, E, F, G and H) or outlying subtype (group 0) including laboratory strains and primary isolates. “HIV-2” means the human immunodeficiency virus type-2. HIV-2 can include but is not limited to extracellular virus particles and the forms of HIV-2 associated with HIV-2 infected cells. The term “SIV” refers to simian immunodeficiency virus, which is an HIV-like virus that infects monkeys, chimpanzees, and other nonhuman primates. SIV can include but is not limited to extracellular virus particles and the forms of SIV associated with SIV infected cells.

In certain embodiments, the compounds, pharmaceutical compositions, and therapeutic methods disclosed herein are useful for preventing, treating, or ameliorating infections caused by HIV-1 and/or HIV-2. In certain embodiments, the compounds, pharmaceutical compositions, and therapeutic methods disclosed herein are useful for preventing, treating or ameliorating infections caused by HIV-1 subtype B.

In certain embodiments, the compounds, pharmaceutical compositions, and therapeutic methods disclosed herein are useful for preventing, treating, or ameliorating infections caused by SIV.

The compounds, pharmaceutical compositions, and therapeutic methods described herein are useful for preventing, treating, or ameliorating infections caused by filoviruses, including but not limited to: Marburg virus, Zaire ebolavirus, Sudan ebolavirus, Cote d'Ivoire ebolavirus, Reston ebolavirus and Bundibugyo ebolavirus.

The compounds, pharmaceutical compositions, and therapeutic methods described herein are useful for preventing, treating, or ameliorating infections caused by coronaviruses including, but not limited to, SARS-CoV-2, SARS-CoV and MERS-CoV.

Antiviral Evaluation Using the “One-Stone-Two-Birds” Pseudo-Type Assay.

Production of HIV Pseudovirions. This protocol is designed to identify potential inhibitors for HIV, coronavirus, Ebola, Marburg and influenza replication (post entry steps). HIV/VSVG or HIV/SARSP or HIV/HA or HIV/EBOV or HIV/MARV virions were produced, respectively, by co-transfecting with either 0.5 μg VSVG (vesicular stomatitis virus glycoprotein) envelope expression plasmid 0.5 μg SARSP (SARS-Cov-2 spike protein) expression plasmid or 0.5 μg hemagglutinin (HA) envelope expression plasmid with 0.5 μg neuraminidase (NA) expression plasmid or 0.5 μg EBVG (Ebola virus glycoprotein) envelope expression plasmid or 0.5 μg MAVG (Marburg virus glycoprotein) envelope expression plasmid and 2 μg replication-defective HIV vector (pNL4-3.Luc.RE) into human embryonic kidney 293T cells (90% confluent) in six-well plates via PEI (polyethylenimine) (Invitrogen, Carlsbad, CA, USA), as previously described with a modified procedure. The HIV vector pNL4-3.Luc.RE was obtained through the AIDS Research and Reference Reagent Program (Division of AIDS, NIAID, NIH). Sixteen hours post-transfection, all media were replaced with fresh, complete DMEM. Eight hours post-transfection, all media were replaced with fresh complete DMEM. Forty-eight hours post-transfection, the supernatants were collected and filtered through a 0.45-μm-pore-size filter (Millipore, Billerica, MA, USA) and the pseudo virions were directly used for infection.

Anti-HIV and anti-H5N1 Influenza Virus Evaluation Assay. This protocol is to identify potential inhibitors for HIV and influenza virus replication (post-entry steps). In this system, the HIV vector pNL4-3.Luc.RE was co-transfected with the VSVG to generate HIV/VSVG virions (HIV virion with VSV glycoprotein on the viral surface), and the same HIV vector was co-transfected with the H5N1 HA and NA constructs to generate HIV virions with bird flu HA on the viral surface [HIV/HA (HIV virion with HA and NA glycoproteins on the viral surface)]. This pNL4-3 was derived from an infectious molecular clone of a SI (syncytium inducing), T-tropic virus, which is replication deficient since the HIV is Env- and Vpr-. In addition, the luciferase gene (luc) carried by this recombinant HIV vector served as the reporter for HIV replication (reverse transcription, integration and HIV gene expression). The infection level was measured as relative light units (RLUs) in the infected cells. The luciferase activities of the 293T cells infected with the HIV vector pNL4-3.Luc.RE reached the range of 10⁵-10⁶ RLUs, approximately 100-fold higher than the background levels when measured using the HIV virions without VSVG. The evaluation principle is that the level of the luciferase activity in the cells should be proportional to the level of viral entry and replication. If a sample can interfere with HIV replication/or HA-mediated viral entry, the level of the luciferase activity in the infected cells will be reduced. Thus, using this protocol, a sample capable of inhibiting HIV or influenza virus replication was identified. The test fractions or compounds were evaluated as follows. Target A549 human lung cells were seeded at 0.5×10⁵ cells per well (24-well plate) in complete DMEM. The lung cell line was used since it is susceptible to HA-mediated viral entry. The stock HIV/VSVG or HIV/HA virions (approximately 2×10⁶ relative light units, or RLUs, on the target cells) were mixed with the individual sample first, and the mixture was incubated with the A549 target cells for 24 hours. Ten microliters of serial concentrations (for example, 20, 10, 5, 2.5, 1.25, 0.625 and 0.3125 μg/mL) and 190 L of the pseudovirus were incubated with target cells. Twenty-four (24) hours post-infection, all media containing sample and virus was removed from target cells and replaced with fresh and complete DMEM. Forty-eight (48) hours post-infection, the target cells were lysed and the luciferase activity was determined. Two different outcomes may occur: 1) It is likely that some samples will “inhibit replication” of both HIV/VSVG and HIV/HA virions (lower Luc for HA and VSVG), since some of these samples can block post-entry steps during viral entry, or some of them are just toxic to the target cells. These samples are classified as anti-HIV. 2) The samples which can specifically inhibit the HIV/HA viral entry (lower Luc for HIV/HA, but not for HIV/VSVG) will be classified as anti-HA inhibitors (influenza virus inhibitors). The concentration of drug inhibiting 50% of virus infectivity (EC₅₀ value) was determined.

Anti-HIV, anti-Ebola and anti-Marburg Virus Evaluation Assay. This protocol was modified from the aforementioned anti-H5N1 influenza virus evaluation assay, which was designed to identify potential inhibitors for HIV, Ebola and Marburg viruses replication (post-entry steps). In this system, the HIV vector pNL4-3.Luc. R. E. was co-transfected with the VSVG to generate HIV/VSVG virions, and the same HIV vector was co-transfected with the Ebola or Marburg glycoprotein (GP) constructs to generate HIV virions with Ebola or Marburg GP on the viral surface (HIV/EBVG or HIV/MAVG). The infection level was measured as relative light units (RLUs) in the infected cells. The luciferase activities of the 293T cells infected with the HIV vector pNL4-3.Luc. R. E. reached the range of 10⁵-10⁶ RLUs, approximately 100-fold higher than the background levels when measured using the HIV virions without VSVG. The evaluation principle is that the level of the luciferase activity in the cells should be proportional to the level of viral entry and replication. If a compound can interfere with HIV replication/or EBVG or MAVG-mediated viral entry, the level of the luciferase activity in the infected cells will be reduced. Thus, using this protocol, compounds capable of inhibiting HIV, EBOV and MARV replication were identified. The test compounds were evaluated as follows. Target A549 human lung cells were seeded at 0.5×10⁵ cells per well (24-well plate) in complete DMEM. The lung cell line was used since it is susceptible to EBVG or MAVG-mediated viral entry. The stock HIV/VSVG or HIV/EBVG or MAVG virions (approximately 2×10⁶ relative light units, or RLUs, on the target cells) were mixed with the individual sample first, and the mixture was incubated with the A549 target cells for 24 hours. Ten microliter of each sample in varying concentrations and 190 μL of the pseudovirus were incubated with target cells. Twenty-four (24) hours post-infection, all media containing sample and virus was removed from target cells and replaced with fresh and complete DMEM. Forty-eight (48) hours post-infection, the target cells were lysed and the luciferase activity was determined.

Anti-SARS-CoV-2 Evaluation Assay. This protocol was modified from the aforementioned anti-H5N1 influenza virus evaluation assay, which was designed to identify potential inhibitors for SARS-CoV-2. In this assay, the HIV vector pNL4-3.Luc. R. E. was co-transfected with SARS-CoV-2 spike protein (SARSP) expression plasmid to generate SARS-CoV-2 pseudovirions (HIV/SARSP). Target Hep G2 liver cancer cells were seeded at 4×10³ cells per well (96-well plate) in complete EMEM. The liver cell line was used because it is susceptible to SARS-CoV-2 mediated viral entry. Ten microliter of each sample in varying concentrations and 190 μL of the HIV/SARSP were incubated with target cells. Forty-eight (48) hours post-infection, the target cells were lysed and the luciferase activity was determined. Arbidol was used as positive control in the experiments. The IC₅₀ value of arbidol against SARS-CoV-2 pseudovirion was measured as 5.23 μM, which was in agreement with the literature reported value (4.11 μM).

Anti-HIV Evaluation Using HIV-1 Clinical Strains.

The HIV-1 clinical strains such as BAL and SF162 (macrophage-tropic: M-tropic), BAL (T-cell line tropic: T-tropic), and 89.6 (a dual tropic strain), HIV-1_{LAV} (wild type), NRTI (nucleoside reverse transcriptase inhibitor)-resistant isolate (HIV-1₁₆₁₇₋₁) (AZT resistant strain from AIDS repository) and NNRTI (non-nucleoside reverse transcriptase inhibitor)-resistant isolate (HIV-1_{N119}) (nevaripine resistant strain from AIDS repository) were used in the study. A standardized human peripheral blood mononuclear cell culture (PBMC) assay was used to determine the compound susceptibility of these HIV-1 strains. AZT, an anti-HIV drug in clinical use, was used as a positive control. All data were generated from three independent experiments, each performed in triplicate. Prior to HIV-1 infection, fresh human PBMCs were used in each experiment. Briefly, donor PBMCs were suspended in R-3 medium [RPMI 1640 medium supplemented with 15-20% FBS (fetal bovine serum), 5% IL-2 (human interleukin-2), 250 U of penicillin per mL, 250 μg of streptomycin per mL and 2 mM L-glutamine] was stimulated with PHA (phytohaemagglutinin, 2-3 μg/mL) for seven days. The preparations (samples) were added to the cultured cells, and the different HIV-1 strains were used to challenge the cultured cells in 96-well plates [1×10⁵ cells per well with 1000 TCID₅₀ (virus 50% tissue culture infectious doses) of HIV strain]. After seven days of incubation, the supernatants were collected and the HIV p24 levels of the infected cells were determined using a p24 antigen ELISA. To measure IC₅₀ values, each drug was tested using a serial of concentrations (for example, 5, 1, 0.2, 0.04, 0.008, 0.016 and 0 μg/mL). The IC₅₀ s were calculated by comparing p24 antigen values for the samples-containing wells with those for no drug control wells. For the p24 assay, the maximum cutoff should be around 120-150 μg/mL.

Antiviral Evaluation Using Infectious Influenza Viruses.

A panel of influenza viruses such as influenza H1N1 (A/HK/415742/09), H3N2 (A/Hong Kong/1/1968), H5N1 (A/Vietnam/1203/2004H), H7N1 (A/Rhea/North Carolina/39482/93), H7N7 (A/Netherlands/219/2003), H7N9 (A/Anhui/1/2013) and H9N2 (A/Chicken/Y280/97) were used in the studies. Samples were evaluated for their antiviral activities against the influenza viruses in A549 cells. Briefly, the preparations (samples) were added to the cultured cells, and the different influenza strains were used to challenge the cultured cells in 24-well plates (1×10⁵ cells per well). After removal of the unbound viruses, the cells were incubated for 48 h. The viral supernatants were collected and viral titers were determined by standard plaque assay in MDCK (Madin-Darby canine kidney) cells.

Cytotoxicity Evaluation Using the SRB Assay.

The cytotoxicity of the sample for A549 cells was measured using the sulforhodamine B (SRB) assay (Vichai V, Kirtikara K. Nature protocols 2006; 1: 1112-1116). Briefly, 190 μL of A549 cells (2×10⁴ cells/mL) was seeded in each well of a 96-well cell culture plate. After 24 hours, 10 μL of DMSO alone, 10 μL of zidovudine (AZT) as positive controlin 10% DMSO, and 10 μL of each sample in 10% (v/v) DMSO were respectively added into wells of a 96-well tissueculture plate. 10 μL of 10% (v/v) DMSO was added into each blank well of a 96-well tissueculture plate as a background calculation plate. After incubation at 37° C. for 2 days, 50 μL cold 50% (w/v) trichloroacetic acid (TCA) were added into each well of the plates, and were further incubated at 4° C. for 1 h. The plates were then washed four times with low-running tap water, and they were allowed to dry at room temperature (r.t.). 50 μL of 0.4% (w/v) SRB solution was added to each well. The plate was left at r.t. for 5-10 mins and were quickly rinsed with 1% (v/v) acetic acid to remove unbound dye. The plates were allowed to dry at r.t. 100 mL of 10 mM Tris base solution (pH 10.5) was added to each well and the plates were shake on a gyratory shaker for at least 30 min to solubilize the protein-bound dye. The OD values were measured at 515 nm in a microplate reader. The CC₅₀ (the concentration of an agent causing 50% cytotoxicity) values were calculated using the GraphPad Prism version 5.0 (GraphPad Software, San Diego, CA).

Toxicity Evaluation in Mice. Repeated-dose toxicity study in mice was applied on the selected samples. The animal study was approved and performed according to Animal Care and Use Guidelines of the Animal Ethics Committee at Hong Kong Baptist University and performed following Animal Care and Use guidelines set by NIH (National Institute of Health, USA). BALB/c nude mice, SPF class, male or female, 6-8 weeks old, were purchased from Charles River Laboratories. Before the experiment, the mice are kept for one week of acclimatization to SPF class laboratory conditions. The mice were then divided into three groups: two dose (25 and 50 mg/kg: 10 mice/each dose group) groups of an ANL compound and one dose of vehicle (negative control: 10 mice). Daily injections at i.p. sites were scheduled for 28 days. Weights of mice were measured twice a week until the end of the experiment. Skin conditions, food intake, water consumption and posture of mice were also inspected. All mice were sacrificed as the end of the experiment to inspect the essential organs

TABLE 1
Anti-HIV activity of tuberculatin analogs.^{a}

| Compound No. | CC₅₀ (nM) | EC₅₀ (nM) | SI |
|---|---|---|---|
| Tuberculatin (1) | 3265 | 220 | 14.8 |
| Diphyllin (7) | 2460 | 170 | 14.6 |
| 9 | 1180 | 34.6 | 34.1 |
| 10 | 188 | 0.97 | 193 |
| 11 | 1260 | 208 | 6.1 |
| 12 | 472 | 26.9 | 17.5 |
| 16 | >20000 | 6582 | >3 |
| 17 | 2245 | 251 | 8.9 |
| 18 | 184 | 7.37 | 25 |
| 19 | 368 | 11.9 | 31 |
| 20 | 2583 | 280 | 9.2 |
| 21 | 328 | 126 | 2.6 |
| 22 | 4632 | 569 | 8.1 |
| 23 | 736 | 43.4 | 17.0 |
| 25 | 1654 | 441 | 3.8 |
| 26 | 4550 | 917 | 5.0 |
| 27 | 2217 | 384 | 5.8 |
| 28 | 2207 | 486 | 4.5 |
| 29 | 381 | 91.8 | 4.2 |
| 30 | 1188 | 18 | 6.5 |
| 31 | 1399 | 177 | 7.9 |
| 32 | 441 | 80.9 | 5.4 |
| 42 | >25000 | >25000 | — |
| 43 | >25000 | >25000 | — |
| 44 | >25000 | >25000 | — |
| 45 | 46800 | 2730 | 17.3 |
| 46 | >50000 | 48600 | — |
| 47 | >25000 | >25000 | — |
| 48 | >25000 | >25000 | — |
| 49 | 23500 | 5450 | 4.3 |
| 50 | >25000 | 23100 | — |
| 51 | >25000 | 15700 | — |
| 52 | >25000 | >25000 | — |
| 53 | >25000 | 14700 | — |
| 54 | >25000 | >25000 | — |
| Nevirapine^{b} | >200 | 35.8 | >5 |

^{a}Results are expressed as CC₅₀ (the concentration caused inhibition of cell growth of host A549 cells by 50%) and EC₅₀ (effective concentration of compound to inhibit viral growth by 50%) values in nM, and data were obtained from triplicate experiments. SI = CC₅₀/EC₅₀.
^{b}Positive control compound.

TABLE 2
Antiviral activities of tuberculatin analogs.^{a}

| | CC₅₀ (nM) | EC₅₀ (nM) | EC₅₀ (nM) | SI | SI |
|---|---|---|---|---|---|
| Compound No. | A549 | AIV | VSV | AIV | VSV |
| Diphyllin (7) | 1983 | 2.36 | 11.2 | 842 | 176 |
| 10 | 400 | 0.75 | 1.50 | 536 | 266 |
| 18 | 208 | 0.71 | 2.43 | 295 | 85.9 |
| 19 | 2101 | 70.0 | 144 | 30.0 | 14.6 |
| 20 | 1566 | 39.9 | 53.8 | 39.2 | 29.1 |
| 21 | 93.0 | 6.25 | 12.0 | 14.9 | 7.8 |
| 22 | 1703 | 39.2 | 75.1 | 43.5 | 22.7 |
| 23 | 158 | 0.30 | 1.24 | 528 | 127 |
| 25 | 648 | 0.72 | 4.52 | 895 | 143 |
| 26 | 2.66 | 0.79 | 0.58 | 3.4 | 4.6 |
| 27 | 379 | 16.9 | 20.8 | 22.4 | 18.2 |
| 28 | 560 | 16.7 | 56.1 | 33.5 | 10.0 |
| 29 | 483 | 8.66 | 19.9 | 55.8 | 24.3 |

^{a}Results are expressed as CC₅₀ (the concentration caused inhibition of cell growth of host A549 cells by 50%) and EC₅₀ (effective concentration of compound to inhibit viral growth by 50%) values in nM, and data were obtained from triplicate experiments.
SI = CC₅₀/EC₅₀.

TABLE 3
Anti-SARS-COV-2 activity (% inhibition) of tuberculatin analogs.^{a}

| | CC₅₀ | EC₅₀ | CC₅₀ | CC₅₀ |
|---|---|---|---|---|
| Compound | (2000 nM) | (2000 nM) | (400 nM) | (400 nM) |
| No. | 293T | SARS-COV-2 | 293T | SARS-COV-2 |
| Diphyllin (7) | 56.1 | 94.6 | 10.0 | 51.7 |
| 9 | 36.5 | 82.4 | −14.2 | 15.9 |
| 10 | 70.0 | 95.6 | 45.3 | 78.7 |
| 11 | 46.7 | 85.1 | 3.47 | 24.3 |
| 12 | 70.5 | 96.3 | 26.8 | 64.5 |
| 16 | −7.71 | 32.4 | −9.37 | 38.5 |
| 17 | 26.2 | 66.2 | −11.2 | 12.8 |
| 18 | 79.9 | 98.3 | 61.8 | 94.9 |
| 19 | 72.7 | 98.3 | 56.7 | 94.9 |
| 20 | 32.6 | 85.2 | 6.90 | 9.80 |
| 21 | 75.0 | 96.3 | 55.9 | 94.9 |
| 22 | 66.3 | 86.0 | −1.25 | 43.8 |
| 23 | 88.6 | 96.7 | 83.5 | 95.2 |
| 25 | 44.9 | 93.6 | −4.42 | 33.5 |
| 26 | 28.4 | 85.5 | −8.23 | 26.0 |
| 27 | 79.5 | 93.0 | 20.6 | 73.2 |
| 28 | 82.8 | 93.0 | 2.48 | 34.2 |
| 29 | 96.4 | 95.6 | 52.0 | 72.2 |
| 30 | 82.5 | 92.7 | 32.6 | 20.6 |
| 31 | 72.1 | 91.9 | 35.1 | 40.4 |
| 32 | 95.6 | 97.4 | 27.2 | 64.0 |

^{a}Results are expressed as % inhibitory effect, and data were obtained from triplicate experiments.

Compound 1 (tuberculatin) was identified as an anti-HIV lead compound from the methanol extract of Justicia procumbens (aerial parts). Tuberculatin (1) showed anti-HIV activities with EC₅₀ values of 220 nM and SI of 14.8 in our present “One-Stone-Two-Birds” evaluation system. In order to improve their antiviral activities and reduce their cytotoxicities, we synthesized numerous analogues of the compound. We designed synthetic route to modify the sugar unit of 5 with different functional groups, which led to the preparation of compounds 8, 9, 10, 11, 12, 16, 17, 18, 19, 20, 21, 22, 23, 24, 25, 26, 27, 28, 29, 30, 31, 32, 42, 43, 44, 45, 46, 47 and 48 (FIGS. 2-6). These compounds were evaluated for their antiviral activities using our developed antiviral screening protocols. The compounds were found to show antiviral activities against human immunodeficiency virus (HIV), avian influenza virus (AIV), vesicular stomatitis virus (VSV) and coronavirus (CoV).

The compounds in Table 1 displayed inhibitory effects against the HIV-1 infection with EC₅₀ values ranging from 0.97 to 6582 nM, and their calculated selectivity indice (SI, CC₅₀/EC₅₀) ranged from 2.6 to 193. The bioactivity result revealed that the ANLs with an apiose sugar unit show strong inhibitory activity against viral replication with low toxicity. Compound 10 was the most potent viral inhibitor among these ANL derivatives with an EC₅₀ value of 0.97 nM and SI of 193. By analyzing the structure and activity relationship (SAR) of these compounds, we found the ANLs with a β-configured apiose moiety exhibited much stronger antiviral activity than those with an α-configured apiose moiety. Acetalization on the 2,3-diol of the apiose moiety with an aldehyde could also significantly alter the antiviral activity. For example, the antiviral activities of the acetonides 9 and 10 were increased around 6 and 12 times in comparison with their corresponding unacetonized counter parts 1 and 19. By including ANL atropisomer analogs, further SAR was disclosed about ANL compounds. Compounds 45-48 are synthetic atropisomer analogs of tuberculatin (1). Compounds 45 and 46 are a pair of atropisomers with the absolute configurations of the C-1 and C-1′ biphenyl groups being determined as R and S, respectively (FIG. 6). Compounds 47 and 48 are determined as another pair of atropisomer with the absolute configurations of the C-1 and C-1′ biphenyl groups being determined as R and S, respectively. Compounds 49-54 are also atropisomer analogs of tuberculatin (1) obtained from Justicia procumbens. The pair of compounds 49 and 50, the pair of compounds 51 and 52, and the pair of compounds 53 and 54 are determined as additional three pairs of atropisomers with the absolute configurations of the C-1 and C-1′ biphenyl groups being determined as R for compounds 49, 51 and 53 and S compounds 50, 52 and 54, respectively. Significant activity discrepancy between a pair of atropisomers was observed between the pair of ANL atropisomers 45 and 46, between the pair of ANL atropisomers 49 and 50, and between the pair of ANL atropisomers 51 and 52, and between the pair of ANL atropisomers 53 and 54. Compounds 45, 49, 51 and 53 with the R configuration demonstrated much more potent antiviral activities than their corresponding S atropisomer compounds 46, 50, 52 and 54 (Table 1).

The compounds in Table 2 displayed inhibitory effects against the AIV infection with EC₅₀ values ranging from 0.30 to 70.0 nM, and their calculated selectivity indice (SI, CC₅₀/EC₅₀) ranged from 3.4 to 895.

The compounds in Table 2 displayed inhibitory effects against the VSV infection with EC₅₀ values ranging from 0.58 to 144 nM, and their calculated selectivity indice (SI, CC₅₀/EC₅₀) ranged from 4.6 to 266.

The compounds in Table 3 displayed inhibitory effects against the SARS-CoV-2 infection measured at the concentrations of 400 and 2000 nM, respectively. Compound 9, 11, 20, 25 and 26 showed over 80% inhibitory effects against SARS-CoV-2 and less than 45% inhibitory effects on the 293T host cells at the concentration of 2000 nM. Compound 12, 27 and 32 showed over 64% inhibitory effects against SARS-CoV-2 and less than 30% inhibitory effects on the 293T host cells at the concentration of 400 nM. The experiments determined that compounds 9, 11, 12, 20, 25, 26, 27 and 32 are the active molecules against SARS-CoV-2.

Materials and Methods

General Experimental Procedures

Optical rotations were measured with a Perkin-Elmer model 241 polarimeter (Maryland, USA). IR spectra were recorded on a Jasco FT/IR-410 spectrometer, equipped with a Specac Silver Gate ATR system by applying a film on a Germanium plate (Maryland, USA). CD spectra were recorded on a JASCO J-1500 CD spectrometer (Maryland, USA). 1D and 2D NMR spectra were recorded on a Bruker DRX-500 MHz or a Bruker DRX-400 MHz or a Bruker DPX-360 MHz spectrometer (Rheinstetten, Germany). Chemical shifts (δ) were expressed in ppm and coupling constants (J) are reported in Hz. All NMR experiments were obtained by using standard pulse sequences supplied by the vendor. Chemical shifts (δ) were expressed in ppm with reference to the solvent signals (CD30D: ¹H: 3.31 ppm, ¹³C: 49.00 ppm; CDCl₃: ¹H: 7.27 ppm, ¹³C: 77.23 ppm; DMSO-d₆: ¹H: 2.50 ppm, ¹³C: 39.51 ppm; acetone-d₆: ¹H: 2.05 ppm, ¹³C: 29.92, 206.68 ppm.), and coupling constants (J) are reported in Hz. Column chromatography was carried out on silica gel (230-400 mesh, Natland International Corporation, Research Triangle Park, NC, USA). Reversed-phase flash chromatography was accomplished with RP-18 silica gel (40-63 mm, EM Science, New Jersey, USA), and reversed-phase preparative HPLC was carried out on a Agilent 1200 series Delivery System pump, equipped with a Agilent 1200 series photodiode detector (California, USA), and a YMC-Pack ODS-A C18 column (120 Å, 5 μm, 250×20 mm², Tokyo, Japan) or a Alltima C18 column (120 Å, 5 μm, 250×10 mm², Chicago, USA). Thin-layer chromatography (TLC) was performed on EMD glass-backed plates coated with 0.25 mm layers of silica gel 60 F254 (Kassel, Germany). HRTOFMS spectra were recorded on a Micromass QTOF-2 (Milford, MA, USA), an Agilent 6540 Q-TOF (Santa Clara, CA, USA), an Agilent 6460 Triple Quadrupole, or a Bruker Q-TOF mass spectrometer (Bremen, Germany). All reagents were purchased from commercial sources and used without further purification.

Collection of Plant Materials

The aerial parts of Justicia procumbens were collected in Ningde City of Fujian Province, China, in July 2013. The identification was conducted by Prof. Hubiao Chen in School of Chinese Medicine, Hong Kong Baptist University. A voucher specimen (δHA00026) is momentarily available for inspection at the Quality Research Laboratory/Phytochemistry Laboratory, School of Chinese Medicine, Hong Kong Baptist University.

Isolation of Tuberculatin (1)

The air-dried aerial parts of the plant materials (18.0 kg) were exhaustively extracted with MeOH for 4 times (4×60 L) at room temperature (12 h each time) and filtered to yield a filtrate. Concentration of the filtrate under vacuum gave a brown residues. The residues were dissolved in H₂O and then successively fractionated with petroleum ether (4×5 L), EtOAc (4×5 L) and n-BuOH (4×5 L) to afford petroleum ether-soluble, EtOAc-soluble, n-BuOH-soluble and H₂O-soluble extracts after condensed to dryness in vacuo. The EtOAc soluble portion (154.0 g) was chromatographed over a silica gel column (100-230 mesh; 10×150 cm), eluting with gradient petroleum ether/Me₂CO (8:1, 10 L; 4:1, 10 L; 3:1, 10 L; 1:1, 10 L), followed by CH₂Cl₂/MeOH (8:2, 10 L; 7:3, 10 L; 0:10 10 L) solutions to afford 140 fractions (F1-140). A portion of the combined fractions F92-126 were chromatographed over a silica gel column (100-230 mesh; 5×100 cm), eluting with gradient CH₂Cl₂/EtOAc (9:1, 5 L; 8:2, 5 L; 7:3, 5 L), followed by CH₂Cl₂/MeOH (8:2, 5 L; 8:2, 5 L; 7:3, 5 L; 0:10, 5 L) solutions to afford 50 sub-fractions (SFI1-50). The combined sub-fractions SFI22-45 were subjected to a RP-18 silica gel column (40-63 m; 3.5×50 cm), eluting with an MeOH/H₂O (8:2) solvent system to yield fraction SFI51 and SFI52. SFI52 was subjected to a further RP-18 silica gel column (40-63 m; 3.5×50 cm) separation, eluting with gradient MeOH/H₂O (1:9, 0.5 L; 2:8, 0.5 L; 3:7, 0.5 L; 4:6, 0.5 L; 5:5, 0.5 L; 6:4, 0.5 L; 7:3, 0.5 L; 1:0, 1 L) solutions to give fractions SFIB1-8, respectively. SFIB7 was subjected to a silica gel column (100-230 mesh; 3.5×50 cm) separation, eluting with gradient CH₂Cl₂/acetone (4:1, 0.5 L; 2:1, 0.5 L; 1:1, 0.5 L) and MeOH (1 L) solutions to afford fractions SFIB9-12, respectively. SFIB10 was further subjected to a silica gel column (100-230 mesh; 3.5×50 cm) separation, eluting with CH₂Cl₂/EtOAc (2:1, 0.5 L), CH₂Cl₂/acetone (1:1, 0.5 L) and MeOH (1 L) solutions to afford fractions SFIB13-15, respectively. SFIB14 was subjected to a preparative HPLC separation on the Phenomenex LUNA-C-18 column (12 m; 250×50 mm), eluting with an isocratic MeCN/H₂O (3:7) at a flow rate of 20 m/min to obtain tuberculatin (1). Another portion of the combined fractions F92-126 were chromatographed over a MCI column chromatography (CC) and eluted with aqueous MeOH (0, 20%, 40%, 60% and 80%) to afford 16 fractions (FA-FP). Fraction FK was subjected to a semipreparative HPLC separation (Solvent system MeCN:H₂O 35:65, flow: 4 mL/min) to yield 11 fractions (FK1-FK11). FK7 and FK6 were pooled and subjected to a semipreparative HPLC separation with a constant gradient of MeCN/H₂O (28%) (flow rate at 2 mL/min, UV detection at λ=210 nm) to give Atrop1. FK5 was separated by a semipreparative HPLC separation, and eluted with a constant gradient of MeCN/H₂O (27.5%) (flow rate at 2 ml/min, UV detection at λ=210 nm) to give Atrop2. FK4 was subjected to a semipreparative HPLC separation with a constant gradient of MeCN/H₂O (27%) (flow rate at 2 ml/min, UV detection at λ=210 nm) to afford Atrop3. Atrop1, Atrop2 and Atrop3 were further chirally separated to obtain the pair compounds 49 and 50, the pair compounds 51 and 52, and the pair compounds 53 and 54, respectively.

Compound 3

To a suspension of L-Ribose (3 g) in dry acetone (150 mL) at 0° C. was added concentrated sulfuric acid (0.088 mL). The resulting solution was stirred at room temperature (r.t.) for 3 h. The reaction mixture was concentrated to remove the solvent and diluted with ethyl acetate (EtOAc) (100 mL), which was washed sequentially with brine (50 mL×3), NaHCO₃(saturated, 50 mL) and water (50 mL). The organic layer was concentrated to give 3 as an oil (3.8 g): ¹H NMR (400 MHz, CDCl₃) δ 1.32 (3H, s), 1.49 (3H, s), 3.70-3.79 (2H, m), 4.41 (1H, brs), 4.58 (1H, d, J=6.0 Hz), 4.83 (1H, d, J=5.9 Hz), 5.42 (1H, s).

Compound 5

A solution of 3 (3.8 g crude, 20 mmol) in methanol (MeOH) (40 mL) was stirred with potassium carbonate (K₂CO₃) (2.33 g, 22.0 mmol) and aqueous formaldehyde (HCHO) solution (20 mL, 39.5% aq.) at reflux for 6 h. The reaction mixture was cooled to r.t., neutralized with hydrochloric acid (HCl solution) (2M in H₂O), filtered through celite and concentrated in vacuo to afford crude 2,3-O-isopropylidene-L-hamamelose 4. Without further purification, compound 4 was dissolved in water (160 mL) and stirred with sodium borohydride (NaBH₄) (3.86 g, 40 mmol) at r.t. After 1.5 h, a new spot was detected on a thin layer chromatography (TLC) plate. The reaction solution was neutralized with glacial acetic acid and stirred with sodium metaperiodate (NaIO₄) (13.4 g, 22.0 mmol) at r.t. for 1 h. TLC analysis (EtOAc) showed the formation of a major product (Rf 0.62). The solution was concentrated to dryness in vacuo and triturated exhaustively with EtOAc. The organic extracts were concentrated in vacuo and purified by a silica gel column (EtOAc/cyclohexane 1:1-4:1 gradient) to afford compound 5 as a colorless oil (3.5 g, overall yield of 79% from L-robise): ¹H NMR (400 MHz, CDCl₃) δ 1.41 (3H, s), 1.50 (3H, s), 2.72 (1H, brs), 3.66 (1H, brs), 3.81 (2H, s), 3.98 (1H, d, J=10 Hz), 4.04 (1H, d, J=10 Hz), 4.37 (1H, s), 5.42 (1H, d, J=3.9 Hz); ¹³C NMR (100 MHz, CDCl₃) δ 27.3, 27.5, 64.1, 74.2, 86.8, 91.5, 101.4, 113.4.

Compound 6

Imidazole (2.20 mmol), tert-butyl(chloro)diphenylsilane (TBDPSCl) (1.1 mmol) and 4-dimethylaminopyridine (DMAP) (0.05 mmol) were added to a stirring solution of 5 (190 mg, 1 mmol) in dichloromethane (CH₂C₂) (3 mL) at 0° C. After 2 h at r.t., the reaction mixture was diluted with EtOAc (50 mL), washed with water (50 mL) and brine (50 mL×2). The organic layer was concentrated and purified by a silica gel column (hexane/EtOAc 10:1-3:1 gradient) to give the desired product 6 as a colorless oil, which was determined as a pair of epimers (the ratio of the α/β about 3:1 according to the NMR data): ¹H NMR (400 MHz, CDCl₃) δ 1.09 (3H, s), 1.10 (δH, s), 1.32 (3H, s), 1.34 (1H, s), 1.50 (3H, s), 1.55 (1H, s), 3.71-3.80 (0.7H, m), 3.82-3.88 (2H, m), 3.95 (0.33H, d, J=12.0 Hz), 3.99 (1H, d, J=10.0 Hz), 4.10-4.16 (0.33H, d, J=9.9 Hz), 4.17 (1H, d, J=9.9 Hz), 4.43 (0.3H, d, J=3.1 Hz), 4.46 (1H, s), 5.05-5.12 (0.33H, dd, J=12.0, 3.1 Hz), 5.42 (1H, d, J=5.8 Hz), 7.39-7.50 (8H, m), 7.65-7.73 (5.3H, m); ¹³C NMR (100 MHz, CDCl₃) δ 19.2, 26.7, 26.8, 27.1, 27.5, 27.6, 64.5, 65.4, 70.2, 74.0, 81.4, 87.2, 91.1, 91.4, 98.1, 101.7, 113. 2, 114.2, 127.8. 127.9, 129.9, 130.0, 132.2, 132.4, 132.5, 132.6, 135.5, 135.7.

Compound 8

An oven-dried 10-mL flask was charged with diphyllin (7) (113 mg, 0.3 mmol), D-apiose derivative 6 (113 mg, 0.3 mmol), and triphenylphosphine (PPh₃) (157 mg, 0.6 mmol) in tetrahydrofuran (THF) was added diisopropylazodicarboxylate (DIAD) (121 mg/118 μL, 0.6 mmol) by syringe at 0° C. under nitrogen atmosphere. The mixture was stirred at r.t. for 2 h-, the reaction was completed. The reaction mixture was diluted with EtOAc (15 mL) and quenched with H₂O (5 mL). The organic layer was separated, washed with brine (10 mL×3) and concentrated in vacuo to give a yellow solid, which was purified by a silica gel column (hexane/EtOAc 10:1) to give the desired product 8 (200 mg) as a pair of epimers (the ratio of α/β is about 1:10): HRMS m/z [M+Na]+(calcd for C₄₅H₄₆O₁₁SiNa⁺, 813.2702; found 813.2695); ¹H NMR (400 MHz, CDCl₃) δ 1.05-1.11 (10H, m), 1.47 (2H, s), 1.73 (4H, d, J=6.90 Hz), 3.80-3.83 (2H, m), 3.89 (1H, s), 4.04 (3H, s), 4.78 (1H, d, J=3.89 Hz), 5.35 (1H, d, J=4.02 Hz), 5.40-5.46 (1H, m), 5.53-5.60 (1H, m), 6.06 (1H, t, J=1.44 Hz), 6.10 (1H, d, J=1.25 Hz), 6.84 (2H, t, J=6.46 Hz), 6.93-7.00 (1H, m), 7.09 (1H, s), 7.34-7.50 (5H, m), 7.61-7.75 (4H, m), 7.78 (1H, d, J=0.88 Hz).

Compounds 9 and 10

To a solution of 8 (100 mg, 0.13 mmol) in THF was added tetrabutylammonium fluoride (TBAF) (0.2 mL, 1M in THF/H₂O at a ratio of 95:5 solution). The mixture was stirred at 0° C. for 2 h. TLC showed 2 spots under the UV and fluorescent detection. Separation of the mixture by prep-TLC (CH₂Cl₂/EtOAc 5:1) afforded the α-configured apioside 9 (5 mg, 7% yield) as a minor product and the p configured apioside 10 (43 mg, 61% yield) as a major product: Compound 9: HRMS m/z [M+Na]+(calcd for C₂₉H₂₈O₁₁Na⁺, 575.1524; found 575.1535); ¹H NMR (400 MHz, CDCl₃) δ 1.51 (3H, s, —C(CH₃)₂), 1.54 (3H, s, —C(CH₃)₂), 3.81 (3H, s, —OCH₃), 3.97 (1H, d, J=12 Hz, H-5″), 4.01 (1H, d, J=12 Hz, H-5″), 4.07 (3H, s, —OCH₃), 4.17 (1H, d, J=10 Hz, H-4″), 4.20 (1H, d, J=10 Hz, H-4″), 4.95 (1H, s, H-2″), 5.48 (1H, dd, J=14.0, 2.0 Hz, H-12), 5.53 (1H, dd, J=14.0, 2.0 Hz, H-12), 5.70 (1H, s, H-1″), 6.05 (1H, d, J=1.4 Hz, H-7′), 6.10 (1H, d, J=1.4 Hz, H-7′), 6.77-6.81 (1H, m, H-6′), 6.81-6.83 (1H, m, H-2′), 6.96 (1H, dd, J=7.8, 1.7 Hz, H-5′), 7.07 (1H, s, H-8), 7.44 (1H, s, H-5); ¹³C NMR (100 MHz, CDCl₃) δ 27.7 (—C(CH₃)₂), 27.9 (—C(CH₃)₂), 55.8 (—OCH₃), 56.0 (—OCH₃), 64.0 (C-12), 67.0 (C-5″), 75.5 (C-4″), 86.8 (C-2″), 92.1 (C-3″) 100.0 (C-1″), 101.2 (C-7′), 106.2 (C-8), 108.2 (C-5), 108.6 (C-5′), 110.6 (C-2′), 114.3 (—C(CH₃)₂), 119.3 (C-6′), 123.5 (C-9), 123.6 (C-10), 126.3 (C-1′), 127.0 (C-2), 128.2 (C-3), 130.6 (C-1), 135.5 (C-4), 143.8 (C-3′), 147.4 (C-4′), 150.2 (C-7), 151.7 (C-6), 169.6 (C-11); Compound 10: HRMS m/z [M+Na]+(calcd for C₂₉H₂₈O₁₁Na⁺, 575.1524; found 575.1529); ¹H NMR (400 MHz, CDCl₃) δ 1.58 (3H, s, —C(CH₃)₂), 1.76 (3H, s, —C(CH₃)₂), 3.74 (1H, d, J=12 Hz, H-5″), 3.81 (3H, s, —OCH₃), 3.85 (1H, d, J=12 Hz, H-5″), 3.90 (1H, d, J=10.0 Hz, H-4″), 4.06 (3H, s, —OCH₃), 4.20 (1H, d, J=10.0 Hz, H-4″), 4.77 (1H, d, J=4.1 Hz, H-2″), 5.38 (1H, d, J=4.1 Hz, H-1″), 5.44 (1H, dd, J=15.6, 1.6 Hz, H-12), 5.56 (1H, dd, J=15.6, 1.6 Hz, H-12), 6.04 (1H, t, J=1.4 Hz, H-7′), 6.09 (1H, d, J=1.1 Hz, H-7′), 6.76-6.84 (2H, m, H-2′ and H-6′), 6.92-6.98 (1H, m, H-4′), 7.06 (1H, s, H-8), 7.79 (1H, s, H-5); ¹³C NMR (100 MHz, CDCl₃) δ 27.8 (—C(CH₃)₂), 28.8 (—C(CH₃)₂), 55.8 (—OCH₃), 56.0 (—OCH₃), 64. (C-12), 67.0 (C-5″), 72.4 (C-4″), 82.4 (C-2″), 91.2 (C-3″), 100.8 (C-1″), 101.2 (C-7′), 104.9 (C-8), 106.0 (C-5), 108.1 (C-5′), 110.4 (C-2′) 110.5 (—C(CH₃)₂), 116.7 (C-6′), 119.2 (C-1′), 123.4 (C-9), 123.5 (C-10), 127.2 (C-2), 129.0 (C-3), 130.5 (C-1), 136.0 (C-4), 145.4 (C-3′), 147.4 (C-4′), 150.2 (C-7), 151.8 (C-6), 169.8 (C-11).

Compound 11

To a solution of 9 (5 mg, 0.009 mmol) in CH₂Cl₂ was added DMAP (cat.), triethylamine (Et₃N) (3 eq.) and Ac₂O (3 eq.). The mixture was stirred at r.t. for 2 h. TLC showed the desired product was formed and the reaction mixture was purified by prep-TLC to give 11 as a yellow solid (4.5 mg, 90% yield): HRMS m/z [M+Na]+(calcd for C₃₁H₃₀O₁₂Na⁺, 617.1629; found 617.1631); ¹H NMR (400 MHz, CDCl₃) δ 1.51 (3H, s, —C(CH₃)₂), 1.53 (3H, s, —C(CH₃)₂), 2.16 (3H, s, —OCOCH₃), 3.82 (3H, s, —OCH₃), 4.09 (3H, s, —OCH₃), 4.21 (1H, d, J=12 Hz, H-5″), 4.24 (1H, d, J=12 Hz, H-5″), 4.47 (1H, d, J=11.8 Hz, H-4″), 4.60 (1H, d, J=11.8 Hz, H-4″), 4.94 (1H, s, H-2″), 5.47 (1H, dd, J=14.4, 1.8 Hz, H-12), 5.53 (1H, dd, J=14.4, 1.8 Hz, H-12), 5.68 (1H, s, H-1″), 6.02-6.11 (2H, m, H-7″), 6.77-6.85 (2H, m, H-2′ and H-6′), 6.97 (1H, dd, J=7.8, 1.9 Hz, H-5′), 7.08 (1H, s, H-8), 7.42 (1H, s, H-5); ¹³C NMR (100 MHz, CDCl₃) δ 20.9 (—OCOCH₃), 27.5 (—C(CH₃)₂), 27.6 (—C(CH₃)₂), 55.8 (—OCH₃), 56.1 (—OCH₃), 65.2 (C-12), 67.0 (C-5″), 75.6 (C-4″), 87.4 (C-2″), 90.1 (C-3″), 99.9 (C-1″), 101.2 (C-7′), 106.3 (C-8), 108.2 (C-5), 108.7 (C-5′), 110.7 (C-2′), 114.7 (—C(CH₃)₂), 119.3 (C-6′), 123.5 (C-9), 123.6 (C-10), 126.2 (C-2), 128.2 (C-3), 130.6 (C-1), 135.6 (C-4), 143.9 (C-3′), 147.4 (C-4′), 150.2 (C-7), 151.8 (C-6), 169.5 (C-11), 170.6 (—OCOCH₃).

Compound 12

To a solution of 10 (10 mg, 0.018 mmol) in CH₂Cl₂ was added DMAP (cat.), Et₃N (3 eq.) and Ac₂O (3 eq.). The mixture was stirred at r.t. for 2 h. Separation of the reaction mixture by prep-TLC afforded 12 as a yellow solid (9 mg, 89% yield): HRMS m/z [M+Na]+(calcd for C₃₁H₃₀O₁₂Na⁺, 617.1629; found 617.1636); ¹H NMR (400 MHz, CDCl₃) δ 1.57 (3H, s, —C(CH₃)₂), 1.75 (3H, s, —C(CH₃)₂), 2.14 (3H, s, —OCOCH₃), 3.81 (3H, s, —OCH₃), 3.93 (1H, d, J=9.9 Hz, H-5″), 4.06 (3H, s, —OCH₃), 4.23 (1H, d, J=9.9 Hz, H-5″), 4.27 (1H, d, J=11.9 Hz, H-4″), 4.38 (1H, d, J=11.9 Hz, H-4″), 4.77 (1H, d, J=4.3 Hz, H-2″), 5.37 (1H, d, J=4.1 Hz, H-1″), 5.43 (1H, dd, J=14.4, 1.9 Hz, H-12), 5.56 (1H, dd, J=14.4, 1.9 Hz, H-12), 6.04 (1H, d, J=1.4 Hz, H-7′), 6.09 (1H, d, J=1.4 Hz, H-7′), 6.75-6.83 (2H, m, H-2′ and H-6′), 6.94 (1H, d, J=8.2 Hz, H-5′), 7.07 (1H, d, J=1.4 Hz, H-8), 7.79 (1H, s, H-5); ¹³C NMR (100 MHz, CDCl₃) δ 20.8 (—OCOCH₃), 27.8 (—C(CH₃)₂), 28.6 (—C(CH₃)₂), 55.8 (—OCH₃), 56.0 (—OCH₃), 65.3 (C-12), 66.9 (C-5″), 72.6 (C-4″), 83.0 (C-2″), 89.1 (C-3″), 100.7 (C-1″), 101.2 (C-7′), 104.6 (C-8), 108.1 (C-5), 110.5 (C-5′), 110.6 (—C(CH₃)₂), 117.6 (C-2′), 119.3 (C-1′), 123.4 (C-9), 123.5 C-10), 127.2 (C-2), 129.0 (C-3), 130.6 (C-1), 136.1 (C-4), 145.3 (C-3′), 147.4 (C-4′), 150.2 (C-7), 151.8 (C-6), 169.6 (C-11), 170.5 (—OCOCH₃).

Compound 13

To a suspension of L-ribose (5.0 g, 33.3 mmol), freshly distilled benzaldehyde (15 mL) and CuSO₄ (12 g) in dry dimethyl formamide (DMF) (15 mL) was added D-camphorsulfonic acid (4 g). After stirring for 24 h at r.t. under nitrogen atmosphere, the reaction mixture was quenched with Et₃N (15 mL) and diluted with CH₂C₂ (30 mL), which was added celite (50 g). After stirring for 15 min, the suspension was filtered through celite. The filter cake was washed with CH₂Cl₂ (50 mL×3), and the filtrate was concentrated in vacuo. The residue was purified by a silica gel column (hexane/EtOAc 5:1-1:1 gradient) to give the desired product 13 (4 g, 50% yield, the ratio of α/β about 7:1 according to NMR): ¹H NMR (400 MHz, CDCl₃) δ 3.72-3.89 (2.2H, m), 4.40-4.45 (0.14H, m), 4.61 (1H, t, J=2.4 Hz), 4.71 (1H, d, J=6.1 Hz), 4.77 (0.14H, dd, J=6.8, 4.2 Hz), 4.85 (0.14H, d, J=1.9 Hz), 4.94 (1H, d, J=6.1 Hz), 5.53 (0.14H, dd, J=9.9, 4.2 Hz), 5.58 (1H, d, J=2.5 Hz), 5.79 (1H, s), 6.00 (0.14H, s), 7.37-7.46 (3.42H, m), 7.49-7.58 (2.28H, m); ¹³C NMR (100 MHz, CDCl₃) δ 63.7, 82.6, 87.5, 102. 7, 105.8, 126.9, 128.4, 129.9, 135.7.

Compound 14 and its Intermediate

To a solution of 13 (1.19 g, 5 mmol) and K₂CO₃ (7.5 mmol) in MeOH (15 mL) was added HCHO (2.7 mL, 39.5% wt aq.). After stirring for 8 h at 85° C., the reaction mixture was neutralized with HCl (1M aq.) and concentrated. The remaining aqueous solution was extracted with CH₂C₂ (10 mL×5), and the combined organic layers were dried over sodium sulfate (Na₂SO₄), filtered and concentrated. The residue was purified by a silica gel column (hexane/EtOAc 5:1-1/1 gradient) to afford 14 as a yellow syrup (1.32 g, 98.5% yield). To a solution of 14 in MeOH (20 mL) was cautiously added NaBH₄ (10 mmol) in small portions. After stirring for 30 mins at r.t., the reaction mixture was cooled to 0° C. and then neutralized to pH of 7 with HCl (1M aq.). To the mixture was then added a solution of NaIO₄ (10 mmol) in H₂O (20 mL), and the reaction was stirred for an additional 1 h at r.t. After the solvent was evaporated, the remaining aqueous solution was extracted with EtOAc (50 mL×3). The combined organic layers were dried over Na₂SO₄, filtered and concentrated. The residue was purified by a silica gel column [petroleum ether (PE)/EtOAc 3:2] to give the intermediate as a colorless syrup (152 mg, 87% yield, α/β 9:1): ¹H NMR (400 MHz, CDCl₃) δ 3.83-3.99 (2.24H, m), 4.07-4.18 (2.24H, m), 4.44 (0.12H, d, J=3.4 Hz), 4.47 (1H, S), 5.14 (0.12H, dd, J=11.7, 3.4 Hz), 5.56 (1H, s), 5.92 (1H, s), 6.09 (0.12H, s), 7.35-7.45 (3.36H, m), 7.49-7.60 (2.24H, m); ¹³C NMR (100 MHz, CDCl₃) δ 63.2, 73.3, 87.2, 91.8, 101.1, 106.1, 127.0, 128.4, 130.0, 135.8.

Compound 15

Imidazole (3 eq.), TBDPSCl (1.2 eq.) and DMAP (0.05 eq.) were added to a solution of apiofuranose intermediate from previous step (190 mg, 0.8 mmol, 1 eq.) in CH₂C₂ (5 mL) at 0° C. After stirring at 20° C. for 12 h, the reaction mixture was diluted with EtOAc (20 mL) and washed with water (20 mL) and brine (20 mL×2). The organic layer was concentrated and purified by a silica gel column (PE/EtOAc 10:1-3:1 gradient) to give the desired product 15 as a colorless oil (240 mg, 63% yield): ¹H NMR (400 MHz, CDCl₃) δ 1.10-1.13 (10.26H, m), 3.65 (0.14H, d, J=10.7 Hz), 3.81 (0.14H, d, J=10.9 Hz), 3.87-3.92 (1H, m), 3.96-4.02 (1H, m), 4.04-4.21 (2.5H, m), 4.58 (0.14H, d, J=3.4 Hz), 4.63 (1H, s), 5.16 (0.14H, dd, J=11.9, 3.1 Hz), 5.59 (1H, d, J=4.1 Hz), 5.99 (1H, s), 6.10 (0.15H, s), 7.40-7.54 (13H, m), 7.71-7.76 (4H, m); ¹³C NMR (100 MHz, CDCl₃) δ 19.2, 26.8, 64.5, 73.0, 87.4, 92.1, 101.6, 106.4, 127.0, 127.8, 128.4, 128.8, 129.9, 1 130.0, 132.5, 132.6, 135.6, 135.7.

Compound 16

To an oven-dried 10-mL flask charging with diphyllin (7) (190 mg, 0.5 mmol), 15 (238 mg, 0.5 mmol) and PPh₃ (262 mg, 1 mmol) in THF (10 mL) was added DIAD (202 mg, 1 mmol) dropwise at 0° C. The reaction was completed in 2 h, and the reaction mixture was diluted with EtOAc (50 mL) and quenched with H₂O (20 mL). The organic layer was washed with brine (20 mL×2) and was then concentrated in vacuo to give a yellow solid which was purified by a silica gel column (PE/EtOAc 5:1-1:1 gradient) to give the α, β-isomers mixed product 16 as a white solid (325 mg, 78% yield).

Compounds 17 and 18

To a solution of 16 (100 mg) in THF (5 mL) was added TBAF (0.5 mL, 1M in THF/H₂O at a ratio of 95:5). The mixture was stirred at r.t. for 1 h. TLC showed the reaction was completed with 2 new spots formed. The mixture was concentrated in vacuo to give an oil and purified by prep-TLC (PE/EtOAc 1:2) to give 17 (6 mg, 8% yield) and 18 (42 mg, 59% yield), respectively. Compound 17: HRMS m/z [M+Na]+(calcd for C₃₃H₂₈O₁₁Na⁺, 623.1524; found 623.1520); ¹H NMR (400 MHz, CDCl₃) δ 3.81 (3H, s, —OCH₃), 4.07 (3H, s, —OCH₃), 4.14 (1H, d, J=12.0 Hz, H-5″), 4.17 (1H, d, J=12.0 Hz, H-5″), 4.28 (1H, d, J=12.0 Hz, H-4″), 4.36 (1H, d, J=12.0 Hz, H-4″), 5.08 (1H, s, H-2″), 5.49 (1H, dd, J=12.0, 1.6 Hz, H-12), 5.55 (1H, dd, J=12.0, 1.6 Hz, H-12), 5.83 (1H, s, H-1″), 6.05 (1H, d, J=1.4 Hz, H-7′), 6.09 (1H, s, —CHC₆H₅), 6.10 (1H, d, J=1.4 Hz, H-7′), 6.77-6.85 (2H, m, H-2′ and H-6′), 6.96 (1H, dd, J=7.8, 1.3 Hz, H-5′), 7.07 (1H, s, H-8), 7.39-7.45 (3H, m, —CHC₆H₅), 7.47 (1H, s, H-5), 7.51-7.55 (2H, m, —CHC₆H₅); ¹³C NMR (100 MHz, CDCl₃) δ 55.8 (—OCH₃), 56.0 (—OCH₃), 63.3 (C-12), 67.0 (C-5″), 74.5 (C-4″), 87.1 (C-2″), 92.2 (C-3″), 100.0 (C-1″), 101.2 (C-7′), 106.3 (—CHC₆H₅), 106.9 (C-8), 108.1 (C-5), 110.6 C-5′), 110.7 (C-2′), 119.3 (C-6′), 123.4 (C-9), 123.5 (C-10), 126.3 (C-1′), 127.0 (—CHC₆H₅), 127.1 (C-2), 128.2 (C-3), 128.6 (—CHC₆H₅), 130.2 (—CHC₆H₅), 130.7 (C-1), 135.6 (—CHC₆H₅), 135.7 (C-4), 143.7 (C-3′), 147.5 (C-4′), 150.3 (C-7), 151.8 (C-6), 169.6 (C-11); Compound 18: HRMS m/z [M+Na]+(calcd for C₃₃H₂₈O₁₁Na⁺, 623.1524; found 623.1523); ¹H NMR (400 MHz, CDCl₃) δ 3.42 (3H, s, —OCH₃), 3.78 (3H, s, —OCH₃), 3.99 (1H, d, J=10.0 Hz, H-5″), 4.07 (1H, d, J=10.0 Hz, H-5″), 4.16 (1H, d, J=9.9 Hz, H-4″), 4.46 (1H, d, J=9.9 Hz, H-4″), 4.84 (1H, d, J=4.4 Hz, H-2″), 5.45 (1H, dd, J=12.0, 1.6 Hz, H-12), 5.51 (1H, d, J=4.39 Hz, H-1″), 5.57 (1H, dd, J=12.0, 1.6 Hz, H-12), 6.05 (1H, s, H-7′), 6.09 (1H, t, J=1.38 Hz, H-7″), 6.42 (1H, s, —CHC₆H₅), 6.75-6.86 (2H, m, H-2′ and H-6′), 6.96 (1H, dd, J=7.9, 2.0 Hz, H-5′), 7.04 (1H, s, H-8), 7.33-7.43 (3H, m, —CHC₆H₅), 7.71-7.81 (3H, m, H-5 and —CHC₆H₅); ¹³C NMR (100 MHz, CDCl₃) δ 55.5 (—OCH₃), 55.7 (—OCH₃), 63.7 (C-12), 67.2 (C-5″), 72.0 (C-4″), 82.6 (C-2″), 91.3 (C-3″), 101.2 (C-1″), 101.4 (C-7′), 104.3 (—CHC₆H₅), 105.2 (C-8), 108.1 (C-5), 110.5 (C-5′), 110.7 (C-2′), 119.2 (C-6′), 123.4 (C-9), 123.5 (C-10), 127.4 (C-1′), 127.6 (—CHC₆H₅), 128.5 (—CHC₆H₅), 130.3 (—CHC₆H₅), 130.4 (C-2), 135.9 (C-3), 136.1 (C-4), 145.6 (C-3′), 147.4 (C-4′), 150.2 (C-7), 151.7 (C-6), 169.9 (C-11).

Compound 1 (tuberculatin)

To a mixture of 17 (6 mg) in THF/MeOH (4 mL, 1:3) was added palladium hydroxide[(Pd(OH)₂] (5 mg, 10% wt on carbon, dry). The mixture was degassed 3 times with H₂ and stirred at r.t. for 6 h under a hydrogen balloon. After TLC showed the the spot of 17 disappeared and a new spot with bigger polarity was formed, the reaction mixture was purified by a silica gel column (PE/EtOAc 3:1 and then CH₂Cl₂/MeOH 30:1) to give the desired product tuberculatin (1) (4.5 mg, 90%): HRMS m/z [M+Na]+(calcd for C₂₆H₂₄O₁₁Na⁺, 535.1211; found 535.1198): ¹H NMR (400 MHz, CDCl₃) δ 3.78 (3H, s, —OCH₃), 3.85 (1H, d, J=11.2 Hz, H-5″), 3.90 (1H, d, J=11.2 Hz, H-5″), 4.04 (3H, s, —OCH₃), 4.06 (1H, d, J=10 Hz, H-4″), 4.19 (1H, d, J=10 Hz, H-4″), 4.51 (1H, d, J=2.8 Hz, H-2″), 5.41-5.55 (3H, m, H-1″ and H-12), 6.05 (1H, s, H-7′), 6.08 (1H, s, H-7′), 6.73-6.82(2H, m, H-2′ and H-6′), 6.93 (1H, dd, J=8.0, 2.4 Hz, H-5′), 7.03 (1H, s, H-8), 7.55 (1H, s, H-5); ¹H NMR (400 MHz, DMSO-d₆) δ 3.44-3.50 (2H, m, H-5″), 3.67 (3H, s, —OCH₃), 3.78 (1H, d, J=9.4 Hz, H-4″), 3.96 (3H, s, —OCH₃), 4.24 (1H, d, J=8.9 Hz, H-4″), 4.41 (1H, dd, J=7.3, 3.8 Hz, H-2″), 4.79 (1H, s, C-3″ OH), 5.04 (1H, t, J=5.5 Hz, C-5″ OH), 5.47 (1H, t, J=4.0 Hz, H-1″), 5.46-5.54 (2H, m, H-12), 5.63 (1H, d, J=7.4 Hz, C-2″ OH), 6.13 (2H, s, H-7′), 6.80 (1H, d, J=7.9 Hz, H-6′), 6.92 (1H, s, H-2′), 7.00 (1H, s, H-8), 7.04 (1H, d, J=7.9 Hz, H-5′), 7.67 (1H, s, H-5); ¹H NMR (400 MHz, CD30D) δ 2.70 (1H, s, OH), 3.60-3.70 (2H, m, H-5″), 3.74 (3H, s, —OCH₃), 3.77 (1H, d, J=12.0 Hz, H-4″), 3.85 (1H, d, J=12.0 Hz, H-4″), 4.03 (3H, s, —OCH₃), 4.33 (1H, d, J=5.1 Hz, H-2″), 4.61 (1H, brs, C-5″ OH), 5.14 (1H, s, C-3″ OH), 5.55 (1H, dd, J=14.8, 4.0 Hz, H-12), 5.59 (1H, d, J=5.1 Hz, H-1″), 5.63 (1H, dd, J=14.8, 4.0 Hz, H-12), 6.05 (1H, d, J=1.1 Hz, H-7′), 6.06 (1H, d, J=1.1 Hz, H-7′), 6.76-6.83 (2H, m, H-2′ and H-6′), 6.97 (1H, d, J=7.8 Hz, H-5′), 7.07 (1H, s, H-8), 8.04 (1H, s, H-5); ¹³C NMR (100 MHz, CDCl₃) δ 55.8 (—OCH₃), 56.1 (—OCH₃), 65.5 (C-12), 67.2 (C-5″), 75.0 (C-4″), 78.7 (C-2″), 78.8 (C-3″), 100.4 (C-1″), 101.2 (C-7′), 106.2 (C-8), 108.2 (C-5), 110.6 (C-5′), 110.7 (C-2′), 111.1 (C-6′), 119.0(C-9), 123.6 (C-10), 126.7 (C-1′), 128.2 (C-2), 128.6 (C-3), 130.6 (C-1), 135.8 (C-4), 144.6 (C-3′), 147.5 (C-4′), 150.2 (C-7), 151.8 (C-6), 170.3 (C-11).

Compound 19

To the solution of 18 (20 mg) in THF/MeOH (4 mL, 1:3), Pd(OH)₂ (5 mg, 10% wt on carbon, dry) was added. The mixture was degassed 3 times with H₂ and stirred at r.t. for 6 h under a hydrogen balloon. TLC showed the 18 disappeared and a new spot was formed, the reaction mixture was purified by a silica gel column (PE/EtOAc 3:1 and then CH₂Cl₂/MeOH 30:1) to give 19 (15 mg, 88%): HRMS m/z [M+Na]+(calcd for C₂₆H₂₄O₁₁Na⁺, 535.1211; found 535.1224); ¹H NMR (400 MHz, DMSO-d₆) δ 3.38-3.46 (2H, m, H-5″), 3.67 (3H, s, —OCH₃), 3.95 (3H, s, —OCH₃), 4.01 (1H, dd, J=9.3, 3.1 Hz, H-4″), 4.10-4.19 (2H, m, H-2″ and H-4″), 4.78 (1H, s, —OH), 5.08 (1H, t, J=5.5 Hz, —OH), 5.26 (1H, d, J=9.2 Hz, H-1″), 5.51 (1H, s, H-12), 5.52 (1H, s, H-12), 5.54 (1H, t, J=4.8 Hz, —OH), 6.12 (2H, s, H-7′), 6.79 (1H, dd, J=8.0, 1.3 Hz, H-6′), 6.91 (1H, d, J=1.3 Hz, H-2′), 6.97 (1H, s, H-8), 7.03 (1H, d, J=7.9 Hz, H-5′), 8.03 (1H, s, H-5); ¹³C NMR (100 MHz, DMSO-d₆) δ 55.2 (—OCH₃), 55.8 (—OCH₃), 62.7 (C-12), 66.8 (C-5″), 71.7 (C-4″), 75.4 (C-2″), 76.0 (C-3″), 101.1 (C-1″), 102.1 (C-7′), 104.2 (C-8), 104.3 (C-5), 105.2 (C-5′), 107.9 (C-2′), 110.9 (C-6′), 118.7 (C-9), 123.6 (C-10), 127.2 (C-1′), 127.9 (C-2), 128.3 (C-3), 129.5 (C-1), 133.8 (C-4), 144.9 (C-3′), 146.9 (C-4′), 150.0 (C-7), 151.2 (C-6), 169.2 (C-11).

Compound 20

To a solution of 17 (15 mg, 0.025 mmol) in CH₂Cl₂ was added Et₃N (3 eq.) and DMAP (0.05 eq.) followed by addition of Ac₂O (2 eq.). The mixture was stirred at r.t. for 2 h and purified by prep-TLC to give the desired product 20 (12 mg) in 78% yield: HRMS m/z [M+Na]+(calcd for C₃₅H₃₀O₁₂Na⁺, 665.1629; found 665.1621); ¹H NMR (400 MHz, CDCl₃) δ 2.20 (3H, s, —COCH₃), 3.82 (3H, s, —OCH₃), 4.09 (3H, s, —OCH₃), 4.28 (1H, d, J=10.3 Hz, H-5″), 4.42 (1H, d, J=10.3 Hz, H-5″), 4.58 (1H, d, J=12.0 Hz, H-4″), 4.72 (1H, d, J=12.0 Hz, H-4″), 5.03 (1H, s, H-2″), 5.46-5.60 (2H, m, H-12), 5.83 (1H, s, H-1″), 6.04-6.08 (2H, m, H-7′), 6.11 (1H, d, J=1.5 Hz, —CHC₆H₅), 6.78-6.86 (2H, m, H-2′ and H-6′), 6.97 (1H, dd, J=7.9, 1.5 Hz, H-5′), 7.09 (1H, s, H-8), 7.38-7.48 (4H, m, H-5 and —CHC₆H₅), 7.50-7.57 (6H, m, —CHC₆H₅); ¹³C NMR (100 MHz, CDCl₃) δ 20.9 (—COCH₃), 55.8 (—OCH₃), 56.1 (—OCH₃), 64.1 (C-12), 67.0 (C-5″), 74.5 (C-4″), 87.5 (C-2″), 90.1 (C-3″), 99.8 (C-1″), 101.2 (C-7′), 106.3 (C-8), 106.9 (—CHC₆H₅), 108.1 (C-5), 108.2 (C-5′), 110.6 (C-2′), 119.3 (C-6′), 123.5 (C-9), 123.6 (C-10), 126.1 (C-1′), 127.0 (—CHC₆H₅), 127.2 (C-2), 128.3 (C-3), 128.5 (—CHC₆H₅), 130.3 (—CHC₆H₅), 130.7 (C-1), 135.2 (—CHC₆H₅), 135.7 (C-4), 143.7 (C-3′), 147.5 (C-4′), 150.2 (C-7), 151.9 (C-6), 169.5 (C-11), 170.6 (—COCH₃).

Compound 21

To a solution of 18 (15 mg, 0.025 mmol) in CH₂C₂ was added Et₃N (3 eq.), DMAP (0.05 eq.) followed by addition of Ac₂O (2 eq.). The mixture was stirred at r.t. for 2 h and was then purified by prep-TLC to give the desired product 21 (13 mg) in 85% yield: HRMS m/z [M+Na]+(calcd for C₃₅H₃₀O₁₂Na⁺, 665.1629; found 665.1625); ¹H NMR (400 MHz, CDCl₃) δ 2.20 (3H, s, —COCH₃), 3.39 (3H, s, —OCH₃), 3.78 (3H, s, —OCH₃), 4.15 (1H, d, J=10.2 Hz, H-5″), 4.42 (1H, d, J=12.0 Hz, H-4″), 4.52 (1H, d, J=10.2 Hz, H-5″), 4.64 (1H, d, J=11.9 Hz, H-4″), 4.82 (1H, d, J=4.4 Hz, H-2″), 5.40-5.48 (1H, m, H-12), 5.51 (1H, d, J=4.4 Hz, H-1″), 5.54-5.61 (1H, m, H-12), 6.05 (1H, d, J=1.4 Hz, H-7′), 6.10 (1H, d, J=1.6 Hz, H-7′), 6.42 (1H, s, —CHC₆H₅), 6.75-6.86 (2H, m, H-2′ and H-6′), 6.96 (1H, dd, J=7.8, 2.9 Hz, H-5′), 7.10 (1H, s, H-8), 7.33-7.43 (3H, m, —CHC₆H₅), 7.73 (1H, s, H-5), 7.74-7.81 (2H, m, —CHC₆H₅); ¹³C NMR (100 MHz, CDCl₃) δ 20.9 (—COCH₃), 55.3 (—OCH₃), 55.7 (—OCH₃), 64.4 (C-12), 67.1 (C-5″), 72.2 (C-4″), 83.0 (C-2″), 89.3 (C-3″), 101.2 (C-1″), 101.3 (C-7′), 104.1 (C-8), 105.7 (—CHC₆H₅), 108.1 (C-5), 110.6 (C-5′), 110.7 (C-2′), 110.9 (C-6′), 119.2 (C-1′), 123.4 (C-9), 123.5 (C-10), 127.4 (C-2), 127.7 (—CHC₆H₅), 127.8 (C-3), 128.5 (—CHC₆H₅), 130.3 (C-1), 130.4 (—CHC₆H₅), 135.7 (—CHC₆H₅), 136.0 (C-4), 145.5 (C-3′), 147.4 (C-4′), 150.2 (C-7), 151.7 (C-6), 169.7 (C-11), 170.6 (—COCH₃).

Compound 22

To a solution of 20 (6.4 mg) in THF/MeOH (1 mL, 1:3) was added Pd(OH)₂(1 mg, 10% wt on carbon, dry). The mixture was degassed 3 times with hydrogen gas and stirred for 12 h at r.t. under a hydrogen balloon. The mixture was then purified by prep-TLC (hexane/EtOAc 1:1) to give 22 in 91% yield (5.5 mg): HRMS m/z [M+Na]+(calcd for C₂₈H₂₆O₁₂Na⁺, 577.1316; found 577.1318); ¹H NMR (400 MHz, CDCl₃) δ 2.18 (3H, s, —COCH₃), 3.51-3.61 (2H, m, H-5″), 3.80 (3H, s, —OCH₃), 4.03-4.09 (4H, m, H-4″ and —OCH₃), 4.28 (1H, d, J=10.0 Hz, H-4″), 4.40-4.44 (1H, m, H-2″), 5.42-5.48 (1H, m, H-1″), 5.49-5.55 (2H, m, H-12″), 6.05 (1H, s, H-7′), 6.09 (1H, s, H-7′), 6.75-6.83 (2H, m, H-2′ and H-6′), 6.94 (1H, dd, J=7.9, 2.0 Hz, H-5′), 7.06 (1H, d, J=1.6 Hz, H-8), 7.57 (1H, s, H-5); ¹³C NMR (100 MHz, CDCl₃) δ 20.8 (—COCH₃), 55.8 (—OCH₃), 56.1 (—OCH₃), 66.5 (C-12), 67.2 (C-5″), 74.7 (C-4″), 77.7 (C-2″), 78.4 (C-3″), 100.4 (C-1″), 101.3 (C-7′), 106.3 (C-8), 108.2 (C-5), 110.6 (C-5′), 111.0 (C-2″), 119.1 (C-6′), 123.5 (C-9), 123.6 (C-10), 126.8 (C-1′), 128.3 (C-2), 128.9 (C-3), 130.7 (C-1), 136.0 (C-4), 144.7 (C-3′), 147.5 (C-4′), 150.3 (C-7), 151.9 (C-6), 170.2 (C-11), 171.8 (—COCH₃).

Compound 23

To a solution of 21 (10 mg) in THF/MeOH (1 mL, 1:3) was added Pd(OH)₂ (1 mg, 10% wt on carbon, dry). The mixture was degassed 3 times with hydrogen gas and stirred for 12 h at r.t. under a hydrogen balloon. The reaction mixture was purified by prep-TLC (hexane/EtOAc 1:1) to give 23 in 83% yield (8 mg): HRMS m/z [M+Na]+(calcd for C₂₈H₂₆O₁₂Na⁺, 577.1316; found 577.1317); ¹H NMR (400 MHz, CDCl₃) δ 2.18 (3H, s, —COCH₃), 3.81 (3H, s, —OCH₃), 4.05 (3H, s, —OCH₃), 4.19-4.24 (2H, m, H-5″), 4.25-4.31 (2H, m, H-4″), 4.34-4.38 (1H, m, H-2″), 5.37-5.44 (1H, m, H-1″), 5.48-5.57 (2H, m, H-12), 6.06 (1H, d, J=1.4 Hz, H-7), 6.10 (1H, d, J=1.3 Hz, H-7), 6.77-6.85 (2H, m, H-2′ and H-6′), 6.96 (1H, dd, J=7.8, 0.9 Hz, H-5′), 7.07 (1H, d, J=4.1 Hz, H-8), 7.77 (1H, s, H-5); ¹³C NMR (100 MHz, CDCl₃) δ 20.8 (—COCH₃), 55.9 (—OCH₃), 56.2 (—OCH₃), 66.8 (C-12), 67.1 (C-5″), 73.7 (C-4″), 75.8 (C-2″), (C-3″), 100.5 (C-1″), 101.3 (C-7′), 104.9 (C-8), 106.3 (C-5), 108.2 (C-5′), 110.7 (C-2′), 119.2 (C-6′), 123.5 (C-9), 123.6 (C-10), 127.2 (C-1′), 128.2 (C-2), 129.5 (C-3), 130.8 (C-1), 136.4 (C-4), 144.3 (C-3′), 147.6 (C-4′), 150.3 (C-7), 152.1 (C-6), 169.8 (C-11), 171.5 (—COCH₃).

Compound 25

Trifluoromethanesulfonic anhydride [(CF₃SO₂)₂₀] (2 eq.) was added dropwise to a solution of the 10 (11 mg, 0.02 mmol) in CH₂C₂ (1 mL) and pyridine (3 eq.) at −30° C. TLC analysis (EtOAc/cyclohexane 1:1) indicated the reaction was completed after stirring of 1 h. The crude mixture was diluted with H₂O and washed with CH₂Cl₂. The combined organic layers were concentrated in vacuo to yield the triflate derivative 24 which was dissolved in DMF (1 mL) without further purification and stirred with sodium azide (NaN₃) (0.1 mmol) at r.t. for 2 h. After TLC analysis (1:1 EtOAc/cyclohexane) showed a major product was formed, the reaction mixture was purified by a silica gel column (PE/EtOAc 5:1-2:1 gradient) to give the desired product 25 (9 mg) in 80% yield: HRMS m/z [M+Na]+(calcd for C₂₉H₂₇N3O₁₀Na⁺, 600.1588; found 600.1589); ¹H NMR (400 MHz, CDCl₃) δ 1.63 (3H, s, —C(CH₃)₂), 1.77 (3H, s, —C(CH₃)₂), 3.52 (1H, d, J=16 Hz, H-5″), 3.66 (1H, d, J=16 Hz, H-5″), 3.82 (3H, s, —OCH₃), 3.89 (1H, d, J=10.0 Hz, H-4″), 4.07 (3H, s, —OCH₃), 4.25 (1H, d, J=10.0 Hz, H₄″), 4.74 (1H, d, J=4.4 Hz, H-2″), 5.38 (1H, d, J=4.3 Hz, H-1″), 5.45 (1H, d, J=2.3 Hz, H-12), 5.55 (1H, d, J=2.3 Hz, H-12), 6.06 (1H, d, J=1.5 Hz, H-7′), 6.11 (1H, d, J=1.5 Hz, H-7′), 6.80-6.90 (2H, m, H-2′ and H-6′), 6.96 (1H, d, J=7.8 Hz, H-5′), 7.09 (1H, s, H-8), 7.79 (1H, s, H-5); ¹³C NMR (100 MHz, CDCl₃) δ 27.9 (—C(CH₃)₂), 28.7 (—C(CH₃)₂), 50.8 (C-5″), 55.0 (—OCH₃), 55.8 (—OCH₃), 56.1 (C-4″), 66.9 (C-12), 73.1 (C-2″), 83.2 (C-3″), 90.4 (C-1″), 100.8 (C-7′), 101.2 (C-8), 104.7 (C-5), 106.1 (C-5′), 108.2 (C-2′), 110.7 (C-6′), 117.8 (—C(CH₃)₂), 119.3 (C-9), 123.5 (C-10), 127.2 (C-1′), 128.1 (C-2), 129.0 (C-3), 130.6 (C-1), 136.2 (C-4), 145.4 (C-3′), 147.5 (C-4′), 150.3 (C-7), 151.9 (C-6), 169.7 (C-11).

Compound 26

Compound 25 (5.5 mg, 0.01 mmol) was dissolved in THF (2 mL) and degassed 3 times with N2. Trimethylphosphine (PMe₃) (0.5 mL, 1M in THF) was then added dropwise by syringe. The mixture was stirred at r.t. for 2 h, and then purified by a silica gel column (EtOAc/hexane 5:1) to give the desired product 26 (4.4 mg) in 80% yield; HRMS m/z [M+Na]+(calcd for C₂₉H₂₉NO₁₀Na⁺, 574.1683; found 574.1687); ¹H NMR (400 MHz, CD30D) δ 1.59 (3H, s, —C(CH₃)₂), 1.73 (3H, s, —C(CH₃)₂), 3.72 (3H, s, —OCH₃), 3.89 (1H, d, J=12.0 Hz, H-5″), 3.94 (1H, d, J=12.0 Hz, H-5″), 3.98-4.04 (4H, m, H-4″ and —OCH₃), 4.22 (2H, d, J=10.0 Hz, H-4″), 4.85-4.89 (1H, m, H-2″), 5.43-5.52 (2H, m, H-1″ and H-12), 5.54-5.62 (1H, m, H-12), 6.05 (2H, dd, J=7.2, 1.1 Hz, H-7′), 6.69-6.77 (2H, m, H-2′ and H-6′), 6.91-6.97 (1H, m, H-5′), 7.03 (1H, d, J=4.3 Hz, H-8), 7.80 (1H, s, H-5); ¹³C NMR (100 MHz, CD₃OD) δ 28.5 (—C(CH₃)₂), 29.0 (—C(CH₃)₂), 47.5 (C-5″), 56.2 (—OCH₃), 56.7 (—OCH₃), 68.7 (C-12), 74.2 (C-4″), 85.0 (C-2″), 92.2 (C-3″), 102.3 (C-1″), 102.8 (C-7′), 106.5 (C-8), 107.1 (C-5), 109.1 (C-5′), 111.9 (C-2′), 118.4 (—C(CH₃)₂), 120.3 (C-6′), 124.7 (C-9), 124.8 (C-10), 128.7 (C-1′), 130.1 (C-2), 130.2 (C-3), 131.9 (C-1), 137.3 (C-4), 147.0 (C-3′), 149.1 (C-4′), 152.0 (C-7), 153.6 (C-6), 172.1 (C-11).

Compound 27

To a solution of 10 (13 mg, 0.023 mmol) in CH₂C₂ (2 mL) was added Et₃N (3 eq.) and DMAP (0.05 eq.). After benzoyl chloride (2 eq.) was added, the reaction mixture was stirred at r.t. TLC showed the reaction was completed after 2 h, and the reaction mixture was purified by prep-TCL to give the desired product 27 in 75% yield (10 mg): HRMS m/z [M+Na]+(calcd for C₃₆H₃₂O₁₂Na⁺, 679.1786; found 679.1785); ¹H NMR (400 MHz, CDCl₃) δ 1.59 (3H, s, —C(CH₃)₂), 1.79 (3H, s, —C(CH₃)₂), 3.82 (3H, s, —OCH₃), 4.00-4.10 (4H, m, H-5″ and —OCH₃), 4.33 (1H, d, J=10.0 Hz, H-5″), 4.50-4.58 (1H, m, H-4″), 4.62-4.70 (1H, m, H-4″), 4.90 (1H, d, J=4.1 Hz, H-2″), 5.39-5.51 (2H, m, H-12), 5.55-5.63 (1H, m, H-1″), 6.06 (1H, s, H-7′), 6.11 (1H, s, H-7′), 6.78-6.89 (2H, m, H-2′ and H-5′), 6.97 (1H, d, J=7.8 Hz, H-5′), 7.09 (1H, s, H-8), 7.45-7.51 (2H, m, —OCOC₆H₅), 7.61 (1H, t, J=6.6 Hz, —OCOC₆H₅), 7.80 (1H, s, H-5), 8.03-8.11 (2H, m, —OCOC₆H₅); ¹³C NMR (100 MHz, CDCl₃) δ 27.9 (—C(CH₃)₂), 28.8 (—C(CH₃)₂), 55.9 (—OCH₃), 56.1 (—OCH₃), 65.7 (C-12), 67.0 (C-5″), 72.9 (C-4″), 83.3 (C-2″), 89.4 (C-3″), 100.8 (C-1″), 101.3 (C-7′), 104.9 (C-8), 106.1 (C-5), 108.2 (C-5′), 110.6 (C-2′), 110.7 (—C(CH₃)₂), 117.7 (C-6′), 119.4 (C-9), 123.5 (C-10), 123.6 (C-1′), 127.3 (C-2), 128.5 (C-3), 128.6 (—OCOC₆H₅), 129.0 (C-1), 129.7 (—OCOC₆H₅), 130.7 (—OCOC₆H₅), 133.6 (—OCOC₆H₅), 136.2 (C-4), 145.4 (C-3′), 147.5 (C-4′), 150.3 (C-7), 151.9 (C-6), 166.1 (—OCOC₆H₅), 169.7 (C-11).

Compound 28

To a solution of pyridine-4-carbonyl chloride (0.069 mmol) in CH₂C₂ (1 mL) was added Et₃N (10 eq.). The mixture was stirred at r.t. for 5 mins (precipitate of salt formed) and was then filtered and added to a solution of 10 (13 mg, 0.023 mmol) in CH₂Cl₂ (1 mL). The mixture was stirred at r.t. for 1 h and purified by prep-TLC to give the desired product 28 (12 mg) in 79% yield: HRMS m/z [M+Na]+(calcd for C₃₅H₃₁NO₁₂Na⁺, 680.1738; found 680.1741); ¹H NMR (400 MHz, CDCl₃) δ 1.58 (3H, s, —C(CH₃)₂), 1.79 (3H, s, —C(CH₃)₂), 3.82 (3H, s, —OCH₃), 4.05-4.11 (4H, m, H-5″-OCH₃), 4.35 (1H, d, J=10.2 Hz, H-5″), 4.56-4.62 (1H, m, H-4″), 4.66-4.72 (1H, m, H-4″), 4.89 (1H, d, J=4.3 Hz, H-2″), 5.41-5.49 (2H, m, H-1″ and H-12), 5.55-5.63 (1H, m, H-12), 6.06 (1H, t, J=1.4 Hz, H-7′), 6.11 (1H, d, J=1.4 Hz, H-7′), 6.78-6.87 (2H, m, H-2′ and H₆′), 6.94-7.00 (1H, m, H-5′), 7.10 (1H, s, H-8), 7.81 (1H, s, H-5), 7.82-7.95 (2H, m, —OCOC₅H₄N), 8.53-9.14 (2H, m, —OCOC₅H₄N); ¹³C NMR (100 MHz, CDCl₃) δ 27.9 (—C(CH₃)₂), 29.0 (—C(CH₃)₂), 55.9 (—OCH₃), 56.1 (—OCH₃), 66.5 (C-12), 66.9 (C-5″), 72.9 (C-4″), 83.3 (C-2″), 89.2 (C-3″), 100.8 (C-1″), 101.2 (C-7′), 104.7 (C-8), 106.1 (C-5), 108.2 (C-5′), 110.6 (C-2′), 110.7 (—C(CH₃)₂), 118.2 (—OCOC₅H₄N), 119.4 (C-6′), 123.5 (C-9), 123.6 (C-10), 127.2 (C-1′), 128.1 (C-2), 129.1 (C-3), 130.7 (C-1), 136.3 (—OCOC₅H₄N), 136.5 (C-4), 145.4 (C-3′), 147.5 (C-4′), 150.3 (—OCOC₅H₄N), 150.7 (C-7), 151.9 (C-6), 164.7 (—OCOC₅H₄N), 169.7 (C-11).

Compound 29

To a solution of pyridine-3-carbonyl chloride (0.069 mmol) in CH₂C₂ (1 Ml) was added Et₃N (10 eq.). The mixture was stirred at r.t. for 5 mins (precipitate of salt formed) and was then filtered and added to a solution of 10 (13 mg, 0.023 mmol) in CH₂C2 (1 Ml). The mixture was stirred at r.t. for 1 h and purified by prep-TLC to give the desired product 29 (14 mg) in 93% yield: HRMS m/z [M+Na]+(calcd for C₃₅H₃₁NO₁₂Na⁺, 680.1738; found 680.1742); ¹H NMR (400 MHz, CDCl₃) δ 1.57 (3H, s, —C(CH₃)₂), 1.78 (3H, s, —C(CH₃)₂), 3.81 (3H, s, —OCH₃), 4.04-4.09 (4H, m, H-5″ and —OCH₃), 4.33 (1H, d, J=10.2 Hz, H-5″), 4.64 (2H, d, J=5.6 Hz, H-4″), 4.92 (1H, dd, J=4.3, 1.1 Hz, H-2″), 5.44 (1H, dd, J=15.1, 2.1 Hz, H-12), 5.49 (1H, d, J=4.4 Hz, H-1″), 5.55-5.63 (1H, m, H-12), 6.03 (1H, dd, J=4.8, 1.4 Hz, H-7′), 6.08 (1H, dd, J=2.7, 1.4 Hz, H-7′), 6.75-6.84 (2H, m, H-2′ and H-6′), 6.91-6.97 (1H, m, H-5′), 7.07 (1H, s, H-8), 7.61 (1H, dd, J=7.7, 5.2 Hz, —OCOC₅H₄N), 7.81 (1H, s, H-5), 8.49 (1H, dt, J=8.0, 1.8 Hz, —OCOC₅H₄N), 8.95 (1H, s, —OCOC₅H₄N), 9.39 (1H, s, —OCOC₅H₄N.); ¹³C NMR (100 MHz, CDCl₃) δ 27.9 (—C(CH₃)₂), 29.0 (—C(CH₃)₂), 55.8 (—OCH₃), 56.1 (—OCH₃), 66.5 (C-12), 67.0 (C-5″), 72.8 (C-4″), 83.3 (C-2″), 89.1 (C-3″), 100.8 (C-1″), 101.2 (C-7′), 104.6 (C-8), 106.1 (C-5), 108.2 (C-5′), 110.7 (C-2′), 118.0 (—C(CH₃)₂), 119.3 (C-6′), 123.5 (C-9), 123.6 (C-10), 127.1 (C-1′), 127.2 (C-2), 128.0 (C-3), 128.9 (—OCOC₅H₄N), 129.0 (—OCOC₅H₄N), 130.6 (C-1), 136.1 (C-4), 139.1 (—OCOC₅H₄N), 145.3 (C-3′), 147.5 (C-4′), 150.3 (C-7), 151.8 (—OCOC₅H₄N), 151.9 (C-6), 153.1 (—OCOC₅H₄N), 163.9 (—OCOC₅H₄N), 169.7 (C-11).

Compound 30

The freshly prepared triflate derivative 24 from 10 (20 mg, 0.036 mmol) was dissolved in acetone (2 mL). Morpholine (3 eq.) and cecium carbonate (Cs₂CO₃) (3 eq.) were then added to the acetone solution. The mixture was stirred at r.t. for 2 h and then was purified by prep-TLC (PE/EtOAc 1:1) to give the desire product 30 (16 mg) in 74% yield: HRMS m/z [M+Na]+(calcd for C₃₃H₃₅NO₁₁Na⁺, 644.2102; found 644.2110); ¹H NMR (400 MHz, CDCl₃) δ 1.57 (3H, s, —C(CH₃)₂), 1.72 (3H, s, —C(CH₃)₂), 2.50-2.61 (2H, m, —NC₄H₈O), 2.71 (2H, s, H-5″), 2.71-2.80 (2H, m, —NC₄H₈O), 3.72 (4H, t, J=4.6 Hz, —NC₄H₈O), 3.82 (3H, s, —OCH₃), 4.07 (3H, s, —OCH₃), 4.09 (1H, d, J=9.8 Hz, H-4″), 4.17-4.22 (1H, m, H-4″), 4.60 (1H, d, J=4.1 Hz, H-2″), 5.39 (1H, d, J=4.1 Hz, H-1″), 5.42-5.49 (1H, m, H-12), 5.54-5.61 (1H, m, H-12), 6.06 (1H, t, J=1.4 Hz, H-7′), 6.10 (1H, d, J=1.5 Hz, H-7′), 6.83 (2H, s, H-2′ and H-5′), 6.94-6.99 (1H, m, H-6′), 7.08 (1H, s, H-8), 7.81 (1H, s, H-5); ¹³C NMR (100 MHz, CDCl₃) δ 27.0 (—C(CH₃)₂), 27.7 (—C(CH₃)₂), 54.2 (—NC₄H₈O), 54.8 (—OCH₃), 55.1 (—OCH₃), 62.7 (C-5″), 65.7 (—NC₄H₈O), 66.0 (C-12), 72.5 (C-4″), 83.3 (C-2″), 90.8 (C-3″), 100.0 (C-1″), 100.2 (C-7′), 103.7 (C-8), 105.1 (C-5), 107.2 (C-5′), 109.65 (C-2′) 109.7 (—C(CH₃)₂), 116.0 (C-6′), 118.4 (C-1′), 122.5 (C-9), 122.6 (C-10), 127.3 (C-2), 127.8 (C-3), 129.6 (C-1), 135.0 (C-4), 144.5 (C-3′), 146.5 (C-4′), 149.3 (C-7), 150.8 (C-6), 168.7 (C-11).

Compound 31

To the freshly prepared triflate derivative 24 from 10 (20 mg, 0.036 mmol) in acetone (2 mL) was added dimethylamine hydrochloride (3 eq.) and Cs₂CO₃ (5 eq.). The mixture was stirred at r.t. for 2 h and then was purified by prep-TLC (PE/EtOAc 1:1, v/v) to give the desire product 31 (15 mg) in 75% yield: HRMS m/z [M+Na]+(calcd for C₃₁H₃₃NO₁₀Na⁺, 602.1996; found 602.2003); ¹H NMR (400 MHz, CDCl₃) δ 1.61 (3H, s, —C(CH₃)₂), 1.77 (3H, s, —C(CH₃)₂), 2.27-2.32 (6H, m, —N(CH₃)₂) 3.74-3.78 (1H, m, H-5″), 3.82 (3H, s, —OCH₃), 3.83-3.86 (1H, m, H-5″), 4.05 (1H, d, J=10.0 Hz, H-4″), 4.07 (3H, s, —OCH₃), 4.28 (1H, d, J=10.0 Hz, H-4″), 4.85 (1H, d, J=4.1 Hz, H-2″), 5.39 (1H, d, J=4.1 Hz, H-1″), 5.42-5.48 (1H, m, H-12), 5.55-5.62 (1H, m, H-12), 6.06 (1H, t, J=1.4 Hz, H-7′), 6.11 (1H, d, J=1.4 Hz, H-7′), 6.79-6.86 (2H, m, H-2′ and H-6′), 6.95-6.99 (1H, m, H-5′), 7.09 (1H, s, H-8), 7.78 (1H, s, H-5); ¹³C NMR (100 MHz, CDCl₃) δ 27.9 (—C(CH₃)₂), 28.8 (—C(CH₃)₂), 46.5 (C-5″), 48.4 (—N(CH₃)₂, 55.9 (—OCH₃), 56.1 (—OCH₃), 66.8 (C-12), 72.9 (C-4″), 83.7 (C-2″), 90.4 (C-3″), 100.8(C-1″), 101.3 (C-7′), 105.1 (C-8), 106.3 (C-5), 108.2 (C-5′), 110.6 (C-2′), 117.7 (—C(CH₃)₂), 119.3 (C-6′), 123.5 (C-9), 123.6 (C-10), 127.2 (C-1′), 128.3 (C-2), 129.2 (C-3), 130.7 (C-1), 136.4 (C-4), 145.4 (C-3′), 147.5 (C-4′), 150.4 (C-7), 151.9 (C-6), 169.6 (C-11).

Compound 32

To a solution of freshly prepared triflate derivative 24 from 10 (20 mg, 0.036 mmol) in acetone (2 mL) was added dimethylamine acetamide (3 eq.) and Cs₂CO₃ (3 eq.). The mixture was stirred at r.t. for 2 h and was purified by prep-TLC (PE/EtOAc 11) to give the desire product in 59% yield (13 mg): HRMS m/z [M+Na]+(calcd for C₃₁H₃₁NO₁₁Na⁺, 616.1789; found 616.1794); H NMR (400 MHz, CDCl₃) δ 1.58 (3H, s, —C(CH₃)₂), 1.76 (3H, s, —C(CH₃)₂), 2.15 (3H, s, —NHCOCH₃), 3.82 (3H, s, —OCH₃), 3.94 (1H, d, J=10.0 Hz, H-5″), 4.07 (3H, s, —OCH₃), 4.22-4.32 (2H, m, H-5″ and H-4″), 4.36-4.43 (1H, m, H-4″), 4.77 (1H, d, J=4.3 Hz, H-2″), 5.38 (1H, d, J=4.1 Hz, H-1″), 5.41-5.48 (1H, m, H-12), 5.54-5.61 (1H, m, H-12), 6.04-6.08 (1H, m, H-7′), 6.11 (1H, d, J=1.3 Hz, H-7′), 6.79-6.86 (2H, m, H-2′ and H-6′), 6.97 (1H, d, J=7.9 Hz, H-5′), 7.09 (1H, s, H-8), 7.80 (1H, m, H-5); ¹³C NMR (100 MHz, CDCl₃) δ 20.8 (—NHCOCH₃), 27.9 (—C(CH₃)₂), 28.7 (—C(CH₃)₂), 55.8 (—OCH₃), 56.1 (—OCH₃), 65.4 (C-12), 66.9 (C-5″), 72.7 (C-4″), 83.1 (C-2″), 89.2 (C-3″), 100.8 (C-1″), 101.2 (C-7′), 104.8 (C-8), 106.1 (C-5), 108.2 (C-5′), 110.7 (C-2′), 117.8 (—C(CH₃)₂), 119.4 (C-6′), 123.5 (C-9), 123.7 (C-10), 127.2 (C-1′), 128.2 (C-2), 129.1 (C-3), 130.7 (C-1), 136.2 (C-4), 145.4 (C-3′), 147.5 (C-4′), 150.3 (C-7), 151.9 (C-6), 169.7 (C-11), 170.6 (—NHCOCH₃).

Compound 34

To a solution of 6-bromoveratraldehyde (33) (3.48 g, 14.20 mmol) in toluene (50 mL) was added ethylene glycol (1.76 g, 28.38 mmol) and β-toluenesulfonic acid (TsOH-H₂O) (0.27 g, 14.21 mmol). A Dean-Stark apparatus filled with toluene was fitted to the round bottom flask and the reaction was refluxed for 8 h. After cooling, the reaction was quenched with Et₃N (0.5 mL), and the mixture was washed with water (2×20 mL) and brine (20 mL). After the organic solvent was removed under vacuum, the obtained mixture was triturated with EtOH. The obtained solid was dried under vacuum to provide 34 as a white solid (3.53 g, 85.8%); HRMS(ESI) m/z: [M+H]⁺ Calcd for C₁₁H₁₄BrO₄, 289.0070; found, 289.0098.

Compound 36

To a solution of sesamol (35, 1.40 g, 10.14 mmol), anhydrous magnesium chloride (MgCl₂)(1.50 g, 15.78 mmol), and Et₃N (5 mL) in anhydrous THF (50 mL) was added parafomaldehyde (1.50 g, 49.95 mmol), and the mixture was refluxed at 80° C. for 10 h. After the mixture was cooled to r.t., the reaction was quenched with 3M HCl (1.5 mL). The mixture was extracted with EtOAc (3×50 mL), which were combined and further washed with brine, dried over anhydrous Na₂SO₄, filtered and concentrated. The residue was purified by column chromatography on a silica gel column by elution with petroleum ether (PE):EtOAc 10:1 to afford compound 36 as a light yellow solid (1.53 g, 90.9%); HRMS(ESI) m/z: [M+H]⁺ Calcd for C8H₇O₄, 167.0339; found, 167.0340.

Compound 37

To a solution of 36 (0.53 g, 3.16 mmol) and Cs₂CO₃ (2.01 g, 6.14 mmol) in anhydrous DMF (15 mL) was added benzyl bromide (BnBr) (1.00 g, 0.72 mL, 5.85 mmol) under argon, and the mixture was refluxed for 5 h (80° C.). After the mixture was cooled to r.t., the reaction was quenched with 3M HCl (1.5 mL), and extracted with EtOAc (3×50 mL). The combined organic extracts were washed with brine, dried over anhydrous Na₂SO₄, filtered and concentrated. The residue was purified by column chromatography on silica gel (PE: EtOAc, 8:1) to afford compound 37 as a light yellow solid (0.74 g, 91.3%); HRMS(ESI) m/z: [M+H]⁺ Calcd for C₁₅H₁₃O₄, 257.0808; found, 257.0756.

Compound 38

Compound 37 (794.90 mg, 2.76 mmol) was dissolved in dry THF (10 mL) under nitrogen, and cooled to −78° C. over 20 min. The solution was added (n-butyllithium) n-BuLi (2.5M in hexanes, 1 mL, 2.50 mmol) dropwisely. The mixture was stirred for another 20 min, followed by the addition of the THF (3 mL) solution of compound 37 (627.42 mg, 2.45 mmol) dropwisely. After stirring for 3 h, the mixture was gradually warmed to r.t., followed by the addition of H₂O (10 mL). The reaction mixture was then extracted with EtOAc (2×50 mL). The combined organic extracts were washed with brine, dried over anhydrous Na₂SO₄, filtered and concentrated to afford 38 as a white solid (1.10 g, 96.3%); HRMS(ESI) m/z: [M+H]⁺ Calcd for C₂₆H₂₇O₈, 467.1700; found, 467.1637.

Compound 39

Compound 38 (1.10 g, 2.36 mmol), dimethyl acetylenedicarboxylate (DMADC) (433.73 mg, 2.97 mmol) and acetic acid (AcOH) (1 mL) were added in CH₂Cl₂ (1.5 mL), and the mixture was heated at 80° C. for 1 h. After a work-up, the mixture was gradually warmed to r.t., followed by addition of H₂O (10 mL). The mixture was then extracted with EtOAc (2×50 mL). The combined organic solutions were washed with brine, dried over anhydrous Na₂SO₄, filtered and concentrated to give a light green solid, which was purified by column chromatography on silica gel (PE: EtOAc 4:1) to give the desired product 39 (550.53 mg, 42.7%); HRMS(ESI) m/z: [M+H]⁺ Calcd for C₃₀H₂₇O₁₀, 547.1599; found, 547.1607; ¹H-NMR (DMSO-d₆, 400 MHz) δ_{H} 11.76 (1H, s), 8.31 (1H, s), 7.64 (1H, s), 7.11-7.17 (3H, m), 6.92-6.94 (2H, m), 6.65 (1H, s), 6.64 (1H, s), 6.04 (1H, d, J=1.0 Hz), 6.05 (1H, d, J=1.0 Hz), 4.93 (2H, s), 3.93 (3H, s), 3.88 (3H, s), 3.60 (3H, s), 3.53 (3H, s).

Compound 40

To a solution of compound 39 (191.15 mg, 0.35 mmol) in dry THF (4 mL) was added NaBH₄ (66.20 mg, 1.75 mmol), and the reaction was allowed to reflux for 4 h. After the solution was cooled to r.t., HCl (3 mol/L) was added to adjust the pH value to 2-3 (approximate 1.5 mL), and the mixture was stirred for additional 1 h. The reaction mixture was extracted with EtOAc (3×50 mL). The combined organic extracts were washed with brine, dried over anhydrous Na₂SO₄, filtered and concentrated, and further purified by a silica gel column (PE: EtOAc 1:1) to give the desired product 20 as a white solid (102.10 mg, 60.0%); HRMS(ESI) m/z: [M+H]⁺ Calcd for C₂₈H₂₃O₈, 487.1387; found, 487.1355; ¹H-NMR (DMSO-d₆, 400 MHz) δ_{H} 10.37 (1H, s, 4-OH), 7.62 (1H, s, H-5), 6.95 (1H, s, H-8), 7.11-7.18 (3H, m, H-11′-13′), 6.88-6.91 (2H, m, H-10′, 14′), 6.85 (1H, s, H-2′), 6.74 (1H, s, H-5′), 6.06 (1H, d, J=1.0 Hz, H-7′), 6.07 (1H, d, J=1.0 Hz, H-7′), 5.35 (2H, s, H-8′), 4.86 (1H, d, J=12.3 Hz, H-12), 4.92 (1H, d, J=12.3 Hz, H-12), 3.94 (3H, s, 6-OCH₃), 3.61 (3H, s, 7-OCH₃).

Compound 42

An oven-dried 25-ml flask charging with 40 (102.10 mg, 0.21 mmol), D-apiose derivative (41) (142.86 mg, 0.30 mmol) and PPh₃ (104.91 mg, 0.40 mmol) in THF (8 mL) was added DIAD (80.88 mg, 0.40 mmol) at 0° C. under nitrogen protection. After the reaction was stirred at r.t. for 2 h, the reaction mixture was extracted with EtOAc (3×50 mL). The combined organic extracts were washed with brine, dried over anhydrous Na₂SO₄, filtered and concentrated, and further purified by a silica gel column (PE: EtOAc 2:3) to give the desired product 21 as a light yellowed solid (190.20 mg, 95.9%, mixture); HRMS(ESI) m/z: [M+H]⁺ Calcd for C₅₆H₅₃O₁₂Si, 945.3301; found, 945.2910; ¹H-NMR (CDCl₃, 400 MHz) δ_{H} 7.43 (1H, s, H-5), 6.98 (1H, s, H-8), 7.13-7.17 (3H, m, H-11′-13′), 6.93-6.99 (2H, m, H-10′, 14′), 6.70 (1H, s, H-5′), 6.72 (1H, s, H-2′), 5.99 (1H, d, J=1.3 Hz, H-7′), 6.04 (1H, d, J=1.3 Hz, H-7′), 5.51 (2H, s, H-8′), 4.23 (1H, d, J=11.0 Hz, H-12), 4.43 (1H, d, J=11.0 Hz, H-12), 3.42 (3H, s, 6-OCH₃), 3.76 (3H, s, 7-OCH₃). 5.42(1H, s, H-1″), 4.04 (1H, d, J=12.0 Hz, H-4″), 4.15(1H, d, J=12.0 Hz, H-4″), 4.14 (1H, s, H-2″), 4.90 (2H, s, H-6″), 6.46 (1H, s, H-5″), 7.40-7.42 (3H, m, H-9″-11″), 7.51-7.53 (2H, m, H-8″, H-12″), 7.42-7.47 (4H, m, H-2′″, H-6′″), 7.70-7.78 (6H, m, H-3′″-5′″), 1.14-1.15 (δ_{H}, s, H-8′″-10′″).

Compounds 43 and 44

To a solution of compound 42 (100.10 mg, 0.10 mmol) in THF (4 mL) was added TBAF (0.8 mL, 1 M in THF/H₂O, 95/5). The mixture was stirred at r.t. for 1 h. TLC showed the reaction was completed, and two new spots with higher polarity were formed. The mixture was concentrated in vacuo to give an oil, which was separated by a silica gel column (PE: EtOAc 1:1) to afford 22a (25.33 mg, 35.8%) and 22b (45.75 mg, 61.1%). Compound 43: HRMS(ESI) m/z: [M+H]⁺ Calcd for C₄₀H₃₅O₁₂, 707.2123; found, 707.1945 ¹H-NMR (CDCl₃, 400 MHz) δ_{H} 7.46 (1H, s, H-5), 7.01 (1H, s, H-8), 7.13-7.16 (3H, m, H-11′-13′), 6.92-6.95 (2H, m, H-10′, 14′), 6.68 (1H, s, H-5′), 6.70 (1H, s, H-2′), 5.98 (1H, d, J=1.3 Hz, H-7′), 6.03 (1H, d, J=1.3 Hz, H-7′), 5.48 (2H, s, H-8′), 4.26 (1H, d, J=11.0 Hz, H-12), 4.35 (1H, d, J=11.0 Hz, H-12), 4.06 (3H, s, 6-OCH₃), 3.77 (3H, s, 7-OCH₃), 5.83 (1H, s, H-1″), 4.13 (1H, d, J=12.0 Hz, H-4″), 4.17 (1H, d, J=12.0 Hz, H-4″), 5.06 (1H, s, H-2″), 4.87 (2H, s, H-6″), 6.08 (1H, s, H-5″), 7.40-7.42 (3H, m, H-9″-11″), 7.52-7.55 (2H, m, H-8″, H-212″); Compound 44: HRMS(ESI) m/z: [M+H]⁺ Calcd for C₄₀H₃₅O₁₂, 707.2123; found, 707.1943 ¹H-NMR (CDCl₃, 400 MHz) δ_{H} 7.72 (1H, s, H-5), 6.96 (1H, s, H-8), 7.12-7.16 (3H, m, H-11′-13′), 6.92-6.95 (2H, m, H-10′, 14′), 6.67 (1H, s, H-5′), 6.70 (1H, s, H-2′), 5.97 (1H, d, J=1.3 Hz, H-7′), 6.02 (1H, d, J=1.3 Hz, H-7′), 5.49 (2H, s, H-8′), 5.54 (1H, d, J=11.0 Hz, H-12), 5.46 (1H, d, J=11.0 Hz, H-12), 3.40 (3H, s, 6-OCH₃), 3.76 (3H, s, 7-OCH₃). 5.49 (1H, d, J=4.3 Hz, H-1″), 4.83 (1H, d, J=4.3 Hz, H-1″), 3.98 (1H, d, J=11.7 Hz, H-4″), 4.04 (1H, d, J=11.7 Hz, H-4″), 4.87 (2H, s, H-6″), 6.41 (1H, s, H-5″), 7.33-7.39 (3H, m, H-9″-11″), 7.74-7.76 (2H, m, H-8″, H-12″).

Compounds 45 and 46

To a solution of 43 (25.13 mg, 0.04 mmol) in the mixed solvents of THF: MeOH (1:3, 4 mL) was added Pd(OH)₂ on carbon (4 mg, 20%). The reaction was degassed 3 times with H₂ and stirred for 6 h under H₂ balloon. TLC showed the 43 disappeared and two new spots with higher polarity were formed. The mixture was purified by a silica gel column (PE: EtOAc 1:1) to provide compounds 45 (7.80 mg, 42.2%) and 46 (8.10 mg, 43.8%). Compound 45: HRMS(ESI) m/z: [M+H]⁺ Calcd for C₂₆H₂₅O₁₂, 529.1346; found, 529.1247; ¹H-NMR (CD₃OD, 400 MHz) & 7.701 (1H, s, H-5), 7.075 (1H, s, H-8), 6.567 (1H, s, H-2′), 6.543 (1H, s, H-5′), 5.946 (1H, d, J=1.0 Hz, H-7′), 5.968 (1H, d, J=1.0 Hz, H-7′), 5.498 (1H, d, J=14.8 Hz, H-12), 5.572 (1H, d, J=14.8 Hz, H-12), 4.019 (3H, s, 6-OCH₃), 3.761 (3H, s, 7-OCH₃), 5.531 (1H, d, J=3.6 Hz, H-1″), 4.516 (1H, d, J=3.6 Hz, H-2″), 4.345 (1H, d, J=9.7 Hz, H-4″), 3.932 (1H, d, J=9.7 Hz, H-4″), 3.668 (1H, d, J=11.4 Hz, H-5″), 3.710 (1H, d, J=11.4 Hz, H-5″); CD (MeOH), (Δε) 200 (−17.73), 212 (0.95), 217 (−0.34), 229 (9.36), 246 (−1.52), 262 (1.89), 276 (−2.36), 293 (−0.41), 315 (−2.03) nm; Compound 46: HRMS(ESI) m/z: [M+H]⁺ Calcd for C₂₆H₂₅O₁₂, 529.1346; found, 529.1287; ¹H-NMR (CD₃OD, 400 MHz) δ_{H}7.698 (1H, s, H-5), 7.076 (1H, s, H-8), 6.561 (1H, s, H-2′), 6.545 (1H, s, H-5′), 5.947 (1H, d, J=1.0 Hz, H-7′), 5.969 (1H, d, J=1.0 Hz, H-7′), 5.495 (1H, d, J=14.8 Hz, H-12), 5.569 (1H, d, J=14.8 Hz, H-12), 4.021 (3H, s, 6-OCH₃), 3.762 (3H, s, 7-OCH₃), 5.538 (1H, d, J=3.6 Hz, H-1″), 4.518 (1H, d, J=3.6 Hz, H-2″), 4.350 (1H, d, J=9.7 Hz, H-4″), 3.934 (1H, d, J=9.7 Hz, H-4″), 3.672 (1H, d, J=11.4 Hz, H-5″), 3.713 (1H, d, J=11.4 Hz, H-5″); CD (MeOH) λ (Δε) 200 (23.17), 211 (−6.35), 218 (−3.32), 229 (−16.06), 246 (2.85), 260 (−4.02), 274 (4.66), 297 (−0.19), 313 (0.76) nm.

Compounds 47 and 48

To a solution of 44 (25.20 mg, 0.04 mmol) in THF: MeOH (1:3, 4 mL) was added Pd(OH)₂ on carbon (4 mg, 20%). The reaction was degassed 3 times with H₂ and stirred for 6 h under H₂ balloon. TLC showed the 44 disappeared, and two new spots with higher polarity were formed. The mixture was purified by a silica gel column (PE: EtOAc 1:1) to afford compound 47 (8.23 mg, 43.3%) and 48 (8.27 mg, 43.5%). Compound 47: HRMS(ESI) m/z: [M+H]⁺ Calcd for C₂₆H₂₅O₁₂, 529.1346; found, 529.1278; ¹H-NMR (CD30D, 400 MHz) δ_{H} 8.001 (1H, s, H-5), 7.074 (1H, s, H-8), 6.560 (1H, s, H-2′), 6.552 (1H, s, H-5′), 5.938 (1H, d, J=1.0 Hz, H-7′), 5.970 (1H, d, J=1.0 Hz, H-7′), 5.585 (1H, d, J=14.8 Hz, H-12), 5.506 (1H, d, J=14.8 Hz, H-12), 4.037 (3H, s, 6-OCH₃), 3.791 (3H, s, 7-OCH₃), 5.482 (1H, d, J=4.7 Hz, H-1″), 4.235 (1H, d, J=4.7 Hz, H-2″), 4.203 (1H, d, J=9.9 Hz, H-4″), 4.232 (1H, d, J=9.9 Hz, H-4″), 3.645 (1H, d, J=11.4 Hz, H-5″), 3.613 (1H, d, J=11.4 Hz, H-5″); CD (MeOH) λ (Δε): 200 (−29.98), 212 (11.95), 219 (6.87), 229 (24.50), 247 (−4.84), 263 (4.99), 275 (−6.53), 293 (−0.34), 313 (−1.44) nm; Compound 48: HRMS(ESI) m/z: [M+H]⁺ Calcd for C₂₆H₂₅O₁₂, 529.1346; found, 529.1287; ¹H-NMR (CD₃OD, 400 MHz), H 7.965 (1H, s, H-5), 7.073 (1H, s, H-8), 6.554 (1H, s, H-2′), 6.548 (1H, s, H-5′), 5.929 (1H, d, J=1.0 Hz, H-7′), 5.965 (1H, d, J=1.0 Hz, H-7′), 5.488 (1H, d, J=14.8 Hz, H-12), 5.577 (1H, d, J=14.8 Hz, H-12), 4.028 (3H, s, 6-OCH₃), 3.793 (3H, s, 7-OCH₃), 5.451 (1H, d, J=4.6 Hz, H-1″), 4.218 (1H, d, J=4.6 Hz, H-2″), 4.221 (1H, d, J=9.9 Hz, H-4″), 4.195 (1H, d, J=9.9 Hz, H-4″), 3.610(1H, d, J=11.2 Hz, H-5″), 3.642 (1H, d, J=11.2 Hz, H-5″); CD (MeOH) λ (Δε): 200 (24.69), 212 (−0.50), 218 (0.808), 229 (−11.93), 246 (1.61), 262 (−3.42), 275 (4.40), 291 (1.36), 314 (2.82) nm.

Compound 49

Colorless power; [α]_{D}²⁰ −39.1 (c 0.05, MeOH); UV (MeOH) λₘₐₓ (log ε): 200 (2.86), 229 (2.63), 266 (2.97), 310 (sh) (2.42), 361 (sh) (2.00) nm; CD (MeOH) λ (Δε): 200 (−22.84), 212 (1.74), 217 (0.40), 229 (11.53), 245 (−0.62), 262 (4.37), 274 (−3.46), 292 (−1.19), 311 (−3.07), 334 (0.95) nm; IR (KBr) vₘₐₓ: 3414, 2932, 1745, 1625, 1508, 1484, 1456, 1435, 1385, 1342, 1264, 1244, 1216, 1167, 1123, 1054, 1037, 993, 934, 858, 769 cm⁻¹; HRMS (ESI) m/z: [M+H]⁺ Calcd for C₃₁H₃₃O₁₆, 661.1769; found, 661.1805; ¹H and ¹³C NMR spectral data (Table 2).

Compound 50

White power; [α]_{D}²⁰ −37.5° (c 0.05, MeOH); UV (MeOH) λₘₐₓ (log ε): 202 (3.09), 229 (2.86), 265 (3.17), 310 (sh) (2.63), 359 (sh) (2.17) nm; CD (MeOH) λ (Δε): 200 (26.93), 211 (−6.27), 218 (−3.28), 229 (−17.54), 248 (5.24), 264 (−2.48), 275 (5.52), 298 (−0.36), 310 (0.47), 332 (−1.56), 348 (−0.21) nm; IR (KBr) vₘₐₓ: 3400, 2927, 1745, 1625, 1508, 1484, 1456, 1436, 1386, 1341, 1264, 1244, 1216, 1168, 1123, 1053, 992, 935, 859, 768 cm¹; HRMS (ESI) m/z: [M+H]⁺ Calcd for C₃₁H₃₃O₁₆, 661.1769; found, 661.1805; ¹H and ¹³C NMR spectral data (Table 2).

Compound 51

White power; [α]_{D}²⁰ −39.6° (c 0.05, MeOH); UV (MeOH) λₘₐₓ (log ε): 200 (2.86), 225 (2.64), 260 (2.97), 307 (sh) (2.44), 355 (sh) (2.19) nm; CD (MeOH) λ (Δε): 200 (−27.24), 211 (3.46), 218 (0.82), 229 (15.28), 246 (−1.05), 261 (4.59), 275 (−4.40), 292 (−1.32), 311 (−3.49), 334 (1.41) nm; IR (KBr) vₘₐₓ: 3410, 2927, 1745, 1625, 1508, 1484, 1454, 1436, 1389, 1344, 1264, 1244, 1216, 1168, 1052, 993, 938, 858, 769 cm⁻¹; HRMS (ESI) m/z: [M+H]⁺ Calcd for C₃₆H₄₁O₂₀, 793.2191; found, 793.2219; ¹H and ¹³C NMR spectral data (Table 3).

Compound 52

Colorless power; [α]_{D}²⁰ −50.2° (c 0.05, MeOH); UV (MeOH) λₘₐₓ (log ε): 202 (2.86), 228 (2.64), 262 (2.97), 309 (sh) (2.44), 362 (sh) (2.19) nm; CD (MeOH) λ (Δε): 200 (29.36), 211 (−6.93), 218 (−4.61), 229 (−19.86), 246 (5.92), 262 (−3.66), 275 (5.62), 296 (0.09), 313 (0.70), 332 (−1.69), 348 (−0.30) nm; IR (KBr) vₘₐₓ: 3410, 2923, 1744, 1625, 1508, 1484, 1455, 1436, 1385, 1342, 1264, 1244, 1216, 1168, 1052, 993, 938, 860, 769 cm⁻¹; HRMS (ESI) m/z: [M+H]⁺ Calcd for C₃₆H₄₁O₂₀, 793.2191; found, 793.2244; ¹H and ¹³C NMR spectral data (Table 3).

Compound 53

White power; [α]_{D}²⁰ −43.7° (c 0.05, MeOH); UV (MeOH) λₘₐₓ (log ε): 200 (3.20), 228 (2.98), 262 (3.31), 309 (sh) (2.78), 362 (sh) (2.27) nm; CD (MeOH) λ (Δε): 200 (−23.39), 210 (2.31), 218 (0.33), 229 (12.88), 248 (−1.19), 260 (3.86), 275 (−3.60), 293 (−0.70), 313 (−2.84), 333 (0.64) nm; IR (KBr) vₘₐₓ: 3427, 2926, 1746, 1626, 1507, 1483, 1436, 1390, 1345, 1264, 1244, 1214, 1168, 1054, 994, 938, 858, 769 cm¹; HRMS (ESI) m/z: [M+Na]⁺ Calcd for C₄₁H₄₈NaO₂₄, 947.2428; found, 947.2494; ¹H and ¹³C NMR spectral data (Table 4).

Compound 54

White power; [α]D (c 0.05, MeOH); UV (MeOH)λₘₐₓ (log ε): 202 (3.00), 228 (2.76), 260 (3.10), 309 (sh) (2.54), 359 (sh) (2.27) nm; CD (MeOH) λ (Δε): 200 (28.16), 210 (−5.2), 219 (−2.85), 229 (−18.81), 248 (5.87), 262 (−3.39), 275 (5.02), 296 (−0.27), 315 (0.99), 331 (−1.74), 343 (−0.48) nm; IR (KBr) vₘₐₓ: 3430, 2964, 1745, 1626, 1508, 1484, 1436, 1384, 1341, 1262, 1245, 1216, 1168, 1053, 994, 939, 801, 769 cm¹; HRMS (ESI) m/z: [M+Na]⁺ Calcd for C₄₁H₄₈NaO₂₄, 947.2428; found, 947.2493; ¹H and ¹³C NMR spectral data (Table 4).

TABLE 2
¹H (400 MHz) and ¹³C (100 MHZ) NMR data of compounds 49 and
50 in CD₃OD (δ in ppm, J in Hz).

| | 49 | | 50 | |
|---|---|---|---|---|
| Position | δ_{H} (J in Hz) | δ_{C}, tpye | δ_{H} (J in Hz) | δ_{C}, tpye |
| 1 | | 133.98, C | | 133.84, C |
| 2 | | 121.05, C | | 120.90, C |
| 3 | | 130.34, C | | 130.66, C |
| 4 | | 146.10, C | | 145.88, C |
| 5 | 7.666, s | 102.09, CH | 7.587, s | 102.47, CH |
| 6 | | 153.25, C | | 153.17, C |
| 7 | | 151.64, C | | 151.52, C |
| 8 | 7.074, s | 107.32, CH | 7.023, s | 153.17, CH |
| 9 | | 132.05, C | | 131.86, C |
| 10 | | 128.26, C | | 128.16, C |
| 11 | | 172.21, C | | 172.15, C |
| 12 | 5.479, d, (15.1) | 68.84, CH₂ | 5.475, d, (15.1) | 68.83, CH₂ |
| | 5.544, d, (15.1) | | 5.516, d, (15.1) | |
| 6-OCH₃ | 4.026,s | 56.81, CH₃ | 4.007, s | 56.81, CH₃ |
| 7-OCH₃ | 3.757, s | 56.03, CH₃ | 3.740, s | 56.00, CH₃ |
| 1′ | | 114.75, C | | 114.63, C |
| 2′ | 6.544, s | 98.93, CH | 6.471, s | 98.90, CH |
| 3′ | | 142.18, C | | 142.05, C |
| 4′ | | 149.79, C | | 149.72, C |
| 5′ | 6.579, s | 111.41, CH | 6.524, s | 111.41, CH |
| 6′ | | 150.78, C | | 150.77, C |
| 7′ | 5.949, d (1.0) | 102.51, CH₂ | 5.959, d (1.0) | 102.47, CH₂ |
| 1″ | 5.664, d (2.7) | 111.35, CH | 5.626, d (2.7) | 111.46, CH |
| 2″ | 4.715, d (2.7) | 85.97, CH | 4.724, d(2.7) | 85.94, CH |
| 3″ | | 81.24, C | | 81.22, C |
| 4″ | 3.911, d (9.7) | 75.69, CH₂ | 3.907, d (9.7) | 75.64, CH₂ |
| | 4.323, d (9.7) | | 4.329, d (9.7) | |
| 5″ | 3.667, d (11.4) | 64.11, CH₂ | 3.776, d (11.4) | 64.13, CH₂ |
| | 3.720, d (11.4) | | 3.729, d (11.4) | |
| 1′′′ | 4.534, d (7.5) | 106.17, CH | 4.543, d (7.5) | 106.10, CH |
| 2′′′ | 3.296, m | 75.20, CH | 3.363, m | 75.19, CH |
| 3′′′ | 3.369, m | 77.67, CH | 3.387, m | 77.65, CH |
| 4′′′ | 3.471, m | 71.08, CH | 3.473, m | 71.07, CH |
| 5′′′ | 3.245, dd (10.5, | 67.29, CH₂ | 3.269, dd (10.5, | 67.25, CH₂ |
| | 11.4) | | 11.4) | |
| | 3.808, dd (5.3, | | 3.845, dd (5.3, | |
| | 11.4) | | 11.4) | |

TABLE 3
¹H (400 MHZ) and ¹³C (100 MHZ) NMR data of compounds
51 and 52 in CD₃OD (δ in ppm, J in Hz).

| | 51 | | 52 | |
|---|---|---|---|---|
| Position | δ_{H} (J in Hz) | δ_{C}, type | δ_{H} (J in Hz) | δ_{C}, type |
| 1 | | 134.14, C | | 133.86, C |
| 2 | | 121.08, C | | 120.79, C |
| 3 | | 130.65, C | | 131.01, C |
| 4 | | 146.18, C | | 145.77, C |
| 5 | 7.634, s | 102.13, CH | 7.457, s | 101.67, CH |
| 6 | | 153.33, C | | 153.13, C |
| 7 | | 151.69, C | | 151.44, C |
| 8 | 7.062, s | 107.38, CH | 6.989, s | 107.19, CH |
| 9 | | 132.11, C | | 131.75, C |
| 10 | | 128.29, C | | 128.04, C |
| 11 | | 172.23, C | | 172.12, C |
| 12 | 5.452, d, (15.1) | 68.89, CH₂ | 5.449, d, (15.1) | 68.83, CH₂ |
| | 5.521, d, (15.1) | | 5.369, d, (15.1) | |
| 6-OCH₃ | 4.013, s | 56.93, CH₃ | 5.958, s | 56.86, CH₃ |
| 7-OCH₃ | 3.748, s | 56.04, CH₃ | 3.726, s | 56.01, CH₃ |
| 1′ | | 114.78, C | | 114.56, C |
| 2′ | 6.543, s | 98.52, CH | 6.520, s | 98.93, CH |
| 3′ | | 142.22, C | | 141.97, C |
| 4′ | | 149.83, C | | 149.71, C |
| 5′ | 6.580, s | 111.42, CH | 6.533, s | 111.60, CH |
| 6′ | | 150.81, C | | 150.78, C |
| 7′ | 5.944, d (1.0) | 102.51, CH₂ | 5.941, d (1.0) | 102.52, CH₂ |
| | 5.966, d (1.0) | | 5.964, d (1.0) | |
| 1″ | 5.610, d (2.9) | 111.58, CH | 5.518, d (2.9) | 111.78, CH |
| 2″ | 4.791, d (2.9) | 86.91, CH | 4.783, d (2.9) | 87.10, CH |
| 3″ | | 80.11, C | | 80.07, C |
| 4″ | 3.931, d (9.7) | 75.65, CH₂ | 3.924, d (9.7) | 75.61, CH₂ |
| | 4.281, d (9.7) | | 4.250, d (9.7) | |
| 5″ | 3.726, d (10.4) | 71.89, CH₂ | 3.709, d (10.4) | 72.37, CH₂ |
| | 4.052, d (10.4) | | 4.070, d (10.4) | |
| 1′′′ | 4.653, d (7.6) | 105. 73, CH | 4.700, d (7.6) | 105.32, CH |
| 2′′′ | 3.292, m | 75.29, CH | 3.293, m | 75.24, CH |
| 3′′′ | 3.362, m | 77.77, C | 3.382, m | 77.79, C |
| 4′′′ | 3.463, m | 71.16, CH₂ | 3.464, m | 71.17, CH₂ |
| 5′′′ | 3.234, dd (10.5, | 67.30, CH₂ | 3.254, dd (10.5, | 67.23, CH₂ |
| | 11.4) | | 11.4) | |
| | 3.797, dd (5.3, | | 3.829, dd (5.3, | |
| | 11.4) | | 11.4) | |
| 1″″ | 4.341, d (7.6) | 105.64, CH | 4.343, d (7.6) | 105.48, CH |
| 2″″ | 3.312, m | 74.93, CH | 3.369, m | 74.91, CH |
| 3″″ | 3.359, m | 77.79, CH | 3.359, m | 77.68, CH |
| 4″″ | 3.520, m | 71.22, CH | 3.550, m | 71.11, CH |
| 5″″ | 3.261, dd (10.5, | 67.15, CH₂ | 3.284, dd (10.5, | 67.09, CH₂ |
| | 11.4) | | 11.4) | |
| | 3.901, dd (5.3, | | 3.923, dd (5.3, | |
| | 11.4) | | 11.4) | |

TABLE 4
¹H (400 MHZ) and ¹³C (100 MHZ) NMR data of compounds
53 and 54 in CD3OD (8 in ppm, J in Hz).

| | 53 | | 54 | |
|---|---|---|---|---|
| Position | δ_{H} (J in Hz) | δ_{C}, type | δ_{H} (J in Hz) | δ_{C}, type |
| 1 | | 134.08, C | | 133.92, C |
| 2 | | 121.08, C | | 120.92, C |
| 3 | | 130.50, C | | 130.79, C |
| 4 | | 146.19, C | | 145.94, C |
| 5 | 7.672, s | 102.17, CH | 7.551, s | 101.88, CH |
| 6 | | 153.31, C | | 153.21, C |
| 7 | | 151.68, C | | 151.55, C |
| 8 | 7.076, s | 107.36, CH | 7.022, s | 107.26, CH |
| 9 | | 132.09, C | | 131.88, C |
| 10 | | 128.25, C | | 128.13, C |
| 11 | | 172.22, C | | 172.15, C |
| 12 | 5.470, d, (15.1) | 68.88, CH₂ | 5.429, d, (15.1) | 68.87, CH₂ |
| | 5.541, d, (15.1) | | 5.493, d, (15.1) | |
| 6-OCH₃ | 4.031, s | 56.94, CH₃ | 3.993, s | 56.91, CH₃ |
| 7-OCH₃ | 3.755, s | 56.03, CH₃ | 3.742, s | 56.02, CH₃ |
| 1′ | | 114.77, C | | 114.64, C |
| 2′ | 6.541, s | 98.94, CH | 6.522, s | 98.94, CH |
| 3′ | | 142.21, C | | 142.06, C |
| 4′ | | 149.82, C | | 149.75, C |
| 5′ | 6.583, s | 111.49, CH | 6.526, s | 111.53, CH |
| 6′ | | 150.79, C | | 150.82, C |
| 7′ | 5.946, d (1.0) | 102.51, CH₂ | 5.945, d (1.0) | 102.51, CH₂ |
| | 5.967, d (1.0) | | 5.967, d (1.0) | |
| 1″ | 5.637, d (2.8) | 111.42, CH | 5.577, d (2.8) | 111.60, CH |
| 2″ | 4.805, d (2.8) | 86.76, CH | 4.795, d (2.8) | 86.85, CH |
| 3″ | | 80.11, C | | 80.51, C |
| 4″ | 3.928, d (10.0) | 75.60, CH₂ | 3.930, d (10.0) | 75.78, CH₂ |
| | 4.305, d (10.0) | | 4.284, d (10.0) | |
| 5″ | 3.739, d (10.5) | 71.72, CH₂ | 3.731, d (10.5) | 71.99, CH₂ |
| | 4.041, d (10.5) | | 4.051, d (10.5) | |
| 1″′ | 4.636, d (7.6) | 105. 79, CH | 4.656, d (7.6) | 105. 58, CH |
| 2″′ | 3.303, m | 75.27, CH | 3.296, m | 75.26, CH |
| 3″′ | 3.441, m | 84.89, C | 3.451, m | 84.95, C |
| 4″′ | 3.447, m | 71.15, CH₂ | 3.468, m | 71.14, CH₂ |
| 5″′ | 3.251, dd (10.5, | 67.29, CH₂ | 3.250, dd (10.5, | 67.26, CH₂ |
| | 11.4) | | 11.4) | |
| | 3.775, dd (5.3, | | 3.825, dd (5.3, | |
| | 11.4) | | 11.4) | |
| 1″″ | 4.362, d (7.6) | 105.48, CH | 4.361, d (7.6) | 105.46, CH |
| 2″″ | 3.435, m | 74.53, CH | 3.462, m | 74.52, CH |
| 3″″ | 3.368, m | 77.75, CH | 3.364, m | 77.89, CH |
| 4″″ | 3.560, m | 69.94, CH | 3.586, m | 69.91, CH |
| 5″″ | 3.270, dd (10.5, | 66.80, CH₂ | 3.271, dd (10.5, | 66.79, CH₂ |
| | 11.4) | | 11.4) | |
| | 3.939, dd (5.3, | | 3.950, dd (5.3, | |
| | 11.4) | | 11.4) | |
| 1″″′ | 5.315, d (2.8) | 111.17, CH | 5.321, d (2.8) | 111.13, CH |
| 2″″′ | 4.016, d (2.8) | 77.91, CH | 4.014, d (2.8) | 77.78, CH |
| 3″″′ | | 80.51, C | | 80.10, C |
| 4″″′ | 3.798, d (10.0) | 75.03, CH₂ | 3.810, d (10.0) | 75.00, CH₂ |
| | 4.140, d (10.0) | | 4.145, d (10.0) | |
| 5″″′ | 3.612, s | 65.18, CH₂ | 3.615, s | 65.14, CH₂ |

Having now fully described the present invention in some detail by way of illustration and examples for purposes of clarity of understanding, it will be obvious to one of ordinary skill in the art that the same can be performed by modifying or changing the invention within a wide and equivalent range of conditions, formulations and other parameters without affecting the scope of the invention or any specific embodiment thereof, and that such modifications or changes are intended to be encompassed within the scope of the appended claims. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention.

All references cited herein are hereby incorporated by reference in their entirety to the extent that there is no inconsistency with the disclosure of this specification. Some references provided herein are incorporated by reference to provide details concerning sources of starting materials, additional starting materials, additional reagents, additional methods of synthesis, additional methods of analysis, additional biological materials, additional cells, and additional uses of the invention. All headings used herein are for convenience only. All patents and publications mentioned in the specification are indicative of the levels of skill of those skilled in the art to which the invention pertains, and are herein incorporated by reference to the same extent as if each individual publication, patent or patent application was specifically and individually indicated to be incorporated by reference. References cited herein are incorporated by reference herein in their entirety to indicate the state of the art as of their publication or filing date and it is intended that this information can be employed herein, if needed, to exclude specific embodiments that are in the prior art. For example, when composition of matter is claimed, it should be understood that compounds known and available in the art prior to applicants' invention, including compounds for which an enabling disclosure is provided in the references cited herein, are not intended to be included in the composition of matter claims herein.

What is claimed is:

1. A compound of Formula I: 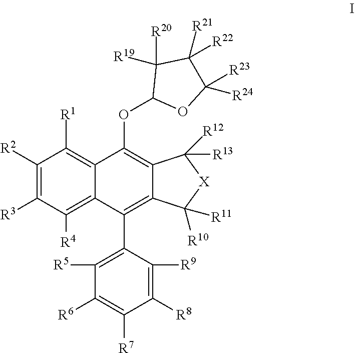
or a pharmaceutically acceptable salt thereof, wherein:
X is oxygen or sulfur;
R¹, R², R³, R⁴, R⁵, R⁶, R⁷, R⁸, and R⁹ are each independently hydrogen, alkyl, akenyl, alkynyl, cycloalkyl, heterocycloalkyl, aryl, aralkyl, heteroaryl, haloalkyl, halogen, cyano, NO₂, —OR²⁶, —C(═O)R²⁷, —C(═O)N(R²⁶)R²⁷, —C(═O)OR²⁶, —OC(═O)R²⁶, —OSi(R²⁵)(R²⁶)R²⁷, —S(═O)₂R²⁶, —S(═O)₂N(R²⁶)R²⁷, —N(R²⁶)R²⁷, —N(R²⁶)N(R²⁶)R²⁷, —N(R²⁶)C(═O)R²⁷, —N(R²⁶)S(═O)₂R²⁷ or a moiety comprising 1 to 30 plural valence atoms selected from the group consisting of carbon, nitrogen, oxygen and sulfur; or R¹ and R², R² and R³, R³ and R⁴, R⁵ and R⁶, R⁶ and R⁷, R⁷ and R⁸, or R⁸ and R⁹ taken together with the carbon atoms to which they are attached to form a cyclic group which is optionally substituted with halogen or a moiety comprising 1 to 30 plural valence atoms selected from the group consisting of carbon, nitrogen, oxygen and sulfur;
R¹⁰ and R¹¹ taken together form oxo; or while one of R¹⁰ and R¹¹ is hydrogen or halogen, the other one of R¹⁰ and R¹¹ is selected from the group consisting of R²⁵, —OR²⁵, —C(═O)R²⁵, and —C(═O)OR²⁵;
R¹² and R¹³ taken together form oxo; or while one of R¹² and R¹³ is hydrogen or halogen, the other one of R¹² and R¹³ is selected from the group consisting of R²⁵, —OR²⁵, —C(═O)R²⁵, and —C(═O)OR²⁵;
R¹⁹ and R²⁰ taken together form oxo; or while one of R¹⁹ and R²⁰ is hydrogen or halogen, the other one of R¹⁹ and R²⁰ is selected from the group consisting of R²⁵, —OR²⁵, —C(═O)R²⁵, —C(═O)OR²⁵, —OC(═O)R²⁵, —OC(═O)N(R²⁵)R²⁵, optionally substituted monosaccharide, optionally substituted disaccharide, optionally substituted trisaccharide, and optionally substituted tetrasaccharide;
R²¹ and R²² taken together form oxo; or while one of R²¹ and R²² is hydrogen, halogen, R²⁵ or —OR²⁵, the other one of R²¹ and R²² is selected from the group consisting of R²⁵, —OR²⁵, —OC(═O)R²⁵, —OC(═O)N(R²⁵)R²⁵, —C(═O)OR²⁵, —CH₂R²⁹, —CH₂OR²⁹, —C(═O)R²⁹; or R¹⁹ and R²¹ taken together with the carbon atoms to which they are attached to form a 5-6 membered heterocyclyl optionally substituted with 1, 2, 3, 4 or 5 group(s) independently selected from R²⁵;
R²³ and R²⁴ taken together form oxo; or while one of R²³ and R²⁴ is hydrogen or halogen, the other one of R²³ and R²⁴ is selected from the group consisting of R²⁵, —OR²⁵, —C(═O)R²⁵, —C(═O)OR²⁵, optionally substituted monosaccharide, optionally substituted disaccharide, optionally substituted trisaccharide, and optionally substituted tetrasaccharide;
R²⁵ for each occurrence is independently selected from hydrogen, halogen, trichloromethyl, trifluoromethyl, cyano, nitro, —OR²⁶, —C(═O)R²⁷, —C(═O)N(R²⁶)R²⁷, —C(═O)OR²⁶, —OC(═O)R²⁶, —OSi(R²⁵)(R²⁶)R²⁷, —S(═O)₂R²⁶, —S(═O)₂N(R²⁶)R²⁷, —N(R²⁶)R²⁷, —N(R²⁶)N(R²⁶)R²⁷, —N(R²⁶)C(═O)R²⁷, —N(R²⁶)S(═O)₂R²⁷, —N₃, —OS(═O)₂CF₃, hydrocarbyl optionally substituted with 1, 2, 3, 4 or 5 group(s) independently selected from the group consisting of R²⁸, heterocyclcyl optionally substituted with 1, 2, 3, 4 or 5 group(s) independently selected from the group consisting of R²⁸, and —(CH₂)ₖ-heterocyclyl optionally substituted with 1, 2, 3, 4 or 5 group(s) independently selected from the group consisting of R²⁸, wherein k is an integer between 1 and 6;
R²⁶ and R²⁷ for each occurrence are each independently hydrogen or selected from hydrocarbyl and heterocyclyl, either of which is optionally substituted with 1, 2, 3, 4 or 5 group(s) independently selected from halogen, cyano, amino, hydroxy, C₁₋₆ alkyl and C₁₋₆ alkoxy;
R²⁸ for each occurrence is independently selected from halogen, trichloromethyl, trifluoromethyl, cyano, nitro, oxo, ═NR²⁶, —OR²⁶, —C(═O)R²⁷, —C(═O)N(R²⁶)R²⁷, —C(═O)OR²⁶, —OC(═O)R²⁶, —OSi(R²⁵)(R²⁶)R²⁷, —S(═O)₂R²⁶, —S(═O)₂N(R²⁶)R²⁷, —N(R²⁶)R²⁷, —N(R²⁶)N(R²⁶)R²⁷, —N(R²⁶)C(═O)R²⁷ and —N(R²⁶)S(═O)₂R²⁷; and
R²⁹ for each occurrence is independently selected from hydrogen, halogen, trichloromethyl, trifluoromethyl, cyano, nitro, —OR²⁶, —C(═O)R²⁷, —C(═O)N(R²⁶)R²⁷, —C(═O)OR²⁶, —OC(═O)R²⁶, —OSi(R²⁵)(R²⁶)R²⁷, —S(═O)₂R²⁶, —S(═O)₂N(R²⁶)R²⁷, —N(R²⁶)R²⁷, —N(R²⁶)N(R²⁶)R²⁷, —N(R²⁶)C(═O)R²⁷, —N(R²⁶)S(═O)₂R²⁷, —N₃, —OS(═O)₂CF₃, optionally substituted monosaccharide, optionally substituted disaccharide, optionally substituted trisaccharide, optionally substituted tetrasaccharide, hydrocarbyl optionally substituted with 1, 2, 3, 4 or 5 group(s) independently selected from the group consisting of R²⁸, and heterocyclyl optionally substituted with 1, 2, 3, 4 or 5 group(s) independently selected from the group consisting of R²⁸, with the proviso that the compound of Formula I does not include a compound selected from the group consisting of 1, 1-Ac, A1, A2, A3, A4, A5, A6, A7, and A8:

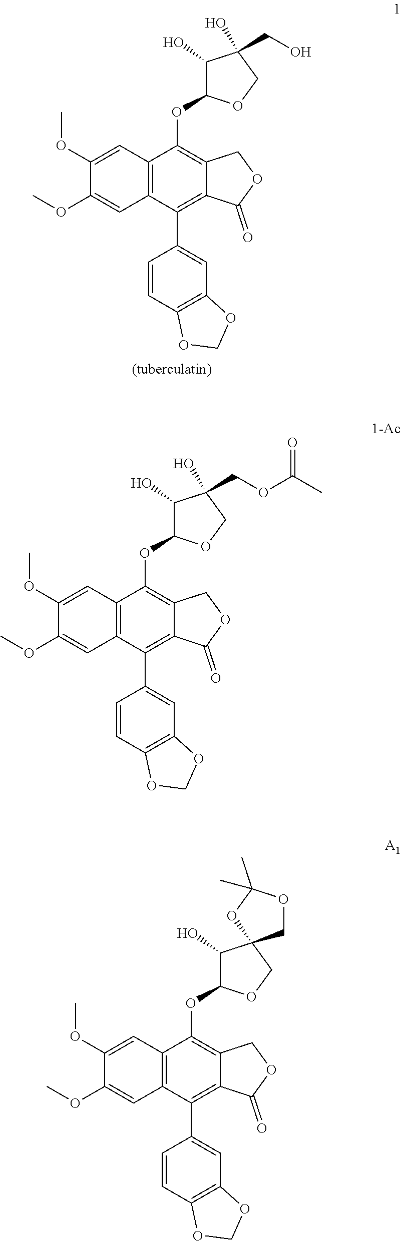

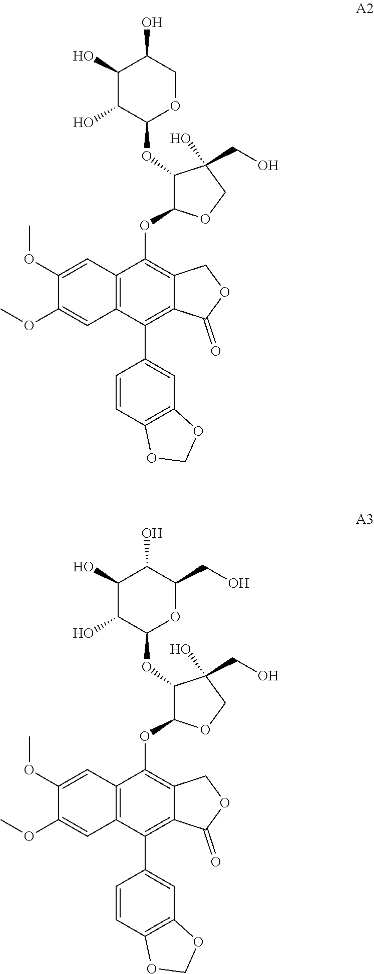 [SMILES: COc1cc2c(O[C@@H]3OC[C@](O)(CO)[C@H]3O[C@@H]3OC[C@H](O)[C@H](O)[C@H]3O)c3c(c(-c4ccc5c(c4)OCO5)c2cc1OC)C(=O)OC3 ; COc1cc2c(O[C@@H]3OC[C@](O)(CO)[C@H]3O[C@@H]3O[C@H](CO)[C@@H](O)[C@H](O)[C@H]3O)c3c(c(-c4ccc5c(c4)OCO5)c2cc1OC)C(=O)OC3]

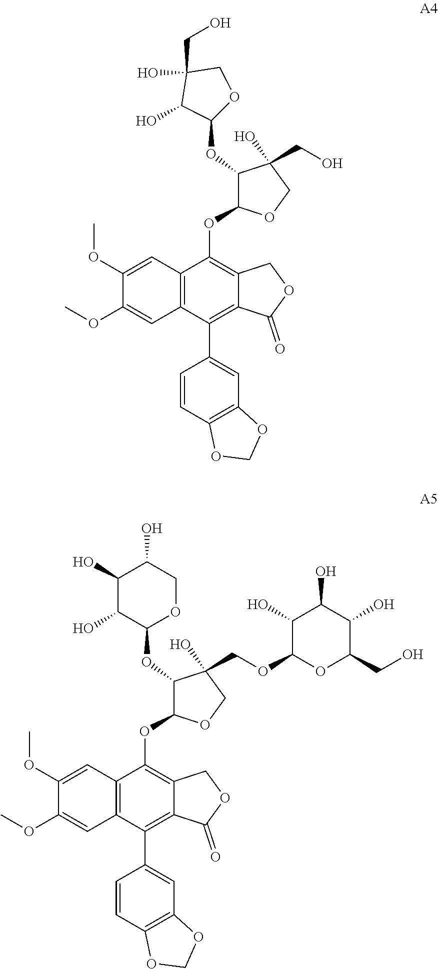 [SMILES: COc1cc2c(O[C@@H]3OC[C@](O)(CO)[C@H]3O[C@@H]3OC[C@](O)(CO)[C@H]3O)c3c(c(-c4ccc5c(c4)OCO5)c2cc1OC)C(=O)OC3 ; COc1cc2c(O[C@@H]3OC[C@](O)(CO[C@@H]4O[C@H](CO)[C@@H](O)[C@H](O)[C@H]4O)[C@H]3O[C@@H]3OC[C@@H](O)[C@H](O)[C@H]3O)c3c(c(-c4ccc5c(c4)OCO5)c2cc1OC)C(=O)OC3]

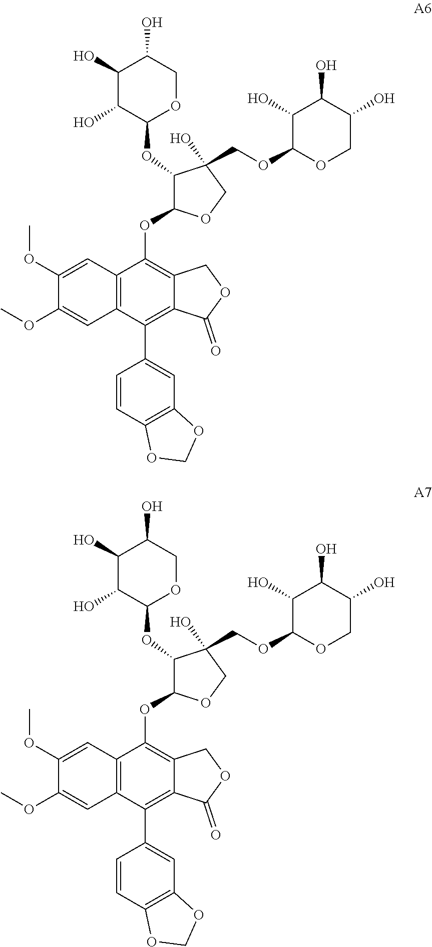 [SMILES: COc1cc2c(O[C@@H]3OC[C@](O)(CO[C@@H]4OC[C@@H](O)[C@H](O)[C@H]4O)[C@H]3O[C@@H]3OC[C@@H](O)[C@H](O)[C@H]3O)c3c(c(-c4ccc5c(c4)OCO5)c2cc1OC)C(=O)OC3 ; COc1cc2c(O[C@@H]3OC[C@](O)(CO[C@@H]4OC[C@@H](O)[C@H](O)[C@H]4O)[C@H]3O[C@@H]3OC[C@H](O)[C@H](O)[C@H]3O)c3c(c(-c4ccc5c(c4)OCO5)c2cc1OC)C(=O)OC3]

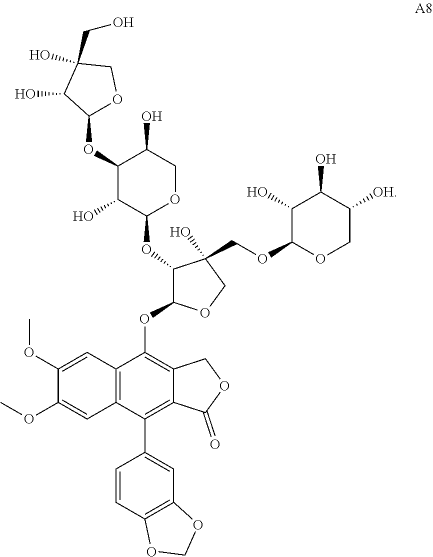 [SMILES: COc1cc2c(O[C@@H]3OC[C@](O)(CO[C@@H]4OC[C@@H](O)[C@H](O)[C@H]4O)[C@H]3O[C@@H]3OC[C@H](O)[C@H](O[C@@H]4OC[C@](O)(CO)[C@H]4O)[C@H]3O)c3c(c(-c4ccc5c(c4)OCO5)c2cc1OC)C(=O)OC3]

2. The compound of claim 1, wherein R²⁰, R²³, and R²⁴ are each hydrogen; R¹⁹ and R²¹ are each independently R²⁹; and R²² is —CH₂R²⁹, —CH₂OR²⁹; or R¹⁹ and R²¹ taken together with the carbon atoms to which they are attached to form a 5-6 membered heterocyclyl optionally substituted with 1, 2, 3, 4 or 5 group(s) independently selected from R²⁵.

3. The compound of claim 1, wherein the compound has Formula II: 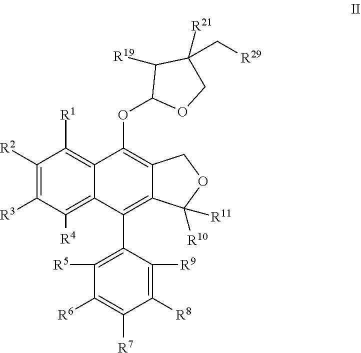
or a pharmaceutically acceptable salt thereof, wherein:
R¹, R², R³, R⁴, R⁵, R⁶, R⁷, R⁸, and R⁹ are each independently hydrogen, alkyl, akenyl, alkynyl, cycloalkyl, heterocycloalkyl, aryl, aralkyl, heteroaryl, haloalkyl, halogen, cyano, NO₂, —OR²⁶, —C(═O)R²⁷, —C(═O)N(R²⁶)R²⁷, —C(═O)OR²⁶, —OC(═O)R²⁶, —OSi(R²⁵)(R²⁶)R²⁷, —S(═O)₂R²⁶, —S(═O)₂N(R²⁶)R²⁷, —N(R²⁶)R²⁷, —N(R²⁶)N(R²⁶)R²⁷, —N(R²⁶)C(═O)R²⁷, —N(R²⁶)S(═O)₂R²⁷ or a moiety comprising 1 to 30 plural valence atoms selected from the group consisting of carbon, nitrogen, oxygen and sulfur; or R¹ and R², R² and R³, R³ and R⁴, R⁵ and R⁶, R⁶ and R⁷, R⁷ and R⁸, or R⁸ and R⁹ taken together with the carbon atoms to which they are attached to form a cyclic group which is optionally substituted with halogen or a moiety comprising 1 to 30 plural valence atoms selected from the group consisting of carbon, nitrogen, oxygen and sulfur;
R¹⁰ and R¹¹ taken together form oxo; or while one of R¹⁰ and R¹¹ is hydrogen, the other one of R¹⁰ and R¹¹ is selected from the group consisting of R²⁵, —OR²⁵, —C(═O)R²⁵, and —C(═O)OR²⁵;
R¹⁹ is selected from the group consisting of R²⁵, —OR²⁵, —OC(═O)R²⁵, —OC(═O)N(R²⁵)R²⁵, optionally substituted monosaccharide, optionally substituted disaccharide, optionally substituted trisaccharide, and optionally substituted tetrasaccharide;
R²¹ is selected from the group consisting of R²⁵, —OR²⁵, —OC(═O)R²⁵, —OC(═O)N(R²⁵)R²⁵, —C(═O)OR²⁵, —CH₂R²⁹, —CH₂OR²⁹, —C(═O)R²⁹; or R¹⁹ and R²¹ taken together with the carbon atoms to which they are attached to form a 5-6 membered heterocyclyl optionally substituted with 1, 2, 3, 4 or 5 group(s) independently selected from R²⁵;
R²⁵ for each occurrence is independently selected from hydrogen, halogen, trichloromethyl, trifluoromethyl, cyano, nitro, —OR²⁶, —C(═O)R²⁷, —C(═O)N(R²⁶)R²⁷, —C(═O)OR²⁶, —OC(═O)R²⁶, —OSi(R²⁵)(R²⁶)R²⁷, —S(═O)₂R²⁶, —S(═O)₂N(R²⁶)R²⁷, —N(R²⁶)R²⁷, —N(R²⁶)N(R²⁶)R²⁷, —N(R²⁶)C(═O)R²⁷, —N(R²⁶)S(═O)₂R²⁷, —N₃, —OS(═O)₂CF₃, hydrocarbyl optionally substituted with 1, 2, 3, 4 or 5 group(s) independently selected from the group consisting of R²⁸, heterocyclcyl optionally substituted with 1, 2, 3, 4 or 5 group(s) independently selected from the group consisting of R²⁸, and —(CH₂)ₖ-heterocyclyl optionally substituted with 1, 2, 3, 4 or 5 group(s) independently selected from the group consisting of R²⁸, wherein k is an integer between 1 and 6;
R²⁶ and R²⁷ for each occurrence are each independently hydrogen or selected from hydrocarbyl and heterocyclyl, either of which is optionally substituted with 1, 2, 3, 4 or 5 group(s) independently selected from halogen, cyano, amino, hydroxy, C₁₋₆ alkyl and C₁₋₆ alkoxy;
R²⁸ for each occurrence is independently selected from halogen, trichloromethyl, trifluoromethyl, cyano, nitro, oxo, ═NR²⁶, —OR²⁶, —C(═O)R²⁷, —C(═O)N(R²⁶)R²⁷, —C(═O)OR²⁶, —OC(═O)R²⁶, —OSi(R²⁵)(R²⁶)R²⁷, —S(═O)₂R²⁶, —S(═O)₂N(R²⁶)R²⁷, —N(R²⁶)R²⁷, —N(R²⁶)N(R²⁶)R²⁷, —N(R²⁶)C(═O)R²⁷ and —N(R²⁶)S(═O)₂R²⁷; and
R²⁹ for each occurrence is independently selected from hydrogen, halogen, trichloromethyl, trifluoromethyl, cyano, nitro, —OR²⁶, —C(═O)R²⁷, —C(═O)N(R²⁶)R²⁷, —C(═O)OR²⁶, —OC(═O)R²⁶, —OSi(R²⁵)(R²⁶)R²⁷, —S(═O)₂R²⁶, —S(═O)₂N(R²⁶)R²⁷, —N(R²⁶)R²⁷, —N(R²⁶)N(R²⁶)R²⁷, —N(R²⁶)C(═O)R²⁷, —N(R²⁶)S(═O)₂R²⁷, —N₃, —OS(═O)₂CF₃, optionally substituted monosaccharide, optionally substituted disaccharide, optionally substituted trisaccharide, optionally substituted tetrasaccharide, hydrocarbyl optionally substituted with 1, 2, 3, 4 or 5 group(s) independently selected from the group consisting of R²⁸, and heterocyclyl optionally substituted with 1, 2, 3, 4 or 5 group(s) independently selected from the group consisting of R²⁸.

4. The compound of claim 3, wherein R¹, R², R³, R⁴, R⁵, R⁶, R⁷, R⁸, and R⁹ are each independently hydrogen, alkyl, akenyl, alkynyl, cycloalkyl, heterocycloalkyl, aryl, aralkyl, heteroaryl, haloalkyl, halogen, cyano, NO₂, —OR²⁶, —C(═O)R²⁷, —C(═O)N(R²⁶)R²⁷, —C(═O)OR²⁶, —OC(═O)R²⁶, —OSi(R²⁵)(R²⁶)R²⁷, —S(═O)₂R²⁶, —S(═O)₂N(R²⁶)R²⁷, —N(R²⁶)R²⁷, —N(R²⁶)N(R²⁶)R²⁷, —N(R²⁶)C(═O)R²⁷, and —N(R²⁶)S(═O)₂R²⁷; or R¹ and R², R² and R³, R³ and R⁴, R⁵ and R⁶, R⁶ and R⁷, R⁷ and R⁸, or R⁸ and R⁹ taken together with the carbon atoms to which they are attached to form a 5-membered heterocyclyl;
R¹⁰ and R¹¹ taken together form oxo;
R¹⁹ is selected from the group consisting of OR²⁵, —OC(═O)R²⁵, —OC(═O)N(R²⁵)R²⁵, optionally substituted monosaccharide, optionally substituted disaccharide, optionally substituted trisaccharide, and optionally substituted tetrasaccharide; and
R²¹ is selected from the group consisting of —OR²⁵, —OC(═O)R²⁵, —OC(═O)N(R²⁵)R²⁵; or R¹⁹ and R²¹ taken together with the carbon atoms to which they are attached to form a 5 membered heterocyclyl optionally substituted with 1, 2, 3, 4 or 5 group(s) independently selected from R²⁵.

5. The compound of claim 1, wherein the compound has Formula III: 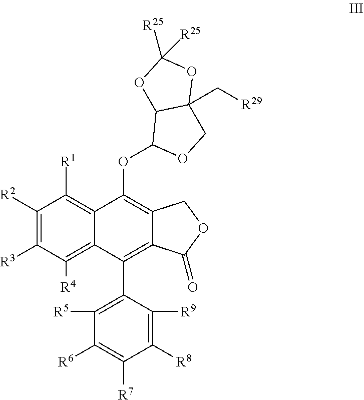
or a pharmaceutically acceptable salt thereof, wherein:
wherein R¹, R², R³, R⁴, R⁵, R⁶, R⁷, R⁸, and R⁹ are each independently hydrogen, alkyl, akenyl, alkynyl, cycloalkyl, heterocycloalkyl, aryl, aralkyl, heteroaryl, haloalkyl, halogen, cyano, NO₂, —OR²⁶, —C(═O)R²⁷, —C(═O)N(R²⁶)R²⁷, —C(═O)OR²⁶, —OC(═O)R²⁶, —OSi(R²⁵)(R²⁶)R²⁷, —S(═O)₂R²⁶, —S(═O)₂N(R²⁶)R²⁷, —N(R²⁶)R²⁷, —N(R²⁶)N(R²⁶)R²⁷, —N(R²⁶)C(═O)R²⁷, —N(R²⁶)S(═O)₂R²⁷; or R⁸ and R², R² and R³, R³ and R⁴, R⁵ and R⁶, R⁶ and R⁷, R⁷ and R⁸, or R⁸ and R⁹ taken together with the carbon atoms to which they are attached to form a 5-membered heterocyclyl;
R²⁵ for each occurrence is independently selected from hydrogen and hydrocarbyl optionally substituted with 1, 2, 3, 4 or 5 group(s) independently selected from the group consisting of R²⁸, heterocyclcyl optionally substituted with 1, 2, 3, 4 or 5 group(s) independently selected from the group consisting of R²⁸, and —(CH₂)ₖ-heterocyclyl optionally substituted with 1, 2, 3, 4 or 5 group(s) independently selected from the group consisting of R²⁸, wherein k is an integer between 1 and 6;
R²⁶ and R²⁷ for each occurrence are each independently hydrogen or selected from hydrocarbyl and heterocyclyl, either of which is optionally substituted with 1, 2, 3, 4 or 5 group(s) independently selected from halogen, cyano, amino, hydroxy, C₁₋₆ alkyl and C₁₋₆ alkoxy;
R²⁸ for each occurrence is independently selected from halogen, trichloromethyl, trifluoromethyl, cyano, nitro, oxo, ═NR²⁶, —OR²⁶, —C(═O)R²⁷, —C(═O)N(R²⁶)R²⁷, —C(═O)OR²⁶, —OC(═O)R²⁶, —OSi(R²⁵)(R²⁶)R²⁷, —S(═O)₂R²⁶, —S(═O)₂N(R²⁶)R²⁷, —N(R²⁶)R²⁷, —N(R²⁶)N(R²⁶)R²⁷, —N(R²⁶)C(═O)R²⁷ and —N(R²⁶)S(═O)₂R²⁷; and
R²⁹ is selected from the group consisting of halogen, cyano, —OR²⁶, —OC(═O)R²⁶, —OSi(R²⁵)(R²⁶)R²⁷, —N(R²⁶)R²⁷, —N(R²⁶)N(R²⁶)R²⁷, —N(R²⁶)C(═O)R²⁷, —N(R²⁶)S(═O)₂R²⁷, —N₃, and —OS(═O)₂CF₃.

6. The compound of claim 5, wherein R¹, R⁴, R⁶, and R⁹ are each hydrogen; R², R³, R⁷, and R⁸ are each independently —OR²⁶; and R⁵ is hydrogen or —OR²⁶; or R² and R³ taken together with the carbon atoms to which they are attached to form a 5-membered heterocyclyl; or R⁷ and R⁸ taken together with the carbon atoms to which they are attached to form a 5-membered heterocyclyl.

7. The compound of claim 1, wherein the compound has Formula IV: 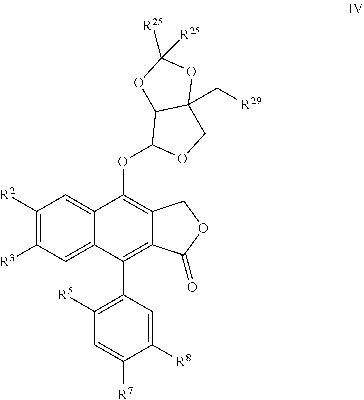
or a pharmaceutically acceptable salt thereof, wherein:
R², R³, R⁷, and R⁸ are each independently —OR²⁶; and R⁵ is hydrogen or —OR²⁶; or R² and R³ taken together form a methylenedioxy group; or R⁷ and R⁸ taken together form a methylenedioxy group;
R²⁵ for each occurrence is independently selected from hydrogen and hydrocarbyl;
R²⁶ for each occurrence is independently hydrogen, hydrocarbyl, or heterocyclyl; and
R²⁹ is selected from the group consisting of halogen, cyano, —OR²⁶, —OC(═O)R²⁶, —OSi(R²⁵)(R²⁶)R²⁷, —N(R²⁶)R²⁷, —N(R²⁶)N(R²⁶)R²⁷, —N(R²⁶)C(═O)R²⁷, —N(R²⁶)S(═O)₂R²⁷, —N₃, and —OS(═O)₂CF₃.

8. The compound of claim 1, wherein the compound has Formula V: 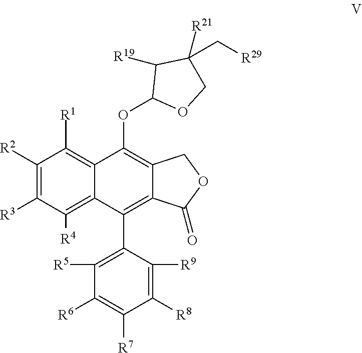
or a pharmaceutically acceptable salt thereof, wherein:
wherein R¹, R², R³, R⁴, R⁵, R⁶, R⁷, R⁸, and R⁹ are each independently hydrogen, alkyl, akenyl, alkynyl, cycloalkyl, heterocycloalkyl, aryl, aralkyl, heteroaryl, haloalkyl, halogen, cyano, NO₂, —OR²⁶, —C(═O)R²⁷, —C(═O)N(R²⁶)R²⁷, —C(═O)OR²⁶, —OC(═O)R²⁶, —OSi(R²⁵)(R²⁶)R²⁷, —S(═O)₂R²⁶, —S(═O)₂N(R²⁶)R²⁷, —N(R²⁶)R²⁷, —N(R²⁶)N(R²⁶)R²⁷, —N(R²⁶)C(═O)R²⁷, —N(R²⁶)S(═O)₂R²⁷; or R¹ and R², R² and R³, R³ and R⁴, R⁵ and R⁶, R⁶ and R⁷, R⁷ and R⁸, or R¹ and R⁹ taken together with the carbon atoms to which they are attached to form a 5-membered heterocyclyl;
R¹⁹ is selected from the group consisting of —OR²⁵, —OC(═O)R²⁵, —OC(═O)N(R²⁵)R²⁵, optionally substituted monosaccharide, optionally substituted disaccharide, optionally substituted trisaccharide, and optionally substituted tetrasaccharide;
R²¹ is selected from the group consisting of —OR²⁵, —OC(═O)R²⁵, —OC(═O)N(R²⁵)R²⁵, and —C(═O)OR²⁵;
R²⁵ for each occurrence is independently selected from hydrogen and hydrocarbyl optionally substituted with 1, 2, 3, 4 or 5 group(s) independently selected from the group consisting of R²⁸, heterocyclcyl optionally substituted with 1, 2, 3, 4 or 5 group(s) independently selected from the group consisting of R²⁸, and —(CH₂)ₖ-heterocyclyl optionally substituted with 1, 2, 3, 4 or 5 group(s) independently selected from the group consisting of R²⁸, wherein k is an integer between 1 and 6;
R²⁶ and R²⁷ for each occurrence are each independently hydrogen or selected from hydrocarbyl and heterocyclyl, either of which is optionally substituted with 1, 2, 3, 4 or 5 group(s) independently selected from halogen, cyano, amino, hydroxy, C₁₋₆ alkyl and C₁₋₆ alkoxy;
R²⁸ for each occurrence is independently selected from halogen, trichloromethyl, trifluoromethyl, cyano, nitro, oxo, ═NR²⁶, —OR²⁶, —C(═O)R²⁷, —C(═O)N(R²⁶)R²⁷, —C(═O)OR²⁶, —OC(═O)R²⁶, —OSi(R²⁵)(R²⁶)R²⁷, —S(═O)₂R²⁶, —S(═O)₂N(R²⁶)R²⁷, —N(R²⁶)R²⁷, —N(R²⁶)N(R²⁶)R²⁷, —N(R²⁶)C(═O)R²⁷ and —N(R²⁶)S(═O)₂R²⁷; and
R²⁹ is selected from the group consisting of halogen, cyano, —OR²⁶, —OC(═O)R²⁶, —OSi(R²⁵)(R²⁶)R²⁷, —N(R²⁶)R²⁷, —N(R²⁶)N(R²⁶)R²⁷, —N(R²⁶)C(═O)R²⁷, —N(R²⁶)S(═O)₂R²⁷, —N₃, and —OS(═O)₂CF₃, optionally substituted monosaccharide, optionally substituted disaccharide, optionally substituted trisaccharide, and optionally substituted tetrasaccharide.

9. The compound of claim 8, wherein R¹, R⁴, R⁶, and R⁹ are each hydrogen; R², R³, R⁷, and R⁸ are each independently —OR²⁶; and R⁵ is hydrogen or —OR²⁶; or R² and R³ taken together with the carbon atoms to which they are attached to form a 5-membered heterocyclyl; or R⁷ and R⁸ taken together with the carbon atoms to which they are attached to form a 5-membered heterocyclyl.

10. The compound of claim 1, wherein the compound has Formula VI: 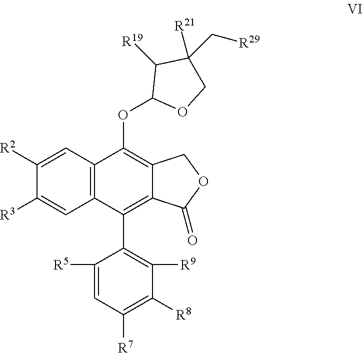
or a pharmaceutically acceptable salt thereof, wherein:
R², R³, R⁷, and R⁸ are each independently —OR²⁶; and R⁵; or R² and R³ taken together form a methylenedioxy group; or R⁷ and R⁸ taken together form a methylenedioxy group;
R⁹ is hydrogen or —OR²⁶;
R¹⁹ is selected from the group consisting of —OR²⁵, —OC(═O)R²⁵, and —OC(═O)N(R²⁵)R²⁵, optionally substituted monosaccharide, optionally substituted disaccharide, optionally substituted trisaccharide, and optionally substituted tetrasaccharide;
R²¹ is selected from the group consisting of —OR²⁵, —OC(═O)R²⁵, —OC(═O)N(R²⁵)R²⁵, and —C(═O)OR²⁵;
R²⁵ for each occurrence is independently selected from hydrogen and hydrocarbyl;
R²⁶ for each occurrence is independently hydrogen, hydrocarbyl, or heterocyclyl; and
R²⁹ is selected from the group consisting of halogen, cyano, —OR²⁶, —OC(═O)R²⁶, —OSi(R²⁵)(R²⁶)R²⁷, —N(R²⁶)R²⁷, —N(R²⁶)N(R²⁶)R²⁷, —N(R²⁶)C(═O)R²⁷, —N(R²⁶)S(═O)₂R²⁷, —N₃, and —OS(═O)₂CF₃, optionally substituted monosaccharide, optionally substituted disaccharide, optionally substituted trisaccharide, and optionally substituted tetrasaccharide.

11. The compound of claim 1, wherein the compound is selected from the group consisting of 8, 9, 10, 11, 12, 16, 17, 18, 19, 20, 21, 23, 24, 25, 26, 27, 28, 29, 30, 31, 32, 42, 43, 44, 45, 46, 47, 48, 49, 50, 51, 52, 53 and 54: 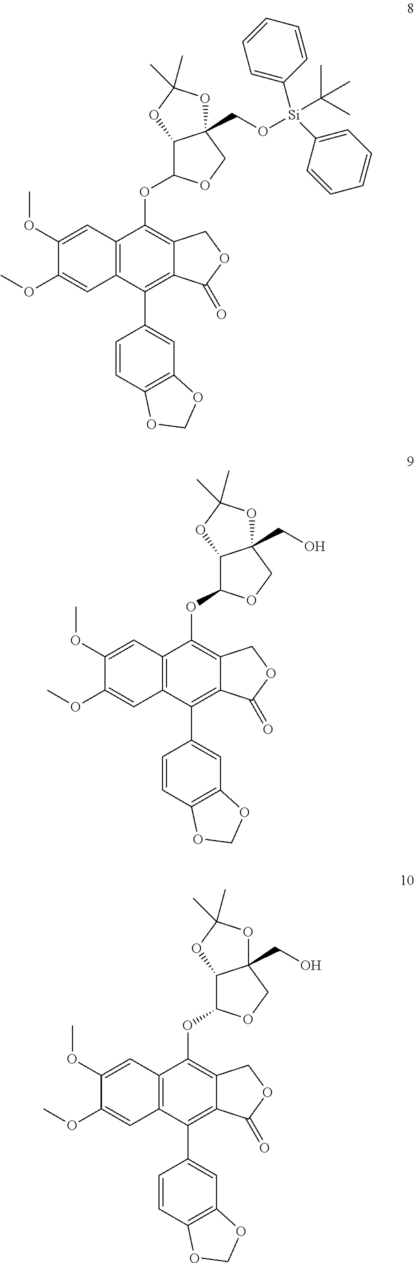 [SMILES: COc1cc2c(OC3OC[C@@]4(CO[Si](c5ccccc5)(c5ccccc5)C(C)(C)C)OC(C)(C)O[C@@H]34)c3c(c(-c4ccc5c(c4)OCO5)c2cc1OC)C(=O)OC3 ; COc1cc2c(O[C@@H]3OC[C@@]4(CO)OC(C)(C)O[C@@H]34)c3c(c(-c4ccc5c(c4)OCO5)c2cc1OC)C(=O)OC3 ; COc1cc2c(O[C@H]3OC[C@@]4(CO)OC(C)(C)O[C@@H]34)c3c(c(-c4ccc5c(c4)OCO5)c2cc1OC)C(=O)OC3] 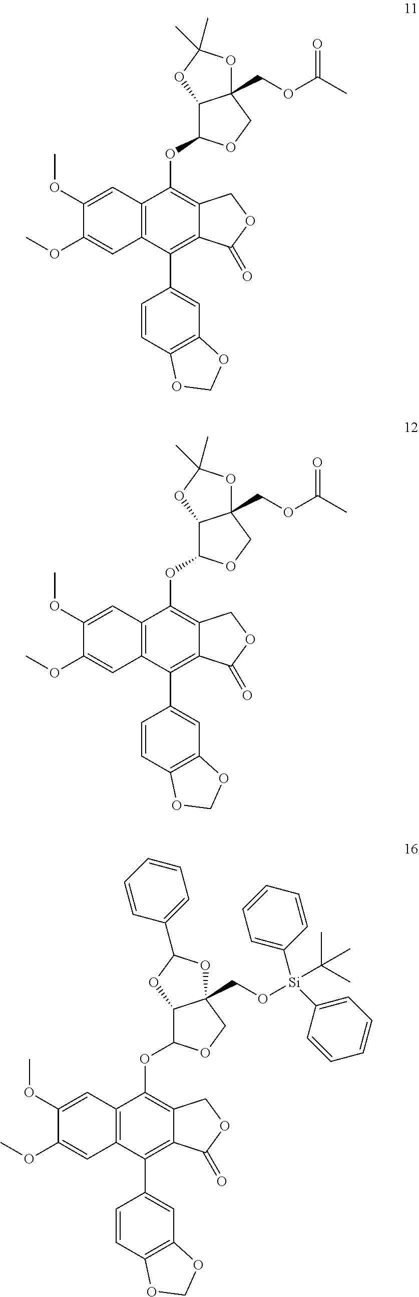 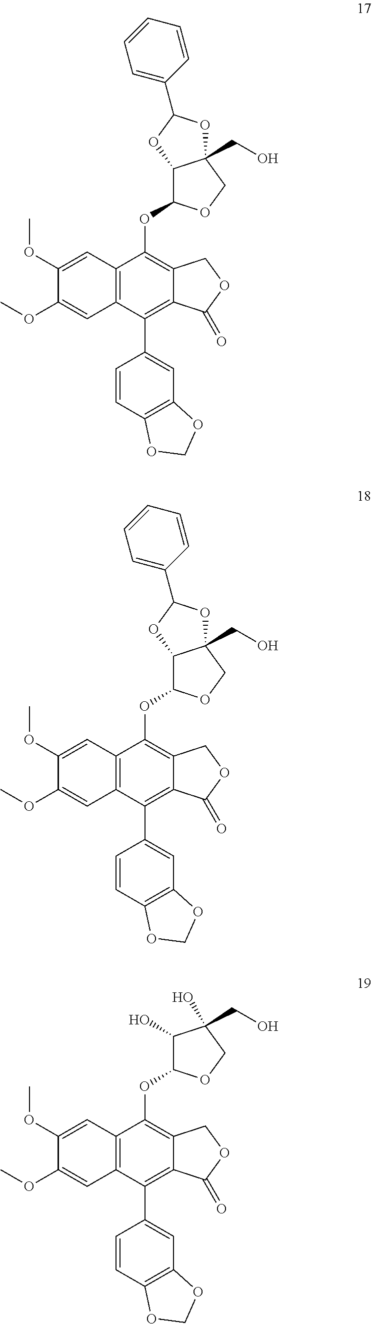 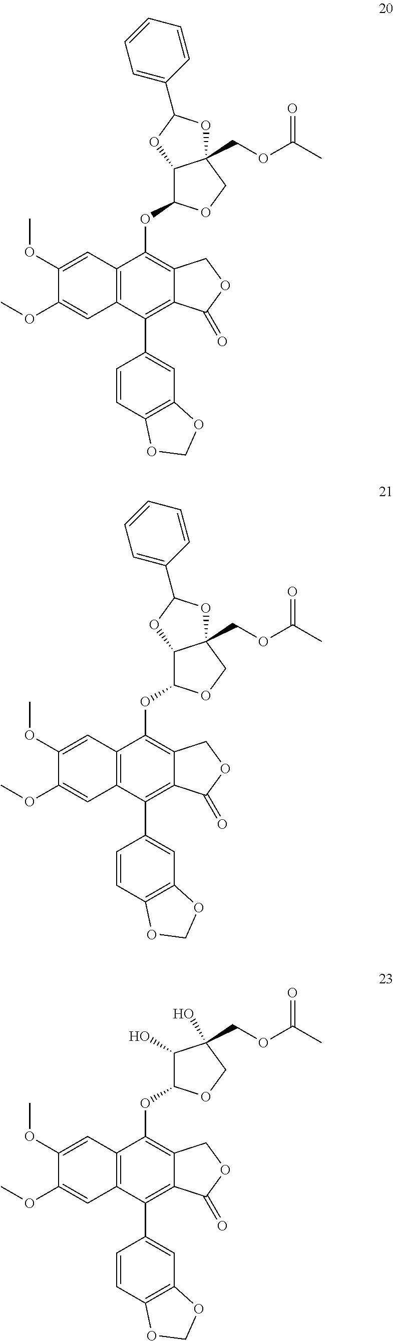 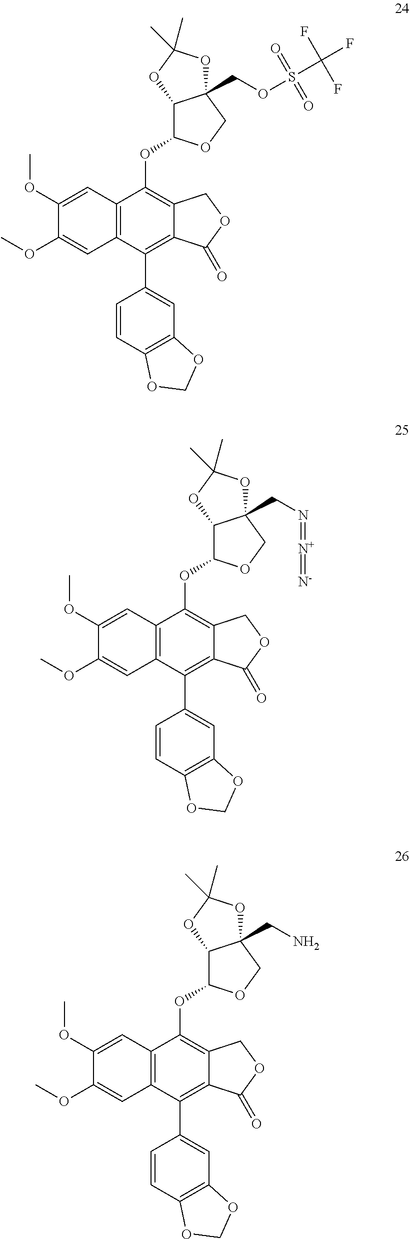 [SMILES: COc1cc2c(O[C@H]3OC[C@@]4(COS(=O)(=O)C(F)(F)F)OC(C)(C)O[C@@H]34)c3c(c(-c4ccc5c(c4)OCO5)c2cc1OC)C(=O)OC3 ; COc1cc2c(O[C@H]3OC[C@@]4(CN=[N+]=[N-])OC(C)(C)O[C@@H]34)c3c(c(-c4ccc5c(c4)OCO5)c2cc1OC)C(=O)OC3 ; COc1cc2c(O[C@H]3OC[C@@]4(CN)OC(C)(C)O[C@@H]34)c3c(c(-c4ccc5c(c4)OCO5)c2cc1OC)C(=O)OC3] 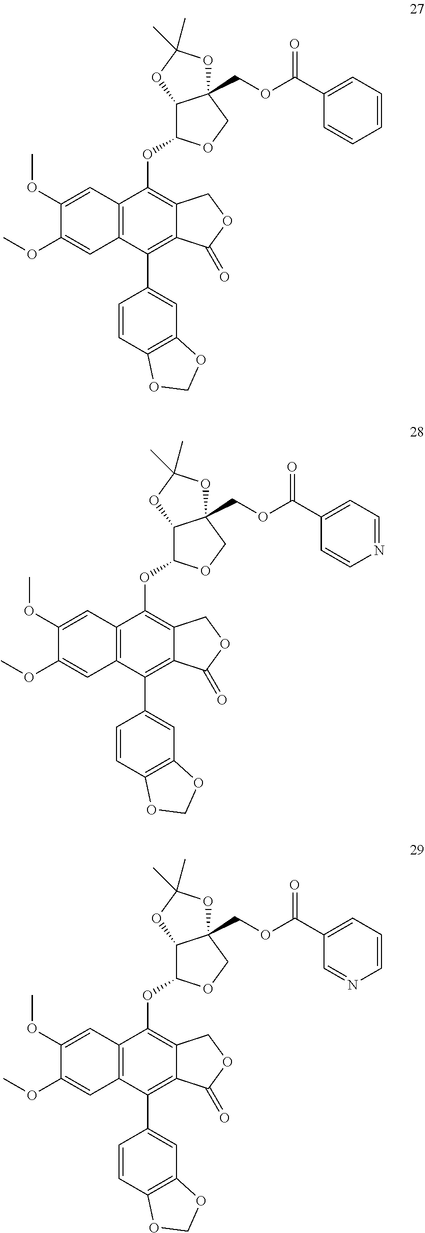 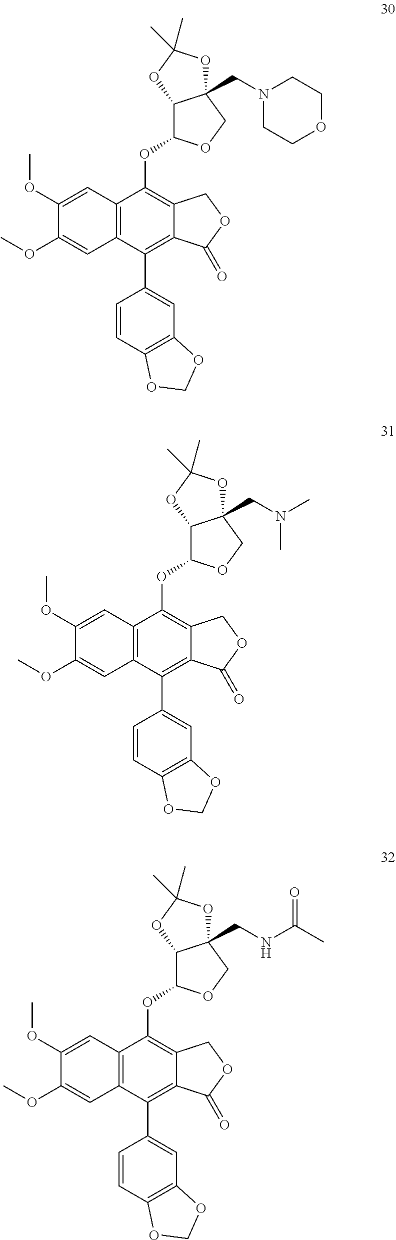 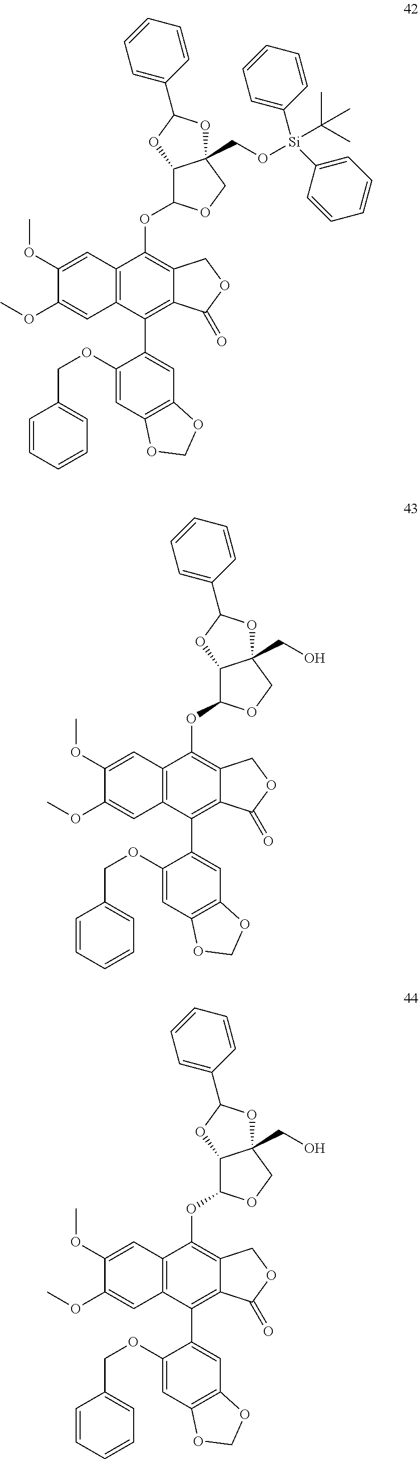 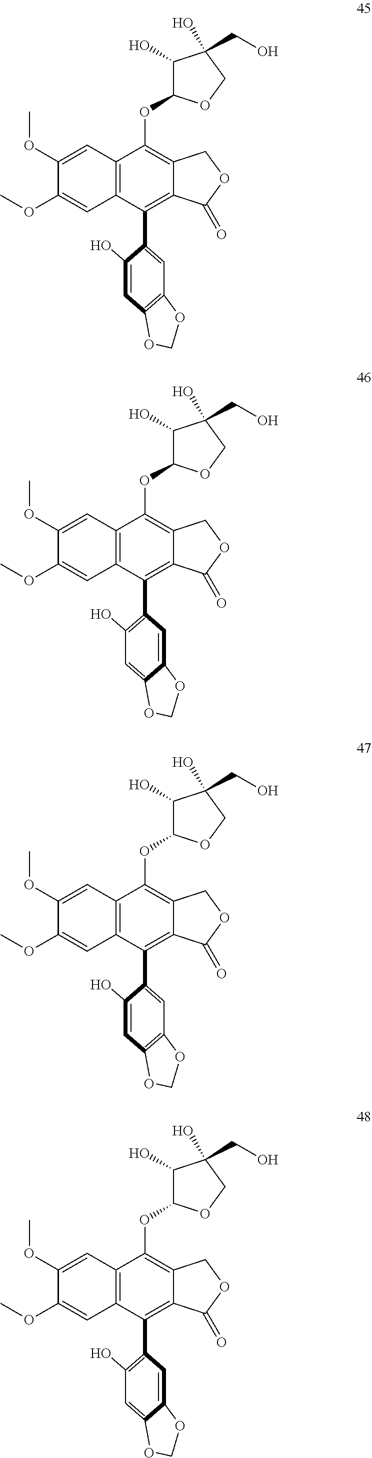 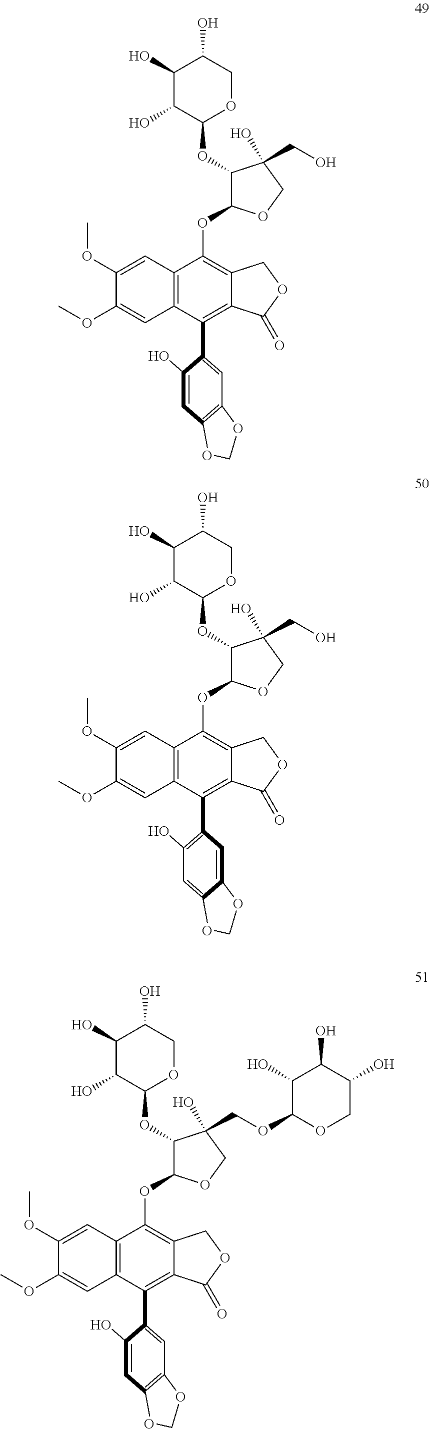 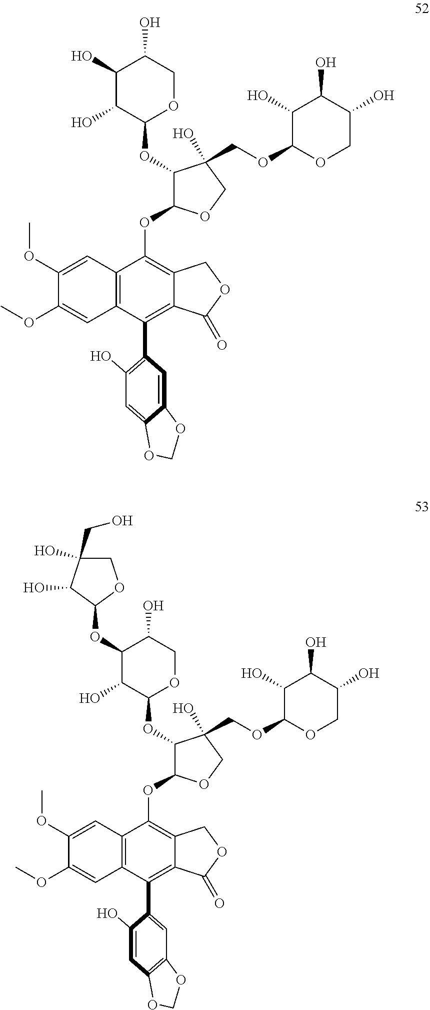 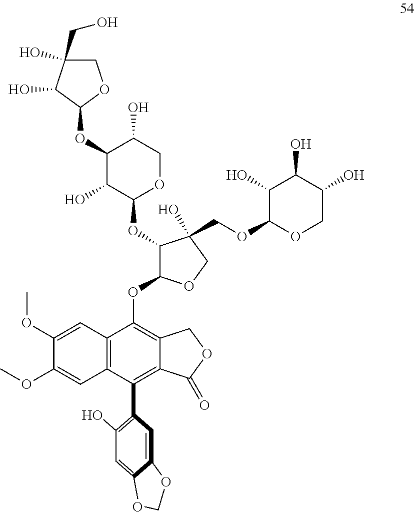
or a pharmaceutically acceptable salt thereof.

12. A pharmaceutical composition comprising a compound of claim 1 and at least one pharmaceutically acceptable excipient.

13. A compound of claim 1 for use in the treatment, prevention or delay of progression of a viral infection in a subject in need thereof.

14. The use of claim 13, wherein the viral infection is human immunodeficiency virus (HIV), influenza, vesicular stomatitis virus (VSV), or coronavirus (CoV).

15. The use of claim 14, wherein the influenza is avian influenza virus (AIV).

16. The use of claim 15, wherein the AIV is influenza A.

17. The use of claim 16, wherein the influenza A is H₅N₁.

18. The use of claim 14, wherein the CoV is severe acute respiratory syndrome coronavirus 2 (SARS-CoV-2).

19. The use of claim 13, wherein the compound inhibits the viral replication.

20. The use of claim 13, wherein the subject is human.

21. The use of claim 13, wherein the subject is an animal.

22. A compound for use in treatment, prevention or delay of progression of a viral infection in a subject in need thereof, wherein the compound has the Formula (I): 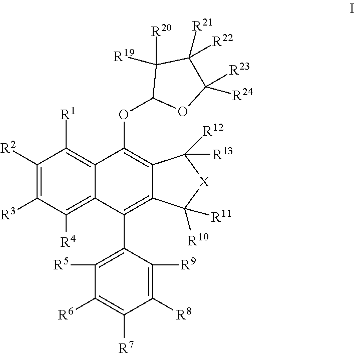
or a pharmaceutically acceptable salt thereof, wherein:
X is oxygen or sulfur;
R¹, R², R³, R⁴, R⁵, R⁶, R⁷, R⁸, and R⁹ are each independently hydrogen, alkyl, akenyl, alkynyl, cycloalkyl, heterocycloalkyl, aryl, aralkyl, heteroaryl, haloalkyl, halogen, cyano, NO₂, —OR²⁶, —C(═O)R²⁷, —C(═O)N(R²⁶)R²⁷, —C(═O)OR²⁶, —OC(═O)R²⁶, —OSi(R²⁵)(R²⁶)R²⁷, —S(═O)₂R²⁶, —S(═O)₂N(R²⁶)R²⁷, —N(R²⁶)R²⁷, —N(R²⁶)N(R²⁶)R²⁷, —N(R²⁶)C(═O)R²⁷, —N(R²⁶)S(═O)₂R²⁷ or a moiety comprising 1 to 30 plural valence atoms selected from the group consisting of carbon, nitrogen, oxygen and sulfur; or R¹ and R², R² and R³, R³ and R⁴, R⁵ and R⁶, R⁶ and R⁷, R⁷ and R⁸, or R⁸ and R⁹ taken together with the carbon atoms to which they are attached to form a cyclic group which is optionally substituted with halogen or a moiety comprising 1 to 30 plural valence atoms selected from the group consisting of carbon, nitrogen, oxygen and sulfur;
R¹⁰ and R¹¹ taken together form oxo; or while one of R¹⁰ and R¹¹ is hydrogen or halogen, the other one of R¹⁰ and R¹¹ is selected from the group consisting of R²⁵, —OR²⁵, —C(═O)R²⁵, and —C(═O)OR²⁵;
R¹² and R¹³ taken together form oxo; or while one of R¹² and R¹³ is hydrogen or halogen, the other one of R¹² and R¹³ is selected from the group consisting of R²⁵, —OR²⁵, —C(═O)R²⁵, and —C(═O)OR²⁵;
R¹⁹ and R²⁰ taken together form oxo; or while one of R¹⁹ and R²⁰ is hydrogen or halogen, the other one of R¹⁹ and R²⁰ is selected from the group consisting of R²⁵, —OR²⁵, —C(═O)R²⁵, —C(═O)OR²⁵, —OC(═O)R²⁵, —OC(═O)N(R²⁵)R²⁵, optionally substituted monosaccharide, optionally substituted disaccharide, optionally substituted trisaccharide, and optionally substituted tetrasaccharide;
R²¹ and R²² taken together form oxo; or while one of R²¹ and R²² is hydrogen, halogen, R²⁵ or —OR²⁵, the other one of R²¹ and R²² is selected from the group consisting of R²⁵, —OR²⁵, —OC(═O)R²⁵, —OC(═O)N(R²⁵)R²⁵, —C(═O)OR²⁵, —CH₂R²⁹, —CH₂OR²⁹, —C(═O)R²⁹; or R¹⁹ and R²¹ taken together with the carbon atoms to which they are attached to form a 5-6 membered heterocyclyl optionally substituted with 1, 2, 3, 4 or 5 group(s) independently selected from R²⁵;
R²³ and R²⁴ taken together form oxo; or while one of R²³ and R²⁴ is hydrogen or halogen, the other one of R²³ and R²⁴ is selected from the group consisting of R²⁵, —OR²⁵, —C(═O)R²⁵, —C(═O)OR²⁵, optionally substituted monosaccharide, optionally substituted disaccharide, optionally substituted trisaccharide, and optionally substituted tetrasaccharide;
R²⁵ for each occurrence is independently selected from hydrogen, halogen, trichloromethyl, trifluoromethyl, cyano, nitro, —OR²⁶, —C(═O)R²⁷, —C(═O)N(R²⁶)R²⁷, —C(═O)OR²⁶, —OC(═O)R²⁶, —OSi(R²⁵)(R²⁶)R²⁷, —S(═O)₂R²⁶, —S(═O)₂N(R²⁶)R²⁷, —N(R²⁶)R²⁷, —N(R²⁶)N(R²⁶)R²⁷, —N(R²⁶)C(═O)R²⁷, —N(R²⁶)S(═O)₂R²⁷, —N₃, —OS(═O)₂CF₃, hydrocarbyl optionally substituted with 1, 2, 3, 4 or 5 group(s) independently selected from the group consisting of R²⁸, heterocyclcyl optionally substituted with 1, 2, 3, 4 or 5 group(s) independently selected from the group consisting of R²⁸, and —(CH₂)ₖ-heterocyclyl optionally substituted with 1, 2, 3, 4 or 5 group(s) independently selected from the group consisting of R²⁸, wherein k is an integer between 1 and 6;
R²⁶ and R²⁷ for each occurrence are each independently hydrogen or selected from hydrocarbyl and heterocyclyl, either of which is optionally substituted with 1, 2, 3, 4 or 5 group(s) independently selected from halogen, cyano, amino, hydroxy, C₁₋₆ alkyl and C₁₋₆ alkoxy;
R²⁸ for each occurrence is independently selected from halogen, trichloromethyl, trifluoromethyl, cyano, nitro, oxo, ═NR²⁶, —OR²⁶, —C(═O)R²⁷, —C(═O)N(R²⁶)R²⁷, —C(═O)OR²⁶, —OC(═O)R²⁶, —OSi(R²⁵)(R²⁶)R²⁷, —S(═O)₂R²⁶, —S(═O)₂N(R²⁶)R²⁷, —N(R²⁶)R²⁷, —N(R²⁶)N(R²⁶)R²⁷, —N(R²⁶)C(═O)R²⁷ and —N(R²⁶)S(═O)₂R²⁷; and
R²⁹ for each occurrence is independently selected from hydrogen, halogen, trichloromethyl, trifluoromethyl, cyano, nitro, —OR²⁶, —C(═O)R²⁷, —C(═O)N(R²⁶)R²⁷, —C(═O)OR²⁶, —OC(═O)R²⁶, —OSi(R²⁵)(R²⁶)R²⁷, —S(═O)₂R²⁶, —S(═O)₂N(R²⁶)R²⁷, —N(R²⁶)R²⁷, —N(R²⁶)N(R²⁶)R²⁷, —N(R²⁶)C(═O)R²⁷, —N(R²⁶)S(═O)₂R²⁷, —N₃, —OS(═O)₂CF₃, optionally substituted monosaccharide, optionally substituted disaccharide, optionally substituted trisaccharide, optionally substituted tetrasaccharide, hydrocarbyl optionally substituted with 1, 2, 3, 4 or 5 group(s) independently selected from the group consisting of R²⁸, and heterocyclyl optionally substituted with 1, 2, 3, 4 or 5 group(s) independently selected from the group consisting of R²⁸.

23. The use of claim 22, wherein the compound is selected from the group consisting of 1, 1-Ac, A1, A2, A3, A4, A5, A6, A7, A8, 8, 9, 10, 11, 12, 16, 17, 18, 19, 20, 21, 22, 23, 24, 25, 26, 27, 28, 29, 30, 31, 32, 42, 43, 44, 45, 46, 47, 48, 49, 50, 51, 52, 53 and 54:

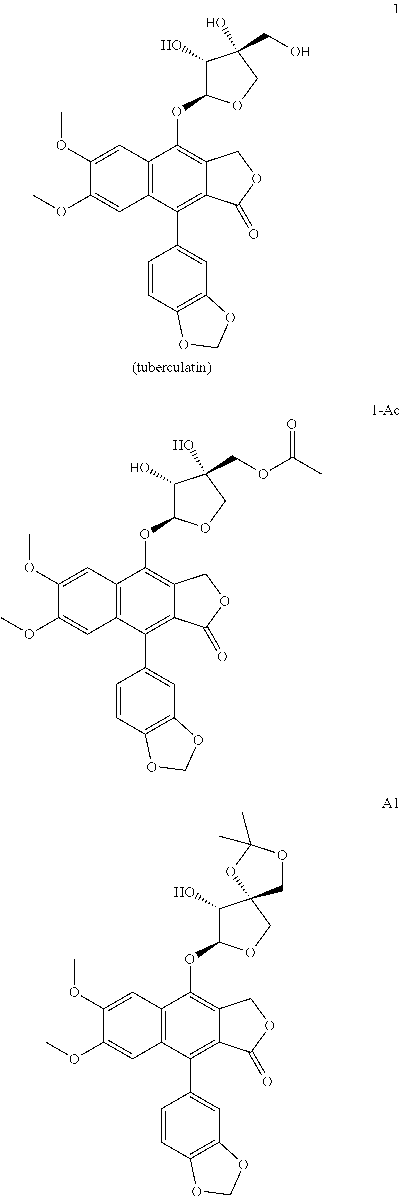

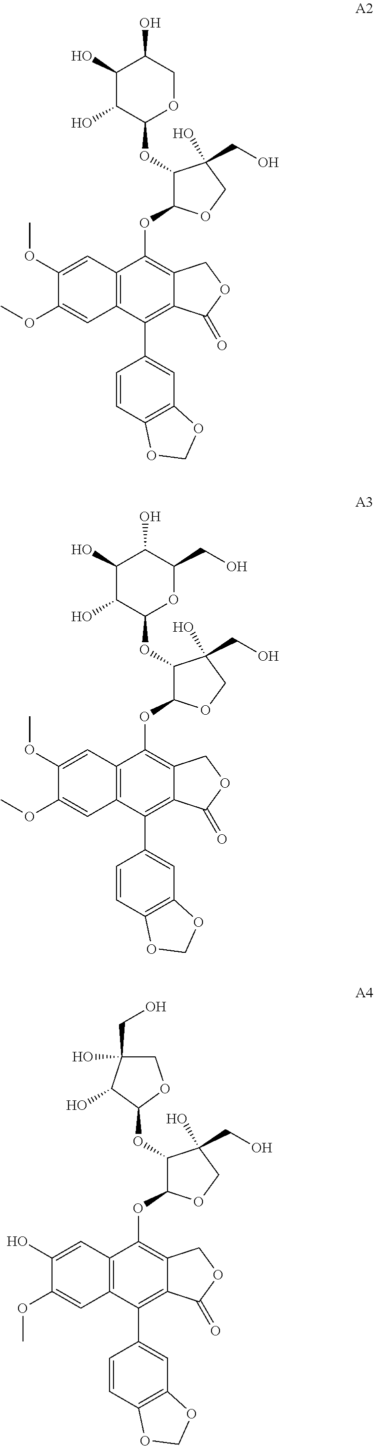

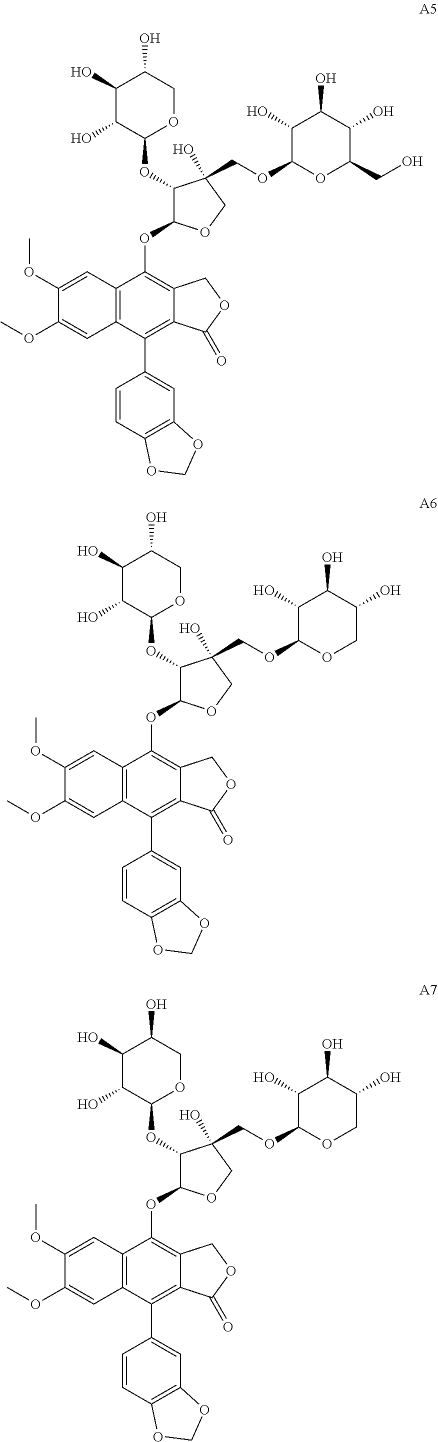

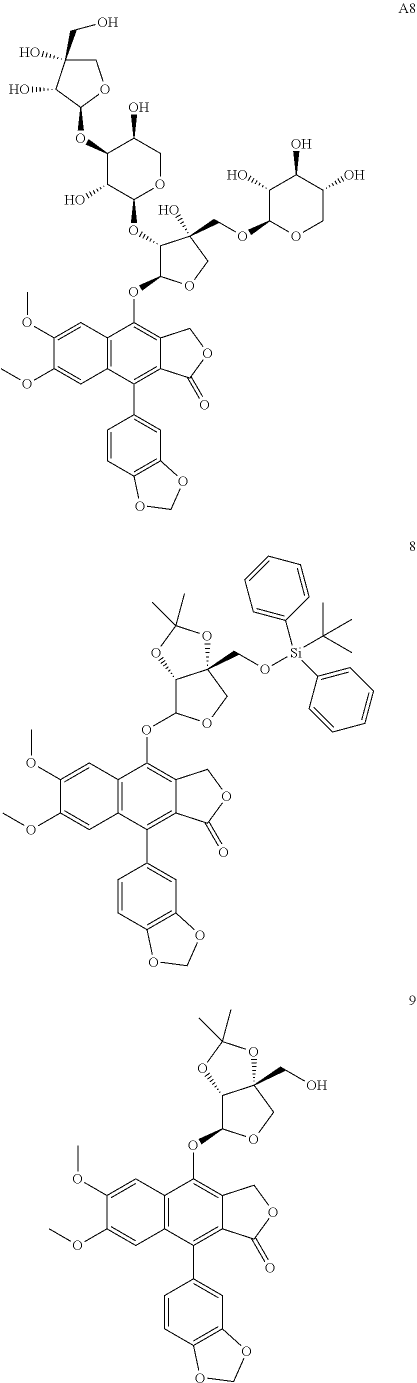

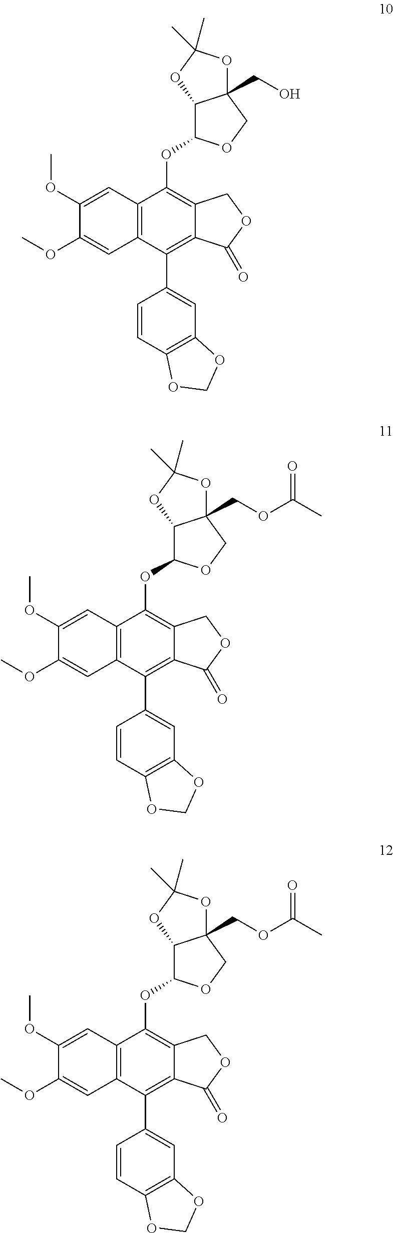

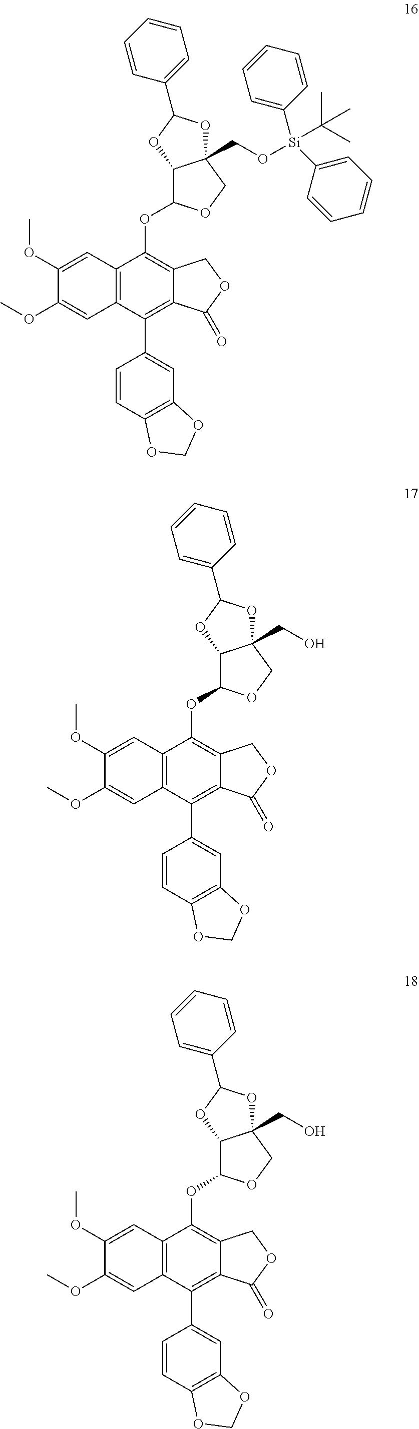

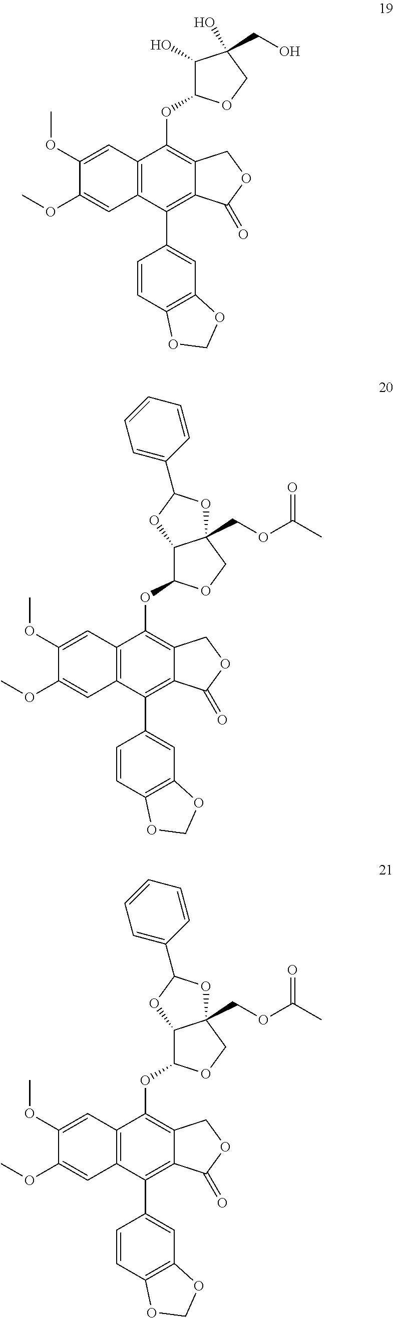

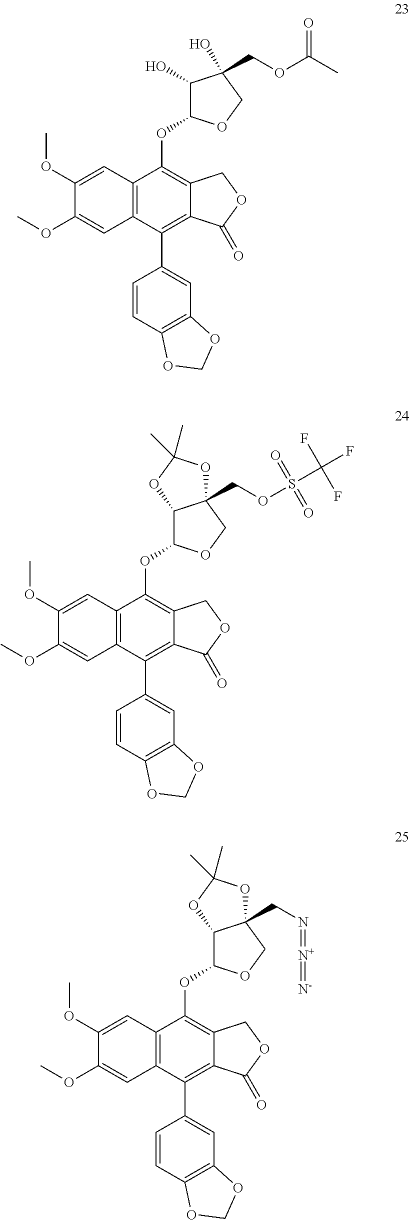

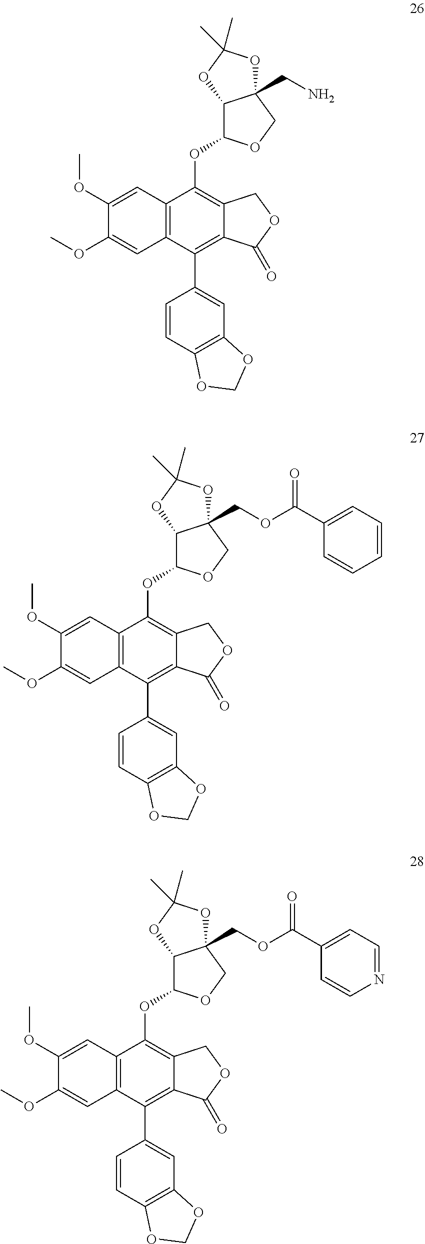 [SMILES: COc1cc2c(O[C@H]3OC[C@@]4(CN)OC(C)(C)O[C@@H]34)c3c(c(-c4ccc5c(c4)OCO5)c2cc1OC)C(=O)OC3 ; COc1cc2c(O[C@H]3OC[C@@]4(COC(=O)c5ccccc5)OC(C)(C)O[C@@H]34)c3c(c(-c4ccc5c(c4)OCO5)c2cc1OC)C(=O)OC3 ; COc1cc2c(O[C@H]3OC[C@@]4(COC(=O)c5ccncc5)OC(C)(C)O[C@@H]34)c3c(c(-c4ccc5c(c4)OCO5)c2cc1OC)C(=O)OC3]

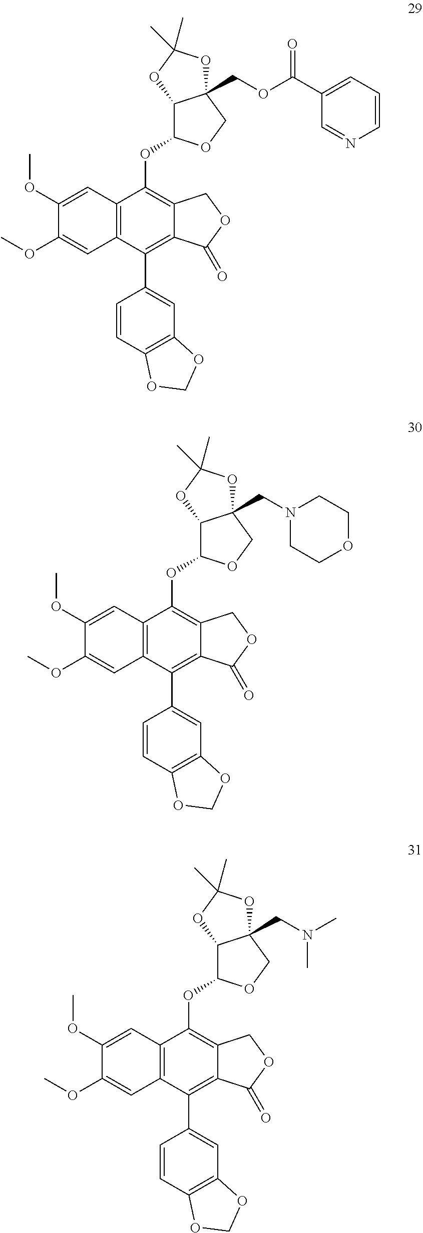

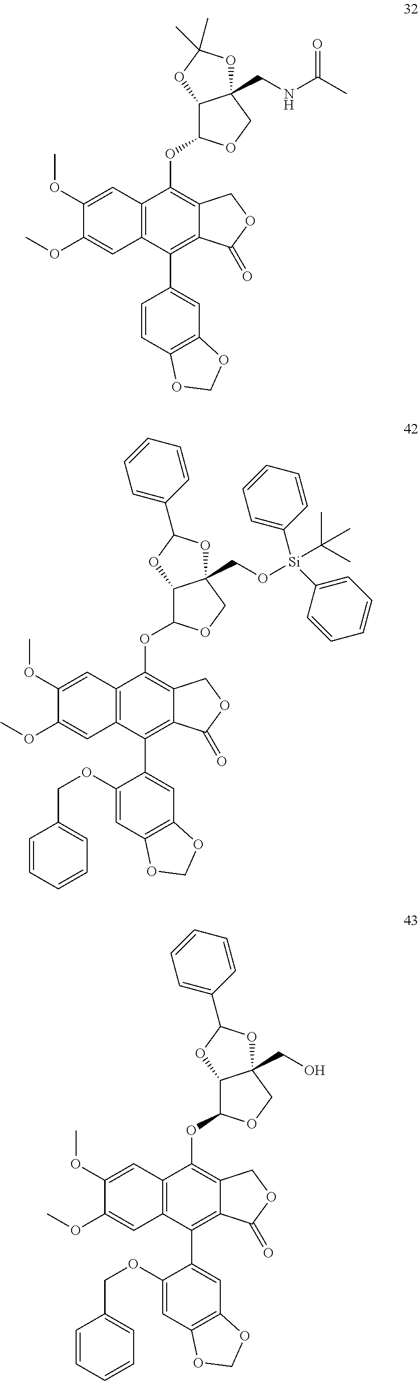

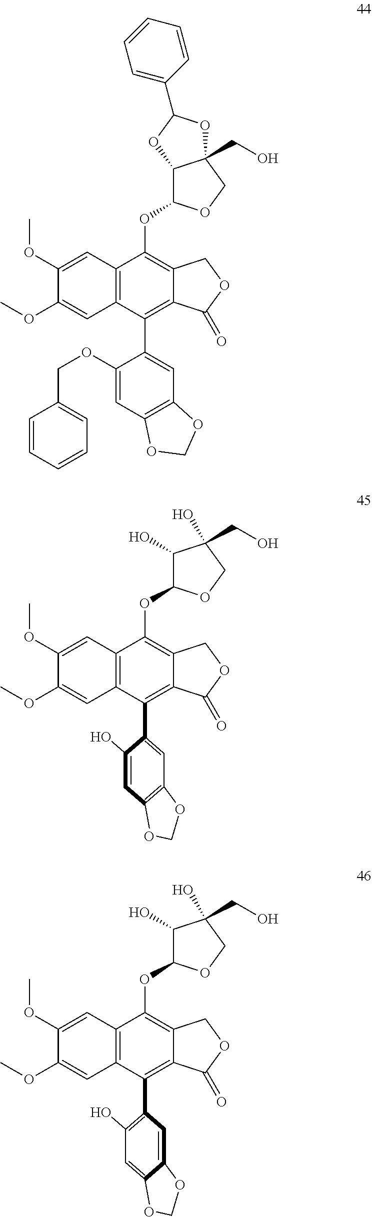

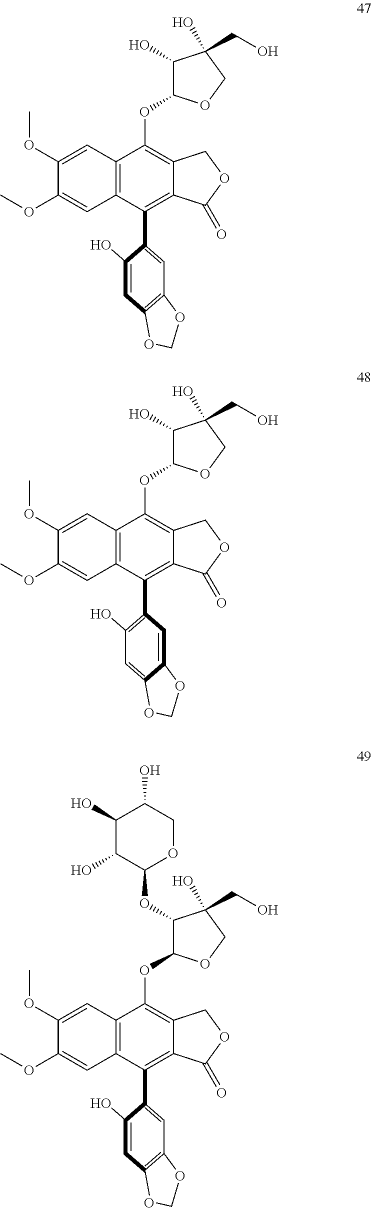

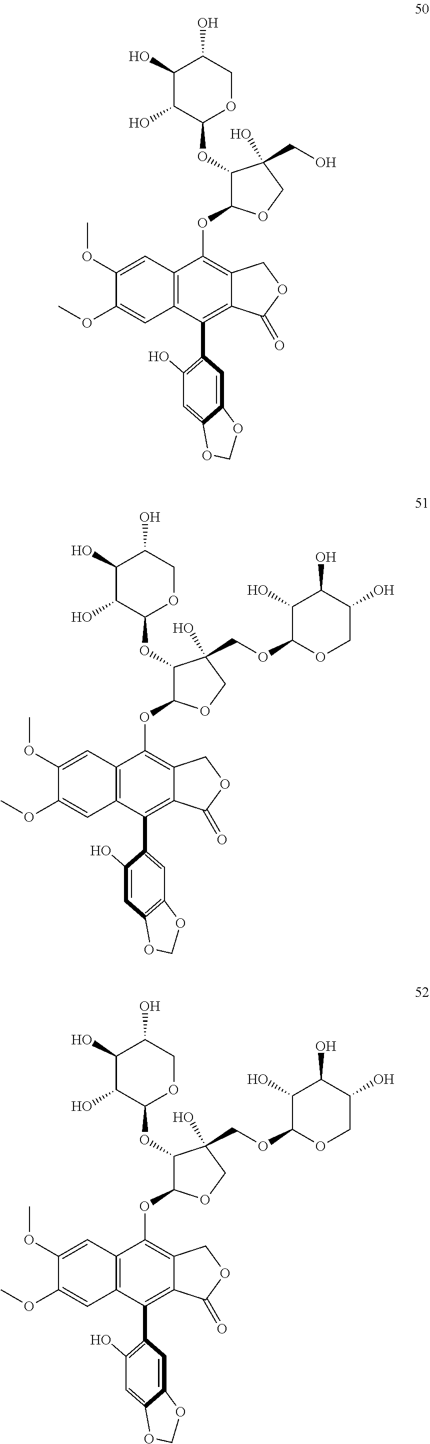

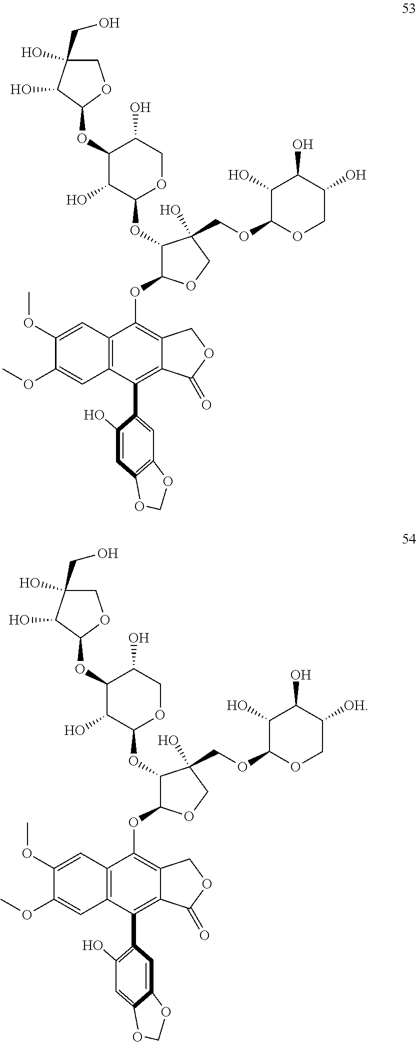

24. The use of claim 22, wherein the viral infection is HIV, influenza, VSV, or CoV.

25. The use of claim 24, wherein the influenza is AIV.

26. The use of claim 25, wherein the AIV is influenza A.

27. The use of claim 26, wherein the influenza A is H5N1.

28. The use of claim 24, wherein the CoV is SARS-CoV-2.

29. The use of claim 23, wherein the compound inhibits the viral replication.

30. The use of claim 23, wherein the subject is human.

31. The use of claim 23, wherein the subject is an animal.

32. The use of claim 24, wherein the compound is present in a separated extract or fraction from a plant material.
